(19) United States
(12) Patent Application Publication
(10) Pub. No.: US 2025/0257041 A1
(43) Pub. Date: Aug. 14, 2025

(54) INDUCERS OF INTEGRATED STRESS RESPONSE TO TREAT CANCER

(71) Applicant: The Board of Regents of The University of Texas System, Austin, TX (US)

(72) Inventors: Joseph M. Ready, Carollton, TX (US); Kevin Luvaga, Dallas, TX (US); Shanhai Xie, Plano, TX (US); Ceclia Morgenstern, Dallas, TX (US); Deepak Nijhawan, Dallas, TX (US); Min Fan, Dallas, TX (US); Jan Erzberger, Dallas, TX (US); Victor Cruz Ruiz, Dallas, TX (US)

(21) Appl. No.: 18/866,939

(22) PCT Filed: May 19, 2023
(86) PCT No.: PCT/US2023/022946

§ 371 Date: Nov. 18, 2024

Related U.S. Application Data
(60) Provisional application No. 63/343,642, filed on May 19, 2022.

(51) Int. Cl. C07D 231/40; A61K 31/167; A61K 31/4155; A61K 31/42; A61K 31/4245; A61K 31/426; A61K 31/4439; A61K 31/496; A61K 31/5377; A61K 45/06; A61P 35/00; A61P 35/02; C07C 233/80; C07D 213/75; C07D 231/42; C07D 261/14; C07D 271/113; C07D 277/46; C07D 401/12; C07D 403/12; C07D 405/12; C07D 405/14; C07D 413/12

(52) U.S. Cl. CPC C07D 231/40; A61K 31/167; A61K 31/4155; A61K 31/42; A61K 31/4245; A61K 31/426; A61K 31/4439; A61K 31/496; A61K 31/5377; A61K 45/06; A61P 35/00; A61P 35/02; C07C 233/80; C07D 213/75; C07D 231/42; C07D 261/14; C07D 271/113; C07D 277/46; C07D 401/12; C07D 403/12; C07D 405/12; C07D 405/14; C07D 413/12

(57) ABSTRACT

Compounds, compositions, and methods for making and using the compounds for treatment of cancer are provided herein. Mechanistically, these compounds were found to bind to RPS23 and were effective in activating the inducible stress response.

### CROSS REFERENCE TO RELATED APPLICATIONS

This application claims the benefit of the U.S. provisional application No. 63/343,642 filed May 19, 2022, the disclosures of each which are herein incorporated by reference in their entirety.

### BACKGROUND

### 1. Field

Provided herein are compositions and methods for treating cancers.

### 2. Discussion of Related Art

Cancer is a group of diseases characterized by abnormal and uncontrolled proliferation of cells. The American Cancer Society had estimated 1.9 million new cancer cases diagnosed and 609,360 cancer deaths in the United States for 2022, making cancer one of the leading causes of death in the US. Cancer cells can form a solid tumor, or the cells can exist as a dispersed mass, as in the case of leukemia.

Thus far, cancer drug therapies tend to rely on cytotoxic agents, selective for dividing cells. These drugs are effective, because cancer cells generally divide more frequently than normal cells. However, such drugs almost inevitably do not kill all the cancer cells in the patient. One reason is that cancer cells can acquire mutations that confer drug resistance. It is therefore important to continue developing new drugs that are mechanistically different from existing drugs and to identify new targets to treat cancer.

Over the last decades, the efficacy of cancer treatment has improved considerably. However, there is still a need for effective drugs against a range of cancer types that do not respond to or partially respond to the available treatment options. New drugs that are effective against multiple cancer types could greatly increase treatment options and enhance outcomes.

### SUMMARY

In some aspects the current disclosure encompasses a compound of any one of Formulae I-VII:

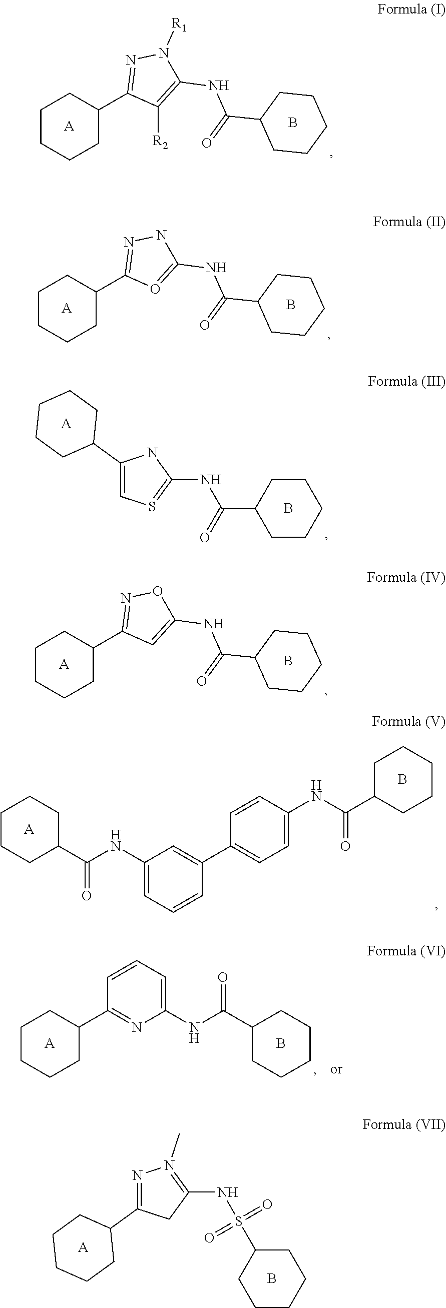

or a pharmaceutically acceptable salt thereof;
wherein,
Cycle A and Cycle B are each independently selected from —(C6-C10)-aryl, —(C6-C10)—substituted aryl, —(C6-C10)-heteroaryl, —(C6-C10)-substituted heteroaryl, —(C3-C10)-alkyl, or —(C3-C10)-substituted alkyl; R₁ is alkyl, aryl, or alkyl-aryl; R₂ is hydrogen, halide or alkyl;
wherein said heterocyclyl has 1-4 heteroatoms independently selected from N, NH, O, S, SO, and SO₂, and said heteroaryl has 1-4 heteroatoms independently selected from N, NH, O, and S.

In some aspects, Cycle A and Cycle B in Formula (I) are a C-6 substituted aryl as shown in Formula (Ia):

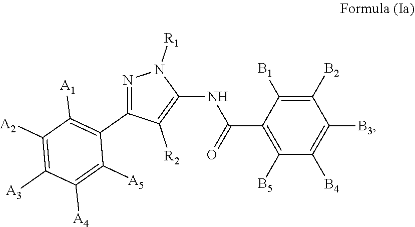

wherein
A₁-A₅ and B₁-B₅ are each independently selected from a halogen, —R, —OR, —NO₂, —NCS, —CN, —CF₃, —OCF₃, —SiR₃, —NH₂, —SR, —SOR, —SO₂R, —SO₂N(R)₂, —SO₃R, —(CR₂)1-3R, —(CR₂)1-3-OR, —(CR2)0-3-C(O)NR(CR2)0-3R, —(CR2)0-3-C(O)NR(CR2)0-30R, —C(O)R, —C(O)C(O)R, —C(O)CH2C(O)R, —C(S)R, —C(S)OR, —C(O)OR, —C(O)C(O)OR, —C(O)C(O)N(R)2, —OC(O)R, —C(O)N(R)2, —OC(O)N(R)2, —C(S)N(R)2, —(CR2)0-3NHC(O)R, —N(R)N(R)COR, —N(R)N(R)C(O)OR, —N(R)N(R)CON(R)2, —N(R)SO₂R, —N(R)SO₂N(R)2, —N(R)C(O)OR, —N(R)C(O)R, —N(R)C(S)R, —N(R)C(O)N(R)2, —N(R)C(S)N(R)2, —N(COR)COR, —N(OR)R, —C(═NH)N(R)2, —C(O)N(OR)R, —C(═NOR)R, or —CF3;
wherein R is hydrogen, alkyl, alkyl amine, alkyl sulfonamide, aryl, substituted aryl, heterocyclic aryl amine, substituted heterocyclic aryl amine, cyclic aliphatic amine, heterocyclic aliphatic amine, substituted heterocyclic aliphatic amine, alkyne; or together with the carbon atom to form a cycloalkyl, a cycloalkenyl, or a heterocyclylalkyl ring;
R₁ is alkyl, aryl, or alkyl-aryl; and
R₂ is hydrogen, halogen, or alkyl.

In some aspects, in Formula (I) Cycle A is a substituted C6-aryl and Cycle B is a (C3-C10) cycloalkyl, a substituted (C3-C10) cycloalkyl, or a (C3-C10) cycloheteroalkyl as shown below:

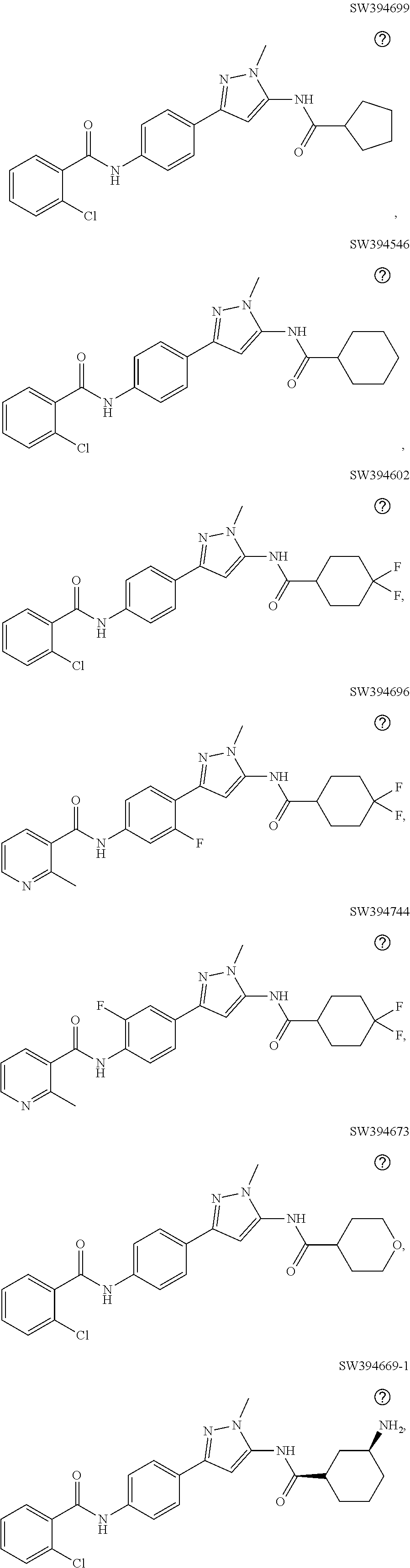

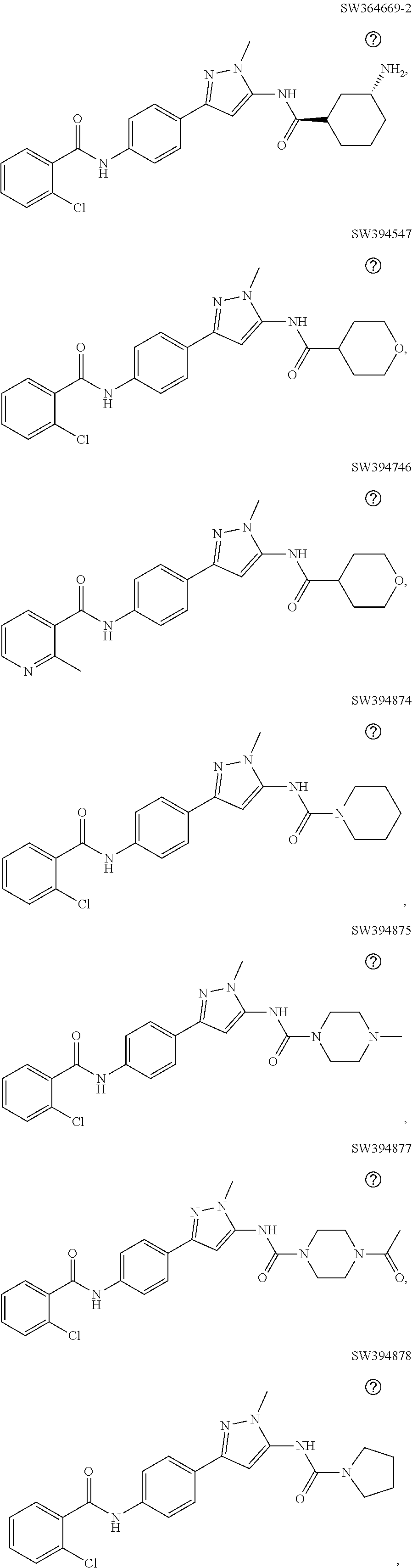

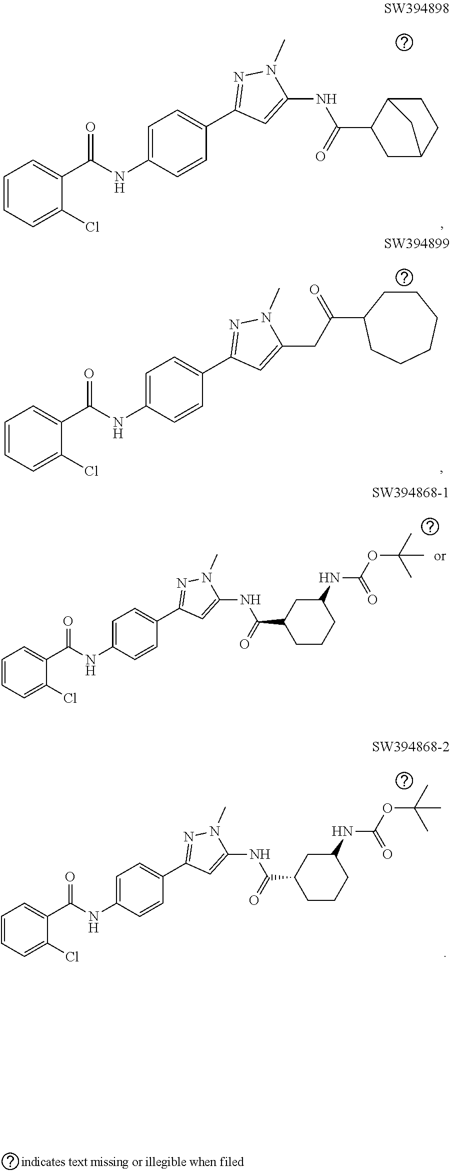

In some aspects, the compound provided herein is Formula (II) or Formula (III), wherein in Formula (II) or Formula (III) cycle A and cycle B are C6-substituted aryl as shown in Formula (IIa)-(IIIa):

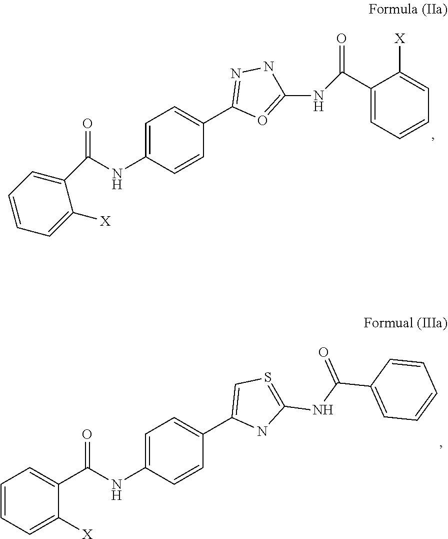

wherein X is a halogen.

In some aspects, the current disclosure encompasses a compound, wherein the compound is Formula (IV) and wherein in Formula (IV) cycle A is a substituted C6-aryl and cycle B is a C-6 aryl as shown in Formula (IVa):

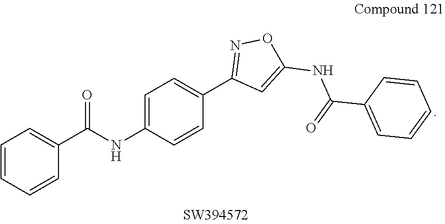 [SMILES: O=C(Nc1ccc(-c2cc(NC(=O)c3ccccc3)on2)cc1)c1ccccc1]

In some aspects, the compound as provided herein is Formula (V), wherein in Formula (V) cycle A is a C6-aryl and cycle B is a substituted C6-aryl as shown in Formula (Va):

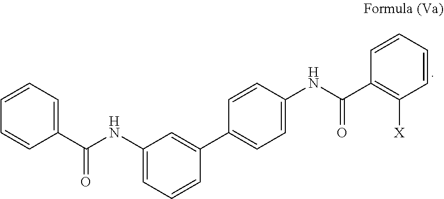

In some aspects, the compound is Formula (VI), wherein in Formula (VI) cycle A is a substituted C6-aryl and cycle B is a C6-aryl as shown below:

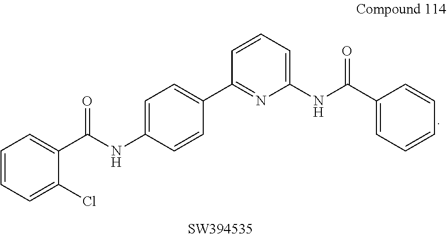 [SMILES: O=C(Nc1cccc(-c2ccc(NC(=O)c3ccccc3Cl)cc2)n1)c1ccccc1]

In some aspects, the compound as provided herein is Formula (VII), wherein in Formula (VII) cycle A is a substituted C6-aryl and cycle B is a C6-aryl as shown below:

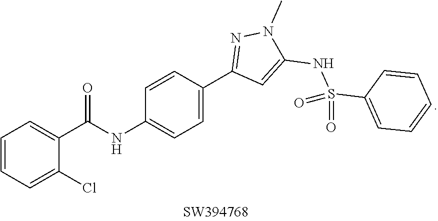 [SMILES: Cn1nc(-c2ccc(NC(=O)c3ccccc3Cl)cc2)cc1NS(=O)(=O)c1ccccc1]

In some aspects, the compound as disclosed herein specifically binds to a site on the ribosome adjacent to RPS23. In some aspects, the compound as disclosed herein can activate the integrated stress response.

In some aspects, the current disclosure also encompasses a pharmaceutical composition comprising: one or more of the compounds as disclosed herein, and at least a pharmaceutically acceptable excipient. In some aspects, the pharmaceutical composition comprises one or more of Compounds 1-188 provided in Table 1 or a pharmaceutically acceptable salt thereof, and at least a pharmaceutically acceptable excipient. In some aspects, the pharmaceutical composition does not comprise the following compounds:

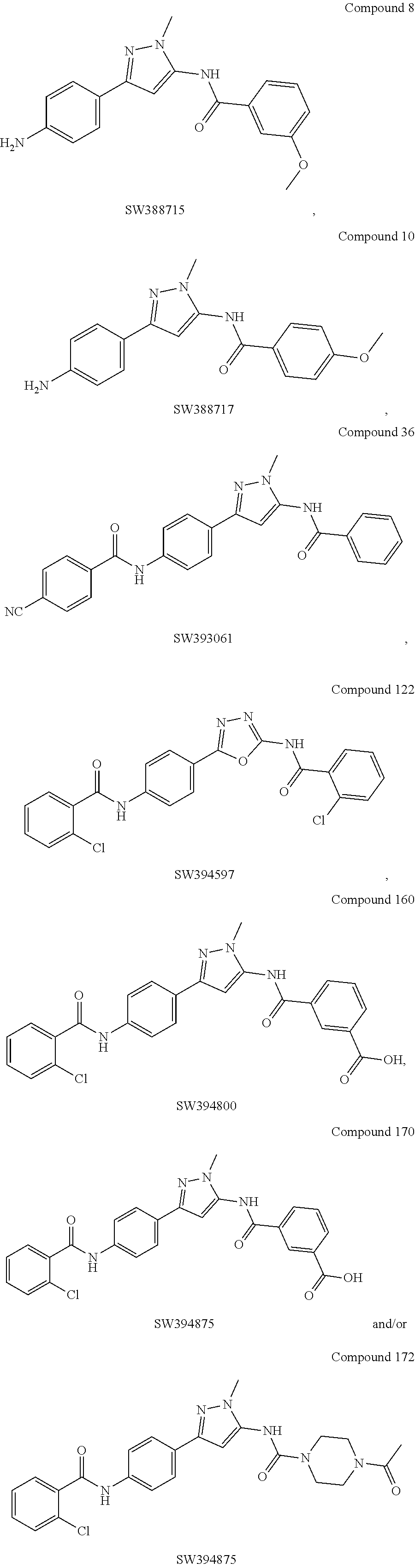

In some aspects of the pharmaceutical composition as disclosed herein, the at least one pharmaceutically acceptable excipient is a liquid or solid filler, a diluent, a binder, a buffering agent, a pH modifying agent, a disintegrant, a dispersant, a preservative, a lubricant or wetting agent, taste-masking agent, an antioxidant, carrier, adjuvant, stabilizing agent, emulsifying agent, solution promoter, salt, solubilizer, antifoaming agent, surfactant, a flavoring agent, a coloring agent, solvent or encapsulating material or any combination thereof. In some aspects, the pharmaceutical composition as provided herein comprises a compound with an IC₅₀ value <20 nM against at least one type of cancer cell line. In some aspects, the compound can arrest the growth of a cancer cell line. In some aspects, the pharmaceutical composition may further comprise additional active agents for example, NSAID, antibiotics, antimicrobial, anti-inflammatory, anticancer, theragnostic agent, or any combination thereof.

In some aspects, administering an effective amount of the pharmaceutical composition as disclosed herein into a subject in need thereof can treat a cancer. In some aspects, the cancer is any cancer, for example cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft tissue sarcoma, squamous cell carcinomas, stomach cancer, testicular cancer, thyroid cancer, trophoblastic neoplasms, uterine cancer, vaginal cancer, cancer of the vulva and Wilma's tumor. In some aspects, the cancer is a leukemia. In some aspects, the cancer is colorectal cancer.

In some aspects, the current disclosure also encompasses a method of treating cancer in a subject in need thereof, comprising: administering an effective amount of one or more of Compounds 1-187 provided in Table 1 or pharmaceutically acceptable salt thereof to the subject.

In some aspects, the current disclosure also encompasses a method of treating cancer in a subject in need thereof, the method comprising: administering an effective amount of one or more of the compounds as provided herein and at least a pharmaceutically acceptable excipient to the subject. In some aspects, the compound is not SW388717, SW388715, SW393061, SW394597, SW394800, SW394875, and/or SW394877. In some aspects, the subject is diagnosed and/or suffering from a leukemia. In some aspects, the subject has a cancer, for example acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, 10 gallbladder cancer, hairy cell leukemia, head and neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft 15 tissue sarcoma, squamous cell carcinomas, stomach cancer, testicular cancer, thyroid cancer, trophoblastic neoplasms, uterine cancer, vaginal cancer, cancer of the vulva and Wilma's tumor. In some aspects, subject is diagnosed and/or suffering from colorectal cancer. In some aspects, the subject is a mammal. In some aspects, the subject is a human. In some aspects, the pharmaceutical composition as disclosed herein can be administered by any mode for example, parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal route.

In some aspects, the current disclosure also encompasses use of a compound comprising as disclosed herein to treat cancer in a subject in need thereof. In some aspects, the compound is used to treat a leukemia. In some aspects, the compound is used to treat colorectal cancer. In some aspects, the compound is used to activate the integrated stress response to treat cancer in a subject in need thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A provides modular synthesis method for an exemplary compound for rapid synthesis and optimization of analogs.

FIG. 1B provides viability data for cancer cell lines, HCT116 and Jurkat. Viability assay was performed using glycyl-phenylalanyl amino fluorocoumarin (GF-AFC) to determine number of viable cells.

FIG. 1C provides cytotoxicity data against leukemia cell for exemplary compound using the CellTox Green Dye to determine number of nonviable cells.

FIG. 2A provides a schematic of the forward genetics approach to determine the mechanism of action of the lead compounds.

FIG. 2B provides data to show the effect of SW388710 (SW710) on cells with shRNA knockdown of the listed genes.

FIG. 3 provides structures of analogs of the first-generation lead compound SW393071 with photocroslinkers, biotin and dyes attached. The analogs are indicated by their short names, with long names as provided: SW212 (SW393212), SW186 (SW393186), SW173 (SW393173), SW242 (SW393242), SW280 (SW393280), SW213 (SW393213), SW185 (SW393185), SW152 (SW393152), SW690 (SW393690), SW345 (SW393345), and SW656 (SW393656).

FIG. 4A provides results of preliminary in vivo trials with SW393071.

FIG. 4B provides results of preliminary in vivo trials with SW394672.

FIG. 4C provides results of preliminary in vivo trials with SW394677.

FIG. 5A provides cell proliferation studies using Jurkat cells using exemplary novel compounds provided in the current disclosure. Jurkat cells, which are derived from a human leukemia, were incubated with varying concentrations of SW388710. Cell proliferation over three days was evaluated using luminescence from Cell Titer Glo (Promega) which reflects ATP levels. SW388710 reduces the proliferation of these cells in a dose dependent manner, (IC₅₀=78.2 nM) and the addition of 10 nM of ISRIB leads to relative resistance demonstrating that the ISR pathway is essential to the anti-proliferative phenotype.

FIG. 5B provides cell proliferation studies using Jurkat cells using exemplary novel compounds provided in the current disclosure. Jurkat cells, which are derived from a human leukemia, were incubated with varying concentrations of SW393071. Cell proliferation over three days was evaluated using luminescence from Cell Titer Glo (Promega) which reflects ATP levels. SW393071 reduces the proliferation of these cells in a dose dependent manner, (IC₅₀=8.2 nM) and the addition of 10 nM of ISRIB leads to relative resistance demonstrating that the ISR pathway is essential to the anti-proliferative phenotype.

FIG. 6A provides cytotoxicity studies for leukemia cells (Jurkat) with compound SW388710. SW388710 induces cell death in leukemic cells. Jurkat (Leukemia) cells were incubated with varying doses of SW388710. Viable cells were quantified by using GF-AFC (a peptide that is cleaved by intracellular proteases revealing a fluorescent molecule) and Cytox (Promega) an impermeable dye that is only able to enter dying cells where it emits a fluorescent signal after binding to DNA.

FIG. 6B provided cytostatic data for colorectal cells, with compound SW388710. SW388710 reduces the proliferation of colorectal cancer cells. HCT-116 (colorectal) cells were incubated with varying doses of SW388710. Viable cells were quantified by using GF-AFC (a peptide that is cleaved by intracellular proteases revealing a fluorescent molecule) and Cytox (Promega) an impermeable dye that is only able to enter dying cells where it emits a fluorescent signal after binding to DNA.

FIG. 7 provides data showing that SW393071 (SW071) can inhibit translation in vitro. Rabbit reticulocyte lysate was incubated in the presence of luciferase mRNA, NTPs, and various concentrations of SW071. Transcription of full-length luciferase was monitored by luminescence, showing dose-dependent inhibition of translation by SW071.

FIG. 8A is a schematic of the experimental set-up to determine the binding site of the compounds on the ribosome.

FIG. 8B provides structures of photocrosslinking analogs SW393065 (SW065), SW393212 (SW212) and SW393213 (SW213).

FIG. 8C provides a western blot analysis of RPS23 bound to the compounds. HCT116 cells were incubated with photo-crosslinkers and irradiated with UV light. The cells were lysed and click-conjugated to biotin-azide. Drug-bound proteins were pulled down with streptavidin coated beads and resolved on an SDS-PAGE gel. Western blotting for RPS23 shows bands with several probes, confirm binding on RPS23 in cells.

FIG. 9 provides data to show that RPS23 mutant populations are strongly resistant to SW393071 (SW071). Wild-type or RPS23 del R107-G109 knock-in mutant cells were incubated with SW071. The RPS23 mutant lines were completely resistant to SW071, confirming RPS23 as the functional target.

FIG. 10 provides a western blot analysis that SW388710 (SW710) does not bind RPS23 mutant cells. Wild type or RPS23 del R107-G109 knock-in mutant cells were HCT116 cells were incubated with photo-crosslinker SW212 (SW393212) and irradiated with UV light. The cells were lysed and click-conjugated to biotin-azide. Drug-bound proteins were pulled down with streptavidin coated beads and resolved on an SDS-PAGE gel. Western blotting for RPS23 shows bands from WT but not RPS23 mutant cells, confirming that mutant cells do not bind SW212 (SW393212).

FIG. 11 provides a schematic model showing that SW393071 activates ISR, leading to elF2α phosphorylation.

FIG. 12 is a western blot analysis showing the RPS23 is required for induction of GCN2 phosphorylation by SW710 (SW388710). Cells were incubated with SW710, lysed, then evaluated via western blot for phospho-GCN2, a marker of activation of the integrated stress response. WT, but not RPS23 mutant cells, showed activation of the ISR.

### DETAILED DESCRIPTION

The present disclosure is based in part on the surprising discovery that the disclosed compounds, such as cytotoxin SW106593 (SW593: identified in an initial high throughput screen for inhibitors of cell proliferation using the HCT116 colorectal carcinoma), are effective in reducing cell proliferation and has potential for being a therapeutic agent. Novel lead compounds and analogs and derivatives thereof provided herein were synthesized and tested in in vitro and in vivo assays and found to be effective anti-cancer agents. Mechanistic and structure studies were conducted, and it was found that these novel compounds bind to the ribosome at a unique binding site adjacent to the ribosomal protein RPS23. No other compound outside of this work has been observed to bind at this site. Binding was found to partially inhibit protein translation. Additionally, these compounds were further found to activate integrated stress response (ISR), a conserved cellular response to a variety of cellular insults. The activated ISR, in conjunction with RPS23 binding, surprisingly and uniquely results in cytotoxicity towards several cancer types, including but not restricted to leukemia and colorectal cancer. Other inducers of the ISR and other inhibitors of translation do not demonstrate this activity, thus making these compounds unique and their properties unexpected.

The current disclosure encompasses compounds and compositions comprising such compounds, wherein the compositions comprise compounds disclosed herein, including those provided in Table 1, or a pharmaceutically acceptable salt thereof, or analogs and derivatives thereof and methods of making and using the disclosed compounds or analogs and derivatives thereof.

### I. Compounds

In some aspects, the current disclosure encompasses compound or salt thereof comprising any one of the compounds disclosed herein. In some aspects, the current disclosure encompasses the compound or salt thereof comprising any one of the compounds 1-188 provided in Table 1. Throughout the current disclosure a given chemical formula or chemical name shall encompass all optical stereoisomers, as well as racemic mixtures where such isomers or mixtures exist, unless the specific isomer or diastereomer is noted.

In some aspects the current disclosure encompasses pharmaceutically acceptable derivatives of the compounds including but not restricted to salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine,1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

The term “alkyl” as used herein means a straight or branched hydrocarbon radical having from 1 to 10 carbon atoms and includes, for example, methyl, ethyl, n-propyl, isopropyl, n-butyl, sec-butyl, isobutyl, tert-butyl, n-pentyl, iso-pentyl, n-hexyl, and the like.

The term “alkenyl” as used means straight and branched hydrocarbon radicals having from 2 to 8 carbon atoms and at least one double bond and includes, but is not limited to, ethenyl, 3-buten-1-yl, 2-ethenylbutyl, 3-hexen-1-yl, and the like. The term “alkenyl” includes cycloalkenyl, and heteroalkenyl in which 1 to 3 heteroatoms selected from O, S, N, or substituted nitrogen may replace carbon atoms.

The term “alkynyl” as used means straight and branched hydrocarbon radicals having from 2 to 8 carbon atoms and at least one triple bond and includes, but is not limited to, ethynyl, 3-butyn-1-yl, propynyl, 2-butyn-1-yl, 3-pentyn-1-yl, and the like.

The term “cycloalkyl” as used means a monocyclic or polycyclic hydrocarbyl group having from 3 to 8 carbon atoms, for instance, cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, amino, alkyl, and dialkylamino, and the like. Also included are rings in which 1 to 3 heteroatoms replace carbons. Such groups are termed “heterocyclyl,” which means a cycloalkyl group also bearing at least one heteroatom selected from O, S, N, or substituted nitrogen. Examples of such groups include, but are not limited to, oxiranyl, pyrrolidinyl, piperidyl, tetrahydropyran, and morpholine.

The term “alkoxy” as used herein means a straight or branched chain alkyl groups having 1-10 carbon atoms and linked through oxygen. Examples of such groups include, but are not limited to, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In addition, alkoxy refers to polyethers such as —O—(CH2)2-O—CH3, and the like.

The alkyl, alkenyl, alkoxy, and alkynyl groups described herein are optionally substituted, prefer-ably by 1 to 3 groups selected from NR4R5, phenyl, substituted phenyl, thio C1-C6 alkyl, C1-C6 alkoxy, hydroxy, carboxy, C1-C6 alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C1-C6 alkyl or (CH2)pPh where p is 1, 2, or 3. Perhalo and polyhalo substitution is also included.

Examples of substituted alkyl groups include, but are not limited to, 2-aminoethyl, 2-hydroxyethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl)ethyl.

Examples of substituted alkynyl groups include, but are not limited to, 2-methoxyethenyl, 2-ethylsulfanylethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include, but are not limited to, dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

The term “anion” as used herein means a negatively charged counterion such as chloride, bromide, trifluoroacetate, and triethylammonium.

The term “acyl” as used herein means an alkyl or aryl (Ar) group having from 1-10 carbon atoms bonded through a carbonyl group, i.e., R—C(O)—. For example, acyl includes, but is not limited to, a C1-C6 alkanoyl, including substituted alkanoyl, wherein the alkyl portion can be substituted by an amine, amide, carboxylic, or heterocyclic group. Typical acyl groups include acetyl, benzoyl, and the like.

The term “aryl” as used herein refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms. The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like.

The terms “halogen” or “halo” as used herein means fluorine, bromine, chlorine, and iodine.

The term “haloalkyl” refers to an alkyl group having one or more halogen substituents. In some embodiments, a haloalkyl group can have 1 to 10 carbon atoms (e.g., from 1 to 8 carbon atoms). Examples of haloalkyl groups include CF₃, C₂F₅, CHF₂, CH₂F, CCl₃, CHCl₂, CH₂Cl, C2Cl₅, and the like. Perhaloalkyl groups, i.e., alkyl groups wherein all of the hydrogen atoms are replaced with halogen atoms (e.g., CF₃ and C₂F₅), are included within the definition of “haloalkyl.” For example, a C₁₋₁₀ haloalkyl group can have the formula —CᵢH₂ᵢ+1−jXⱼ, wherein X is F, Cl, Br, or I, i is an integer in the range of 1 to 10, and j is an integer in the range of 0 to 21, provided that j is less than or equal to 2i+1.

The term “heteroaryl” as used herein refers to an aromatic monocyclic ring system containing at least one ring heteroatom selected from O, N, and S or a polycyclic ring system where at least one of the rings in the ring system is aromatic and contains at least one ring heteroatom. A heteroaryl group, as a whole, can have from 5 to 14 ring atoms and contain 1-5 ring heteroatoms. In some embodiments, heteroaryl groups can include monocyclic heteroaryl rings fused to one or more aromatic carbocyclic rings, non-aromatic carbocyclic rings, or nonaromatic cycloheteroalkyl rings. The heteroaryl group can be covalently attached to the defined chemical structure at any heteroatom or carbon atom that results in a stable structure. Generally, het-eroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyrdazinyl, thienothiazolyl, thienooxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like.

The term “lower alkenyl” as used herein refers to alkenyl groups which contains 2 to 6 carbon atoms. An alkenyl group is a hydrocarbyl group containing at least one carbon-carbon double bond. As defined herein, it may be unsubstituted or substituted with the substituents described herein. The carbon-carbon double bonds may be between any two carbon atoms of the alkenyl group. It is preferred that it contains 1 or 2 carbon-carbon double bonds and more preferably one carbon-carbon double bond. The alkenyl group may be straight chained or branched. Examples include but are not limited to ethenyl, 1-propenyl, 2-propenyl, 1-butenyl, 2-butenyl, 2-methyl-1-propenyl, 1,3-butadienyl, and the like.

The term “lower alkynyl” as used herein, refers to an alkynyl group containing 2-6 carbon atoms. An alkynyl group is a hydrocarbyl group containing at least one carbon-carbon triple bond. The carbon-carbon triple bond may be between any two carbon atom of the alkynyl group. In an embodiment, the alkynyl group contains 1 or 2 carbon-carbon triple bonds and more preferably one carbon-carbon triple bond. The alkynyl group may be straight chained or branched. Examples include but are not limited to ethynyl, 1-propynyl, 2-propynyl, 1-butynyl, 2-butynyl and the like.

The term “carbalkoxy” as used herein refers to an alkoxycarbonyl group, where the attachment to the main chain is through the carbonyl group, e.g., —C(O)—. Examples include but are not limited to methoxy carbonyl, ethoxy carbonyl, and the like.

The term “oxo” as used herein refers to a double-bonded oxygen (i.e., ═O). It is also to be understood that the terminology C(O) refers to a —C═O group, whether it be ketone, aldehyde or acid or acid derivative. Similarly, S(O) refers to a —S═O group.

The term “cycloalkyl” as used herein refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like.

The term “heteroatom” as used herein refers to an atom of any element other than carbon or hydrogen and includes, for example, nitrogen, oxygen, sulfur, phosphorus, and selenium.

The term “cycloheteroalkyl” as used herein refers to a non-aromatic cycloalkyl group that contains at least one (e.g., one, two, three, four, or five) ring heteroatom selected from O, N, and S, and optionally contains one or more (e.g., one, two, or three) double or triple bonds. A cycloheteroalkyl group, as a whole, can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). Cycloheteroalkyl groups can also contain one or more oxo groups, such as phthalimidyl, piperidonyl, oxazolidinonyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, azetidine, and the like.

The compounds described herein may contain chiral centers and therefore may exist in different enantiomeric and diastereomeric forms. One aspect described herein encompasses all optical isomers or stereoisomers of the compounds described herein both as racemic mixtures and as individual enantiomers or diastereoisomers, or mixtures thereof, and to all pharmaceutical compositions or methods of treatment described herein that contain or employ them, respectively. Individual isomers can be obtained by known methods, such as optical resolution, optically selective reaction, or chiral chromatographic separation in the preparation of the final product or its intermediate.

The compounds described herein can exist in unsolvated forms as well as solvated forms, including hydrated forms. In general, the solvated forms, including hydrated forms, are equivalent to unsolvated forms and are intended to be encompassed within the scope described herein.

Compounds described herein also includes isotopically labelled compounds, which are identical to those described herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ³¹P, ³²P, ³⁵S, ¹⁸F, and ³⁶Cl, respectively. Compounds described herein, prodrugs thereof, and pharmaceutically acceptable salts of said compounds or of said prodrugs which contain the aforementioned isotopes and/or other isotopes of other atoms are within the scope described herein. Certain isotopically labelled compounds described herein, for example those into which radioactive isotopes such as ³H and ¹⁴C are incorporated, are useful in drug and/or substrate tissue distribution assays. Tritiated, i.e., ³H, and carbon-14 i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds described herein and prodrugs thereof can generally be prepared by carrying out the procedures disclosed in the Schemes and/or in the Examples and Preparations below, by substituting a readily available isotopically labelled reagent for a non-isotopically labelled reagent.

Compounds described herein also includes compounds conjugated with probes including but not restricted to photocrosslinkers, dyes, biotin etc.

The term “analog” as used herein refers to a small organic compound, a nucleotide, a protein, or a polypeptide that possesses similar or identical activity or function(s) as the compound, nucleotide, protein or polypeptide or compound having the desired activity and therapeutic effect (e.g., inhibition of tumor growth), but need not necessarily comprise a sequence or structure that is similar or identical to the sequence or structure of the preferred embodiment.

The term “derivative” as used herein refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein, or polypeptide possesses a similar or identical function as the parent polypeptide.

In some aspects, of the current disclosure, the compounds provided herein may bind to a site on the ribosome adjacent to RPS23. In some aspects, the compounds provided herein can activate the integrated stress response.

In an aspect, the disclosure provides compounds comprising Formulae (I)-(VII) or a pharmaceutically acceptable salt thereof:

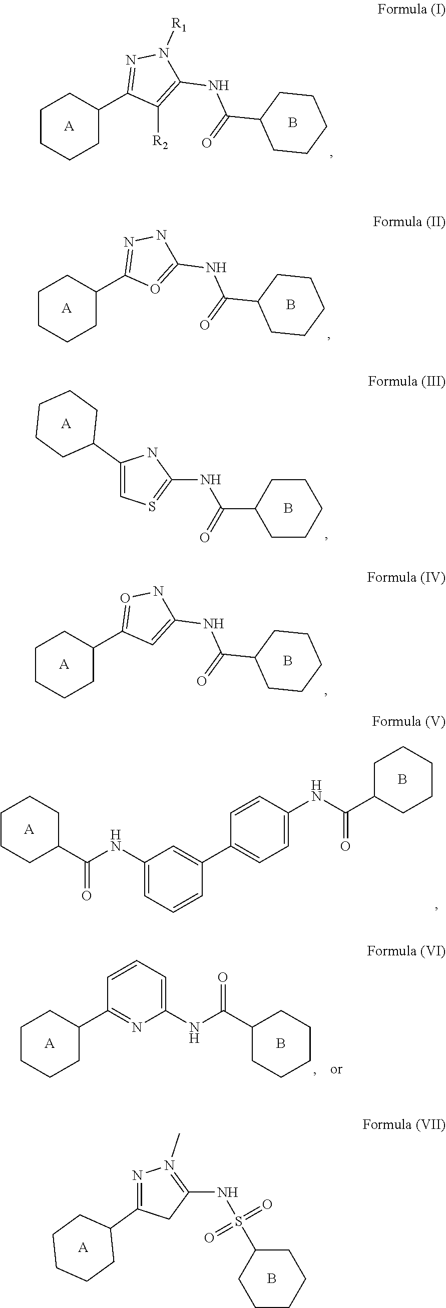

wherein:
cycle A and cycle B are each independently selected from —(C6-C10)-aryl or substituted aryl, —(C6-C10)-heteroaryl or substituted hetero aryl, —(C3-C10)-alkyl or substituted alkyl;
R₁ is alkyl, aryl, or alkyl-aryl;
R₂ is hydrogen, halide, or alkyl; and
wherein said heterocyclyl has 1-4 heteroatoms independently selected from N, NH, O, S, SO, and SO₂, and said heteroaryl has 1-4 heteroatoms independently selected from N, NH, O, and S

In an aspect, the disclosure provides compounds comprising Formula (Ia) wherein cycle A and cycle B are a C-6 substituted aryl in Formula (I) or a pharmaceutically acceptable salt thereof:

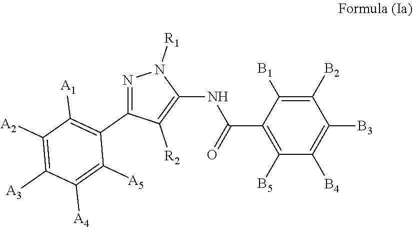

wherein:
A₁-A₅ and B₁-B₅ are each independently selected from a halogen, —R, —OR, —NO₂, —NCS, —CN, —CF₃, —OCF3, —SiR3, —NH2, —SR, —SOR, —SO2R, —SO2N(R)2, —SO3R, —(CR2)1-3R, —(CR2)1-3-OR, —(CR2)0-3-C(O)NR(CR2)0-3R, —(CR2)0-3-C(O)NR(CR2)0-30R, —C(O)R, —C(O)C(O)R, —C(O)CH2C(O)R, —C(S)R, —C(S)OR, —C(O)OR, —C(O)C(O)OR, —C(O)C(O)N(R)2, —OC(O)R, —C(O)N(R)2, —OC(O)N(R)2, —C(S)N(R)2, —(CR2)0-3NHC(O)R, —N(R)N(R)COR, —N(R)N(R)C(O)OR, —N(R)N(R)CON(R)2, —N(R)SO2R, —N(R)SO2N(R)2, —N(R)C(O)OR, —N(R)C(O)R, —N(R)C(S)R, —N(R)C(O)N(R)2, —N(R)C(S)N(R)2, —N(COR)COR, —N(OR)R, —C(═NH)N(R)2, —C(O)N(OR)R, —C(═NOR)R, —CF3, or —NHC(O)R;
wherein R is hydrogen, alkyl, alkyl amine, alkyl sulfonamide, aryl, substituted aryl, heterocyclic aryl amine, substituted heterocyclic aryl amine, cyclic aliphatic amine, heterocyclic aliphatic amine, substituted heterocyclic aliphatic amine, alkyne; or together with the carbon atom to form a cycloalkyl, a cycloalkenyl, or a heterocyclylalkyl ring;
R₁ is alkyl, aryl, or alkyl-aryl; and
R₂ is hydrogen, halide, or alkyl.

In certain embodiments, the disclosure provides compounds comprising Formula (Ia) or a pharmaceutically acceptable salt thereof wherein A₁-A₅ are each independently selected from

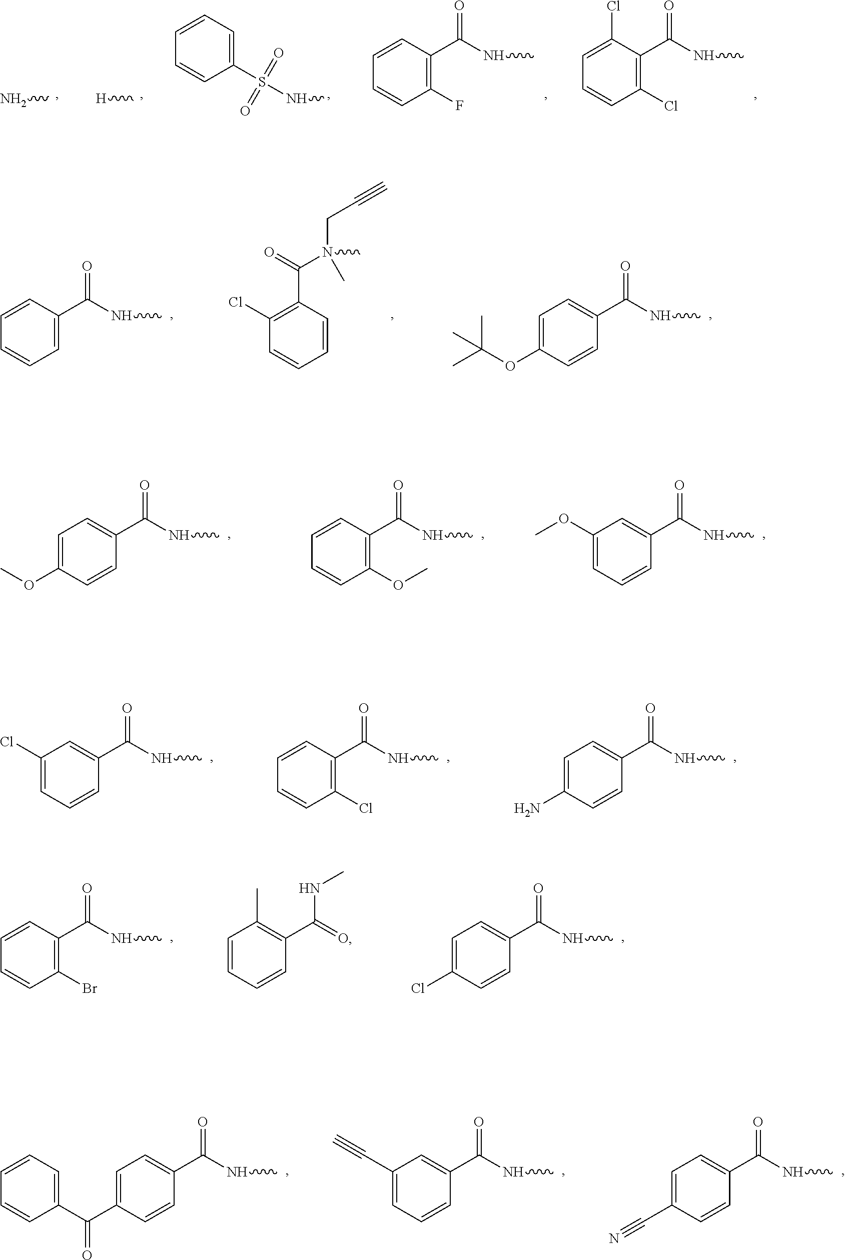

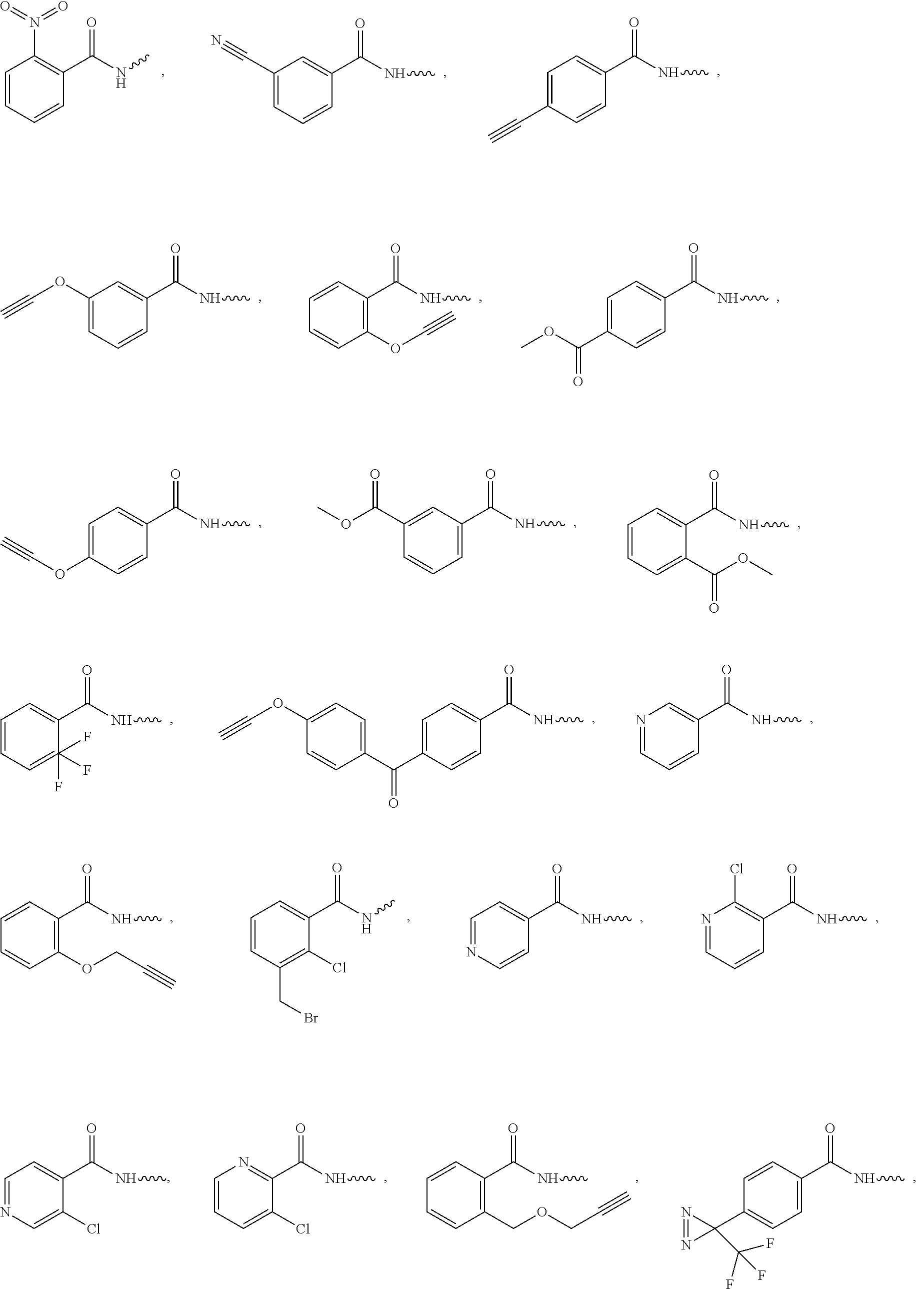

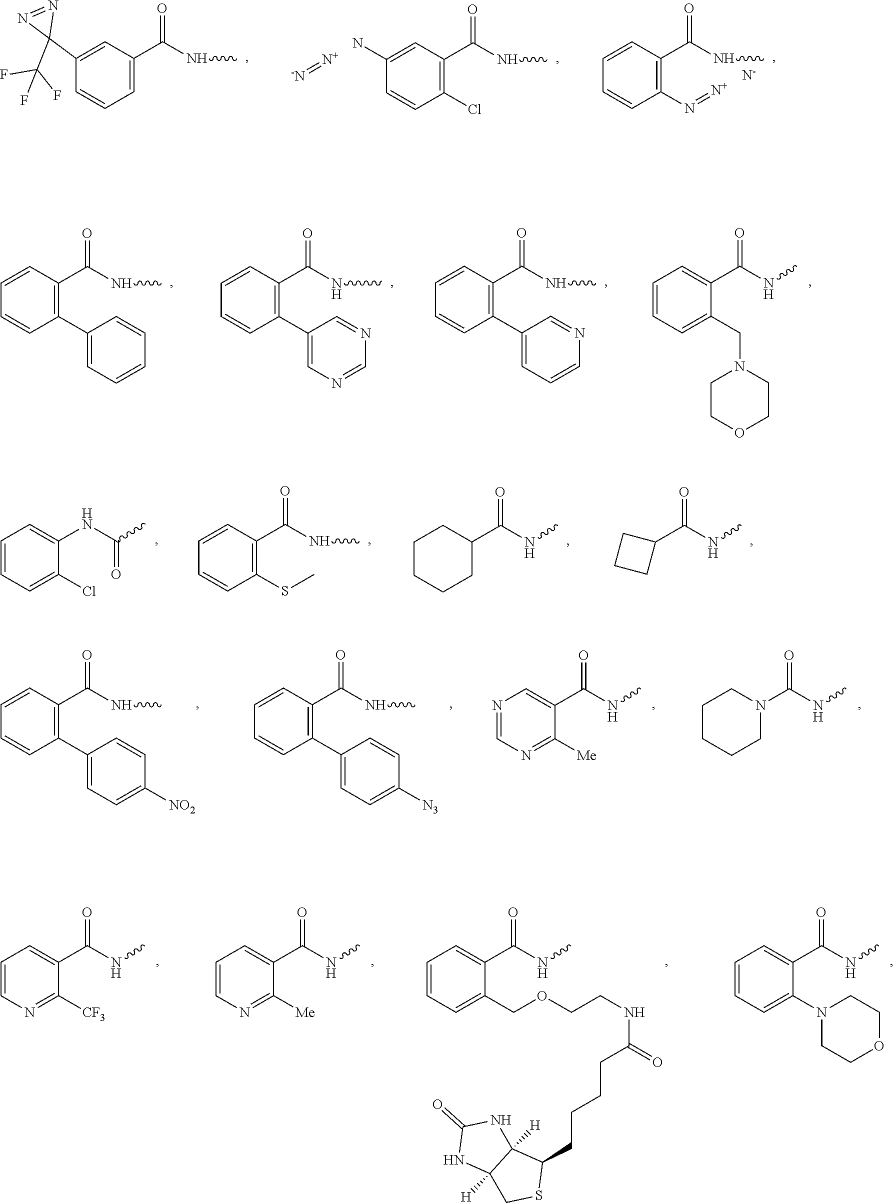

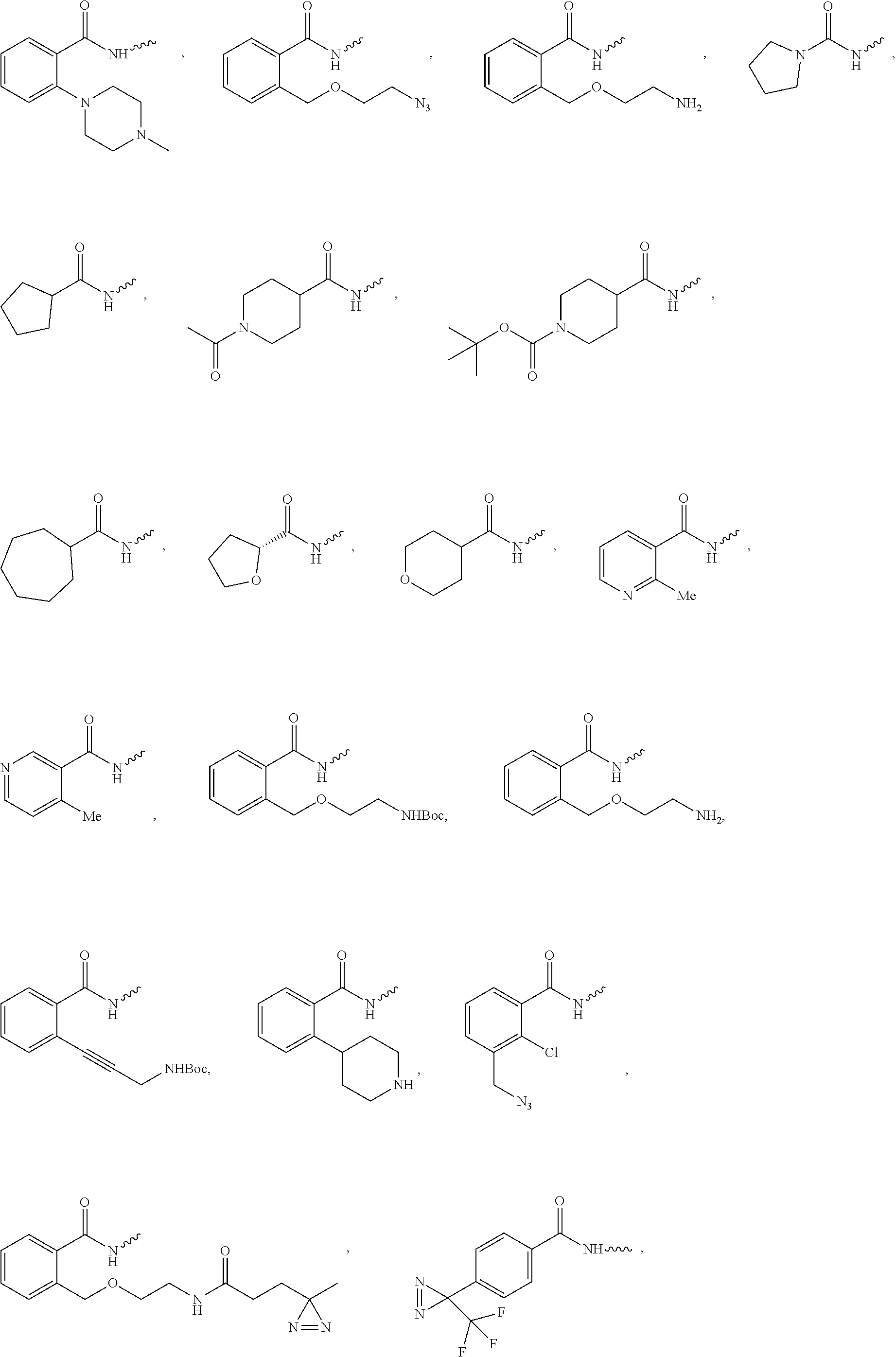

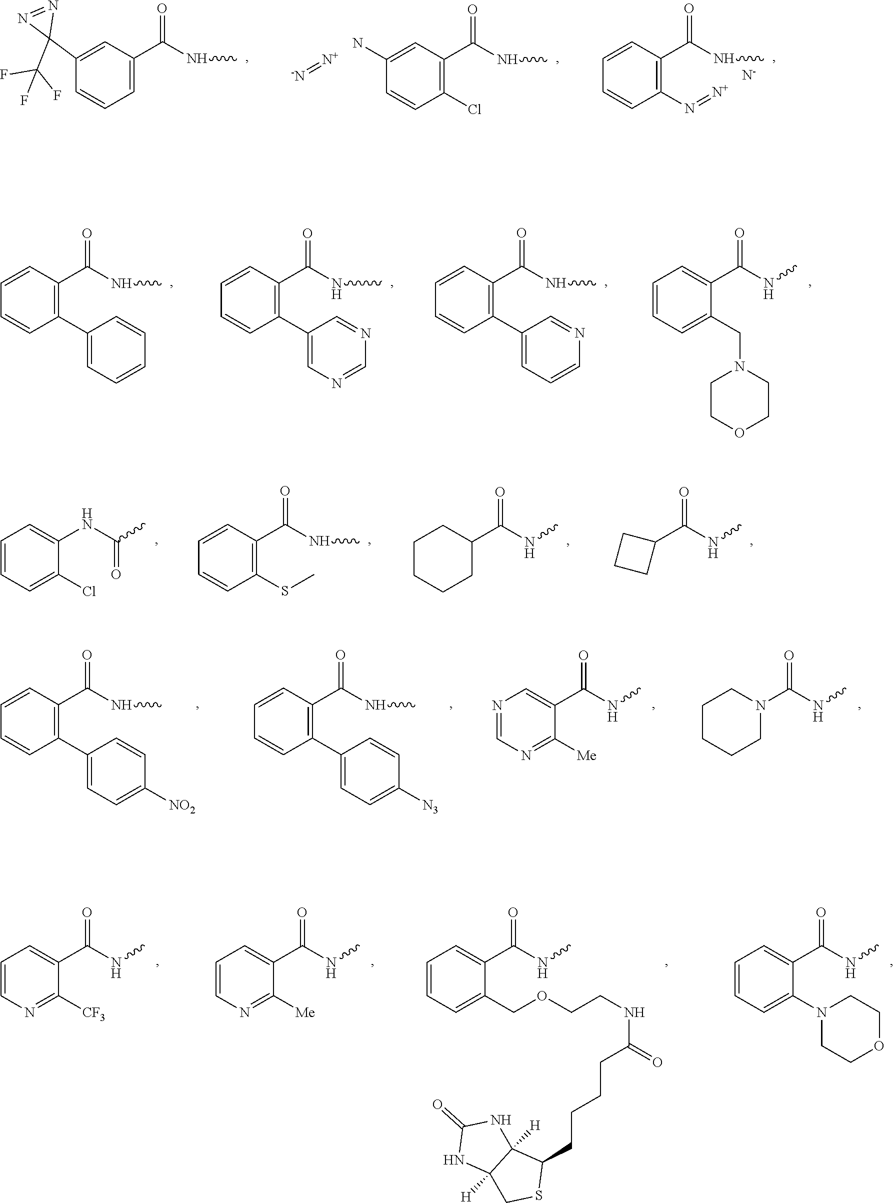

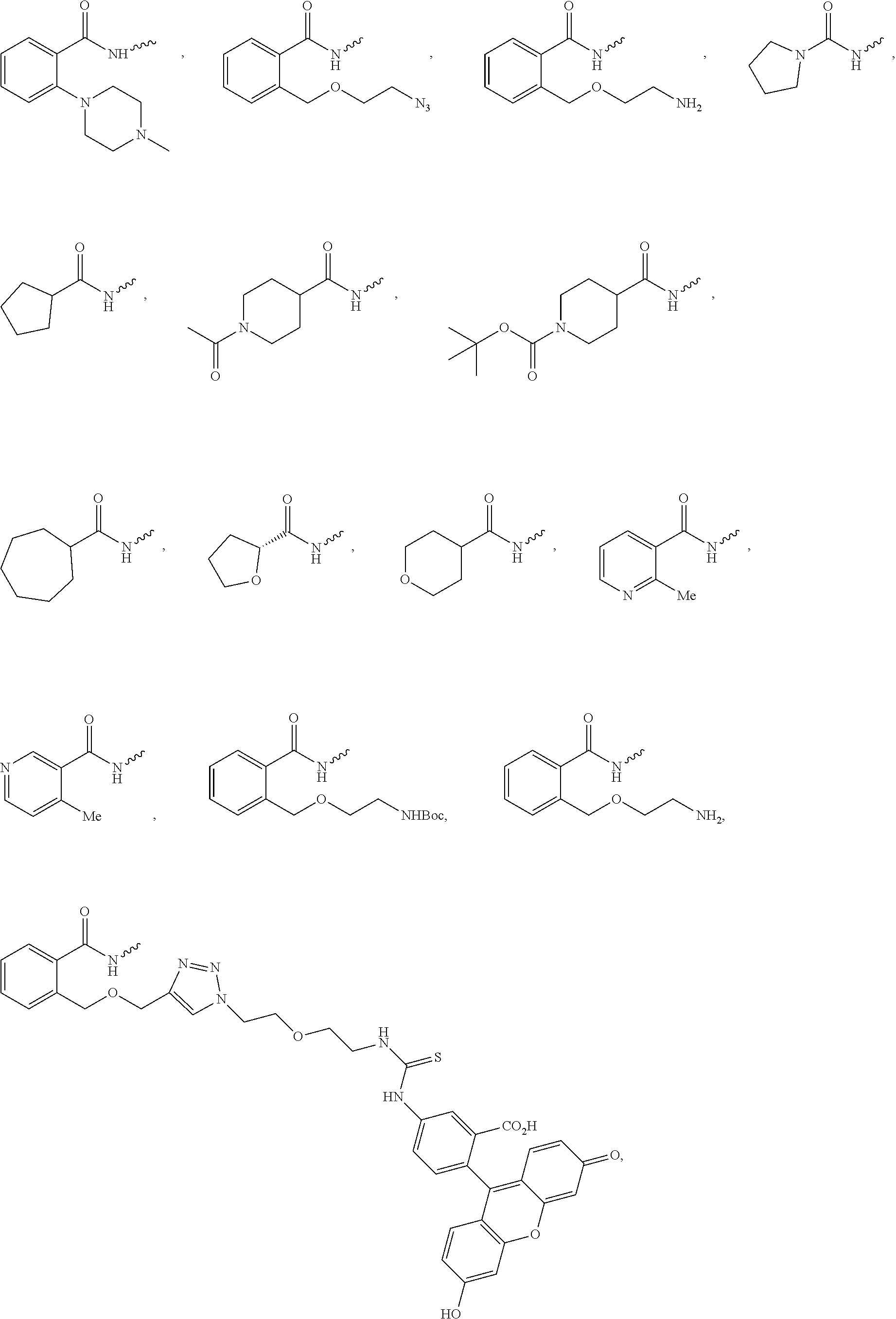

and B₁-B₅ are each independently selected from

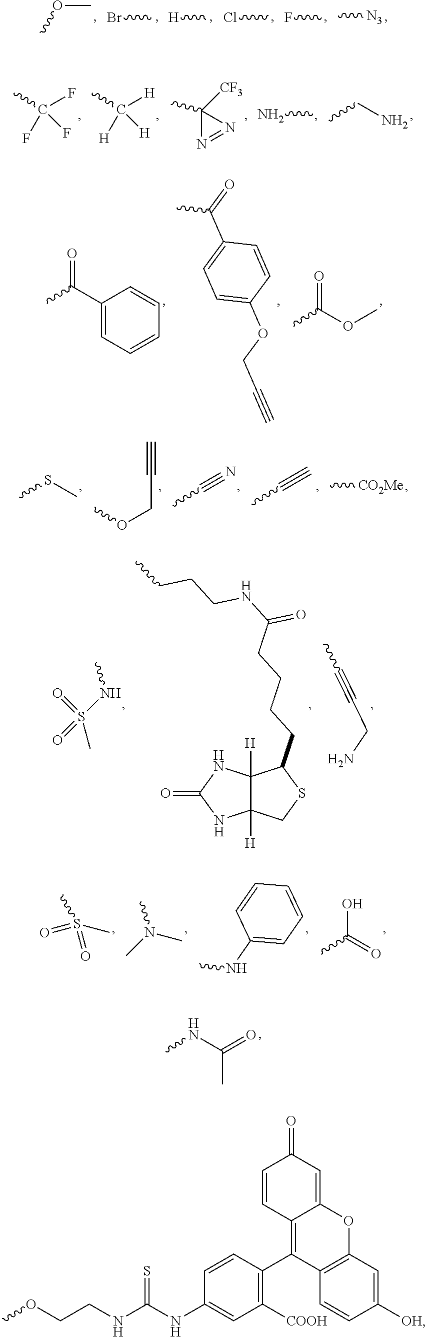

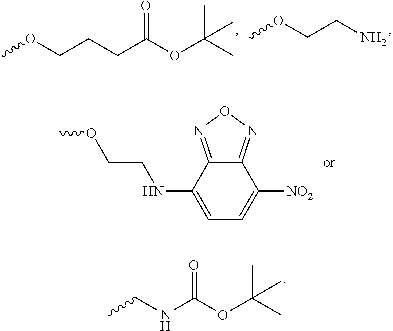

In some embodiments, the compound is a compound with Formula (Ia), wherein the compound is

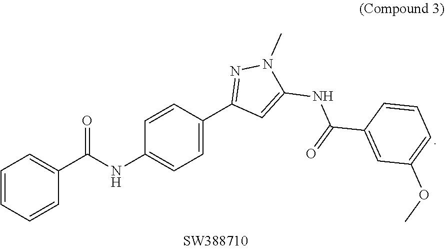 [SMILES: COc1cccc(C(=O)Nc2cc(-c3ccc(NC(=O)c4ccccc4)cc3)nn2C)c1]

In other embodiments, the compound is a compound with Formula (Ia), wherein the compound is

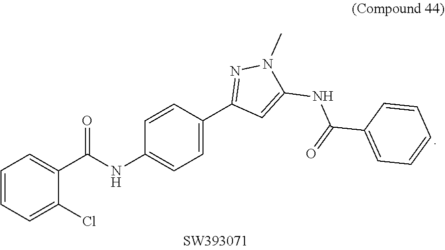 [SMILES: Cn1nc(-c2ccc(NC(=O)c3ccccc3Cl)cc2)cc1NC(=O)c1ccccc1]

In other embodiments, the compound is a compound with Formula (Ia), wherein the compound is

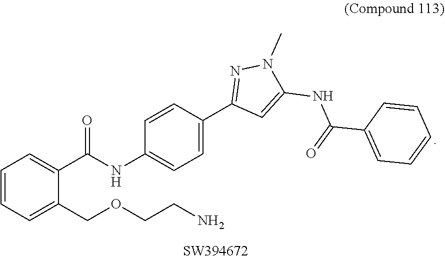 [SMILES: Cn1nc(-c2ccc(NC(=O)c3ccccc3COCCN)cc2)cc1NC(=O)c1ccccc1]

In certain embodiments, the compounds are compounds of Formula (I) wherein cycle A is a substituted aryl are described above and cycle B is cycloalkyl, substituted cycloalkyl, heterocycloalkyl, or substituted heteroalkyl as shown if Formula (Ib) below

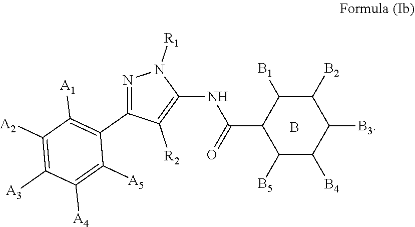

In some embodiments, the compound of Formula (Ib) is any one of the compounds below

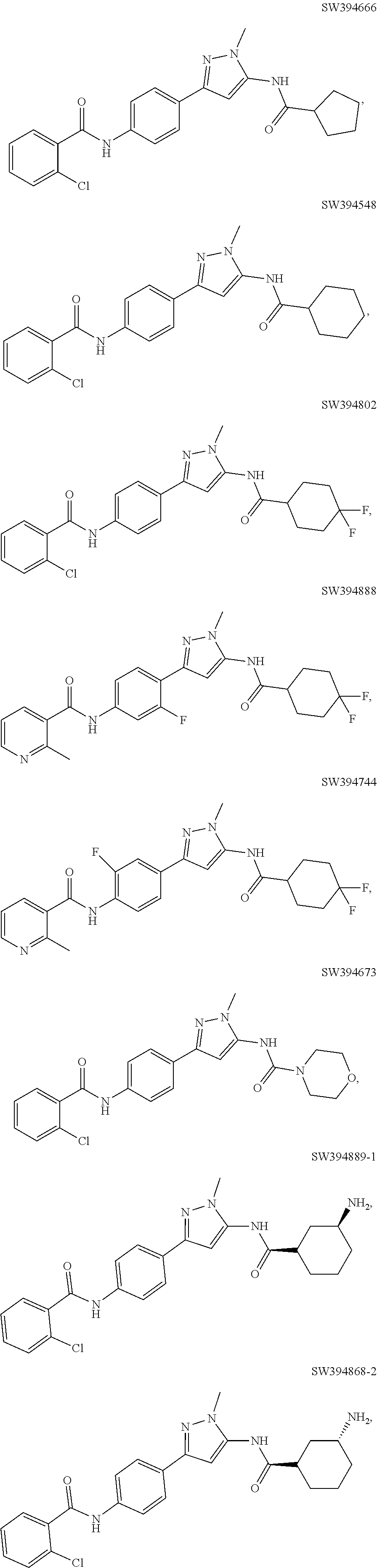

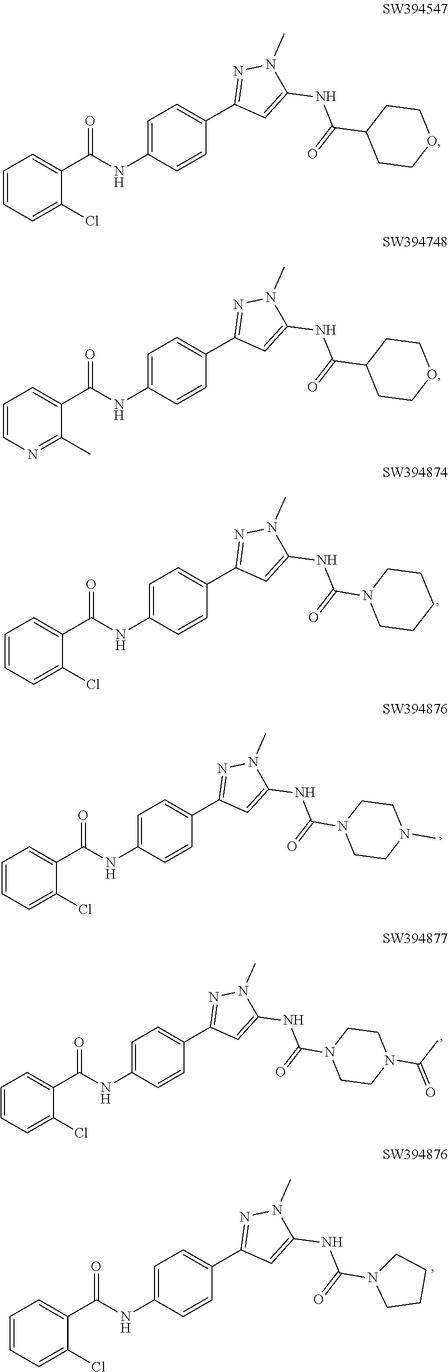 [SMILES: Cn1nc(-c2ccc(NC(=O)c3ccccc3Cl)cc2)cc1NC(=O)C1CCOCC1 ; Cc1ncccc1C(=O)Nc1ccc(-c2cc(NC(=O)C3CCOCC3)n(C)n2)cc1 ; Cn1nc(-c2ccc(NC(=O)c3ccccc3Cl)cc2)cc1NC(=O)N1CCCCC1 ; CN1CCN(C(=O)Nc2cc(-c3ccc(NC(=O)c4ccccc4Cl)cc3)nn2C)CC1 ; CC(=O)N1CCN(C(=O)Nc2cc(-c3ccc(NC(=O)c4ccccc4Cl)cc3)nn2C)CC1 ; Cn1nc(-c2ccc(NC(=O)c3ccccc3Cl)cc2)cc1NC(=O)N1CCCC1]

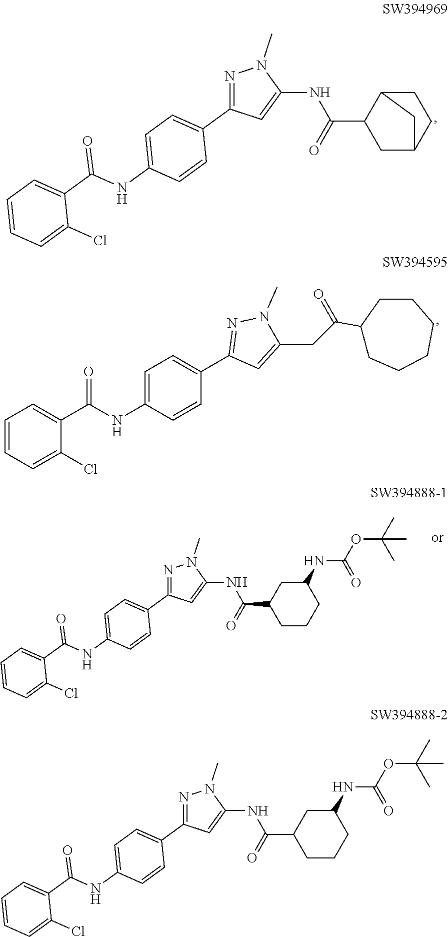

or a pharmaceutically acceptable salt thereof.

In other embodiments, the disclosure provides compounds of Formula (II) or Formula (III) and wherein in Formula (II) or Formula (III) cycle A and cycle B are C6-substituted aryl as shown in Formula (IIa)-(IIIa) and wherein X is a halogen. The halogen is fluorine, bromine, chloride, and/or iodine.

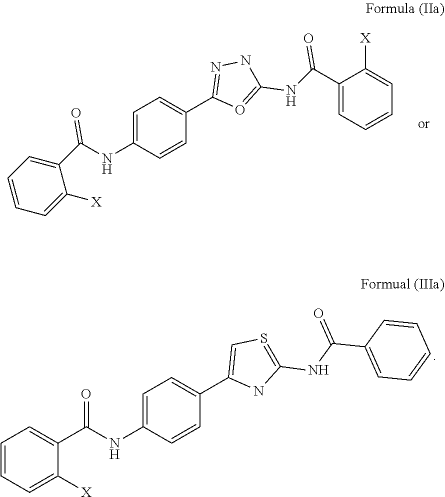

In other embodiments, the disclosure provides compounds of Formula (IV) wherein in Formula (IV) cycle A is a substituted C6-aryl and cycle B is a C-6 aryl as shown below:

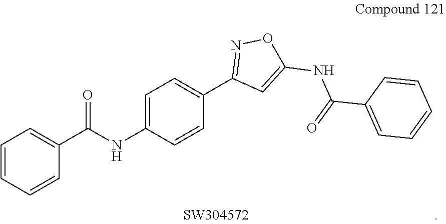 [SMILES: O=C(Nc1ccc(-c2cc(NC(=O)c3ccccc3)on2)cc1)c1ccccc1]

In other embodiments, the disclosure provides compounds of Formula (V) wherein in Formula (Va) cycle A is a substituted C6-aryl and cycle B is a C-6 aryl as shown below:

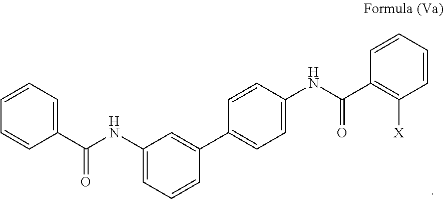

In some embodiments of the disclosure, X in Formula (Va) is chlorine and the compound is

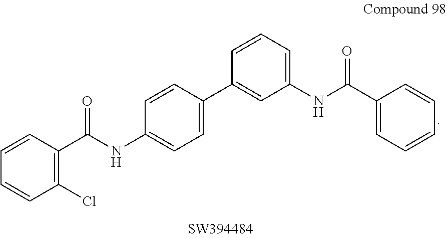 [SMILES: O=C(Nc1cccc(-c2ccc(NC(=O)c3ccccc3Cl)cc2)c1)c1ccccc1]

In other embodiments, the disclosure provides compounds of Formula (VI) wherein in Formula (VI) cycle A is a substituted C6-aryl and cycle B is a C-6 aryl as shown below:

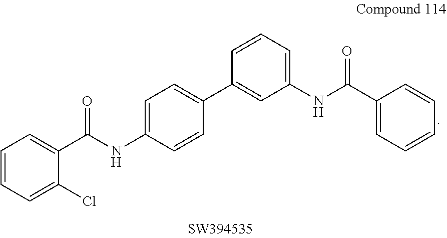 [SMILES: O=C(Nc1cccc(-c2ccc(NC(=O)c3ccccc3Cl)cc2)c1)c1ccccc1]

In other embodiments, the disclosure provides compounds of Formula (VII) wherein in Formula (VII) cycle A is a substituted C6-aryl and cycle B is a C-6 aryl as shown below:

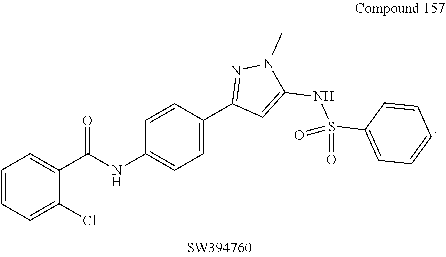 [SMILES: Cn1nc(-c2ccc(NC(=O)c3ccccc3Cl)cc2)cc1NS(=O)(=O)c1ccccc1]

In other embodiments, the disclosure provides the compounds of Table 1, listed as compounds 1-188:

TABLE 1

| Num- | | | | |
|---|---|---|---|---|
| ber | Name | Structure | ¹H NMR | ¹³C NMR |
| 1 | SW106593 | 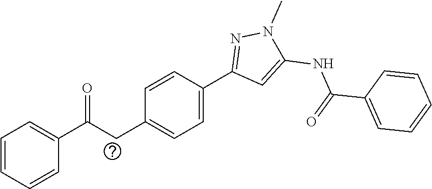 [SMILES: Cn1nc(-c2ccc(CC(=O)c3ccccc3)cc2)cc1NC(=O)c1ccccc1] | (600 MHz, DMSO-d₆) δ 10.51 (s, 1H), 10.36 (s, 1H), 8.05-8.01 (m, 2H), 8.00-7.96 (m, 2H), 7.85 (d, J = 8.7 Hz, 2H), 7.78 (d, J = 8.7 Hz, 2H), 7.67-7.62 (m, 1H), 7.62-7.52 (m, 5H), 6.70 (s, 1H), 3.76 (s, 3H) | 165.7, 165.5, 148.0, 138.6, 137.6, 135.0, 133.4, 132.2, 131.6, 128.9, 128.6, 128.4, 128.0, 127.7, 125.01, 120.4, 97.6, 35.9 |
| 2 | SW388709 | 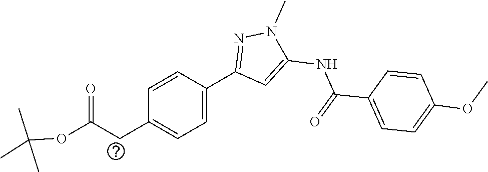 [SMILES: COc1ccc(C(=O)Nc2cc(-c3ccc(CC(=O)OC(C)(C)C)cc3)nn2C)cc1] | (600 MHz, Methanol-d₄) δ 7.93 (d, J = 8.8 Hz, 2H), 7.63 (d, J = 8.7 Hz, 2H), 7.40 (d, J = 8.2 Hz, 2H), 7.01-6.96 (m, 2H), 6.50 (s, 1H), 3.86 (s, 3H), 3.77 (s, 3H), 1.50 (s, 9H); | (151 MHz, MeOD) δ 167.8, 163.7, 154.4, 150.6, 139.3, 138.3, 130.4, 128.2, 126.4, 125.7, 119.3, 114.4, 98.5, 55.8, 35.8, 28.6; |
| 3 | SW388710 | 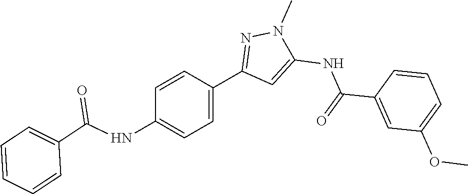 [SMILES: COc1cccc(C(=O)Nc2cc(-c3ccc(NC(=O)c4ccccc4)cc3)nn2C)c1] | (600 MHz, Methanol-d₄) δ 7.97-7.85 (m, 2H), 7.73 (s, 4H), 7.60-7.49 (m, 3H), 7.46 (dd, J = 8.4, 6.9 Hz, 2H), 7.40 (t, J = 8.0 Hz, 1H), 7.12 (dd, J = 8.3, 2.6 Hz, 1H), 6.57 (s, 1H), 3.86 (s, 3H), 3.80 (s, 3H); | (151 MHz, Methanol-d₄) δ 168.2, 168.1, 160.5, 150.5, 138.8, 138.1, 135.5, 135.0, 132.3, 130.3, 129.9, 129.0, 128.0, 126.4, 121.6, 120.5, 119.0, 113.6, 98.8, 55.7, 35.9; |
| 4 | SW388711 | 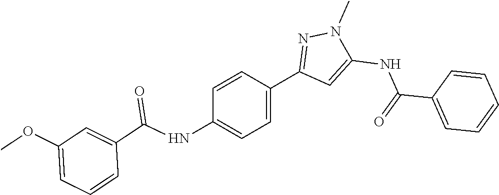 [SMILES: COc1cccc(C(=O)Nc2ccc(-c3cc(NC(=O)c4ccccc4)n(C)n3)cc2)c1] | (600 MHz, DMSO-d₆) δ 10.49 (s, 1H), 10.31 (s, 1H), 8.04-8.00 (m, 2H), 7.84 (d, J = 8.4 Hz, 2H), 7.78 (d, J = 8.6 Hz, 2H), 7.66-7.62 (m, 1H), 7.57 (t, J = 7.6 Hz, 3H), 7.51 (t, J = 2.1 Hz, 1H), 7.45 (t, J = 7.9 Hz, 1H), 7.16 (dd, J = 8.2, 2.6 Hz, 1H), 6.70 (s, 1H), 3.85 (s, 3H), 3.76 (s, 3H); | (151 MHz, DMSO) δ 165.7, 165.2, 159.2, 148.0, 138.49, 137.6, 136.4, 133.3, 132.2, 129.6, 128.6, 128.0, 125.1, 120.5, 120.0, 117.4, 112.9, 97.6, 55.4, 35.9; |
| 5 | SW388712 | 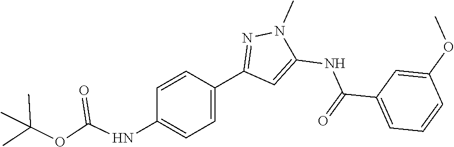 [SMILES: COc1cccc(C(=O)Nc2cc(-c3ccc(NC(=O)OC(C)(C)C)cc3)nn2C)c1] | (400 MHz, Methanol-d₄) δ 7.64-7.59 (m, 2H), 7.54-7.48 (m, 2H), 7.44- 7.35 (m, 3H), 7.10 (ddd, J = 8.3, 2.6, 1.0 Hz, 1H), 6.50 (s, 1H), 3.83 (s, 3H), 3.76 (s, 3H), 1.50 (s, 9H); | (101 MHz, MeOD) δ 168.2, 160.6, 154.5, 150.7, 139.5, 138.1, 135.0, 130.3, 128.3, 126.5, 120.5, 119.4, 119.0, 113.7, 98.6, 80.7, 55.8, 35.8, 28.6; |
| 6 | SW388713 | 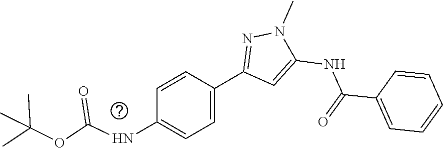 [SMILES: Cn1nc(-c2ccc(NC(=O)OC(C)(C)C)cc2)cc1NC(=O)c1ccccc1] | (600 MHz, Methanol-d₄) δ 7.95 (d, J = 7.7 Hz, 2H), 7.66-7.61 (m, 2H), 7.60- 7.55 (m, 1H), 7.49 (t, J = 7.3 Hz, 2H), 7.41 (d, J = 8.2 Hz, 2H), 6.53 (d, J = 1.6 Hz, 1H), 3.78 (d, J = 1.7 Hz, 3H), 1.51 (d, J = 1.6 Hz, 9H); | (151 MHz, Methanol-d₄) δ 168.5, 154.5, 150.8, 139.5, 138.2, 133.7, 133.1, 129.2, 128.38, 126.5, 119.3, 98.6, 35.8, 28.6; |
| 7 | SW388714 | 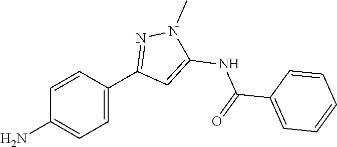 [SMILES: Cn1nc(-c2ccc(N)cc2)cc1NC(=O)c1ccccc1] | (400 MHz, Chloroform-d) δ 8.37 (s, 1H), 7.85-7.79 (m, 2H), 7.55-7.46 (m, 3H), 7.41 (t, J = 7.7 Hz, 2H), 6.65- 6.60 (m, 2H), 6.35 (s, 1H), 3.64 (s, 3H); | (101 MHz, CDCl₃) δ 166.4, 150.4, 146.3, 136.3, 133.1, 132.5, 128.9, 127.6, 126.7, 124.2, 115.3, 97.4, 35.7; |
| 8 | SW388715 | 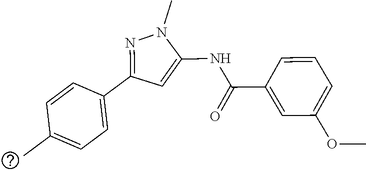 | 600 MHz, DMSO-d₆) δ 10.34 (s, 1H), 7.59 (d, J = 7.6 Hz, 1H), 7.55 (t, J = 2.2 Hz, 1H), 7.46 (dd, J = 8.4, 2.3 Hz, 3H), 7.20 (ddd, J = 8.3, 2.7, 0.9 Hz, 1H), 6.60-6.57 (m, 2H), 6.48 (s, 1H), 5.18 (s, 2H), 3.84 (s, 3H), 3.69 (s, 3H); | (151 MHz, DMSO) δ 165.4, 159.3, 149.2, 148.3, 137.0, 134.8, 129.8, 125.8, 121.4, 120.1, 118.0, 113.9, 113.0, 96.6, 55.4, 35.6; |
| 9 | SW388716 | 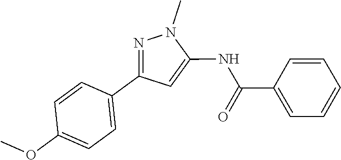 [SMILES: COc1ccc(-c2cc(NC(=O)c3ccccc3)n(C)n2)cc1] | (600 MHz, DMSO-d₆) δ 10.45 (s, 1H), 10.17 (s, 1H), 8.02 (d, J = 7.6 Hz, 2H), 7.99 (d, J = 8.8 Hz, 2H), 7.83 (d, J = 8.5 Hz, 2H), 7.77 (d, J = 8.6 Hz, 2H), 7.66-7.62 (m, 1H), 7.57 (t, J = 7.6 Hz, 2H), 7.10-7.05 (m, 2H), 6.70 (s, 1H), 3.84 (s, 3H), 3.76 (s, 3H); | (151 MHz, DMSO) δ 165.7, 164.9, 161.9, 148.1, 138.8, 137.6, 133.4, 132.2, 129.7, 128.7, 128.6, 128.0, 127.0, 125.0, 120.4, 113.6, 97.6, 55.5, 35.9; |
| 10 | SW388717 | 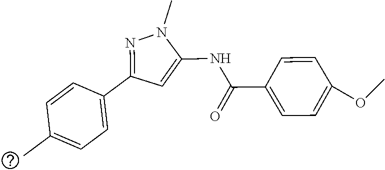 | (400 MHz, Methanol-d₄) δ 7.92 (d, J = 8.8 Hz, 2H), 7.49 (d, J = 8.5 Hz, 2H), 6.97 (d, J = 8.9 Hz, 2H), 6.72 (d, J = 8.5 Hz, 2H), 6.42 (s, 1H), 3.84 (s, 3H), 3.74 (s, 3H); | (101 MHz, Methanol-d₄) δ 167.8, 163.7, 151.3, 147.4, 138.1, 130.3, 127.1, 125.8, 124.2, 116.0, 114.4, 98.1, 55.8, 35.6; |
| 11 | SW388718 | 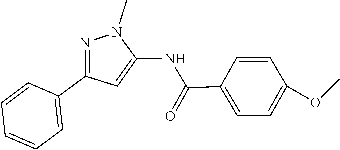 [SMILES: COc1ccc(C(=O)Nc2cc(-c3ccccc3)nn2C)cc1] | (600 MHz, DMSO-d₆) δ 10.33 (s, 1H), 10.27 (s, 1H), 8.00 (d, J = 8.8 Hz, 2H), 7.99-7.92 (m, 2H), 7.84 (d, J = 8.8 Hz, 2H), 7.81-7.73 (m, 2H), 7.64-7.57 (m, 1H), 7.57-7.49 (m, 2H), 7.20-7.02 (m, 2H), 6.67 (s, 1H), 3.85 (s, 3H), 3.74 (s, 3H); | (151 MHz, DMSO-d₆) δ 165.5, 165.1, 162.4, 148.0, 138.5, 137.7, 135.0, 131.6, 129.9, 128.9, 128.4, 127.7, 125.4, 125.0, 120.4, 113.8, 97.7, 69.8, 55.5, 35.8; |
| 12 | SW389001 | 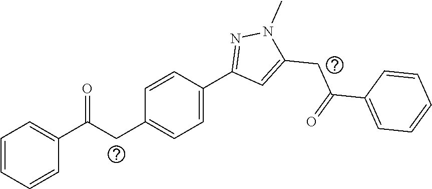 [SMILES: Cn1nc(-c2ccc(CC(=O)c3ccccc3)cc2)cc1CC(=O)c1ccccc1] | (400 MHz, Methanol-d₄) δ 8.05 (dd, J = 7.7, 1.8 Hz, 1H), 8.00-7.94 (m, 2H), 7.77-7.67 (m, 4H), 7.63-7.57 (m, 1H), 7.55-7.47 (m, 3H), 7.17- 7.01 (m, 2H), 6.59 (s, 1H), 4.05 (s, 3H), 3.81 (s, 3H); | (101 MHz, MeOD) δ 168.5, 165.3, 158.1, 150.5, 138.5, 138.3, 134.1, 133.8, 133.1, 132.1, 130.1, 129.3, 128.5, 126.6, 122.5, 121.9, 121.4, 112.4, 98.8, 56.6, 36.0; |
| 13 | SW389002 | 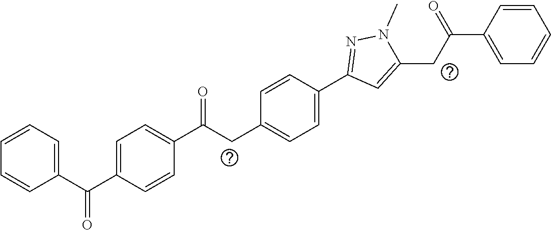 [SMILES: Cn1nc(-c2ccc(CC(=O)c3ccc(C(=O)c4ccccc4)cc3)cc2)cc1CC(=O)c1ccccc1] | (400 MHz, Methanol-d₄) δ 8.04 (d, J = 8.0 Hz, 1H), 7.96 (d, J = 7.6 Hz, 1H), 7.85 (d, J = 8.0 Hz, 1H), 7.80-7.69 (m, 3H), 7.60 (dt, J = 21.3, 7.4 Hz, 1H), 7.50 (q, J = 7.3 Hz, 2H), 6.58 (s, 0H), 3.80 (s, 1H); | (101 MHz, Methanol-d₄) δ 197.4, 168.3, 166.8, 150.4, 140.5, 139.0, 138.5, 138.1, 137.4, 133.7, 133.6, 133.0, 130.6, 130.5, 130.1, 129.1, 129.0, 128.3, 128.1, 126.4, 121.6, 98.7, 35.9; |
| 14 | SW389003 | 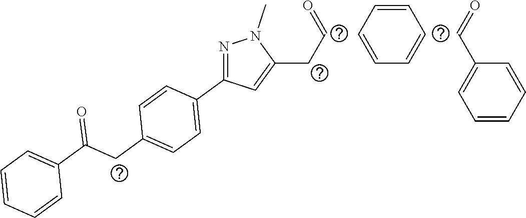 | (600 MHz, DMSO-d₆) δ 10.66 (s, 1H), 10.33 (s, 1H), 8.18 (d, J = 7.9 Hz, 2H), 7.97 (d, J = 7.6 Hz, 2H), 7.90 (d, J = 7.9 Hz, 2H), 7.85 (d, J = 8.3 Hz, 2H), 7.79 (d, J = 8.0 Hz, 4H), 7.73 (t, J = 7.4 Hz, 1H), 7.65-7.58 (m, 3H), 7.54 (t, J = 7.5 Hz, 2H), 6.74 (s, 1H), 3.79 (s, 3H); | (151 MHz, DMSO-d₆) δ 195.4, 165.5, 165.0, 148.1, 140.0, 138.6, 137.3, 136.6, 136.6, 135.0, 133.2, 131.6, 128.8, 128.8, 128.4, 128.2, 127.7, 125.1, 120.4, 97.6, 36.0; |
| 15 | SW389004 | 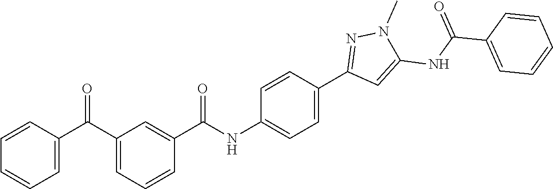 [SMILES: Cn1nc(-c2ccc(NC(=O)c3cccc(C(=O)c4ccccc4)c3)cc2)cc1NC(=O)c1ccccc1] | (400 MHz, Chloroform-d) δ 9.16 (s, 1H), 9.11 (s, 1H), 8.18 (s, 1H), 7.96 (d, J = 7.7 Hz, 1H), 7.81 (d, J = 7.7 Hz, 2H), 7.76 (d, J = 7.7 Hz, 1H), 7.67-7.61 (m, 2H), 7.52 (t, J = 7.4 Hz, 1H), 7.46-7.33 (m, 7H), 7.31-7.23 (m, 3H), 6.21 (s, 1H), 3.52 (s, 3H); | (101 MHz, Chloroform-d) δ 196.2, 167.0, 165.7, 149.3, 137.8, 137.1, 136.8, 136.6, 135.2, 133.0, 133.0, 132.9, 132.5, 131.4, 130.1, 130.0, 128.7, 128.6, 127.8, 125.8, 121.3, 98.5, 35.7; |
| 16 | SW389005 | 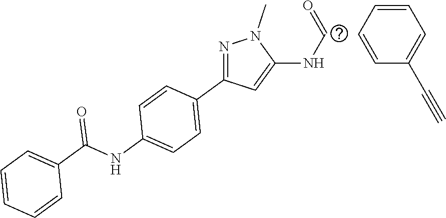 | (600 MHz, DMSO-d₆) δ 10.54 (s, 1H), 10.34 (s, 1H), 8.13 (d, J = 1.8 Hz, 1H), 8.02 (t, J = 6.5 Hz, 1H), 7.99- 7.96 (m, 2H), 7.85 (d, J = 8.5 Hz, 2H), 7.79 (d, J = 8.8 Hz, 2H), 7.75 (dd, J = 7.7, 1.4 Hz, 1H), 7.60 (dq, J = 7.7, 3.6 Hz, 2H), 7.54 (dd, J = 8.3, 6.8 Hz, 2H), 6.71 (d, J = 2.5 Hz, 1H), 4.36 (s, 1H), 3.77 (s, 3H); | (151 MHz, DMSO) δ 165.6, 164.8, 148.1, 138.6, 137.4, 135.2, 135.0, 133.9, 131.6, 131.0, 129.2, 128.9, 128.6, 128.6, 128.5, 128.0, 127.7, 97.6, 82.7, 81.9, 35.9; |
| 17 | SW389006 | 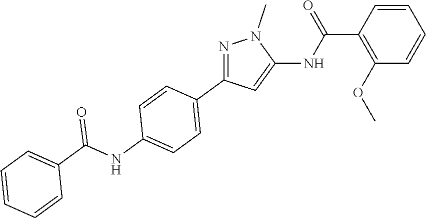 [SMILES: COc1ccccc1C(=O)Nc1cc(-c2ccc(NC(=O)c3ccccc3)cc2)nn1C] | (400 MHz, Methanol-d₄) δ 8.15 (dd, J = 8.0, 1.9 Hz, 1H), 8.03-7.98 (m, 1H), 7.93-7.86 (m, 2H), 7.74 (s, 4H), 7.61-7.38 (m, 3H), 7.14 (dt, J = 7.4, 3.2 Hz, 2H), 6.77 (s, 1H), 4.09 (s, 3H), 3.85 (s, 3H); | (151 MHz, DMSO) δ 165.5, 163.6, 157.1, 148.0, 138.5, 137.5, 135.0, 133.0, 131.6, 130.4, 128.4, 127.7, 125.3, 125.1, 122.7, 120.7, 120.4, 112.2, 96.2, 56.1, 35.6; |
| 18 | SW389007 | 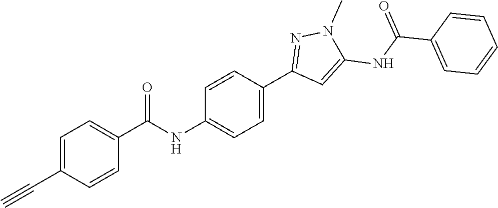 [SMILES: C#Cc1ccc(C(=O)Nc2ccc(-c3cc(NC(=O)c4ccccc4)n(C)n3)cc2)cc1] | (600 MHz, DMSO-d₆) δ 10.45 (s, 1H), 10.40 (s, 1H), 8.03-8.00 (m, 2H), 8.00-7.98 (m, 2H), 7.85-7.82 (m, 2H), 7.80-7.77 (m, 2H), 7.64 (dd, J = 8.4, 6.7 Hz, 3H), 7.57 (dd, J = 8.3, 7.0 Hz, 2H), 6.71 (s, 1H), 4.43 (s, 1H), 3.76 (s, 3H); | (151 MHz, DMSO) δ 165.7, 164.7, 148.0, 138.4, 137.6, 135.0, 133.3, 132.2, 131.8, 129.1, 128.6, 128.0, 128.0, 125.1, 124.8, 120.5, 97.6, 83.2, 82.9, 35.8; |
| 19 | SW389008 | 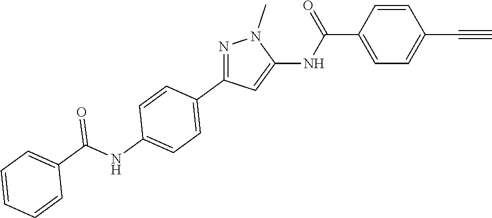 [SMILES: C#Cc1ccc(C(=O)Nc2cc(-c3ccc(NC(=O)c4ccccc4)cc3)nn2C)cc1] | (600 MHz, DMSO-d₆) δ 10.56 (s, 1H), 10.34 (s, 1H), 8.03 (d, J = 8.0 Hz, 2H), 7.99-7.96 (m, 2H), 7.86-7.82 (m, 2H), 7.80-7.76 (m, 2H), 7.69- 7.65 (m, 2H), 7.62-7.58 (m, 1H), 7.54 (dd, J = 8.3, 6.8 Hz, 2H), 6.70 (s, 1H), 4.47 (s, 1H), 3.76 (s, 3H); | (151 MHz, DMSO) δ 165.5, 164.9, 148.1, 138.6, 137.4, 135.0, 133.3, 131.9, 131.6, 128.8, 128.4, 128.3, 127.7, 125.4, 125.1, 120.4, 97.6, 83.5, 82.8, 35.9; |
| 20 | SW389009 | 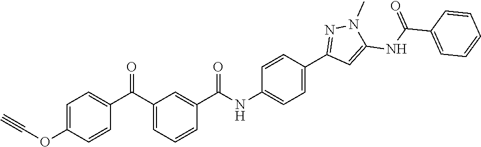 [SMILES: C#COc1ccc(C(=O)c2cccc(C(=O)Nc3ccc(-c4cc(NC(=O)c5ccccc5)n(C)n4)cc3)c2)cc1] | (600 MHz, Chloroform-d) δ 9.03 (s, 1H), 8.97 (s, 1H), 8.13 (d, J = 1.8 Hz, 1H), 7.98 (dt, J = 7.8, 1.4 Hz, 1H) 7.86 (d, J = 7.6 Hz, 2H), 7.80-7.72 (m, 1H), 7.70 (d, J = 8.8 Hz, 2H), 7.46 (h, J = 5.8 Hz, 5H), 7.39 (t, J = 7.7 Hz, 1H), 7.32 (t, J = 7.7 Hz, 2H), 6.96 (d, J = 8.8 Hz, 2H), 6.26 (s, 1H), 4.71 (d, J = 2.4 Hz, 2H), 3.58 (s, 3H), 2.56 (s, 1H); | (151 MHz, Chloroform-d) δ 194.9, 166.8, 165.6, 161.5, 149.2, 138.4, 137.2, 136.8, 135.1, 132.9, 132.7, 132.6, 132.6, 131.0, 130.2, 129.8, 128.8, 128.4, 127.8, 125.9, 121.2, 114.7, 98.4, 77.8, 76.5, 56.0, 35.8; |
| 21 | SW389010 | 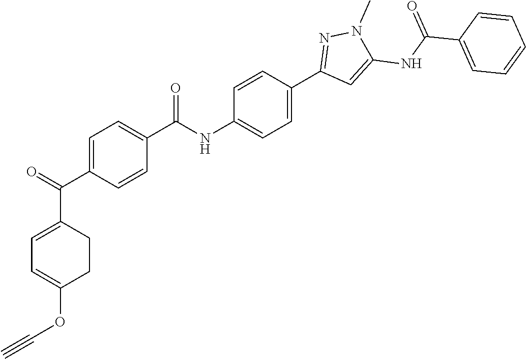 [SMILES: C#COC1=CC=C(C(=O)c2ccc(C(=O)Nc3ccc(-c4cc(NC(=O)c5ccccc5)n(C)n4)cc3)cc2)CC1] | (600 MHz, DMSO-d₆) δ 10.51 (s, 1H), 10.44 (s, 1H), 8.12 (d, J = 7.8 Hz, 2H), 8.02 (d, J = 7.5 Hz, 2H), 7.83 (dt, J = 27.9, 8.4 Hz, 8H), 7.65 (t, J = 7.5 Hz, 1H), 7.57 (t, J = 7.6 Hz, 2H), 7.18 (d, J = 8.4 Hz, 2H), 6.71 (s, 1H), 4.95 (s, 2H), 3.76 (s, 3H), 3.67 (s, 1H); | (151 MHz, DMSO-d₆) δ 194.0, 165.7, 164.8, 161.1, 148.0, 140.2, 138.4, 137.9, 137.5, 133.3, 132.2, 129.8, 129.3, 129.1, 128.6, 128.0, 127.8, 125.1, 120.5, 114.9, 97.6, 78.9, 78.7, 55.8, 35.9; |
| 22 | SW389012 | 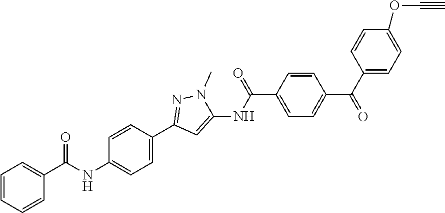 [SMILES: C#COc1ccc(C(=O)c2ccc(C(=O)Nc3cc(-c4ccc(NC(=O)c5ccccc5)cc4)nn3C)cc2)cc1] | 600 MHz, DMSO-d6) δ 10.64 (s, 1H), 10.33 (s, 1H), 8.16 (d, J = 7.8 Hz, 2H), 7.97 (d, J = 7.5 Hz, 2H), 7.85 (dd, J = 8.2, 6.1 Hz, 4H), 7.83-7.77 (m, 4H), 7.60 (t, J = 7.3 Hz, 1H), 7.55 (t, J = 7.5 Hz, 2H), 7.18 (d, J = 8.5 Hz, 2H), 6.74 (s, 1H), 4.96 (d, J = 2.5 Hz, 2H), 3.79 (s, 3H), 3.68 (d, J = 2.5 Hz, 1H); | (151 MHz, DMSO-d6) δ 194.0, 165.5, 165.1, 161.2, 148.1, 140.7, 138.6, 137.4, 136.2, 135.0, 132.2, 131.6, 129.7, 129.3, 128.9, 128.4, 128.1, 127.7, 125.1, 120.4, 114.9, 97.6, 78.9, 78.7, 55.8, 35.9; |
| 23 | SW389013 | 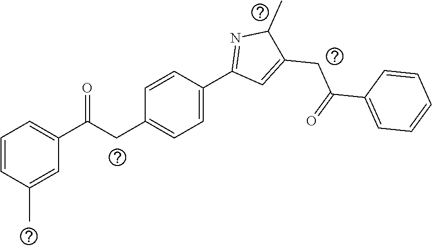 [SMILES: Cc1cccc(C(=O)Cc2ccc(C3=NC(C)C(CC(=O)c4ccccc4)=C3)cc2)c1] | (600 MHz, DMSO-d₆) δ 10.45 (s, 1H), 10.41 (s, 1H), 8.09 (t, J = 1.8 Hz, 1H), 8.04-7.98 (m, 3H), 7.86-7.82 (m, 2H), 7.81-7.77 (m, 2H), 7.70 (dt, J = 7.7, 1.5 Hz, 1H), 7.66-7.62 (m, 1H), 7.57 (t, J = 7.7 Hz, 3H), 6.71 (s, 1H), 4.35 (s, 1H), 3.76 (s, 3H); | (151 MHz, DMSO) δ 165.7, 164.5, 148.0, 138.4, 137.6, 135.4, 134.6, 133.3, 132.2, 130.7, 129.1, 129.1, 128.6, 128.4, 128.0, 125.1, 121.9, 120.5, 97.6, 82.9, 81.7, 35.9; |
| 24 | SW389014 | 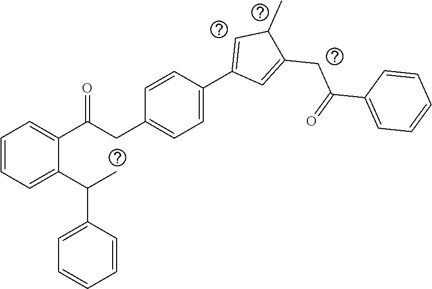 [SMILES: CC1C=C(c2ccc(CC(=O)c3ccccc3C(C)c3ccccc3)cc2)C=C1CC(=O)c1ccccc1] | (600 MHz, DMSO-d₆) δ 10.41 (s, 1H), 8.00 (d, J = 7.6 Hz, 2H), 7.85 (d, J = 7.4 Hz, 1H), 7.73 (s, 1H), 7.68 (d, J = 8.3 Hz, 2H), 7.65-7.53 (m, 7H), 7.39 (d, J = 7.7 Hz, 2H), 7.31-7.26 (m, 3H), 7.22 (t, J = 7.3 Hz, 1H), 6.66 (s, 1H), 3.73 (s, 3H); | (151 MHz, DMSO) δ 166.9, 166.2, 149.9, 148.2, 140.4, 138.0, 136.3, 133.8, 133.7, 132.7, 131.0, 130.1, 130.0, 129.1, 128.9, 128.4, 128.4, 126.4, 125.6, 125.1, 123.6, 123.3, 98.4, 92.9, 36.3; |
| 25 | SW389152 | 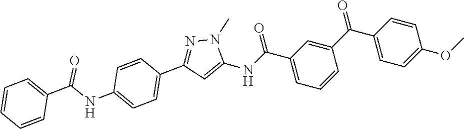 [SMILES: COc1ccc(C(=O)c2cccc(C(=O)Nc3cc(-c4ccc(NC(=O)c5ccccc5)cc4)nn3C)c2)cc1] | (600 MHz, DMSO-d₆) δ 10.61 (s, 1H), 8.32 (s, 1H), 8.28 (d, J = 7.9 Hz, 1H), 7.96 (t, J = 9.3 Hz, 3H), 7.90-7.81 (m, 4H), 7.76 (dd, J = 17.5, 8.2 Hz, 3H), 7.60 (d, J = 7.3 Hz, 1H), 7.54 (t, J = 7.6 Hz, 2H), 7.18 (d, J = 8.4 Hz, 2H), 6.71 (s, 1H), 4.95 (d, J = 2.5 Hz, 2H), 3.76 (s, 3H), 3.67 (d, J = 2.7 Hz, 1H); | (151 MHz, Methanol-d₄) δ 195.9, 168.1, 167.3, 162.4, 150.5, 139.1, 138.8, 138.0, 135.4, 134.1, 133.9, 133.1, 132.4, 132.0, 130.5, 129.9, 129.6, 129.4, 129.2, 129.1, 128.4, 128.0, 126.4, 121.7, 115.3, 101.1, 98.8, 77.1, 56.4, 36.0; |
| 26 | SW389153 | 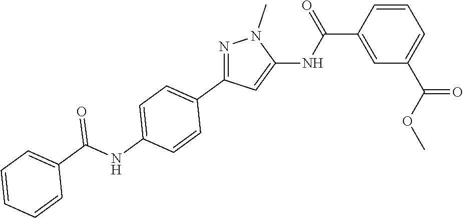 [SMILES: COC(=O)c1cccc(C(=O)Nc2cc(-c3ccc(NC(=O)c4ccccc4)cc3)nn2C)c1] | (600 MHz, Chloroform-d) δ 9.25 (s, 1H), 8.58-8.49 (m, 2H), 8.11 (dt, J = 7.8, 1.4 Hz, 1H), 8.06 (d, J = 7.7 Hz, 1H), 7.79-7.73 (m, 2H), 7.58-7.49 (m, 2H), 7.49-7.45 (m, 1H), 7.44- 7.40 (m, 2H), 7.39 (d, J = 7.8 Hz, 1H), 7.34 (t, J = 7.7 Hz, 2H), 6.29 (s, 1H), 3.81 (s, 3H), 3.64 (s, 3H); | (151 MHz, Chloroform-d) δ 166.6, 166.3, 166.1, 149.3, 136.9, 136.4, 134.6, 133.7, 133.2, 132.4, 132.0, 130.6, 130.2, 129.0, 128.81, 128.7, 127.3, 125.9, 121.3, 98.4, 52.5, 35.9; |
| 27 | SW389154 | 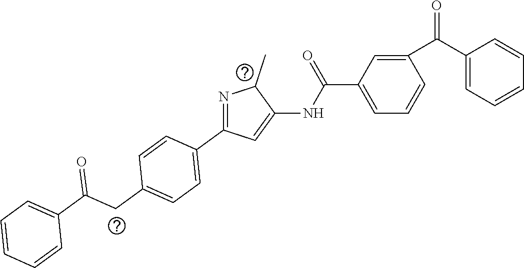 [SMILES: CC1N=C(c2ccc(CC(=O)c3ccccc3)cc2)C=C1NC(=O)c1cccc(C(=O)c2ccccc2)c1] | (600 MHz, DMSO-d₆) δ 10.64 (s, 1H), 10.33 (s, 1H), 8.37 (s, 1H), 8.31 (d, J = 7.7 Hz, 1H), 8.00-7.95 (m, 3H), 7.87-7.83 (m, 2H), 7.79 (ddd, J = 18.8, 9.4, 7.4 Hz, 5H), 7.75-7.71 (m, 1H), 7.61 (q, J = 7.3 Hz, 3H), 7.55 (dd, J = 8.3, 6.8 Hz, 2H), 6.72 (s, 1H), 3.76 (s, 3H); | (151 MHz, DMSO-d₆) δ 195.3, 165.5, 164.9, 148.1, 138.6, 137.4, 137.3, 136.6, 135.0, 133.6, 133.1, 131.9, 131.6, 129.8, 129.1, 128.9, 128.8, 128.4, 127.7, 125.1, 120.4, 97.6, 35.9; |
| 28 | SW389155 | 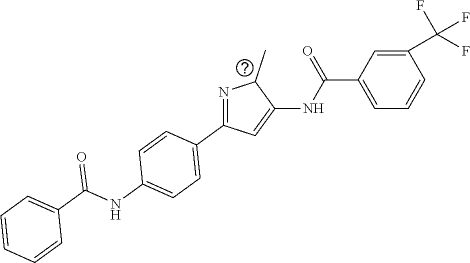 [SMILES: CC1N=C(c2ccc(NC(=O)c3ccccc3)cc2)C=C1NC(=O)c1cccc(C(F)(F)F)c1] | (600 MHz, DMSO-d₆) δ 10.73 (s, 1H), 10.36 (s, 1H), 8.37 (d, J = 2.1 Hz, 1H), 8.33 (d, J = 7.8 Hz, 1H), 8.00 (ddt, J = 12.0, 7.0, 1.4 Hz, 3H), 7.88 (d, J = 8.7 Hz, 2H), 7.84-7.78 (m, 3H), 7.62-7.57 (m, 1H), 7.57-7.52 (m, 2H), 6.75 (s, 1H), 3.80 (s, 3H); | (151 MHz, DMSO) δ 165.6, 164.4, 148.2, 138.7, 137.2, 135.1, 134.3, 132.1, 131.6, 129.9, 129.5, 129.3, 128.9, 128.8, 128.8, 128.4, 127.7, 125.2, 124.9, 124.7, 124.6, 123.1, 120.5, 97.7, 36.0; |
| 29 | SW389156 | 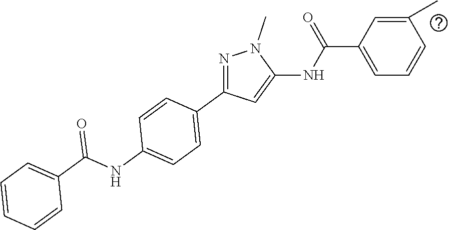 [SMILES: Cc1cccc(C(=O)Nc2cc(-c3ccc(NC(=O)c4ccccc4)cc3)nn2C)c1] | (600 MHz, DMSO-d₆) δ 10.40 (s, 1H), 10.33 (s, 1H), 8.00-7.95 (m, 2H), 7.86-7.82 (m, 3H), 7.83-7.75 (m, 3H), 7.63-7.57 (m, 1H), 7.54 (dd, J = 8.2, 6.6 Hz, 2H), 7.45 (dd, J = 4.8, 1.4 Hz, 2H), 6.69 (s, 1H), 3.75 (s, 3H), 2.41 (s, 3H); | (151 MHz, DMSO) δ 165.9, 165.6, 148.0, 138.6, 138.0, 137.6, 135.0, 133.4, 132.8, 131.6, 128.9, 128.5, 128.5, 128.5, 127.7, 125.1, 120.4, 97.6, 35.9, 21.0; |
| 30 | SW389157 | 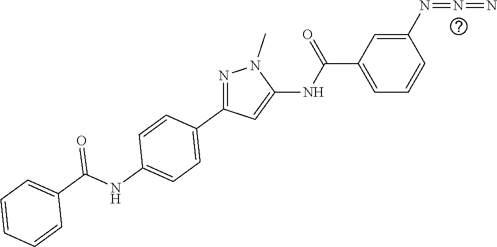 | (600 MHz, Chloroform-d) δ 7.90 (d, J = 7.3 Hz, 2H), 7.71 (d, J = 4.7 Hz, 4H), 7.64 (d, J = 5.7 Hz, 1H), 7.55- 7.40 (m, 5H), 7.23 (dt, J = 7.1, 3.5 Hz, 1H), 6.56 (d, J = 4.9 Hz, 1H), 3.79 (d, J = 4.6 Hz, 3H); | (151 MHz, DMSO) δ 165.6, 164.8, 148.1, 140.0, 138.6, 137.3, 135.0, 135.0, 135.0, 131.6, 130.4, 128.9, 128.5, 127.7, 125.1, 124.7, 122.8, 120.4, 118.5, 97.7, 35.9; |
| 31 | SW389158 | 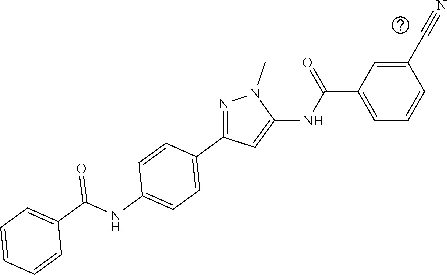 [SMILES: Cn1nc(-c2ccc(NC(=O)c3ccccc3)cc2)cc1NC(=O)c1cccc(C#N)c1] | (600 MHz, DMSO-d₆) δ 10.67 (s, 1H), 10.33 (s, 1H), 8.46 (d, J = 1.8 Hz, 1H), 8.30 (dt, J = 8.0, 1.5 Hz, 1H), 8.12 (dt, J = 7.7, 1.4 Hz, 1H), 7.99- 7.96 (m, 2H), 7.87-7.83 (m, 2H), 7.81-7.77 (m, 3H), 7.62-7.58 (m, 1H), 7.54 (dd, J = 8.2, 6.7 Hz, 2H), 6.74 (s, 1H), 3.79 (s, 3H); | (151 MHz, DMSO) δ 165.6, 165.5, 163.9, 148.1, 138.6, 138.5, 137.2, 135.5, 135.0, 135.0, 134.6, 132.8, 131.7, 131.6, 130.0, 128.8, 128.4, 128.4, 127.7, 127.7, 125.1, 120.4, 120.3, 118.3, 111.7, 97.3, 36.0; |
| 32 | SW389159 | 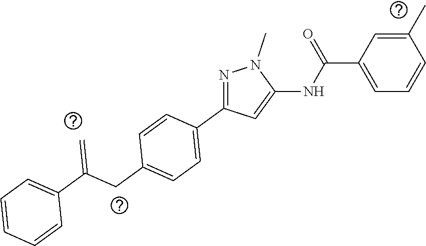 [SMILES: C=C(Cc1ccc(-c2cc(NC(=O)c3cccc(C)c3)n(C)n2)cc1)c1ccccc1] | (600 MHz, Methanol-d₄) δ 7.90 (dd, J = 8.3, 1.3 Hz, 2H), 7.78 (d, J = 7.7 Hz, 1H), 7.73 (s, 4H), 7.71-7.64 (m, 1H), 7.55-7.51 (m, 1H), 7.51-7.48 (m, 1H), 7.46 (dd, J = 8.4, 7.0 Hz, 2H), 7.30 (td, J = 8.4, 2.7 Hz, 1H), 6.58 (s, 1H), 3.80 (s, 3H); | (151 MHz, Methanol-d₄) δ 168.2, 167.0, 164.2, 162.5, 150.5, 138.8, 138.0, 135.9 (d, J = 7.1 Hz), 135.5, 132.3, 131.1 (d, J = 7.7 Hz), 129.9, 129.1, 128.0, 126.4, 121.7, 120.0, 119.9, 115.6, 115.5, 98.8, 35.9; |
| 33 | SW389160 | 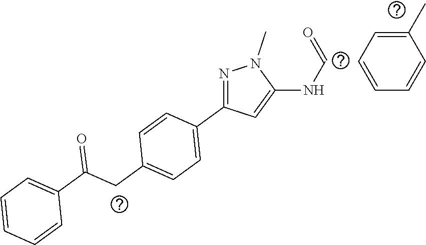 | (600 MHz, DMSO-d₆) δ 10.50 (s, 1H), 10.34 (s, 1H), 8.00-7.96 (m, 2H), 7.87-7.84 (m, 3H), 7.79 (d, J = 8.7 Hz, 2H), 7.78-7.74 (m, 1H), 7.62- 7.57 (m, 1H), 7.56-7.47 (m, 4H), 6.71 (s, 1H), 3.76 (s, 3H), 2.56 (s, 3H); | (151 MHz, DMSO) δ 165.6, 165.4, 148.1, 139.1, 138.6, 137.5, 135.0, 134.0, 131.6, 129.3, 129.2, 128.9, 128.5, 127.7, 125.1, 124.9, 124.4, 120.5, 97.7, 35.9, 14.6; |
| 34 | SW389161 | 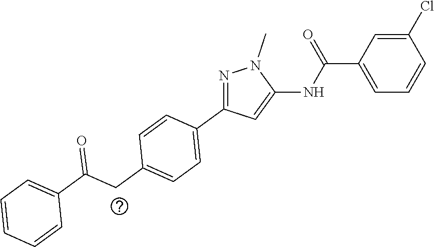 [SMILES: Cn1nc(-c2ccc(CC(=O)c3ccccc3)cc2)cc1NC(=O)c1cccc(Cl)c1] | (400 MHz, MeOD) δ 7.91 (t, J = 1.9 Hz, 1H), 7.82 (ddt, J = 15.6, 7.8, 1.3 Hz, 3H), 7.66 (s, 4H), 7.52-7.35 (m, 5H), 6.51 (s, 1H), 3.73 (s, 3H). | |
| 35 | SW389162 | 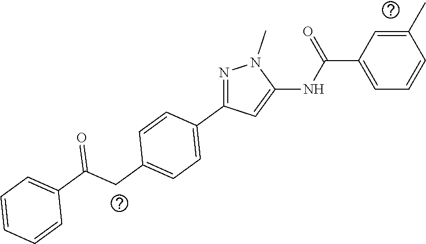 [SMILES: Cc1cccc(C(=O)Nc2cc(-c3ccc(CC(=O)c4ccccc4)cc3)nn2C)c1] | (600 MHz, DMSO) δ 10.56 (s, 1H), 10.33 (s, 1H), 8.20 (t, J = 1.9 Hz, 1H), 8.02-7.95 (m, 3H), 7.88-7.81 (m, 3H), 7.81-7.76 (m, 2H), 7.62-7.59 (m, 1H), 7.58-7.52 (m, 3H), 6.71 (s, 1H), 3.77 (s, 3H). | (151 MHz, DMSO) δ 131.31, 131.02, 128.88, 128.13, 127.57, 125.52, 122.28, 120.84, 120.74, 98.00, 40.88, 36.37. |
| 36 | SW393061 | 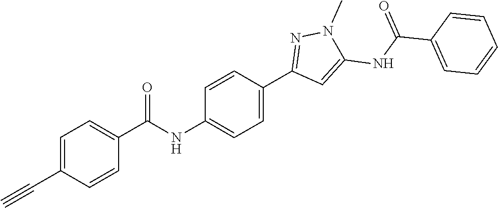 [SMILES: C#Cc1ccc(C(=O)Nc2ccc(-c3cc(NC(=O)c4ccccc4)n(C)n3)cc2)cc1] | (600 MHz, DMSO) δ 10.56 (s, 1H), 10.45 (s, 1H), 8.15-8.10 (m, 2H), 8.07-8.04 (m, 2H), 8.03-8.00 (m, 3H), 7.82 (q, J = 8.8 Hz, 4H), 7.68- 7.62 (m, 1H), 7.58 (dd, J = 8.3, 7.0 Hz, 2H), 6.72 (s, 1H), 3.76 (s, 3H). | (151 MHz, DMSO) δ 166.17, 164.55, 148.37, 139.46, 138.56, 138.01, 133.77, 132.96, 132.67, 129.76, 129.03 (d, J = 2.6 Hz), 128.40, 125.58, 120.96, 98.10, 36.33.; |
| 37 | SW393062 | 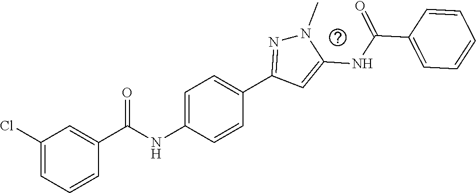 [SMILES: Cn1nc(-c2ccc(NC(=O)c3cccc(Cl)c3)cc2)cc1NC(=O)c1ccccc1] | (600 MHz, DMSO) δ 10.44 (d, J = 10.4 Hz, 2H), 8.05-8.00 (m, 3H), 7.94 (dt, J = 7.8, 1.4 Hz, 1H), 7.85- 7.78 (m, 4H), 7.70-7.67 (m, 1H), 7.67-7.63 (m, 1H), 7.62-7.56 (m, 3H), 6.71 (s, 1H), 3.77 (s, 3H). | (151 MHz, DMSO) δ 164.46, 148.40, 138.69, 137.98, 137.39, 133.77, 133.69, 132.68, 131.88, 130.92, 129.58, 129.05, 128.39, 127.89, 126.99, 125.54, 120.92, 98.08, 36.32. |
| 38 | SW393063 | 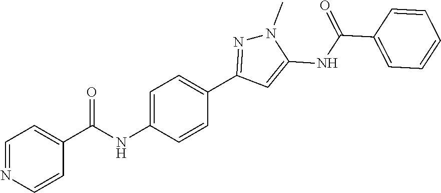 [SMILES: Cn1nc(-c2ccc(NC(=O)c3ccncc3)cc2)cc1NC(=O)c1ccccc1] | (600 MHz, MeOD) δ 8.63-8.58 (m, 2H), 7.91-7.87 (m, 2H), 7.79-7.75 (m, 2H), 7.66 (s, 4H), 7.54-7.48 (m, 1H), 7.45-7.39 (m, 2H), 6.51 (s, 1H), 3.73 (s, 3H). | (151 MHz, MeOD) δ 167.56, 164.59, 149.70, 149.68, 142.94, 137.51, 133.01, 132.46, 129.84, 128.62, 127.75, 125.90, 121.86, 121.01, 98.14, 49.55, 35.45.; |
| 39 | SW393065 | 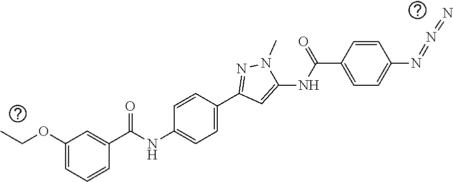 | (600 MHz, Methanol-d₄) δ 7.58-7.53 (m, 2H), 7.29 (s, 4H), 7.10 (d, J = 1.8 Hz, 2H), 6.99-6.94 (m, 1H), 6.74- 6.68 (m, 3H), 6.13 (s, 1H), 4.33 (d, J = 2.4 Hz, 2H), 4.00 (s, 4H); | (151 MHz, Methanol-d₄) δ 167.2, 166.9, 158.1, 150.2, 144.8, 138.4, 137.9, 136.7, 130.1, 130.0, 129.8, 129.7, 126.2, 121.4, 120.8, 119.4, 118.9, 114.3, 98.5, 78.5, 76.4, 56.3, 35.8; |
| 40 | SW393066 | 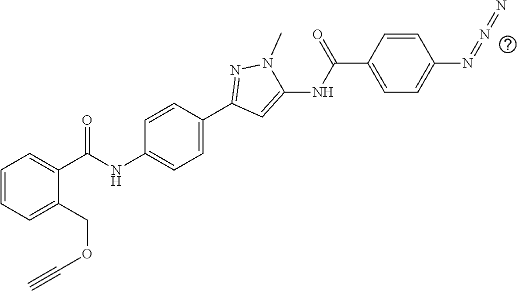 | (600 MHz, DMSO-d₆) δ 10.43 (s, 1H), 10.21 (s, 1H), 8.06 (d, J = 8.6 Hz, 2H), 7.83-7.74 (m, 4H), 7.65 (dd, J = 7.6, 1.9 Hz, 1H), 7.52 (ddd, J = 8.8, 7.4, 1.8 Hz, 1H), 7.41-7.29 (m, 2H), 7.25 (d, J = 8.4 Hz, 1H), 7.12 (t, J = 7.5 Hz, 1H), 6.69 (s, 1H), 4.97 (d, J = 2.4 Hz, 2H), 3.75 (s, 3H), 3.67 (s, 1H); | (151 MHz, DMSO-d₆) δ 164.7, 164.3, 154.5, 148.0, 143.4, 138.4, 137.5, 131.7, 129.9, 129.7, 128.8, 125.7, 125.2, 121.3, 119.7, 119.2, 113.5, 97.6, 79.0, 78.9, 56.4, 35.9; |
| 41 | SW393068 | 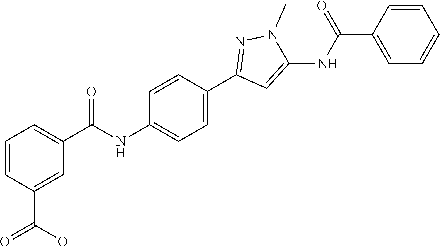 [SMILES: Cn1nc(-c2ccc(NC(=O)c3cccc(C(=O)O)c3)cc2)cc1NC(=O)c1ccccc1] | (600 MHz, DMSO) δ 10.54 (s, 1H), 10.45 (s, 1H), 8.56 (t, J = 1.8 Hz, 1H), 8.26 (dt, J = 7.9, 1.4 Hz, 1H), 8.18 (dt, J = 7.8, 1.4 Hz, 1H), 8.06-8.00 (m, 2H), 7.87-7.79 (m, 4H), 7.72 (t, J = 7.7 Hz, 1H), 7.68-7.62 (m, 1H), 7.58 (dd, J = 8.3, 7.0 Hz, 2H), 6.72 (s, 1H), 3.93 (s, 3H), 3.77 (s, 3H). | (151 MHz, DMSO) δ 166.26, 164.97, 148.40, 138.74, 135.93, 132.88, 132.62, 132.50, 130.33, 129.58, 129.02, 128.81, 128.39, 125.53, 120.99, 52.92, 36.30. |
| 42 | SW393069 | 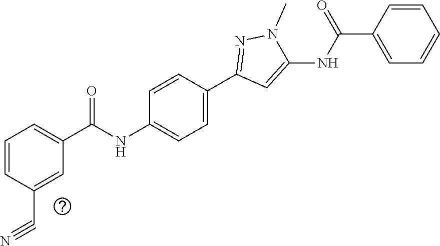 [SMILES: Cn1nc(-c2ccc(NC(=O)c3cccc(C#N)c3)cc2)cc1NC(=O)c1ccccc1] | (600 MHz, DMSO) δ 10.50 (s, 1H), 10.45 (s, 1H), 8.43 (t, J = 1.7 Hz, 1H), 8.27 (dt, J = 8.0, 1.5 Hz, 1H), 8.09 (dt, J = 7.8, 1.4 Hz, 1H), 8.04-8.00 (m, 2H), 7.80 (s, 4H), 7.78 (t, J = 7.8 Hz, 1H), 7.68-7.62 (m, 1H), 7.58 (dd, J = 8.3, 6.9 Hz, 2H), 6.72 (s, 1H), 3.77 (s, 3H). | (151 MHz, DMSO) δ 166.16, 164.06, 148.37, 138.57, 138.03, 136.43, 135.47, 133.79, 133.02, 132.67, 131.78, 130.34, 129.71, 129.04, 128.39, 125.59, 120.88, 118.83, 111.99, 98.07, 36.32. |
| 43 | SW393070 | 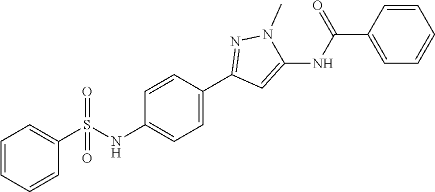 [SMILES: Cn1nc(-c2ccc(NS(=O)(=O)c3ccccc3)cc2)cc1NC(=O)c1ccccc1] | (600 MHz, DMSO) δ 10.39 (d, J = 26.6 Hz, 2H), 8.03-7.96 (m, 2H), 7.82-7.76 (m, 2H), 7.67-7.62 (m, 3H), 7.62-7.60 (m, 1H), 7.56 (td, J = 7.5, 7.0, 1.5 Hz, 4H), 7.16-7.10 (m, 2H), 6.63 (s, 1H), 3.72 (s, 3H). | (151 MHz, DMSO) δ 166.11, 150.10, 148.07, 139.95, 137.97, 137.35, 133.73, 133.39, 132.67, 129.88, 129.75, 129.03, 128.88, 128.37, 128.14, 127.13, 126.06, 120.77, 98.05, 36.29. |
| 44 | SW393071 | 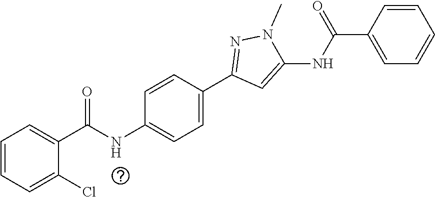 [SMILES: Cn1nc(-c2ccc(NC(=O)c3ccccc3Cl)cc2)cc1NC(=O)c1ccccc1] | (600 MHz, DMSO) δ 8.04-8.00 (m, 2H), 7.81-7.74 (m, 4H), 7.67-7.63 (m, 1H), 7.61 (dd, J = 7.5, 1.8 Hz, 1H), 7.60-7.55 (m, 3H), 7.53 (td, J = 7.7, 1.8 Hz, 1H), 7.48 (td, J = 7.4, 1.3 Hz, 1H), 6.70 (s, 1H), 3.76 (s, 3H). | (151 MHz, DMSO) δ 166.15, 165.32, 148.37, 138.71, 137.46, 131.58, 130.40, 130.13, 129.52, 129.42, 129.03, 128.39, 127.75, 125.65, 120.11, 98.01, 36.29. |
| 45 | SW393072 | 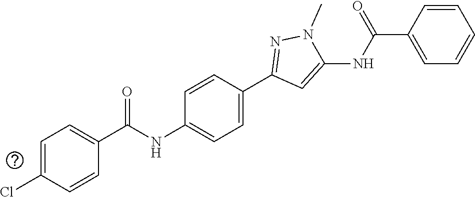 [SMILES: Cn1nc(-c2ccc(NC(=O)c3ccc(Cl)cc3)cc2)cc1NC(=O)c1ccccc1] | (600 MHz, DMSO) δ 10.44 (s, 1H), 10.39 (s, 1H), 8.04-7.99 (m, 4H), 7.84-7.77 (m, 4H), 7.66-7.62 (m, 3H), 7.60-7.55 (m, 2H), 6.71 (s, 1H), 3.76 (s, 3H). | (151 MHz, MeOD) δ 128.92, 128.73, 128.65, 127.75, 125.95, 120.85, 98.02. |
| 46 | SW393073 | 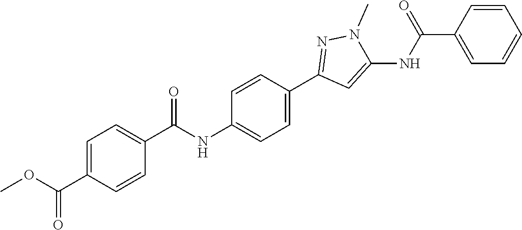 [SMILES: COC(=O)c1ccc(C(=O)Nc2ccc(-c3cc(NC(=O)c4ccccc4)n(C)n3)cc2)cc1] | (600 MHz, DMSO) δ 10.52 (s, 1H), 10.44 (s, 1H), 8.14-8.08 (m, 4H), 8.04-8.00 (m, 2H), 7.88-7.77 (m, 4H), 7.68-7.62 (m, 1H), 7.58 (t, J = 7.7 Hz, 2H), 6.72 (s, 1H), 3.91 (s, 3H), 3.77 (s, 3H). | (151 MHz, DMSO) δ 166.18, 166.14, 165.11, 165.02, 148.41, 139.55, 139.49, 138.71, 138.61, 137.97, 133.76, 132.67, 132.48, 129.68, 129.63, 129.04, 128.88, 128.58, 128.39, 128.14, 125.56, 120.94, 120.84, 98.08, 52.93, 49.07, 36.32. |
| 47 | SW393124 | 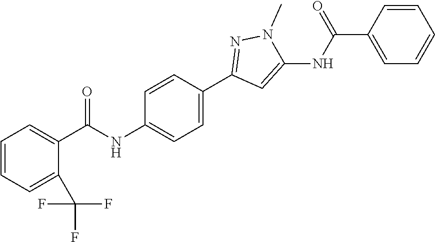 [SMILES: Cn1nc(-c2ccc(NC(=O)c3ccccc3C(F)(F)F)cc2)cc1NC(=O)c1ccccc1] | (600 MHz, DMSO) δ 10.64 (s, 1H), 10.44 (s, 1H), 8.02 (dt, J = 7.1, 1.3 Hz, 2H), 7.89-7.86 (m, 1H), 7.82 (t, J = 7.6 Hz, 1H), 7.80 (m, 2H), 7.76- 7.71 (m, 4H), 7.67-7.63 (m, 1H), 7.58 (dd, J = 8.3, 7.0 Hz, 2H), 6.71 (s, 1H), 3.77 (s, 3H). | (151 MHz, DMSO) δ 166.16, 165.97, 148.38, 138.71, 137.98, 136.68, 133.77, 133.13, 132.67, 130.54, 129.58, 129.04, 128.88, 128.39, 128.14, 126.84, 126.81, 126.40, 126.19, 125.67, 125.51, 125.18, 123.37, 120.84, 120.20, 98.06, 55.40, 36.31. |
| 48 | SW393125 | 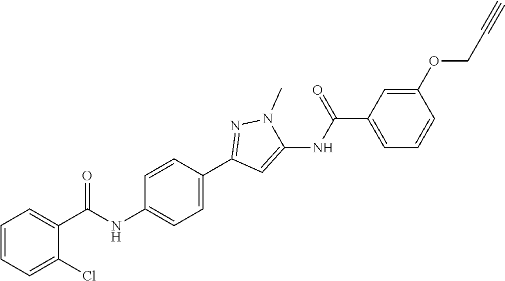 [SMILES: C#CCOc1cccc(C(=O)Nc2cc(-c3ccc(NC(=O)c4ccccc4Cl)cc3)nn2C)c1] | (600 MHz, Chloroform-d) δ 8.92 (s, 1H), 8.49 (s, 1H), 7.48 (d, J = 8.3 Hz, 2H), 7.44-7.31 (m, 5H), 7.27-7.17 (m, 3H), 7.13 (t, J = 7.9 Hz, 2H), 7.01 (dd, J = 8.3, 2.6 Hz, 1H), 6.24 (s, 1H), 4.55 (d, J = 2.4 Hz, 2H), 3.56 (s, 3H), 2.45 (t, J = 2.4 Hz, 1H); | (151 MHz, Chloroform-d) δ 166.4, 165.4, 157.8, 149.3, 136.8, 136.6, 135.3, 134.4, 131.6, 130.8, 130.3, 130.3, 129.8, 129.7, 127.2, 126.1, 120.8, 120.6, 119.4, 114.1, 98.3, 78.2, 76.2, 56.0, 35.9; |
| 49 | SW393126 | 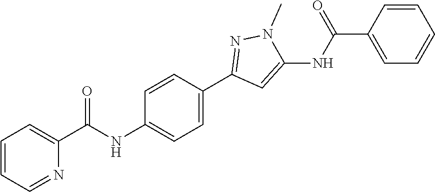 [SMILES: Cn1nc(-c2ccc(NC(=O)c3ccccn3)cc2)cc1NC(=O)c1ccccc1] | (400 MHz, MeOD) δ 9.09 (dd, J = 2.3, 0.9 Hz, 1H), 8.68 (dd, J = 4.9, 1.7 Hz, 1H), 8.35-8.28 (m, 1H), 8.02-7.94 (m, 2H), 7.64-7.55 (m, 1H), 7.55- 7.45 (m, 3H), 6.59 (s, 1H), 3.81 (s, 3H). | (151 MHz, MeOD) δ 149.37, 137.95, 127.80, 77.47, 77.26, 77.04. |
| 50 | SW393127 | 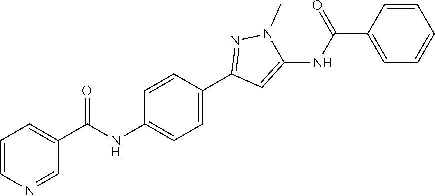 [SMILES: Cn1nc(-c2ccc(NC(=O)c3cccnc3)cc2)cc1NC(=O)c1ccccc1] | (600 MHz, MeOD) δ 9.09 (dd, J = 2.2, 0.8 Hz, 1H), 8.68 (dd, J = 4.9, 1.6 Hz, 1H), 8.32 (dt, J = 8.0, 1.9 Hz, 1H), 8.00-7.95 (m, 2H), 7.75 (s, 4H), 7.62-7.56 (m, 1H), 7.54-7.47 (m, 3H), 6.60 (s, 1H), 3.81 (s, 3H). | (151 MHz, MeOD) δ 167.78, 164.82, 151.38, 149.71, 148.17, 137.77, 137.57, 136.12, 132.99, 132.43, 131.26, 129.68, 128.57, 127.73, 125.81, 123.81, 121.03, 98.16, 49.23, 35.29.; |
| 51 | SW393128 | 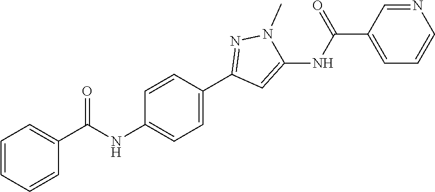 [SMILES: Cn1nc(-c2ccc(NC(=O)c3ccccc3)cc2)cc1NC(=O)c1cccnc1] | (400 MHz, MeOD) δ 9.06 (d, J = 2.2 Hz, 1H), 8.66 (dd, J = 4.9, 1.7 Hz, 1H), 8.29 (dt, J = 8.0, 2.0 Hz, 1H), 7.89-7.81 (m, 2H), 7.67 (d, J = 1.3 Hz, 4H), 7.50-7.44 (m, 2H), 7.41 (dd, J = 8.2, 6.5 Hz, 2H), 6.55 (s, 1H), 3.76 (s, 3H). | |
| 52 | SW393129 | 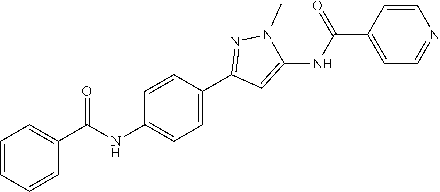 [SMILES: Cn1nc(-c2ccc(NC(=O)c3ccccc3)cc2)cc1NC(=O)c1ccncc1] | (400 MHz, MeOD) δ 8.68-8.62 (m, 2H), 7.86-7.78 (m, 4H), 7.65 (s, 4H), 7.49-7.43 (m, 1H), 7.43-7.36 (m, 2H), 6.53 (s, 1H), 3.74 (s, 3H). | (151 MHz, MeOD) δ 77.27, 77.24, 77.05, 77.03, 76.84, 76.82, 49.34. |
| 53 | SW393130 | 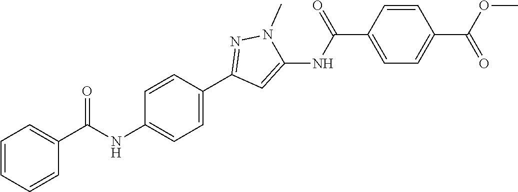 [SMILES: COC(=O)c1ccc(C(=O)Nc2cc(-c3ccc(NC(=O)c4ccccc4)cc3)nn2C)cc1] | (400 MHz, MeOD) δ 8.10-8.03 (m, 2H), 7.97 (d, J = 8.2 Hz, 2H), 7.87- 7.80 (m, 2H), 7.65 (s, 4H), 7.50-7.44 (m, 1H), 7.44-7.36 (m, 2H), 6.52 (s, 1H), 3.89 (s, 3H), 3.74 (s, 3H). | (151 MHz, MeOD) δ 129.78, 128.56, 127.95, 127.31, 126.05, 120.76, 98.01, 77.38, 77.17, 76.96, 35.56. |
| 54 | SW393132 | 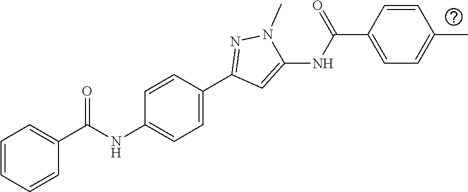 [SMILES: Cc1ccc(C(=O)Nc2cc(-c3ccc(NC(=O)c4ccccc4)cc3)nn2C)cc1] | (600 MHz, DMSO) δ 10.56 (s, 1H), 10.37 (s, 1H), 8.12-8.06 (m, 2H), 8.06-7.99 (m, 2H), 7.92- 7.87 (m, 2H), 7.86-7.81 (m, 2H), 7.73-7.68 (m, 2H), 7.67-7.63 (m, 1H), 7.62-7.57 (m, 2H), 6.76 (s, 1H), 3.81 (s, 3H). | (151 MHz, DMSO) δ 165.97, 165.13, 148.51, 139.02, 137.75, 137.52, 135.45, 132.52, 132.04, 130.36, 129.28, 129.14, 128.88, 128.14, 125.52, 120.85, 98.03, 36.34. |
| 55 | SW393133 | 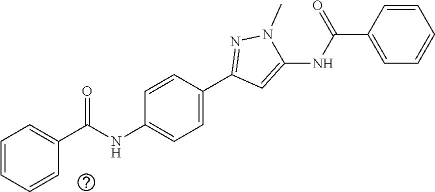 [SMILES: Cn1nc(-c2ccc(NC(=O)c3ccccc3)cc2)cc1NC(=O)c1ccccc1] | (600 MHz, DMSO) δ 10.56 (s, 1H), 10.44 (s, 1H), 8.04-8.00 (m, 2H), 7.81-7.75 (m, 4H), 7.74 (dd, J = 8.1, 1.1 Hz, 1H), 7.68-7.62 (m, 1H), 7.61- 7.54 (m, 3H), 7.52 (td, J = 7.5, 1.2 Hz, 1H), 7.44 (td, J = 7.7, 1.8 Hz, 1H), 6.71 (s, 1H), 3.77 (s, 3H). | (151 MHz, DMSO) δ 166.22, 166.15, 148.40, 139.62, 139.57, 138.75, 138.01, 133.80, 133.20, 132.66, 131.67, 129.50, 129.35, 129.04, 128.88, 128.39, 128.22, 125.65, 120.14, 120.05, 119.49, 98.04, 49.08, 36.30. |
| 56 | SW393134 | 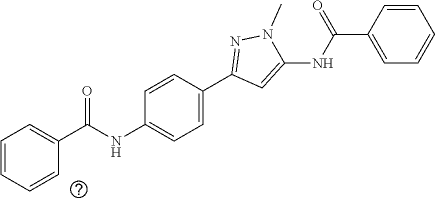 [SMILES: Cn1nc(-c2ccc(NC(=O)c3ccccc3)cc2)cc1NC(=O)c1ccccc1] | (600 MHz, DMSO) δ 10.44 (s, 1H), 10.38 (s, 1H), 8.04-8.00 (m, 2H), 7.79 (q, J = 8.8 Hz, 4H), 7.68-7.62 (m, 1H), 7.58 (dd, J = 8.3, 7.0 Hz, 2H), 7.48 (dd, J = 7.4, 1.5 Hz, 1H), 7.40 (td, J = 7.5, 1.4 Hz, 1H), 7.32 (ddd, J = 9.0, 7.5, 1.8 Hz, 2H), 6.70 (s, 1H), 3.76 (s, 3H), 2.41 (s, 3H). | (151 MHz, DMSO) δ 168.26, 166.16, 148.47, 139.11, 137.95, 137.73, 135.69, 133.78, 132.67, 131.00, 130.10, 129.21, 129.05, 128.88, 128.39, 128.14, 127.70, 126.13, 125.57, 120.84, 120.15, 120.06, 98.03, 36.30, 19.80., |
| 57 | SW393141 | 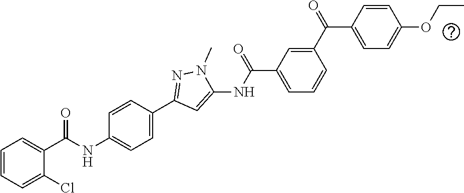 [SMILES: CCOc1ccc(C(=O)c2cccc(C(=O)Nc3cc(-c4ccc(NC(=O)c5ccccc5Cl)cc4)nn3C)c2)cc1] | (600 MHz, DMSO-d₆) δ 10.62 (s, 1H), 10.58 (s, 1H), 8.32 (d, J = 1.9 Hz, 1H), 8.28 (dt, J = 7.9, 1.4 Hz, 1H), 7.95 (dt, J = 7.6, 1.5 Hz, 1H), 7.85-7.80 (m, 2H), 7.80-7.73 (m, 5H), 7.59 (ddd, J = 15.9, 7.7, 1.5 Hz, 2H), 7.52 (td, J = 7.7, 1.8 Hz, 1H), 7.47 (td, J = 7.4, 1.3 Hz, 1H), 7.21-7.15 (m, 2H), 6.71 (s, 1H), 4.95 (d, J = 2.4 Hz, 2H), 3.76 (s, 3H), 3.67 (t, J = 2.4 Hz, 1H); | (151 MHz, DMSO-d₆) δ 193.9, 165.0, 164-9, 161.1, 148.0, 138.3, 138.0, 137.4, 137.0, 132.8, 132.3, 131.5, 131.2, 130.0, 129.7, 129.0, 129.0, 128.5, 127.3, 125.2, 119.69, 114.9, 97.7, 78.9, 78.7, 55.8, 35.9; |
| 58 | SW393142 | 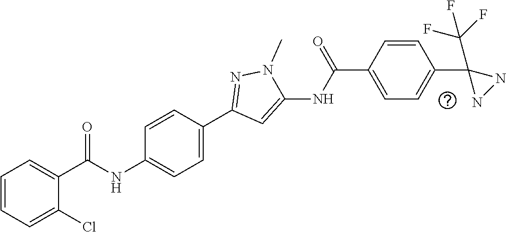 [SMILES: Cn1nc(-c2ccc(NC(=O)c3ccccc3Cl)cc2)cc1NC(=O)c1ccc(C2(C(F)(F)F)NN2)cc1] | (600 MHz, Methanol-d₄) δ 8.02 (d, J = 8.2 Hz, 22H), 7.76-7.69 (m, 4H), 7.58- 7.51 (m, 1H), 7.45 (d, J = 8.0 Hz, 1H), 7.40 (td, J = 7.7, 1.8 Hz, 1H), 7.36 (td, J = 7.4, 1.3 Hz, 1H), 7.32 (d, J = 8.2 Hz, 2H), 6.58 (s, 1H), 3.80 (s, 3H); | (151 MHz, MeOD) δ 167.2, 166.9, 150.4, 138.5, 137.9, 136.9, 134.8, 133.6, 131.7, 131.5, 130.5, 130.0, 129.3, 128.9, 127.4, 127.1, 126.5, 121.0, 98.7, 35.9; |
| 59 | SW393143 | 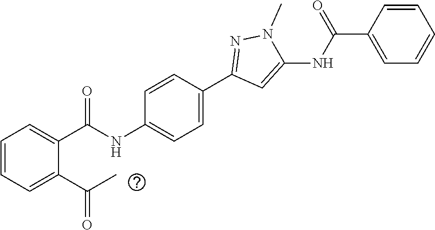 [SMILES: CC(=O)c1ccccc1C(=O)Nc1ccc(-c2cc(NC(=O)c3ccccc3)n(C)n2)cc1] | (600 MHz, MeOD) δ 8.09 (dd, J = 8.6, 1.3 Hz, 1H), 7.92-7.88 (m, 2H), 7.72 (td, J = 7.5, 1.2 Hz, 1H), 7.70-7.66 (m, 2H), 7.64-7.58 (m, 4H), 7.55- 7.51 (m, 1H), 7.50 (s, 1H), 7.45 (t, J = 7.8 Hz, 2H), 6.54 (s, 1H), 3.76 (s, 3H). | (151 MHz, MeOD) δ 133.87, 128.81, 128.49, 127.66, 125.74, 124.28, 120.40, 98.07, 77.95, 77.77, 77.74, 77.52, 48.30, 48.13, 48.04, 47.90, 47.76, 47.70, 47.62, 47.56, 47.41. |
| 60 | SW393144 | 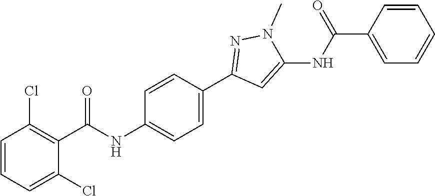 [SMILES: Cn1nc(-c2ccc(NC(=O)c3c(Cl)cccc3Cl)cc2)cc1NC(=O)c1ccccc1] | (600 MHz, DMSO) δ 10.83 (s, 1H), 10.49 (s, 1H), 8.03 (dt, J = 7.1, 1.4 Hz, 2H), 7.84-7.77 (m, 2H), 7.77-7.71 (m, 2H), 7.67-7.63 (m, 1H), 7.62- 7.44 (m, 8H), 6.71 (d, J = 2.3 Hz, 1H), 3.77 (s, 3H). | (151 MHz, DMSO) δ 166.16, 165.84, 162.43, 148.30, 138.33, 138.05, 136.82, 135.09, 133.76, 132.67, 131.91, 131.89, 131.70, 130.41, 129.77, 129.34, 129.03, 128.94, 128.84, 128.74, 128.69, 128.42, 125.78, 120.12, 120.04, 98.09, 36.33. |
| 61 | SW393168 | 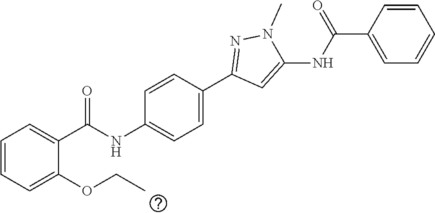 [SMILES: CCOc1ccccc1C(=O)Nc1ccc(-c2cc(NC(=O)c3ccccc3)n(C)n2)cc1] | (600 MHz, DMSO) δ 10.43 (s, 1H), 10.22 (s, 1H), 8.04-8.00 (m, 2H), 7.78 (d, J = 1.0 Hz, 4H), 7.68-7.62 (m, 2H), 7.58 (dd, J = 8.4, 7.0 Hz, 2H), 7.53 (ddt, J = 8.6, 7.2, 1.5 Hz, 1H), 7.26 (d, J = 8.4 Hz, 1H), 7.13 (t, J = 7.4 Hz, 1H), 6.70 (s, 1H), 4.97 (dd, J = 2.4, 1.3 Hz, 2H), 3.76 (s, 3H), 3.68 (dt, J = 3.4, 1.6 Hz, 1H). | (151 MHz, DMSO) δ 166.15, 154.93, 148.44, 138.86, 137.95, 133.77, 130.16, 129.24, 129.05, 128.39, 125.62, 121.76, 120.14, 120.05, 113.94, 98.03, 79.44, 79.33, 56.86, 36.30. |
| 62 | SW393169 | 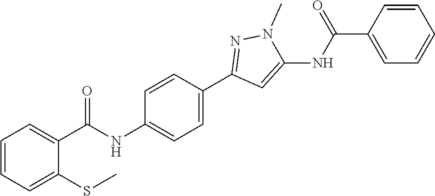 [SMILES: CSc1ccccc1C(=O)Nc1ccc(-c2cc(NC(=O)c3ccccc3)n(C)n2)cc1] | (600 MHz, MeOD) δ 7.93-7.88 (m, 2H), 7.70-7.63 (m, 4H), 7.56-7.50 (m, 1H), 7.49-7.41 (m, 3H), 7.37 (td, J = 7.7, 1.5 Hz, 1H), 7.32 (dd, J = 8.1, 1.2 Hz, 1H), 7.17 (td, J = 7.5, 1.2 Hz, 1H), 6.53 (s, 1H), 3.75 (s, 3H), 2.41 (s, 3H). | (151 MHz, MeOD) δ 137.60, 135.75, 132.38, 130.58, 129.33, 128.52, 127.70, 127.64, 126.63, 125.74, 124.71, 120.48, 98.07, 77.81, 77.59, 77.38, 35.15, 15.67. |
| 63 | SW393170 | 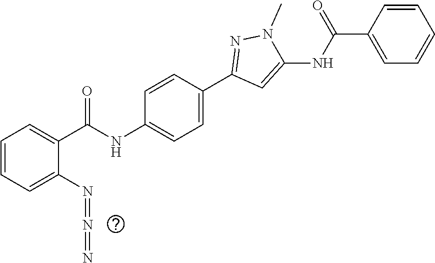 | (600 MHz, DMSO) δ 10.44 (s, 1H), 10.40 (s, 1H), 8.04-8.00 (m, 2H), 7.78 (d, J = 1.3 Hz, 4H), 7.68-7.62 (m, 1H), 7.62-7.55 (m, 4H), 7.45 (dd, J = 8.5, 1.0 Hz, 1H), 7.32 (td, J = 7.5, 1.1 Hz, 1H), 6.71 (s, 1H), 3.76 (s, 3H). | (151 MHz, DMSO) δ 166.16, 164.96, 164.86, 148.42, 138.80, 138.69, 137.97, 137.16, 133.77, 132.67, 131.99, 129.86, 129.68, 129.62, 129.37, 129.05, 128.39, 125.63, 125.49, 120.09, 120.04, 120.00, 98.05, 36.30. |
| 64 | SW393171 | 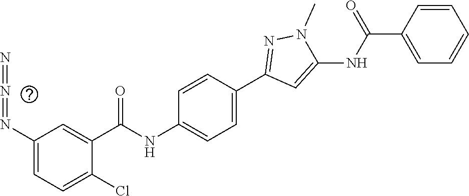 | (600 MHz, MeOD) δ 7.89 (d, J = 7.3 Hz, 2H), 7.69-7.60 (m, 4H), 7.54- 7.48 (m, 1H), 7.43 (t, J = 7.8 Hz, 2H), 7.37-7.32 (m, 1H), 7.17 (d, J = 2.7 Hz, 1H), 6.98 (dd, J = 8.6, 2.8 Hz, 1H), 6.51 (s, 1H), 3.74 (s, 3H). | (151 MHz, MeOD) δ 167.47, 165.01, 149.72, 139.26, 137.52, 137.48, 137.36, 133.00, 132.44, 131.35, 129.66, 128.62, 127.74, 126.78, 125.95, 121.50, 120.41, 119.52, 98.09, 35.46, 29.61.; |
| 65 | SW393172 | 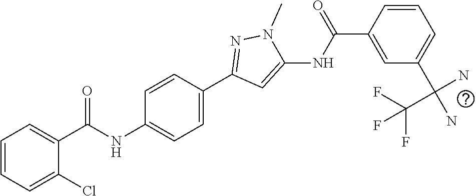 [SMILES: Cn1nc(-c2ccc(NC(=O)c3ccccc3Cl)cc2)cc1NC(=O)c1cccc(C(N)(N)C(F)(F)F)c1] | (600 MHz, Methanol-d₄) δ 8.02 (d, J = 8.2 Hz, 2H), 7.76-7.68 (m, 4H), 7.55 (dd, J = 7.5, 1.8 Hz, 1H), 7.44 (dd, J = 8.0, 1.3 Hz, 1H), 7.39 (td, J = 7.7, 1.8 Hz, 1H), 7.35 (td, J = 7.4, 1.3 Hz, 1H), 7.31 (d, J = 8.2 Hz, 2H), 6.58 (s, 1H), 3.79 (s, 3H). | (151 MHz, Methanol-d₄) δ 167.0, 166.7, 150.3, 138.3, 137.7, 136.8, 134.7, 133.6, 131.6, 131.4, 130.4, 129.9, 129.3, 128.8, 127.3, 127.0, 126.4, 120.9, 98.6, 35.9. |
| 66 | SW393173 | 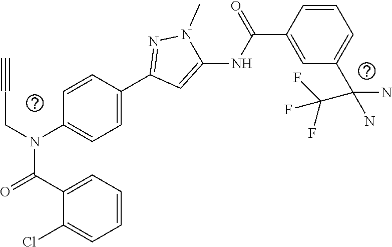 [SMILES: C#CCN(C(=O)c1ccccc1Cl)c1ccc(-c2cc(NC(=O)c3cccc(C(N)(N)C(F)(F)F)c3)n(C)n2)cc1] | (600 MHz, Chloroform-d) δ 8.95 (s, 1H), 8.02 (d, J = 6.7 Hz, 1H), 7.72 (s, 1H), 7.55-7.47 (m, 4H), 7.19 (d, J = 8.6 Hz, 2H), 7.00 (dd, J = 7.7, 1.7 Hz, 1H), 6.93 (dd, J = 8.1, 1.1 Hz, 1H), 6.81 (td, J = 7.5, 1.1 Hz, 1H), 6.70 (td, J = 7.7, 1.7 Hz, 1H), 6.17 (s, 1H), 4.69 (d, J = 2.4 Hz, 2H), 3.72 (s, 3H), 2.27 (t, J = 2.5 Hz, 1H). | (151 MHz, Chloroform-d) δ 168.3, 165.1, 148.5, 140.0, 136.4, 135.4, 134.0, 133.3, 130.6, 130.5, 130.1, 130.1, 129.5, 129.5, 128.9, 128.4, 127.8, 126.2, 126.0, 125.9, 123.0, 121.2, 98.4, 78.4, 72.9, 38.9, 36.1. |
| 67 | SW393174 | 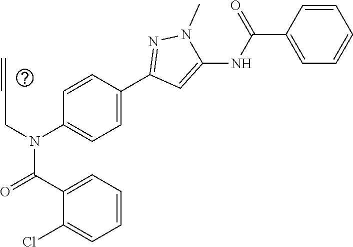 [SMILES: C=CCN(C(=O)c1ccccc1Cl)c1ccc(-c2cc(NC(=O)c3ccccc3)n(C)n2)cc1] | (600 MHz, Chloroform-d) δ 8.84 (s, 1H), 7.92 (d, J = 7.4 Hz, 2H), 7.57- 7.48 (m, 3H), 7.43 (t, J = 7.8 Hz, 2H), 7.21-7.16 (m, 2H), 7.01 (ddd, J = 13.9, 7.8, 1.5 Hz, 2H), 6.88 (td, J = 7.5, 1.2 Hz, 1H), 6.83 (td, J = 7.7, 1.7 Hz, 1H), 6.27 (s, 1H), 4.65 (d, J = 2.5 Hz, 2H), 3.69 (s, 3H), 2.28 (t, J = 2.5 Hz, 1H). | (151 MHz, Chloroform-d) δ 168.1, 166.3, 148.5, 140.0, 136.9, 135.5, 130.5, 130.2, 129.5, 128.8, 128.5, 127.8, 127.7, 126.3, 125.9, 98.2, 78.5, 72.8, 38.8, 36.0. |
| 68 | SW393185 | 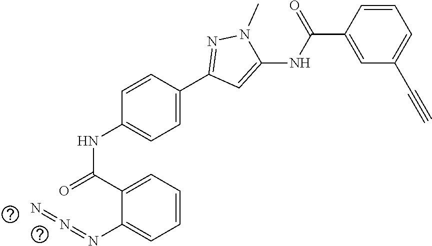 | (400 MHz, Chloroform-d) δ 9.40 (s, 1H), 8.52 (s, 1H), 8.18 (dd, J = 7.9, 1.6 Hz, 1H), 8.05 (d, J = 1.8 Hz, 1H), 7.93-7.87 (m, 1H), 7.71-7.66 (m, 2H), 7.62 (dt, J = 7.8, 1.4 Hz, 1H), 7.60 7.51 (m, 3H), 7.38 (t, J = 7.8 Hz, 1H), 7.30-7.21 (m, 3H), 6.48 (s, 1H), 3.77 (s, 3H), 3.13 (s, 1H). | (101 MHz, Chloroform-d) δ 165.4, 163.0, 149.5, 137.1, 136.9, 136.4, 135.8, 133.7, 133.0, 132.7, 131.3, 130.3, 129.0, 128.1, 126.1, 125.6, 124.9, 123.0, 121.1, 118.6, 98.1, 82.6, 78.9, 36.0. |
| 69 | SW393186 | 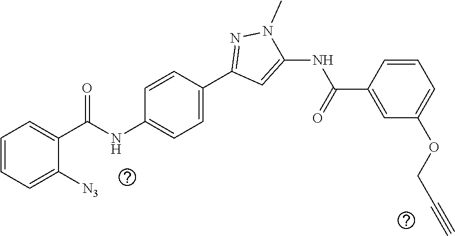 [SMILES: C#CCOc1cccc(C(=O)Nc2cc(-c3ccc(NC(=O)c4ccccc4N=[N+]=[N-])cc3)nn2C)c1] | (400 MHz, Chloroform-d) δ 9.38 (s, 1H), 8.69 (s, 1H), 8.11 (dd, J = 7.9, 1.6 Hz, 1H), 7.68-7.61 (m, 2H), 7.58- 7.47 (m, 5H), 7.33-7.17 (m, 3H), 7.11 (ddd, J = 8.3, 2.6, 0.9 Hz, 1H), 6.43 (s, 1H), 4.68 (d, J = 2.4 Hz, 2H), 3.71 (s, 3H), 2.54 (t, J = 2.4 Hz, 1H). | (101 MHz, Chloroform-d) δ 166.0, 163.1, 157.9, 149.4, 137.1, 136.9, 136.6, 134.7, 133.0, 132.5, 130.3, 129.9, 126.0, 125.5, 125.0, 121.0, 120.5, 119.3, 118.6, 114.1, 98.0, 78.2, 76.2, 56.1, 35.9. |
| 70 | SW393187 | 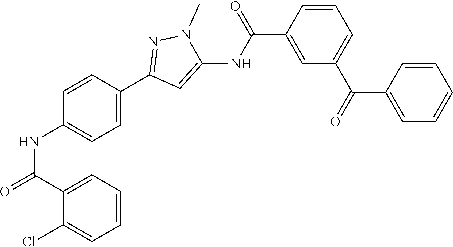 [SMILES: Cn1nc(-c2ccc(NC(=O)c3ccccc3Cl)cc2)cc1NC(=O)c1cccc(C(=O)c2ccccc2)c1] | (600 MHz, DMSO) δ 10.64 (s, 1H), 10.59 (s, 1H), 8.37 (d, J = 1.9 Hz, 1H), 8.31 (d, J = 7.7 Hz, 1H), 7.99 (dt, J = 7.8, 1.4 Hz, 1H), 7.81 (dd, J = 7.9, 1.4 Hz, 2H), 7.80-7.76 (m, 5H), 7.76- 7.72 (m, 1H), 7.62 (td, J = 7.5, 1.6 Hz, 3H), 7.59 (dd, J = 8.1, 1.2 Hz, 1H), 7.53 (td, J = 7.7, 1.8 Hz, 1H), 7.48 (td, J = 7.5, 1.3 Hz, 1H), 6.71 (s, 1H), 3.77 (s, 3H). | (151 MHz, DMSO) δ 195.74, 165.38, 165.32, 148.44, 138.75, 137.84, 137.74, 137.46, 137.07, 134.04, 133.57, 132.33, 131.58, 130.40, 130.26, 130.14, 129.50, 129.45, 129.42, 129.29, 129.24, 127.75, 125.67, 120.12, 98.12, 49.07, 36.37. |
| 71 | SW393188 | 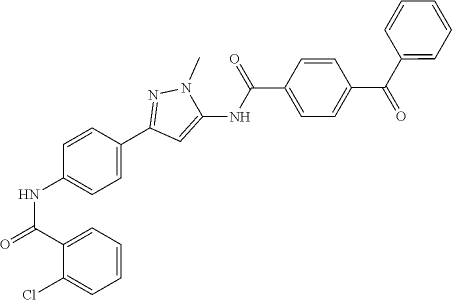 [SMILES: Cn1nc(-c2ccc(NC(=O)c3ccccc3Cl)cc2)cc1NC(=O)c1ccc(C(=O)c2ccccc2)cc1] | (600 MHz, MeOD) δ 8.13 (d, J = 8.1 Hz, 2H), 7.94-7.89 (m, 2H), 7.84- 7.80 (m, 2H), 7.78-7.72 (m, 3H), 7.69- 7.64 (m, 1H), 7.59-7.52 (m, 5H), 7.48 (dd, J = 7.9, 1.3 Hz, 1H), 7.43 (td, J = 7.7, 1.8 Hz, 1H), 7.39 (td, J = 7.4, 1.3 Hz, 1H), 6.65 (s, 1H), 3.86 (s, 3H). | (151 MHz, MeOD) δ 130.02, 129.98, 128.49, 127.89, 120.40, 77.67, 77.45, 77.24, 35.36. |
| 72 | SW393189 | 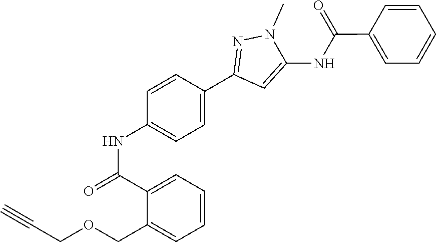 [SMILES: C#CCOCc1ccccc1C(=O)Nc1ccc(-c2cc(NC(=O)c3ccccc3)n(C)n2)cc1] | (600 MHz, CDCl₃) δ 9.06 (s, 1H), 8.47 (s, 1H), 7.84 (d, J = 7.4 Hz, 3H), 7.71 (dd, J = 7.5, 1.5 Hz, 1H), 7.67-7.61 (m, 2H), 7.59-7.54 (m, 2H), 7.46 (ddt, J = 8.7, 7.2, 1.6 Hz, 1H), 7.41 (td, J = 7.4, 1.5 Hz, 1H), 7.39-7.34 (m, 2H), 7.34-7.30 (m, 2H), 6.41 (s, 1H), 4.64 (s, 2H), 4.20 (d, J = 2.5 Hz, 2H), 3.70 (s, 3H), 2.38 (t, J = 2.4 Hz, 1H). | (151 MHz, CDCl₃) δ 166.76, 166.24, 149.43, 137.45, 137.18, 136.55, 133.05, 132.99, 132.46, 131.46, 130.90, 129.93, 129.85, 129.25, 128.77, 127.62, 125.99, 120.31, 98.06, 78.48, 75.96, 70.51, 57.73, 35.94, 29.73. |
| 73 | SW393212 | 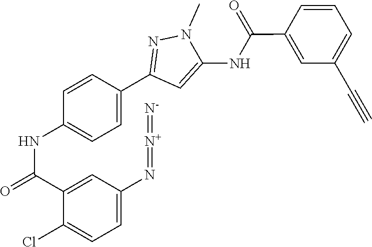 [SMILES: C#Cc1cccc(C(=O)Nc2cc(-c3ccc(NC(=O)c4cc(N=[N+]=[N-])ccc4Cl)cc3)nn2C)c1] | (600 MHz, Methanol-d₄) δ 9.91 (s, 1H), 8.06 (d, J = 1.8 Hz, 1H), 7.93 (d, J = 7.8 Hz, 1H), 7.74-7.64 (m, 5H), 7.46 (t, J = 7.8 Hz, 1H), 7.41 (d, J = 8.8 Hz, 2H), 7.21 (d, J = 2.7 Hz, 1H), 7.04 (dd, J = 8.7, 2.8 Hz, 1H), 6.57 (s, 1H), 3.79 (s, 3H), 3.26 (s, 1H). | (151 MHz, Methanol-d₄) δ 166.9, 165.6, 165.5, 150.1, 139.6, 138.0, 138.0, 137.8, 137.7, 136.0, 133.8, 131.7, 130.0, 129.1, 128.4, 127.1, 126.3, 123.2, 121.8, 120.9, 120.8, 119.9, 98.5, 82.7, 78.9, 35.8. |
| 74 | SW393213 | 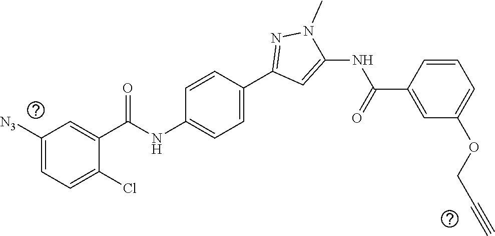 [SMILES: C#CCOc1cccc(C(=O)Nc2cc(-c3ccc(NC(=O)c4cc(N=[N+]=[N-])ccc4Cl)cc3)nn2C)c1] | (600 MHz, Methanol-d₄) δ 7.75-7.67 (m, 4H), 7.58 (d, J = 8.2 Hz, 2H), 7.42 (dt, J = 7.8, 3.6 Hz, 2H), 7.23-7.18 (m, 2H), 7.07 (dd, J = 8.6, 2.7 Hz, 1H), 6.57 (s, 1H), 4.77 (d, J = 2.4 Hz, 2H), 3.80 (s, 3H), 2.77 (t, J = 2.4 Hz, 1H). | (151 MHz, Methanol-d₄) δ 168.0, 166.1, 158.5, 150.4, 140.0, 138.4, 138.2, 138.2, 135.1, 131.9, 130.4, 130.3, 127.5, 126.5, 122.1, 121.3, 121.1, 119.9, 119.8, 114.9, 98.8, 78.6, 76.7, 56.5, 36.0; |
| 75 | SW393214 | 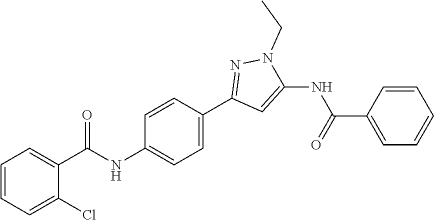 [SMILES: CCn1nc(-c2ccc(NC(=O)c3ccccc3Cl)cc2)cc1NC(=O)c1ccccc1] | (600 MHz, Methanol-d₄) δ 7.97-7.91 (m, 2H), 7.77-7.68 (m, 4H), 7.59- 7.56 (m, 1H), 7.54 (dd, J = 7.5, 1.8 Hz, 1H), 7.51-7.47 (m, 2H), 7.44 (dd, J = 7.9, 1.3 Hz, 1H), 7.39 (td, J = 7.7, 1.8 Hz, 1H), 7.35 (td, J = 7.5, 1.4 Hz, 1H), 6.56 (s, 1H), 4.12 (q, J = 7.3 Hz, 2H), 1.45 (t, J = 7.2 Hz, 3H). | (151 MHz, Methanol-d₄) δ 168.5, 167.0, 150.5, 138.3, 137.0, 136.8, 133.6, 132.9, 131.6, 131.4, 130.4, 130.2, 129.3, 129.1, 128.2, 127.4, 126.4, 120.9, 99.2, 44.0, 15.3. |
| 76 | SW393215 | 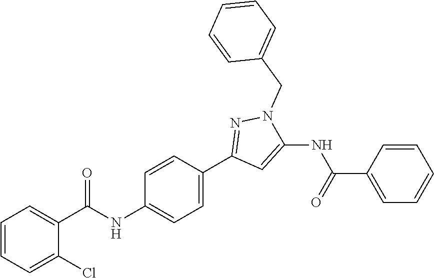 [SMILES: O=C(Nc1cc(-c2ccc(NC(=O)c3ccccc3Cl)cc2)nn1Cc1ccccc1)c1ccccc1] | (600 MHz, DMSO-d₆) δ 10.59 (s, 1H), 10.48 (s, 1H), 7.95-7.87 (m, 2H), 7.83-7.73 (m, 4H), 7.65-7.59 (m, 2H), 7.58 (dd, J = 8.1, 1.2 Hz, 1H), 7.56-7.50 (m, 3H), 7.46 (td, J = 7.4, 1.2 Hz, 1H), 7.32 (t, J = 7.5 Hz, 2H), 7.26 (t, J = 7.4 Hz, 1H), 7.23-7.16 (m, 2H), 6.79 (s, 1H), 5.38 (s, 2H). | (151 MHz, DMSO-d₆) δ 165.8, 164.9, 138.4, 137.6, 137.3, 137.0, 133.4, 132.2, 131.1, 130.0, 129.7, 129.0, 128.9, 128.6, 128.5, 127.9, 127.4, 127.3, 125.4, 119.7, 98.2, 51.8. |
| 77 | SW393216 | 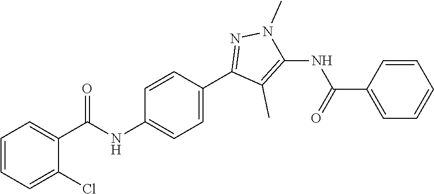 [SMILES: Cc1c(-c2ccc(NC(=O)c3ccccc3Cl)cc2)nn(C)c1NC(=O)c1ccccc1] | (600 MHz, Methanol-d₄) δ 8.00 (dd, J = 7.8, 1.5 Hz, 2H), 7.75 (d, J = 8.6 Hz, 2H), 7.65-7.57 (m, 3H), 7.54 (dd, J = 7.5, 1.8 Hz, 1H), 7.50 (t, J = 7.7 Hz, 2H), 7.45 (dd, J = 8.0, 1.3 Hz, 1H), 7.41 (td, J = 7.7, 1.8 Hz, 1H), 7.36 (td, J = 7.4, 1.3 Hz, 1H), 3.75 (s, 3H), 2.10 (s, 3H). | (151 MHz, Methanol-d₄) δ 168.9, 167.4, 149.0, 138.3, 137.1, 135.5, 133.5, 133.1, 131.7, 131.6, 130.8, 129.4, 129.3, 128.5, 128.4, 127.5, 120.9, 109.9, 35.7, 9.3. |
| 78 | SW393217 | 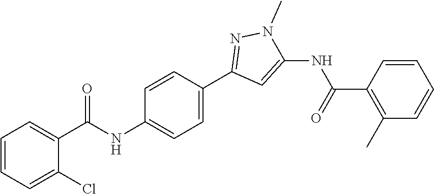 [SMILES: Cc1ccccc1C(=O)Nc1cc(-c2ccc(NC(=O)c3ccccc3Cl)cc2)nn1C] | (600 MHz, MeOD) δ 7.67 (d, J = 8.3 Hz, 2H), 7.62 (d, J = 8.5 Hz, 2H), 7.52 (dd, J = 7.5, 1.8 Hz, 1H), 7.46 (d, J = 7.7 Hz, 1H), 7.37 (dd, J = 7.9, 1.3 Hz, 1H), 7.34-7.25 (m, 3H), 7.22-7.17 (m, 2H), 6.60 (s, 1H), 3.74 (s, 3H), 2.44 (s, 3H). | (151 MHz, MeOD) δ 169.17, 165.78, 149.77, 137.54, 137.26, 136.60, 135.89, 134.84, 131.28, 131.14, 130.86, 130.64, 130.09, 129.57, 129.28, 127.16, 126.98, 126.01, 125.73, 120.32, 97.29, 35.51, 19.75. |
| 79 | SW393218 | 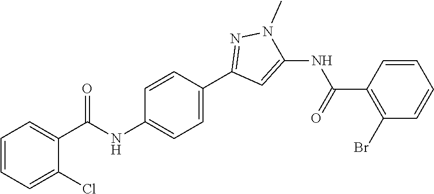 [SMILES: Cn1nc(-c2ccc(NC(=O)c3ccccc3Cl)cc2)cc1NC(=O)c1ccccc1Br] | (600 MHz, CDCl₃) δ 8.07 (s, 1H), 7.98 (s, 1H), 7.73-7.65 (m, 2H), 7.61 (td, J = 7.9, 1.7 Hz, 2H), 7.58-7.53 (m, 3H), 7.39-7.30 (m, 3H), 7.27 (tdd, J = 7.8, 5.9, 1.6 Hz, 2H), 6.58 (s, 1H), 3.81 (s, 3H). | (151 MHz, CDCl₃) δ 165.50, 164.50, 149.53, 136.90, 136.24, 135.76, 134.99, 133.65, 132.21, 131.75, 130.64, 130.41, 130.32, 130.17, 127.88, 127.30, 126.11, 120.40, 119.25, 97.49, 53.47, 36.19, 29.73. |
| 80 | SW393241 | 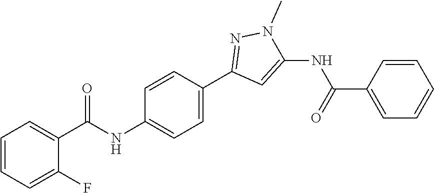 [SMILES: Cn1nc(-c2ccc(NC(=O)c3ccccc3F)cc2)cc1NC(=O)c1ccccc1] | (600 MHz, DMSO-d₆) δ 10.51 (s, 1H), 10.45 (s, 1H), 8.05-8.01 (m, 2H), 7.79 (d, J = 1.2 Hz, 4H), 7.70 (td, J = 7.4, 1.8 Hz, 1H), 7.67-7.63 (m, 1H), 7.59 (dq, J = 8.7, 7.3 Hz, 3H), 7.40- 7.32 (m, 2H), 6.71 (s, 1H), 3.77 (s, 3H). | (151 MHz, DMSO-d₆) δ 165.7, 162.7, 159.7, 158.1, 148.0, 138.3, 137.6, 133.3, 132.5 (d, J = 8.2 Hz), 132.2, 130.0 (d, J = 3.1 Hz), 129.1, 128.6, 128.0, 125.2, 125.2, 125.1, 124.6 (d, J = 3.3 Hz), 119.8, 116.3, 116.1, 97.6, 35.9. |
| 81 | SW393242 | 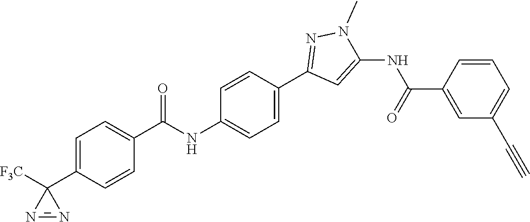 [SMILES: C#Cc1cccc(C(=O)Nc2cc(-c3ccc(NC(=O)c4ccc(C5(C(F)(F)F)N=N5)cc4)cc3)nn2C)c1] | (600 MHz, Methanol-d₄) δ 8.08 (d, J = 1.8 Hz, 1H), 7.99 (d, J = 8.5 Hz, 2H), 7.95 (dt, J = 7.8, 1.5 Hz, 1H), 7.73 (d, J = 1.2 Hz, 4H), 7.68 (dt, J = 7.7, 1.4 Hz, 1H), 7.49 (t, J = 7.8 Hz, 1H), 7.31 (d, J = 8.2 Hz, 2H), 6.60 (s, 1H), 3.81 (s, 3H), 3.39 (s, 1H). | (151 MHz, MeOD) δ 167.5, 166.8, 150.5, 138.7, 138.1, 136.9, 136.3, 134.1, 132.9, 132.0, 130.2, 129.4, 128.7, 128.7, 127.1, 126.5, 121.7, 98.8, 82.9, 79.4, 36.0; |
| 82 | SW393243 | 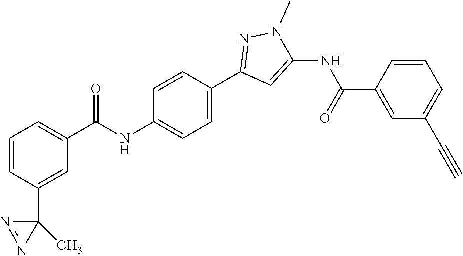 [SMILES: C#Cc1cccc(C(=O)Nc2cc(-c3ccc(NC(=O)c4cccc(C5(C)N=N5)c4)cc3)nn2C)c1] | (600 MHz, Methanol-d₄) δ 8.08 (t, J = 1.8 Hz, 1H), 7.99 (dt, J = 7.8, 1.3 Hz, 1H), 7.97-7.92 (m, 1H), 7.76-7.70 (m, 5H), 7.68 (dt, J = 7.8, 1.4 Hz, 1H), 7.55 (t, J = 7.8 Hz, 1H), 7.50-7.46 (m, 2H), 6.59 (s, 1H), 3.81 (s, 3H), 3.36 (s, 1H). | (151 MHz, Methanol-d₄) δ 167.4, 166.7, 150.5, 138.6, 138.0, 136.5, 136.2, 134.1, 132.0, 130.2, 130.2, 130.1, 129.8, 129.4, 129.3, 128.7, 126.4, 123.6, 121.7, 98.8, 82.9, 79.9, 36.0. |
| 83 | SW393244 | 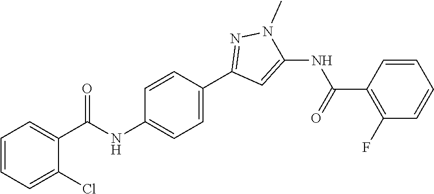 [SMILES: Cn1nc(-c2ccc(NC(=O)c3ccccc3Cl)cc2)cc1NC(=O)c1ccccc1F] | (600 MHz, CDCl₃) (600 MHz, CDCl₃) δ 8.36 (d, J = 16.5 Hz, 1H), 8.12 (td, J = 7.9, 1.9 Hz, 1H), 7.94 (s, 1H), 7.73 (d, J = 8.5 Hz, 2H), 7.69 (dd, J = 7.6, 1.9 Hz, 1H), 7.61 (d, J = 8.5 Hz, 2H), 7.52 (tdd, J = 7.5, 5.2, 1.8 Hz, 1H), 7.38 (dd, J = 7.9, 1.4 Hz, 1H), 7.34 (td, J = 7.6, 1.8 Hz, 1H), 7.32-7.26 (m, 2H), 7.16 (dd, J = 12.4, 8.3 Hz, 1H), 6.68 (s, 1H), 3.79 (s, 3H). | (151 MHz, CDCl₃) δ 164.39, 161.57, 161.01, 160.99, 159.94, 149.62, 137.10, 136.17, 135.20, 134.69, 134.63, 132.59, 131.73, 130.64, 130.41, 130.05, 127.33, 126.15, 125.43, 125.41, 120.18, 119.69, 119.62, 116.38, 116.21, 97.02, 35.70, 29.73, 22.72. |
| 84 | SW393270 | 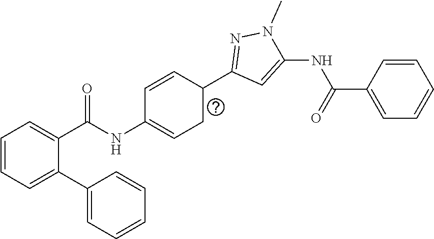 [SMILES: Cn1nc(C2C=CC(NC(=O)c3ccccc3-c3ccccc3)=CC2)cc1NC(=O)c1ccccc1] | (600 MHz, DMSO-d₆) δ 10.44 (s, 1H), 10.33 (s, 1H), 8.02 (d, J = 7.7 Hz, 2H), 7.71 (d, J = 8.2 Hz, 2H), 7.64 (d, J = 7.4 Hz, 1H), 7.63-7.55 (m, 6H), 7.49 (dd, J = 16.7, 8.0 Hz, 4H), 7.40 (t, J = 7.6 Hz, 2H), 7.32 (t, J = 7.4 Hz, 1H), 6.68 (s, 1H), 3.76 (s, 3H). | (151 MHz, DMSO-d₆) δ 167.85, 165.76, 148.05, 140.10, 139.31, 138.49, 137.53, 137.13, 133.35, 132.26, 130.07, 129.87, 128.80, 128.37, 127.96, 127.90, 127.36, 127.30, 125.10, 119.73, 97.60, 35.87. |
| 85 | SW393279 | 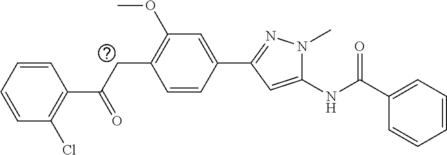 [SMILES: COc1cc(-c2cc(NC(=O)c3ccccc3)n(C)n2)ccc1CC(=O)c1ccccc1Cl] | ¹H NMR (400 MHz, cdcl₃) δ 8.57 (s, 1H), 8.44-8.37 (m, 1H), 7.95-7.89 (m, 2H), 7.73-7.67 (m, 1H), 7.52 (tt, J = 7.6, 1.7 Hz, 1H), 7.48-7.32 (m, 6H), 7.32-7.27 (m, 1H), 6.58-6.45 (m, 1H), 3.92 (d, J = 1.6 Hz, 3H), 3.88-3.72 (m, 3H). | |
| 86 | SW393280 | 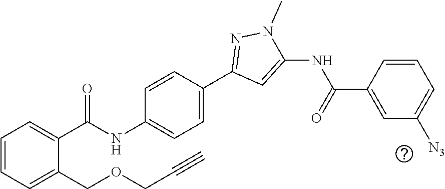 [SMILES: C#CCOCc1ccccc1C(=O)Nc1ccc(-c2cc(NC(=O)c3cccc(N=[N+]=[N-])c3)n(C)n2)cc1] | (600 MHz, CDCl₃) δ 9.08 (s, 1H), 8.65 (s, 1H), 7.70 (dd, J = 7.5, 1.5 Hz, 1H), 7.65-7.61 (m, 2H), 7.59 (d, J = 7.7 Hz, 1H), 7.56-7.51 (m, 3H), 7.42 (td, J = 7.5, 1.5 Hz, 1H), 7.40-7.34 (m, 2H), 7.27 (t, J = 7.9 Hz, 1H), 7.09 (dd, J = 7.9, 2.3 Hz, 1H), 6.40 (s, 1H), 4.64 (s, 2H), 4.21 (d, J = 2.4 Hz, 2H), 3.70 (s, 3H), 2.10 (s, 1H). | (151 MHz, CDCl₃) δ 166.82, 165.38, 149.39, 141.00, 137.34, 137.10, 136.30, 134.86, 132.99, 131.53, 130.95, 130.11, 129.94, 129.89, 129.27, 126.06, 125.97, 123.73, 122.70, 120.43, 118.56, 98.14, 78.43, 76.01, 70.52, 57.72, 35.98, 31.95, 30.99, 30.19, 29.73. |
| 87 | SW393293 | 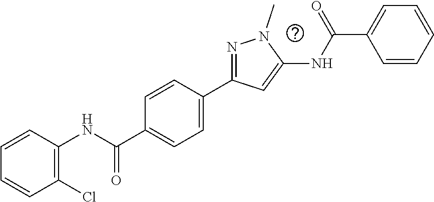 [SMILES: Cn1nc(-c2ccc(C(=O)Nc3ccccc3Cl)cc2)cc1NC(=O)c1ccccc1] | 1H NMR (400 MHz, MeOD) δ 8.12 (dd, J = 8.2, 1.6 Hz, 1H), 8.02-7.90 (m, 7H), 7.62-7.58 (m, 1H), 7.57- 7.49 (m, 4H), 7.49-7.44 (m, 2H), 7.34 (td, J = 7.7, 1.6 Hz, 2H), 7.17 (td, J = 7.7, 1.7 Hz, 1H), 6.72 (s, 1H), 3.87 (d, J = 3.6 Hz, 3H). | |
| 88 | SW393294 | 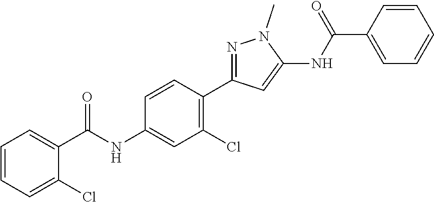 [SMILES: Cn1nc(-c2ccc(NC(=O)c3ccccc3Cl)cc2Cl)cc1NC(=O)c1ccccc1] | (600 MHz, DMSO) δ 10.79 (s, 1H), 10.51 (s, 1H), 8.07-7.99 (m, 3H), 7.83 (d, J = 8.5 Hz, 1H), 7.69 (dd, J = 8.5, 2.2 Hz, 1H), 7.67-7.52 (m, 7H), 7.49 (td, J = 7.4, 1.3 Hz, 1H), 6.78 (s, 1H), 3.80 (s, 3H). | (151 MHz, DMSO) δ 166.20, 165.68, 145.96, 139.64, 137.31, 137.00, 133.72, 132.70, 131.85, 131.23, 130.72, 130.40, 130.20, 129.46, 129.04, 128.41, 127.81, 120.85, 118.75, 101.67, 36.44. |
| 89 | SW393296 | 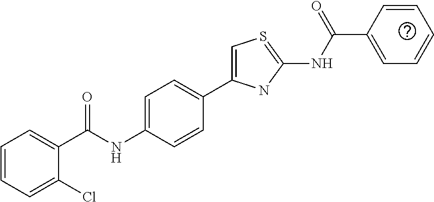 | (600 MHz, DMSO-d₆) δ 10.66 (s, 1H), 8.15 (d, J = 7.6 Hz, 2H), 7.96 (d, J = 8.4 Hz, 2H), 7.83 (d, J = 8.3 Hz, 2H), 7.64 (td, J = 13.3, 11.8, 7.4 Hz, 3H), 7.57 (q, J = 7.5 Hz, 3H), 7.52 (t, J = 7.6 Hz, 1H), 7.47 (t, J = 7.4 Hz, 1H). | (151 MHz, DMSO-d₆) δ 165.4, 165.1, 158.7, 149.0, 138.6, 137.0, 132.7, 132.1, 131.3, 130.1, 130.0, 129.8, 129.1, 128.7, 128.3, 127.4, 126.4, 119.8, 107.8; |
| 90 | SW393298 | 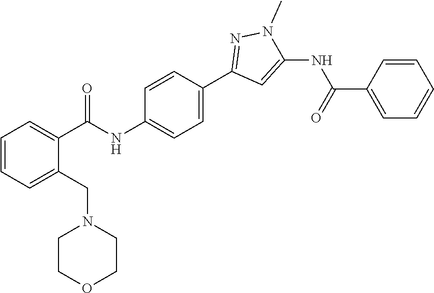 [SMILES: Cn1nc(-c2ccc(NC(=O)c3ccccc3CN3CCOCC3)cc2)cc1NC(=O)c1ccccc1] | (600 MHz, Chloroform-d) δ 11.80 (s, 1H), 8.83 (s, 1H), 7.96 (dd, J = 6.9, 4.2 Hz, 3H), 7.70 (d, J = 8.3 Hz, 2H), 7.57 (d, J = 8.3 Hz, 2H), 7.51 (t, J = 7.4 Hz, 1H), 7.44 (dd, J = 5.7, 3.3 Hz, 2H), 7.38 (t, J = 7.6 Hz, 2H), 7.22 (dd, J = 5.6, 3.3 Hz, 1H), 6.47 (s, 1H), 3.77 (s, 3H), 3.74 (t, J = 4.9 Hz, 4H), 3.61 (s, 2H), 2.54 (d, J = 5.6 Hz, 4H). | (151 MHz, Chloroform-d) δ 166.9, 166.4, 149.4, 137.8, 137.3, 136.8, 133.2, 132.7, 132.4, 132.4, 131.6, 130.7, 130.2, 128.9, 128.8, 127.8, 126.1, 120.9, 98.1, 66.8, 62.7, 52.8, 36.0. |
| 91 | SW393345 | 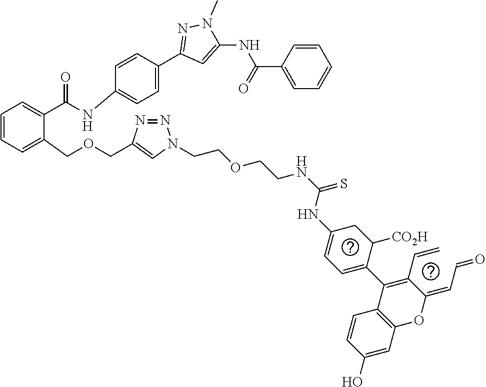 [SMILES: C=CC1=C(C2=CC=C(NC(=S)NCCOCCn3cc(COCc4ccccc4C(=O)Nc4ccc(-c5cc(NC(=O)c6ccccc6)n(C)n5)cc4)nn3)CC2C(=O)O)c2ccc(O)cc2O/C1=C/C=O] | (600 MHz, MeOD) δ 7.95 (d, J = 6.8 Hz, 3H), 7.73 (dd, J = 8.3, 2.1 Hz, 1H), 7.70-7.63 (m, 3H), 7.62-7.53 (m, 4H), 7.47 (t, J = 7.8 Hz, 2H), 7.41-7.34 (m, 3H), 6.98 (d, J = 8.2 Hz, 1H), 6.64 (d, J = 2.4 Hz, 2H), 6.59 (dd, J = 8.7, 5.7 Hz, 2H), 6.55 (s, 1H), 6.48 (dt, J = 8.8, 1.8 Hz, 2H), 4.73 (s, 2H), 4.38 (t, J = 4.9 Hz, 2H), 3.82- 3.71 (m, 9H), 3.57 (t, J = 5.1 Hz, 6H). | |
| 92 | SW394442 | 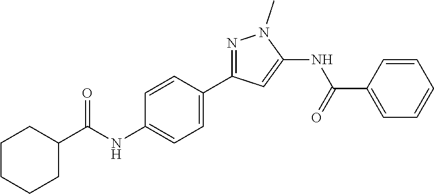 [SMILES: Cn1nc(-c2ccc(NC(=O)C3CCCCC3)cc2)cc1NC(=O)c1ccccc1] | (600 MHz, DMSO-d₆) δ 10.42 (s, 1H), 9.87 (s, 1H), 8.03-7.99 (m, 2H), 7.73-7.69 (m, 2H), 7.67-7.62 (m, 3H), 7.56 (dd, J = 8.3, 6.9 Hz, 2H), 6.66 (s, 1H), 3.74 (s, 3H), 2.34 (tt, J = 11.7, 3.5 Hz, 1H), 1.84-1.73 (m, 4H), 1.42 (qd, J = 12.6, 3.2 Hz, 2H), 1.32-1.14 (m, 4H). | (151 MHz, DMSO-d₆) δ 174.3, 165.7, 148.1, 138.9, 137.5, 133.3, 132.2, 128.6, 128.2, 127.9, 125.1, 119.1, 97.5, 44.9, 35.8, 29.2, 25.3 |
| 93 | SW394443 | 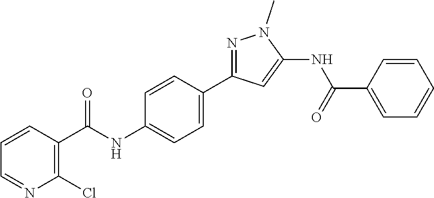 [SMILES: Cn1nc(-c2ccc(NC(=O)c3cccnc3Cl)cc2)cc1NC(=O)c1ccccc1] | (600 MHz, DMSO-d₆) δ 10.73 (s, 1H), 10.44 (s, 1H), 8.55 (dd, J = 4.8, 1.9 Hz, 1H), 8.11 (dd, J = 7.5, 1.9 Hz, 1H), 8.03-8.00 (m, 2H), 7.82-7.79 (m, 2H), 7.77-7.74 (m, 2H), 7.66- 7.62 (m, 1H), 7.57 (ddd, J = 8.7, 6.1, 4.2 Hz, 3H), 6.71 (s, 1H), 3.76 (s, 3H). | (151 MHz, DMSO-d₆) δ 165.8, 163.5, 150.6, 147.9, 146.5, 138.3, 138.0, 137.6, 133.3, 133.2, 132.3, 129.3, 128.6, 128.0, 125.3, 123.3, 119.72, 97.7, 35.9; |
| 94 | SW394444 | 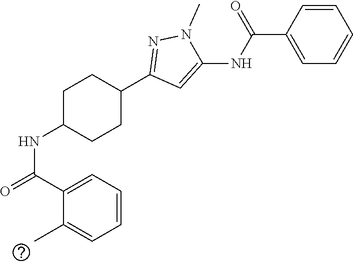 [SMILES: Cc1ccccc1C(=O)NC1CCC(c2cc(NC(=O)c3ccccc3)n(C)n2)CC1] | (400 MHz, cd₃od) δ 7.93 (t, J = 6.1 Hz, 2H), 7.63-7.25 (m, 7H), 6.12 (d, J = 9.3 Hz, 1H), 3.91 (tt, J = 11.8, 8.4, 3.8 Hz, 1H), 3.69 (d, J = 2.8 Hz, 3H), 3.30 (s, 1H), 2.57 (tt, J = 12.5, 3.6 Hz, 1H), 2.21-1.97 (m, 3H), 1.97-1.70 (m, 3H), 1.67-1.37 (m, 3H). | (101 MHz, cd₃od) δ 167.84, 167.75, 155.83, 155.53, 136.60, 136.40, 133.05, 132.26, 130.62, 129.63, 129.60, 128.65, 128.46, 128.45, 127.63, 127.61, 126.70, 126.68, 98.43, 97.70, 78.00, 77.68, 77.35, 48.85, 47.83, 47.61, 47.40, 46.21, 36.84, 35.50, 34.64, 31.99, 31.66, 28.96, 27.47. |
| 95 | SW394445 | 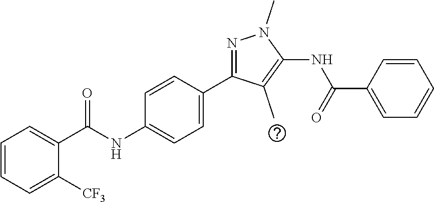 [SMILES: Cc1c(-c2ccc(NC(=O)c3ccccc3C(F)(F)F)cc2)nn(C)c1NC(=O)c1ccccc1] | (600 MHz, MeOD) δ 8.02 (d, J = 7.5 Hz, 2H), 7.86 (dd, J = 8.3, 5.8 Hz, 2H), 7.78-7.71 (m, 2H), 7.70-7.59 (m, 4H), 7.53 (t, J = 7.6 Hz, 2H), 2.01 (s, 3H). | |
| 96 | SW394446 | 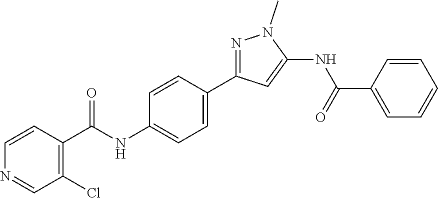 [SMILES: Cn1nc(-c2ccc(NC(=O)c3ccncc3Cl)cc2)cc1NC(=O)c1ccccc1] | (600 MHz, DMSO-d₆) δ 10.80 (s, 1H), 10.46 (s, 1H), 8.80 (s, 1H), 8.68 (d, J = 4.7 Hz, 1H), 8.04-7.99 (m, 2H), 7.83-7.79 (m, 2H), 7.77-7.73 (m, 2H), 7.68 (d, J = 4.8 Hz, 1H), 7.66- 7.62 (m, 1H), 7.57 (t, J = 7.6 Hz, 2H), 6.71 (s, 1H), 3.76 (s, 3H). | (151 MHz, DMSO-d₆) δ 165.8, 162.8, 149.5, 148.6, 147.9, 143.4, 137.7, 137.6, 133.3, 132.3, 129.5, 128.6, 128.0, 127.5, 125.3, 122.9, 119.8, 97.7, 35.9; |
| 97 | SW394447 | 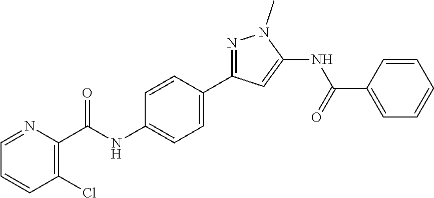 [SMILES: Cn1nc(-c2ccc(NC(=O)c3ncccc3Cl)cc2)cc1NC(=O)c1ccccc1] | (600 MHz, Methanol-d₄) δ 8.50 (d, J = 4.5 Hz, 1H), 7.95 (d, J = 7.6 Hz, 2H), 7.86 (d, J = 8.2 Hz, 1H), 7.77-7.68 (m, 4H), 7.56 (t, J = 7.5 Hz, 1H), 7.50-7.41 (m, 3H), 6.56 (s, 1H), 3.79 (s, 3H), 2.78 (s, 3H). | (151 MHz, Methanol-d₄) δ 168.2, 162.9 (d, J = 2.7 Hz), 150.2, 147.9, 146.929, 140.7, 138.1, 137.8, 133.5, 133.0, 131.8, 130.1, 129.1, 128.3, 127.3, 126.4, 120.7, 98.7, 38.8, 35.9. |
| 98 | SW394484 | 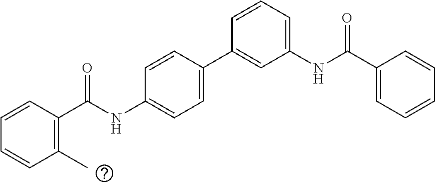 [SMILES: Cc1ccccc1C(=O)Nc1ccc(-c2cccc(NC(=O)c3ccccc3)c2)cc1] | (600 MHz, MeOD) δ 7.87-7.81 (m, 3H), 7.69-7.63 (m, 2H), 7.61-7.56 (m, 1H), 7.55-7.50 (m, 2H), 7.50- 7.43 (m, 2H), 7.41-7.36 (m, 2H), 7.36- 7.34 (m, 1H), 7.34-7.24 (m, 4H). | (151 MHz, MeOD) δ 167.24, 166.16, 141.21, 138.68, 137.40, 137.05, 136.03, 134.80, 131.78, 131.22, 130.91, 130.04, 129.28, 129.08, 128.53, 127.50, 127.36, 126.94, 122.97, 120.67, 120.57, 119.61, 119.26, 77.46, 77.25, 77.04, 60.65, 29.63, 13.96. |
| 99 | SW394485 | 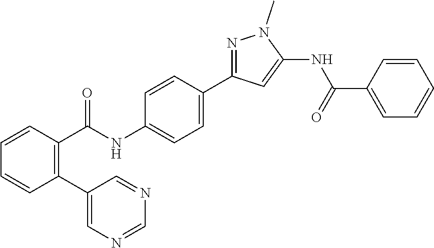 [SMILES: Cn1nc(-c2ccc(NC(=O)c3ccccc3-c3cncnc3)cc2)cc1NC(=O)c1ccccc1] | (600 MHz, Methanol-d₄) δ 9.07 (s, 1H), 8.82 (s, 2H), 7.97-7.93 (m, 2H), 7.73 (dd, J = 7.6, 1.4 Hz, 1H), 7.68- 7.64 (m, 2H), 7.62 (td, J = 7.6, 1.4 Hz, 1H), 7.59-7.55 (m, 2H), 7.55- 7.51 (m, 2H), 7.50-7.44 (m, 3H), 6.54 (s, 1H), 3.78 (s, 3H). | (151 MHz, Methanol-d₄) δ 172.6, 168.7, 168.4, 157.3, 156.6, 150.2, 138.3, 138.2, 137.1, 135.3, 133.6, 133.3, 133.1, 131.5, 130.2, 129.8, 129.2, 128.9, 128.4, 126.4, 121.0, 98.8, 36.0; |
| 100 | SW394486 | 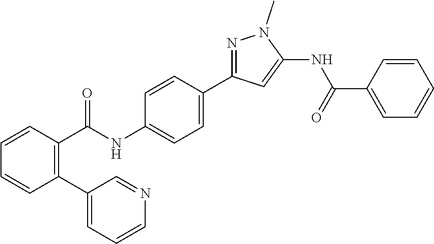 [SMILES: Cn1nc(-c2ccc(NC(=O)c3ccccc3-c3cccnc3)cc2)cc1NC(=O)c1ccccc1] | (600 MHz, DMSO-d₆) δ 10.46 (s, 1H), 10.44 (s, 1H), 8.65 (d, J = 2.4 Hz, 1H), 8.52 (dd, J = 4.8, 1.7 Hz, 1H), 8.02 (d, J = 7.6 Hz, 2H), 7.85 (dt, J = 7.9, 2.0 Hz, 1H), 7.71 (d, J = 8.3 Hz, 2H), 7.68 (d, J = 7.5 Hz, 1H), 7.63 (dtd, J = 7.5, 5.1, 2.4 Hz, 2H), 7.61-7.52 (m, 6H), 7.43 (dd, J = 7.9, 4.8 Hz, 1H), 6.67 (s, 1H), 3.75 (s, 3H). | (151 MHz, DMSO-d₆) δ 167.4, 165.7, 148.7, 148.4, 148.0, 138.3, 137.5, 137.2, 136.0, 135.8, 135.8, 133.3, 132.2, 130.2, 130.2, 129.0, 128.6, 128.1, 128.1, 128.0, 125.1, 123.4, 119.7, 97.6, 35.9. |
| 101 | SW394487 | 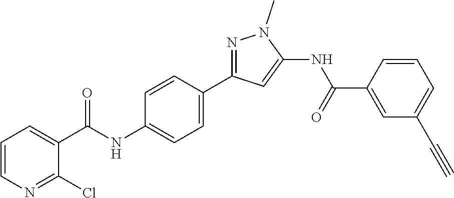 [SMILES: C#Cc1cccc(C(=O)Nc2cc(-c3ccc(NC(=O)c4cccnc4Cl)cc3)nn2C)c1] | (600 MHz, DMSO-d₆) δ 10.72 (s, 1H), 10.53 (s, 1H), 8.55 (dd, J = 4.8, 1.9 Hz, 1H), 8.13-8.09 (m, 2H), 8.02 (d, J = 7.8 Hz, 1H), 7.80 (d, J = 8.4 Hz, 2H), 7.75 (dd, J = 8.6, 2.4 Hz, 3H), 7.62-7.55 (m, 2H), 6.71 (s, 1H), 4.36 (s, 1H), 3.77 (s, 3H). | (151 MHz, DMSO-d₆) δ 164.8, 163.5, 150.6, 147.9, 146.5, 138.3, 138.1, 137.4, 135.2, 133.8, 133.2, 131.0, 129.3, 129.2, 128.6, 125.3, 123.3, 122.1, 119.7, 97.6, 82.7, 81.9, 35.9. |
| 102 | SW394488 | 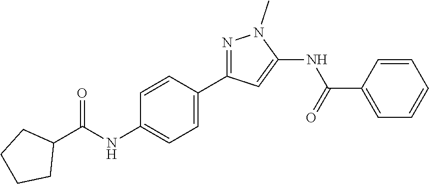 [SMILES: Cn1nc(-c2ccc(NC(=O)C3CCCC3)cc2)cc1NC(=O)c1ccccc1] | (600 MHz, DMSO-d₆) δ 10.42 (s, 1H), 9.93 (s, 1H), 8.01 (d, J = 7.3 Hz, 2H), 7.71 (d, J = 8.6 Hz, 2H), 7.64 (dd, J = 13.0, 7.9 Hz, 3H), 7.56 (t, J = 7.6 Hz, 2H), 6.66 (s, 1H), 3.74 (s, 3H), 2.78 (p, J = 8.0 Hz, 1H), 1.85 (ddt, J = 11.7, 8.6, 5.1 Hz, 2H), 1.77-1.64 (m, 4H), 1.56 (dq, J = 10.0, 6.5, 4.8 Hz, 2H). | (151 MHz, DMSO-d₆) δ 174.4, 165.7, 148.1, 138.9, 137.5, 133.4, 132.2, 128.6, 128.2, 127.9, 125.1, 119.2, 97.5, 45.3, 35.8, 30.2, 25.7. |
| 103 | SW394489 | 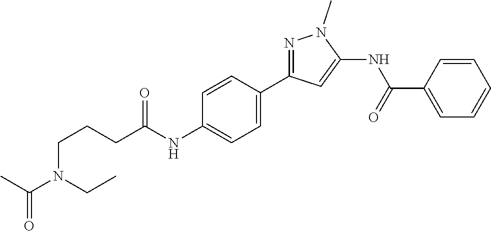 [SMILES: CCN(CCCC(=O)Nc1ccc(-c2cc(NC(=O)c3ccccc3)n(C)n2)cc1)C(C)=O] | (600 MHz, DMSO) δ 10.43 (s, 1H), 10.00 (s, 1H), 8.05-7.97 (m, 2H), 7.76-7.69 (m, 2H), 7.64 (dd, J = 8.1, 6.2 Hz, 3H), 7.57 (t, J = 7.7 Hz, 2H), 6.67 (s, 1H), 4.01 (s, 2H), 3.75 (s, 3H), 2.80 (s, 3H), 1.79 (dt, J = 13.6, 3.3 Hz, 3H), 1.50 (qd, J = 12.5, 4.3 Hz, 2H), 1.42 (s, 9H). | (151 MHz, DMSO) δ 173.46, 166.15, 154.33, 148.47, 139.10, 137.92, 133.76, 132.66, 129.03, 128.79, 128.38, 125.58, 119.67, 97.93, 79.13, 79.09, 43.13, 40.52, 36.27, 28.68, 28.56, 28.53, 28.21. |
| 104 | SW394490 | 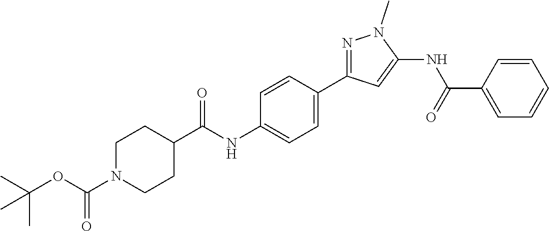 [SMILES: Cn1nc(-c2ccc(NC(=O)C3CCN(C(=O)OC(C)(C)C)CC3)cc2)cc1NC(=O)c1ccccc1] | (600 MHz, MeOD) δ 7.98-7.94 (m, 2H), 7.70-7.66 (m, 2H), 7.61-7.57 (m, 3H), 7.50 (t, J = 7.7 Hz, 2H), 6.58 (s, 1H), 3.81 (s, 3H), 3.65 (hept, J = 6.6 Hz, 2H), 3.19-3.08 (m, 3H), 2.71 (td, J = 12.9, 2.9 Hz, 1H), 2.60 (tt, J = 11.4, 3.9 Hz, 1H), 2.11 (s, 3H), 1.96- 1.88 (m, 2H). | (151 MHz, MeOD) δ 173.72, 169.96, 167.59, 149.78, 138.13, 137.60, 132.99, 132.46, 128.80, 128.62, 127.76, 125.86, 120.17, 120.07, 98.01, 77.53, 77.47, 77.32, 77.26, 77.10, 54.44, 54.39, 45.90, 43.27, 43.22, 42.54, 41.14, 35.39, 28.66, 28.37, 20.96, 18.22, 18.19, 16.82, 12.23. |
| 105 | SW394491 | 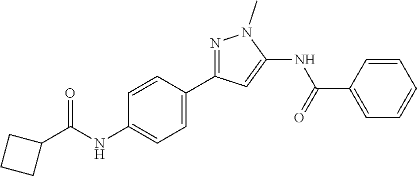 [SMILES: Cn1nc(-c2ccc(NC(=O)C3CCC3)cc2)cc1NC(=O)c1ccccc1] | (600 MHz, DMSO-d₆) δ 10.42 (s, 1H), 9.79 (s, 1H), 8.03-7.99 (m, 2H), 7.74- 7.69 (m, 2H), 7.67-7.61 (m, 3H), 7.56 (dd, J = 8.4, 7.0 Hz, 2H), 6.66 (s, 1H), 3.74 (s, 3H), 3.27-3.20 (m, 1H), 2.24 (pd, J = 9.1, 2.4 Hz, 2H), 2.11 (dtd, J = 11.9, 8.7, 2.6 Hz, 2H), 1.94 (dp, J = 11.0, 9.0 Hz, 1H), 1.85-1.77 (m, 1H). | (151 MHz, DMSO-d₆) δ 172.9, 165.7, 148.1, 138.8, 137.5, 133.3, 132.2, 128.6, 128.2, 127.9, 125.1, 119.2, 97.5, 53.6, 35.8, 24.7, 17.8 |
| 106 | SW394515 | 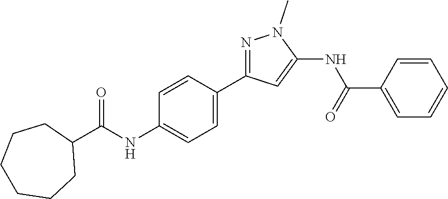 [SMILES: Cn1nc(-c2ccc(NC(=O)C3CCCCCC3)cc2)cc1NC(=O)c1ccccc1] | (600 MHz, DMSO-d₆) δ 10.42 (s, 1H), 9.85 (s, 1H), 8.01 (d, J = 7.7 Hz, 2H), 7.71 (d, J = 8.2 Hz, 2H), 7.64 (d, J = 7.9 Hz, 3H), 7.56 (t, J = 7.6 Hz, 2H), 6.66 (s, 1H), 3.74 (s, 3H), 1.88-1.81 (m, 2H), 1.78-1.70 (m, 3H), 1.68- 1.42 (m, 8H). | (151 MHz, DMSO) δ 175.3, 165.7, 148.1, 138.9, 137.5, 133.3, 132.2, 128.6, 128.1, 127.9, 125.1, 119.2, 97.5, 46.4, 35.8, 31.1, 28.0, 26.1; |
| 107 | SW394516 (isomers-1) | 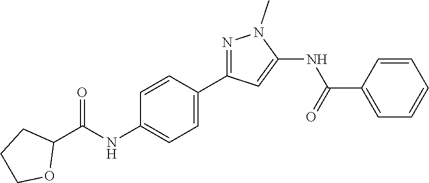 [SMILES: Cn1nc(-c2ccc(NC(=O)C3CCCO3)cc2)cc1NC(=O)c1ccccc1] | (600 MHz, Methanol-d₄) δ 7.94 (d, J = 7.3 Hz, 2H), 7.70-7.67 (m, 2H), 7.62- 7.58 (m, 2H), 7.58-7.53 (m, 1H), 7.47 (t, J = 7.8 Hz, 2H), 6.55 (s, 1H), 4.42 (dd, J = 8.4, 6.0 Hz, 1H), 4.05 (dt, J = 8.3, 6.5 Hz, 1H), 3.93 (dt, J = 8.4, 6.8 Hz, 1H), 3.77 (s, 3H), 2.32 (dq, J = 13.1, 7.6 Hz, 1H), 2.09 (ddt, J = 12.7, 8.1, 6.3 Hz, 1H), 1.93 (pd, J = 6.7, 2.5 Hz, 2H). | (151 MHz, MeOD) δ 172.9, 168.2, 150.14, 138.0, 137.2, 133.5, 132.9, 130.1, 129.1, 128.2, 126.4, 120.8, 98.6, 78.9, 70.1, 35.8, 30.8, 25.9. |
| 108 | SW394516 (isomers-2) | 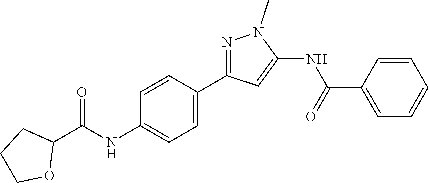 [SMILES: Cn1nc(-c2ccc(NC(=O)C3CCCO3)cc2)cc1NC(=O)c1ccccc1] | (600 MHz, Methanol-d₄) δ 7.94 (d, J = 7.3 Hz, 2H), 7.70-7.67 (m, 2H), 7.62- 7.58 (m, 2H), 7.58-7.53 (m, 1H), 7.47 (t, J = 7.8 Hz, 2H), 6.55 (s, 1H), 4.42 (dd, J = 8.4, 6.0 Hz, 1H), 4.05 (dt, J = 8.3, 6.5 Hz, 1H), 3.93 (dt, J = 8.4, 6.8 Hz, 1H), 3.77 (s, 3H), 2.32 (dq, J = 13.1, 7.6 Hz, 1H), 2.09 (ddt, J = 12.7, 8.1, 6.3 Hz, 1H), 1.93 (pd, J = 6.7, 2.5 Hz, 2H). | (151 MHz, MeOD) δ 172.9, 168.2, 150.14, 138.0, 137.2, 133.5, 132.9, 130.1, 129.1, 128.2, 126.4, 120.8, 98.6, 78.9, 70.1, 35.8, 30.8, 25.9. |
| 109 | SW394517 | 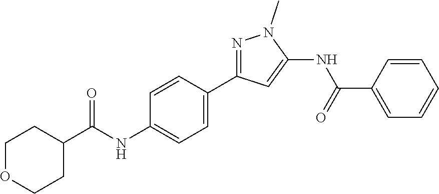 [SMILES: Cn1nc(-c2ccc(NC(=O)C3CCOCC3)cc2)cc1NC(=O)c1ccccc1] | (600 MHz, DMSO-d₆) δ 10.43 (s, 1H), 9.98 (s, 1H), 8.01 (d, J = 7.7 Hz, 2H), 7.72 (d, J = 8.3 Hz, 2H), 7.64 (dd, J = 11.9, 7.8 Hz, 3H), 7.56 (t, J = 7.6 Hz, 2H), 6.66 (s, 1H), 3.91 (dt, J = 11.7, 3.2 Hz, 2H), 3.74 (s, 3H), 3.38-3.31 (m, 2H), 2.61 (tt, J = 10.3, 4.8 Hz, 1H), 1.69 (qt, J = 12.9, 6.1 Hz, 4H). | (151 MHz, DMSO) δ 173.0, 165.7, 148.0, 138.7, 137.5, 133.3, 132.2, 128.6, 128.3, 128.0, 125.1, 119.2, 97.5, 66.4, 41.8, 35.8, 28.9; |
| 110 | SW394518 (HCl Salt: SW394676) | 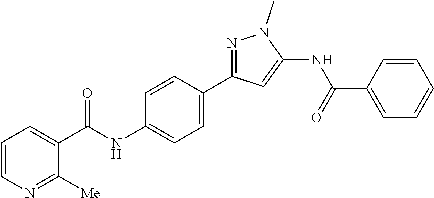 [SMILES: Cc1ncccc1C(=O)Nc1ccc(-c2cc(NC(=O)c3ccccc3)n(C)n2)cc1] | (600 MHz, DMSO-d₆) δ 10.53 (s, 1H), 10.44 (s, 1H), 8.57 (dd, J = 4.9, 1.8 Hz, 1H), 8.04-8.00 (m, 2H), 7.89 (dd, J = 7.7, 1.8 Hz, 1H), 7.79 (s, 4H), 7.66-7.62 (m, 1H), 7.57 (t, J = 7.6 Hz, 2H), 7.35 (dd, J = 7.6, 4.9 Hz, 1H), 6.71 (s, 1H), 3.76 (s, 3H), 2.59 (s, 3H). | (151 MHz, DMSO) δ 166.6, 165.8, 155.1, 149.8, 148.0, 138.4, 137.59, 135.3, 133.4, 132.3, 132.3, 129.1, 128.6, 128.0, 125.2, 121.0, 119.8, 97.7, 35.9, 22.7; |
| 111 | SW394519 | 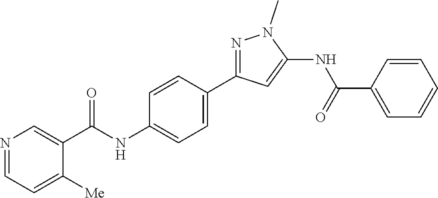 [SMILES: Cc1ccncc1C(=O)Nc1ccc(-c2cc(NC(=O)c3ccccc3)n(C)n2)cc1] | (600 MHz, DMSO-d₆) δ 10.57 (s, 1H), 10.44 (s, 1H), 8.67 (s, 1H), 8.55 (d, J = 5.0 Hz, 1H), 8.03-8.00 (m, 2H), 7.79 (s, 4H), 7.66-7.62 (m, 1H), 7.57 (t, J = 7.6 Hz, 2H), 7.37 (d, J = 5.0 Hz, 1H), 6.71 (s, 1H), 3.76 (s, 3H), 2.43 (s, 3H). | (151 MHz, DMSO) δ 165.7, 165.5, 150.4, 148.0, 147.7, 145.1, 138.3, 137.6, 133.4, 133.0, 132.2, 129.1, 128.6, 128.0, 125.7, 125.2, 119.9, 97.6, 35.9, 18.8. |
| 112 | SW394532 | 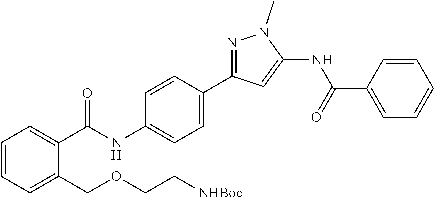 [SMILES: Cn1nc(-c2ccc(NC(=O)c3ccccc3COCCNC(=O)OC(C)(C)C)cc2)cc1NC(=O)c1ccccc1] | (600 MHz, CDCl₃) δ 9.10 (s, 1H), 8.61 (s, 1H), 7.83 (d, J = 7.6 Hz, 2H), 7.67 (d, J = 7.5 Hz, 1H), 7.62 (d, J = 8.3 Hz, 2H), 7.52 (d, J = 8.2 Hz, 2H), 7.48-7.43 (m, 1H), 7.39 (td, J = 7.5, 1.5 Hz, 1H), 7.36-7.28 (m, 4H), 6.38 (s, 1H), 4.57 (s, 2H), 3.68 (s, 3H), 3.50 (t, J = 5.4 Hz, 2H), 3.25 (q, J = 5.6 Hz, 2H), 1.32 (s, 9H). | (151 MHz, CDCl₃) δ 166.99, 166.33, 156.00, 149.35, 137.46, 136.64, 136.60, 134.21, 132.97, 132.45, 130.85, 130.78, 129.98, 129.49, 128.89, 128.75, 127.65, 126.10, 120.25, 98.08, 79.57, 71.66, 69.43, 40.29, 35.91, 29.72, 28.38.; |
| 113 | SW394534 (HCl salt: SW394672) | 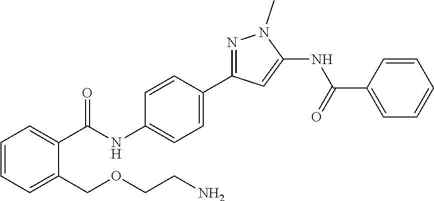 [SMILES: Cn1nc(-c2ccc(NC(=O)c3ccccc3COCCN)cc2)cc1NC(=O)c1ccccc1] | (600 MHz, MeOD) δ 8.08-8.02 (m, 2H), 7.92-7.84 (m, 5H), 7.72-7.67 (m, 1H), 7.64 (dd, J = 7.6, 1.4 Hz, 1H), 7.62-7.58 (m, 4H), 7.56 (td, J = 7.5, 1.4 Hz, 1H), 7.50 (td, J = 7.5, 1.4 Hz, 1H), 7.02 (s, 1H), 4.83 (s, 2H), 4.00 (d, J = 1.4 Hz, 3H), 3.74-3.71 (m, 2H), 3.13 (t, J = 5.0 Hz, 3H). | (151 MHz, MeOD) δ 169.42, 147.94, 141.08, 140.14, 135.98, 135.19, 133.00, 132.34, 130.58, 129.94, 128.72, 128.42, 128.13, 127.71, 127.03, 120.71, 98.25, 77.68, 77.46, 77.25, 71.13, 65.90, 39.52, 35.74, 29.56. |
| 114 | SW394535 | 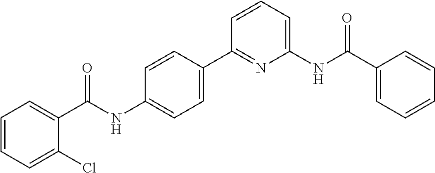 [SMILES: O=C(Nc1cccc(-c2ccc(NC(=O)c3ccccc3Cl)cc2)n1)c1ccccc1] | (600 MHz, CDCl₃) δ 8.69 (s, 1H), 8.36 (s, 1H), 8.05 (d, J = 8.5 Hz, 3H), 8.01 (d, J = 7.2 Hz, 2H), 7.87 (s, 1H), 7.85- 7.79 (m, 3H), 7.62 (t, J = 7.4 Hz, 1H), 7.56 (t, J = 8.1 Hz, 3H), 7.50 (d, J = 7.9 Hz, 1H), 7.46 (td, J = 7.6, 1.8 Hz, 1H), 7.42 (t, J = 7.4 Hz, 1H). | (151 MHz, CDCl₃) δ 165.82, 164.45, 151.22, 134.96, 132.34, 131.95, 130.63, 130.55, 130.50, 128.91, 127.71, 127.43, 127.31, 120.09, 116.36, 112.39. |
| 115 | SW394544 | 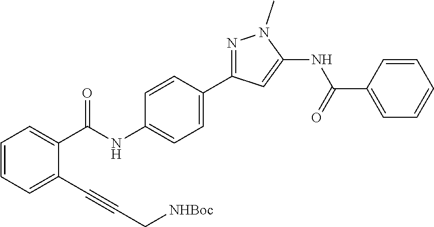 [SMILES: Cn1nc(-c2ccc(NC(=O)c3ccccc3C#CCNC(=O)OC(C)(C)C)cc2)cc1NC(=O)c1ccccc1] | (600 MHz, CDCl₃) δ 8.96 (s, 1H), 8.68 (s, 1H), 7.88-7.81 (m, 3H), 7.59 (d, J = 8.2 Hz, 2H), 7.52 (d, J = 8.3 Hz, 2H), 7.48-7.42 (m, 1H), 7.42-7.38 (m, 1H), 7.33 (t, J = 8.0 Hz, 4H), 6.35 (s, 1H), 4.07 (d, J = 5.8 Hz, 2H), 3.66 (s, 3H), 1.35 (s, 9H). | (151 MHz, CDCl₃) δ 166.35, 164.87, 155.48, 149.28, 137.04, 136.63, 136.21, 133.79, 132.99, 132.41, 130.81, 130.02, 129.74, 128.99, 128.72, 127.69, 126.08, 120.31, 119.29, 98.05, 93.11, 80.81, 80.29, 60.46, 35.88, 35.35, 31.16, 29.72, 28.35, 21.09, 14.21. |
| 116 | SW394545 | 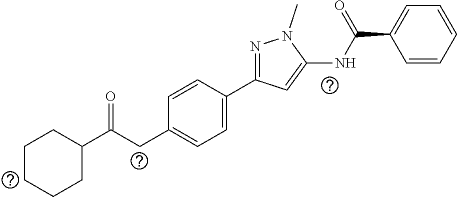 | | |
| 117 | SW394546 | 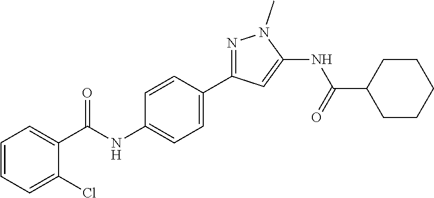 [SMILES: Cn1nc(-c2ccc(NC(=O)c3ccccc3Cl)cc2)cc1NC(=O)C1CCCCC1] | (600 MHz, DMSO-d₆) δ 10.56 (s, 1H), 9.86 (s, 1H), 7.76-7.71 (m, 4H), 7.59 (dd, J = 13.8, 7.7 Hz, 2H), 7.51 (t, J = 7.7 Hz, 1H), 7.46 (t, J = 7.4 Hz, 1H), 6.60 (s, 1H), 3.68 (s, 3H), 2.43 (td, J = 11.6, 9.8, 5.8 Hz, 1H), 1.84 (d, J = 12.6 Hz, 2H), 1.79-1.74 (m, 2H), 1.66 (d, J = 12.4 Hz, 1H), 1.42 (qd, J = 12.4, 3.3 Hz, 2H), 1.34-1.25 (m, 2H), 1.24-1.15 (m, 1H); | (151 MHz, DMSO) δ 174.3, 164.9, 147.8, 138.2, 137.8, 137.0, 131.1, 130.0, 129.7, 129.1, 129.0, 127.3, 125.2, 119.6, 95.9, 43.8, 35.7, 29.1, 25.4, 25.1; |
| 118 | SW394547 | 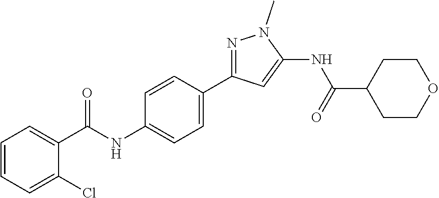 [SMILES: Cn1nc(-c2ccc(NC(=O)c3ccccc3Cl)cc2)cc1NC(=O)C1CCOCC1] | (600 MHz, DMSO-d₆) δ 10.56 (s, 1H), 9.96 (s, 1H), 7.74 (d, J = 1.5 Hz, 4H), 7.59 (ddd, J = 13.5, 7.7, 1.4 Hz, 2H), 7.51 (td, J = 7.7, 1.8 Hz, 1H), 7.46 (td, J = 7.5, 1.3 Hz, 1H), 6.62 (s, 1H), 3.92 (ddd, J = 11.5, 4.4, 2.1 Hz, 2H), 3.69 (s, 3H), 3.44-3.31 (m, 2H), 2.70 (ddt, J = 11.4, 7.3, 4.1 Hz, 1H), 1.75 (dd, J = 12.7, 3.5 Hz, 2H), 1.69 (td, J = 12.0, 4.1 Hz, 2H). | (151 MHz, DMSO) δ 173.1, 164.9, 147.9, 138.2, 137.6, 137.0, 131.1, 130.0, 129.7, 129.1, 129.0, 127.3, 125.2, 119.7, 96.0, 66.3, 40.8, 35.7, 28.8. |
| 119 | SW394548 | 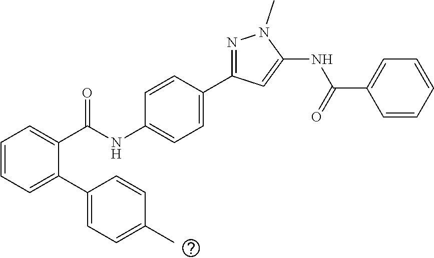 [SMILES: Cc1ccc(-c2ccccc2C(=O)Nc2ccc(-c3cc(NC(=O)c4ccccc4)n(C)n3)cc2)cc1] | (600 MHz, DMSO-d₆) δ 10.52 (s, 2H), 8.26 (d, J = 8.3 Hz, 2H), 8.02 (d, J = 7.7 Hz, 2H), 7.77-7.47 (m, 13H), 6.68 (s, 1H), 3.75 (s, 3H). | (151 MHz, DMSO) δ 167.2, 165.7, 147.9, 147.1, 146.7, 138.4, 137.7, 137.5, 137.0, 133.3, 132.3, 130.3, 130.2, 129.7, 128.8, 128.6, 128.3, 128.0, 125.2, 123.5, 119.8, 97.6, 35.9. |
| 120 | SW394549 | 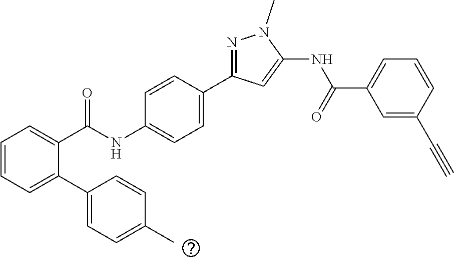 [SMILES: C#Cc1cccc(C(=O)Nc2cc(-c3ccc(NC(=O)c4ccccc4-c4ccc(C)cc4)cc3)nn2C)c1] | (600 MHz, DMSO-d₆) δ 10.52 (s, 1H), 10.37 (s, 1H), 8.11 (d, J = 1.8 Hz, 1H), 8.02 (dt, J = 7.8, 1.5 Hz, 1H), 7.74 (dt, J = 7.8, 1.4 Hz, 1H), 7.73- 7.69 (m, 2H), 7.62-7.55 (m, 5H), 7.52-7.45 (m, 4H), 7.17-7.13 (m, 2H), 6.67 (s, 1H), 4.36 (s, 1H), 3.75 (s, 3H). | (151 MHz, DMSO) δ 167.7, 164.8, 138.6, 138.5, 138.3, 137.3, 137.0, 137.0, 135.1, 133.8, 131.0, 129.9, 129.9, 129.2, 128.8, 128.6, 128.0, 127.4, 125.1, 122.1, 119.7, 119.12, 97.5, 82.7, 81.9, 35.9. |
| 121 | SW394572 | 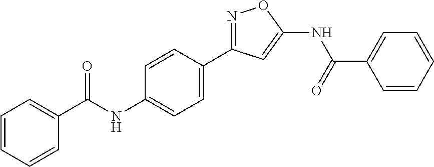 [SMILES: O=C(Nc1ccc(-c2cc(NC(=O)c3ccccc3)on2)cc1)c1ccccc1] | (600 MHz, MeOD) δ 8.01-7.97 (m, 2H), 7.92 (dq, J = 8.1, 1.6, 1.2 Hz, 2H), 7.86-7.79 (m, 4H), 7.59 (td, J = 7.3, 1.3 Hz, 1H), 7.57-7.45 (m, 5H), 6.88 (s, 1H). | (151 MHz, MeOD) δ 163.18, 162.18, 134.72, 132.68 (d, J = 11.4 Hz), 131.87, 128.69, 128.52, 127.84, 127.36 (d, J = 13.7 Hz), 120.75, 87.32, 77.46, 77.24, 77.03, 48.83 (dd, J = 43.1, 21.9 Hz), 48.47. |
| 122 | SW394597 | 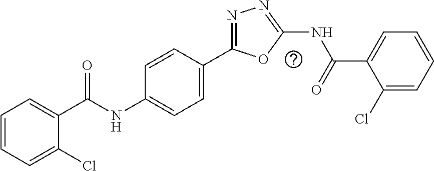 [SMILES: O=C(Nc1ccc(-c2nnc(NC(=O)c3ccccc3Cl)o2)cc1)c1ccccc1Cl] | (600 MHz, CDCl₃) δ 8.09 (s, 1H), 7.99 (d, J = 8.3 Hz, 2H), 7.81 (d, J = 7.6 Hz, 1H), 7.76 (d, J = 8.4 Hz, 1H), 7.74-7.71 (m, 1H), 7.45-7.40 (m, 3H), 7.38 (td, J = 7.6, 1.7 Hz, 1H), 7.35 (qd, J = 7.7, 7.3, 4.0 Hz, 4H). | (151 MHz, CDCl₃) δ 132.19, 130.87, 130.61, 130.56, 127.88, 127.51, 120.07, 29.73. |
| 123 | SW394598 | 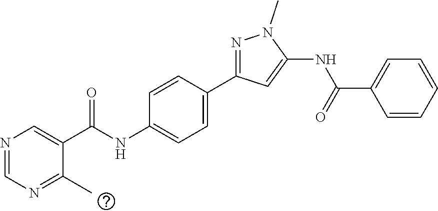 [SMILES: Cc1ncncc1C(=O)Nc1ccc(-c2cc(NC(=O)c3ccccc3)n(C)n2)cc1] | (600 MHz, DMSO-d₆) δ 10.70 (s, 1H), 10.44 (s, 1H), 9.16 (s, 1H), 8.91 (s, 1H), 8.03-8.00 (m, 2H), 7.81 (d, J = 8.8 Hz, 2H), 7.77 (d, J = 8.8 Hz, 2H), 7.66-7.63 (m, 1H), 7.57 (dd, J = 8.4, 7.0 Hz, 2H), 6.71 (s, 1H), 3.76 (s, 3H), 2.61 (s, 3H). | (151 MHz, DMSO) δ 165.8, 164.6, 164.0, 158.5, 155.1, 147.9, 138.1, 137.6, 133.3, 132.3, 130.1, 129.4, 128.6, 128.0, 125.3, 119.9, 97.7, 35.9, 22.3. |
| 124 | SW394599 | 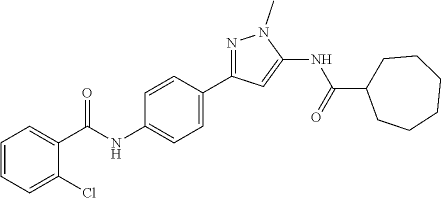 [SMILES: Cn1nc(-c2ccc(NC(=O)c3ccccc3Cl)cc2)cc1NC(=O)C1CCCCCC1] | (600 MHz, DMSO-d₆) δ 10.56 (s, 1H), 9.85 (s, 1H), 7.76-7.71 (m, 4H), 7.59 (ddd, J = 14.0, 7.7, 1.5 Hz, 2H), 7.51 (td, J = 7.7, 1.8 Hz, 1H), 7.46 (td, J = 7.4, 1.3 Hz, 1H), 6.59 (s, 1H), 3.68 (s, 3H), 2.60 (tt, J = 9.4, 4.2 Hz, 1H), 1.88 (ddd, J = 14.0, 7.0, 3.3 Hz, 2H), 1.73 (ddt, J = 13.4, 6.7, 3.6 Hz, 2H), 1.68-1.44 (m, 8H). | (151 MHz, DMSO) δ 175.4, 164.9, 147.8, 138.2, 137.8, 137.0, 131.1, 130.0, 129.7, 129.1, 129.0, 127.3, 125.2, 119.6, 96.0, 45.3, 35.7, 31.0, 27.9, 26.1. |
| 125 | SW394600 | 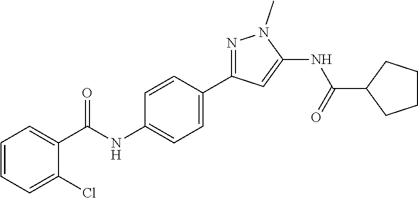 [SMILES: Cn1nc(-c2ccc(NC(=O)c3ccccc3Cl)cc2)cc1NC(=O)C1CCCC1] | (600 MHz, DMSO-d₆) δ 10.57 (s, 1H), 9.99 (s, 1H), 7.77-7.71 (m, 4H), 7.59 (dd, J = 15.2, 7.7 Hz, 2H), 7.51 (t, J = 7.6 Hz, 1H), 7.46 (t, J = 7.5 Hz, 1H), 6.61 (s, 1H), 3.70 (s, 3H), 2.95- 2.85 (m, 1H), 1.92-1.85 (m, 2H), 1.71 (dq, J = 35.0, 7.1 Hz, 4H), 1.61- 1.53 (m, 2H), 1.26 (dd, J = 18.4, 6.9 Hz, 2H). | (151 MHz, DMSO) δ 174.5, 164.9, 147.8, 138.2, 137.9, 137.0, 131.1, 130.0., 129.7, 129.1, 129.0, 127.3, 125.2, 119.7, 95.9, 53.4, 44.3, 35.7, 30.1, 25.7. |
| 126 | SW394601 | 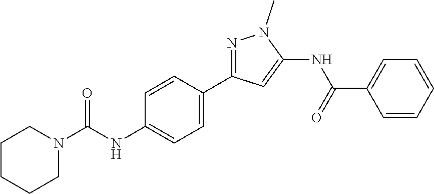 [SMILES: Cn1nc(-c2ccc(NC(=O)N3CCCCC3)cc2)cc1NC(=O)c1ccccc1] | (600 MHz, CDCl₃) δ 8.09 (s, 1H), 7.99 (d, J = 8.3 Hz, 2H), 7.81 (d, J = 7.6 Hz, 1H), 7.76 (d, J = 8.4 Hz, 1H), 7.74-7.71 (m, 1H), 7.45-7.40 (m, 3H), 7.38 (td, J = 7.6, 1.7 Hz, 1H), 7.35 (qd, J = 7.7, 7.3, 4.0 Hz, 4H). | (151 MHz, CDCl₃) δ 169.22, 166.85, 149.23, 136.93, 136.69, 133.01, 132.42, 129.80, 128.66, 127.80, 125.94, 119.84, 98.09, 62.48, 55.18, 54.88, 43.20, 35.85, 29.71, 26.11, 23.49, 17.62, 12.61. |
| 127 | SW394602 | 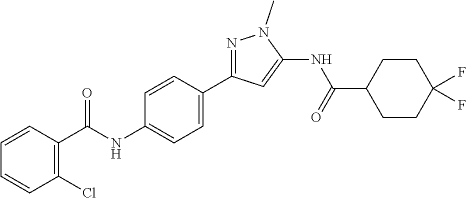 [SMILES: Cn1nc(-c2ccc(NC(=O)c3ccccc3Cl)cc2)cc1NC(=O)C1CCC(F)(F)CC1] | (600 MHz, DMSO-d₆) δ 10.57 (s, 1H), 10.04 (s, 1H), 7.74 (s, 4H), 7.61- 7.56 (m, 2H), 7.51 (td, J = 7.7, 1.8 Hz, 1H), 7.46 (t, J = 7.5 Hz, 1H), 6.62 (s, 1H), 3.70 (s, 3H), 2.64-2.56 (m, 1H), 2.15-2.07 (m, 2H), 2.00-1.81 (m, 4H), 1.71 (dh, J = 14.7, 3.7 Hz, 2H); | (151 MHz, DMSO) δ 172.9, 164.9, 147.9, 138.2, 137.6, 137.0, 131.1, 130.0, 129.7, 129.1, 129.0, 127.3, 125.2, 119.7, 96.0, 41.0, 35.7, 32.3, 32.2, 32.0, 25.6, 25.5; |
| 128 | SW394603 | 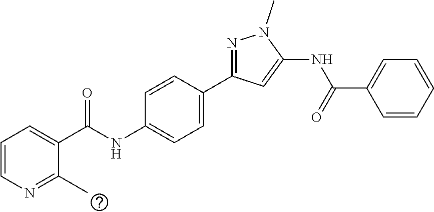 [SMILES: Cc1ncccc1C(=O)Nc1ccc(-c2cc(NC(=O)c3ccccc3)n(C)n2)cc1] | (600 MHz, DMSO-d₆) δ 10.77 (s, 1H), 10.44 (s, 1H), 8.87 (dd, J = 4.7, 1.6 Hz, 1H), 8.25 (dd, J = 7.8, 1.6 Hz, 1H), 8.01 (dd, J = 8.2, 1.3 Hz, 2H), 7.87 (dd, J = 7.8, 4.7 Hz, 1H), 7.82- 7.79 (m, 2H), 7.73-7.70 (m, 2H), 7.67-7.62 (m, 1H), 7.57 (dd, J = 8.3, 7.0 Hz, 2H), 6.71 (s, 1H), 3.76 (s, 3H). | (151 MHz, DMSO) δ 166.17, 164.24, 150.59, 148.32, 142.98, 142.76, 142.54, 138.40, 138.02, 137.93, 133.77, 132.79, 132.68, 129.86, 129.05, 128.39, 127.75, 125.77, 122.84, 121.02, 120.22, 98.10, 36.33. |
| 129 | SW394628 (HCl salt: SW394677) | 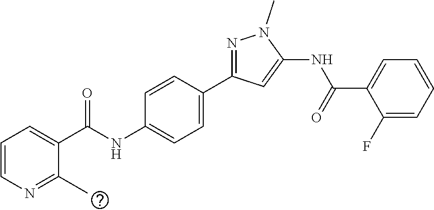 [SMILES: Cc1ncccc1C(=O)Nc1ccc(-c2cc(NC(=O)c3ccccc3F)n(C)n2)cc1] | (600 MHz, DMSO-d₆) δ 10.54 (s, 1H), 10.53 (s, 1H), 8.57 (dd, J = 4.9, 1.8 Hz, 1H), 7.89 (dd, J = 7.7, 1.8 Hz, 1H), 7.81-7.75 (m, 5H), 7.66-7.61 (m, 1H), 7.42-7.34 (m, 3H), 6.77 (s, 1H), 3.78 (s, 3H), 2.59 (s, 3H). | (151 MHz, DMSO) δ 166.6, 162.8, 160.1, 158.4, 155.1, 149.8, 148.1, 138.4, 137.1, 135.2, 133.3, 133.3, 132.3, 130.3, 130.3, 129.0, 125.3, 124.7, 124.7, 123.5, 123.4, 121.0, 119.8, 116.4, 116.3, 96.8, 35.8, 22.7. |
| 130 | SW394629 | 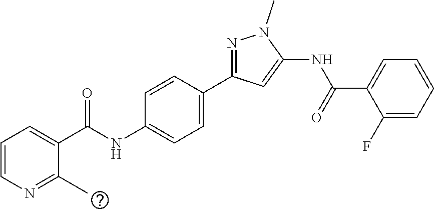 [SMILES: Cc1ncccc1C(=O)Nc1ccc(-c2cc(NC(=O)c3ccccc3F)n(C)n2)cc1] | (600 MHz, DMSO-d₆) δ 10.77 (s, 1H), 10.55 (s, 1H), 8.87 (dd, J = 4.8, 1.5 Hz, 1H), 8.25 (dd, J = 7.9, 1.6 Hz, 1H), 7.87 (dd, J = 7.8, 4.8 Hz, 1H), 7.81 (d, J = 8.7 Hz, 2H), 7.78 (td, J = 7.5, 1.8 Hz, 1H), 7.74-7.70 (m, 2H), 7.64 (tdd, J = 8.2, 6.2, 1.8 Hz, 1H), 7.43-7.35 (m, 2H), 6.77 (s, 1H), 3.78 (s, 3H). | (151 MHz, DMSO) δ 163.8, 162.7, 160.1, 158.4, 150.2, 148.0, 142.5, 142.3, 138.0, 137.5, 137.1, 133.3, 133.3, 132.4, 130.3, 130.3, 129.3, 127.3, 125.4, 124.7, 124.7, 123.5, 123.4, 122.4, 120.6, 119.8, 116.4, 116.3, 96.8, 35.8. |
| 131 | SW394630 | 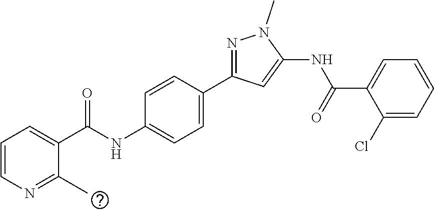 [SMILES: Cc1ncccc1C(=O)Nc1ccc(-c2cc(NC(=O)c3ccccc3Cl)n(C)n2)cc1] | (400 MHz, DMSO-d₆) δ 10.71 (s, 1H), 10.53 (s, 1H), 8.57 (dd, J = 5.0, 1.8 Hz, 1H), 7.89 (dd, J = 7.7, 1.8 Hz, 1H), 7.79 (s, 4H), 7.68 (dd, J = 7.4, 1.8 Hz, 1H), 7.61 (dd, J = 8.0, 1.4 Hz, 1H), 7.55 (td, J = 7.6, 1.8 Hz, 1H), 7.49 (td, J = 7.3, 1.4 Hz, 1H), 7.36 (dd, J = 7.7, 4.9 Hz, 1H), 6.77 (s, 1H), 3.80 (s, 3H), 2.59 (s, 3H). | (101 MHz, dmso) δ 166.6, 165.0, 155.1, 149.8, 148.1, 138.4, 137.1, 135.9, 135.2, 132.3, 131.6, 130.1, 129.8, 129.3, 129.0, 127.4, 125.3, 121.0, 119.8, 96.5, 36.0, 22.7. |
| 132 | SW394631 | 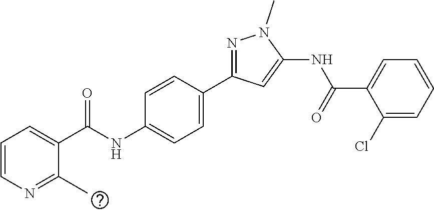 [SMILES: Cc1ncccc1C(=O)Nc1ccc(-c2cc(NC(=O)c3ccccc3Cl)n(C)n2)cc1] | (600 MHz, DMSO-d₆) δ 10.77 (s, 1H), 10.72 (s, 1H), 8.88 (dd, J = 4.9, 1.5 Hz, 1H), 8.25 (dd, J = 8.0, 1.6 Hz, 1H), 7.87 (dd, J = 7.8, 4.7 Hz, 1H), 7.82-7.80 (m, 2H), 7.74-7.70 (m, 2H), 7.68 (dd, J = 7.5, 1.7 Hz, 1H), 7.61 (dd, J = 8.0, 1.2 Hz, 1H), 7.55 (td, J = 7.7, 1.8 Hz, 1H), 7.50 (td, J = 7.4, 1.3 Hz, 1H), 6.78 (s, 1H), 3.81 (s, 3H). | (151 MHz, DMSO) δ 165.0, 163.8, 150.2, 148.0, 142.6, 142.3, 138.0, 137.5, 137.0, 135.9, 132.4, 131.6, 130.1, 129.8, 129.2, 129.3, 127.4, 127.3, 125.4, 122.4, 120.6, 119.8, 96.5, 36.0. |
| 133 | SW394656 | 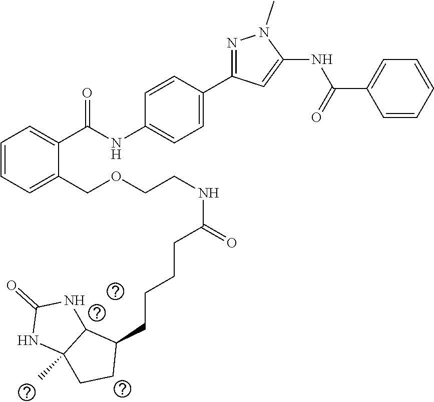 [SMILES: Cn1nc(-c2ccc(NC(=O)c3ccccc3COCCNC(=O)CCCC[C@H]3CC[C@@]4(C)NC(=O)NC34)cc2)cc1NC(=O)c1ccccc1] | (600 MHz, CDCl₃) δ 7.93 (d, J = 7.6 Hz, 2H), 7.71 (dd, J = 8.6, 2.2 Hz, 2H), 7.64-7.59 (m, 3H), 7.52 (dd, J = 8.5, 6.4 Hz, 1H), 7.44 (td, J = 7.8, 1.9 Hz, 2H), 7.39 (t, J = 7.9 Hz, 2H), 7.33 (d, J = 7.1 Hz, 2H), 6.85 (t, J = 5.6 Hz, 1H), 6.55 (d, J = 2.1 Hz, 1H), 4.65 (d, J = 2.1 Hz, 2H), 4.34 (dd, J = 7.3, 4.3 Hz, 1H), 4.10 (ddd, J = 7.4, 4.5, 2.0 Hz, 1H), 3.77 (d, J = 2.1 Hz, 3H), 3.53 (d, J = 5.5 Hz, 2H), 2.99 (dt, J = 10.1, 5.8 Hz, 1H), 2.78 (ddd, J = 13.0, 5.0, 2.0 Hz, 1H), 2.58 (d, J = 12.9 Hz, 1H), 1.89 (d, J = 9.1 Hz, 3H), 1.42 (dtq, J = 28.8, 14.9, 8.2, 7.6 Hz, 4H), 0.83-0.74 (m, 3H). | (151 MHz, CDCl₃) δ 174.25, 167.10, 163.93, 139.54, 137.86, 137.44, 136.53, 132.43, 130.27, 130.03, 129.46, 128.64, 127.72, 126.97, 126.07, 125.87, 120.33, 98.00, 77.15, 76.94, 61.75, 59.96, 55.22, 49.43, 49.28, 49.14, 49.04, 49.00, 48.90, 48.86, 48.71, 48.57, 40.26, 38.30, 35.94, 35.58, 30.46, 29.69, 28.28, 27.80, 25.27. |
| 134 | SW394657 | 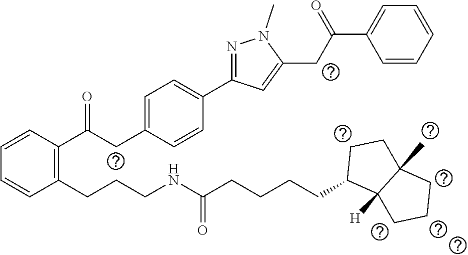 [SMILES: Cn1nc(-c2ccc(CC(=O)c3ccccc3CCCNC(=O)CCCC[C@@H]3CC[C@]4(C)CCC[C@H]34)cc2)cc1CC(=O)c1ccccc1] | | |
| 135 | SW394692 | 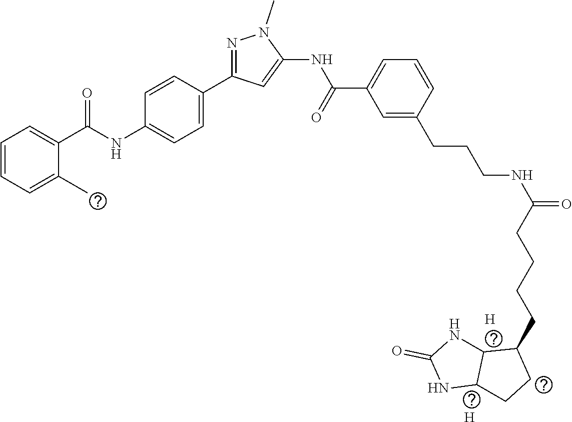 | (600 MHz, MeOD) δ 7.75 (s, 1H), 7.73 (d, J = 7.5 Hz, 1H), 7.71-7.68 (m, 2H), 7.66-7.63 (m, 2H), 7.48 (dd, J = 7.4, 1.8 Hz, 1H), 7.43 (dd, J = 8.0, 1.3 Hz, 1H), 7.42-7.36 (m, 3H), 7.34 (td, J = 7.4, 1.4 Hz, 1H), 6.56 (s, 1H), 4.35 (dd, J = 7.9, 4.9 Hz, 1H), 4.17 (dd, J = 7.9, 4.5 Hz, 1H), 3.73 (s, 3H), 3.14 (td, J = 6.8, 3.2 Hz, 2H), 3.11-3.07 (m, 1H), 2.80 (dd, J = 12.8, 5.0 Hz, 1H), 2.67 (t, J = 7.7 Hz, 2H), 2.58 (d, J = 12.7 Hz, 1H), 2.12 (td, J = 7.3, 1.6 Hz, 2H), 1.79 (q, J = 7.1 Hz, 3H), 1.39-1.26 (m, 3H). | |
| 136 | SW394693 | 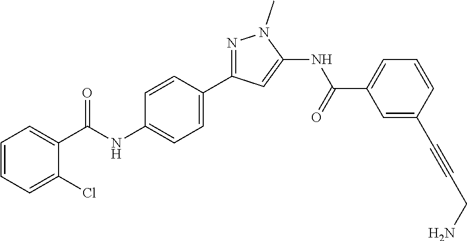 [SMILES: Cn1nc(-c2ccc(NC(=O)c3ccccc3Cl)cc2)cc1NC(=O)c1cccc(C#CCN)c1] | (600 MHz, MeOD) δ 8.05 (d, J = 1.7 Hz, 1H), 7.96 (dd, J = 9.3, 7.6 Hz, 2H), 7.67 (d, J = 7.7 Hz, 1H), 7.54- 7.45 (m, 3H), 7.43 (dd, J = 8.1, 1.3 Hz, 2H), 7.41-7.37 (m, 2H), 7.34 (td, J = 7.4, 1.4 Hz, 2H), 6.79 (s, 1H), 3.99 (s, 3H), 3.83 (d, J = 1.5 Hz, 5H), 3.68-3.61 (m, 2H). | (151 MHz, MeOD) δ 166.84, 166.26, 139.60- 139.14 (m), 136.52, 135.32, 133.48, 131.20, 131.06, 130.64, 129.71, 128.91, 128.53, 128.33, 126.89, 126.15, 122.21, 120.22, 97.94, 85.19, 81.38, 72.17, 71.05, 60.78, 42.35, 34.83, 29.31. |
| 137 | SW394694 | 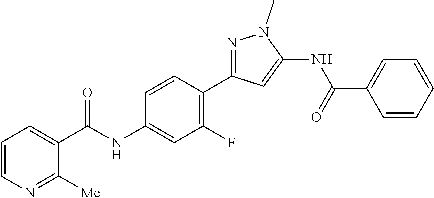 [SMILES: Cc1ncccc1C(=O)Nc1ccc(-c2cc(NC(=O)c3ccccc3)n(C)n2)c(F)c1] | (600 MHz, Methanol-d₄) δ 8.49 (dd, J = 5.0, 1.7 Hz, 1H), 7.97-7.94 (m, 2H), 7.88-7.82 (m, 2H), 7.75 (dd, J = 13.1, 2.1 Hz, 1H), 7.60-7.56 (m, 1H), 7.50 (t, J = 7.8 Hz, 2H), 7.37 (dd, J = 8.5, 2.1 Hz, 1H), 7.30 (dd, J = 7.7, 5.0 Hz, 1H), 6.67 (d, J = 3.4 Hz, 1H), 3.82 (s, 3H), 2.67 (s, 3H). | (151 MHz, MeOD) δ 168.5, 168.1, 161.3, 159.7, 156.4, 150.1, 145.0, 139.8, 139.8, 137.8, 136.4, 133.5, 133.1, 129.2, 128.6, 128.5, 128.3, 121.8, 117.7, 117.6, 116.3, 116.3, 108.6, 108.4, 102.1, 102.1, 36.0, 22.4; |
| 138 | SW394695 | 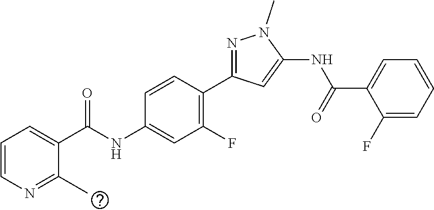 [SMILES: Cc1ncccc1C(=O)Nc1ccc(-c2cc(NC(=O)c3ccccc3F)n(C)n2)c(F)c1] | (600 MHz, Methanol-d₄) δ 8.47 (dd, J = 5.0, 1.8 Hz, 1H), 7.90 (td, J = 7.6, 1.9 Hz, 1H), 7.86-7.81 (m, 2H), 7.74 (dd, J = 13.0, 2.1 Hz, 1H), 7.58-7.53 (m, 1H), 7.36 (dd, J = 8.5, 2.1 Hz, 1H), 7.31-7.25 (m, 2H), 7.23-7.18 (m, 1H), 6.75 (d, J = 3.4 Hz, 1H), 3.83 (s, 3H), 2.66 (s, 3H). | (151 MHz, MeOD) δ 167.8, 163.9, 163.8, 161.6, 161.1, 159.9, 159.5, 156.3, 149.9, 145.0, 139.6, 139.6, 136.9, 136.9, 136.1, 134.5, 134.5, 132.8, 131.6, 131.6, 128.4, 128.4, 125.2, 125.2, 121.6, 121.6, 121.5, 117.4, 117.3, 116.8, 116.6, 116.1, 116.1, 108.4, 108.2, 101.4, 101.4, 35.8, 22.4. |
| 139 | SW394696 | 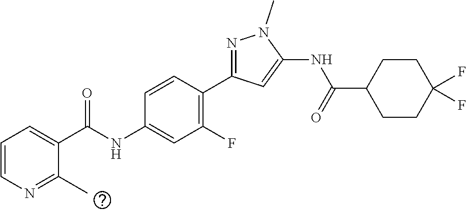 [SMILES: Cc1ncccc1C(=O)Nc1ccc(-c2cc(NC(=O)C3CCC(F)(F)CC3)n(C)n2)c(F)c1] | (600 MHz, DMSO-d₆) δ 10.71 (s, 1H), 10.08 (s, 1H), 8.57 (dd, J = 4.9, 1.7 Hz, 1H), 7.91-7.85 (m, 2H), 7.77 (dd, J = 13.7, 2.1 Hz, 1H), 7.51 (dd, J = 8.5, 2.0 Hz, 1H), 7.36 (dd, J = 7.7, 4.9 Hz, 1H), 6.57 (d, J = 3.9 Hz, 1H), 3.73 (s, 3H), 2.66-2.55 (m, 4H), 2.16- 2.07 (m, 2H), 2.00-1.81 (m, 4H), 1.74-1.66 (m, 2H). | (151 MHz, DMSO) δ 172.9, 166.8, 159.8, 158.1, 155.2, 150.0, 142.6, 137.4, 135.3, 131.9, 127.7, 127.7, 125.3, 123.7, 121.0, 116.3, 116.3, 115.6, 107.0, 106.9, 98.8, 98.7, 41.0, 35.8, 32.3, 32.2, 32.0, 25.6, 25.5, 22.7. |
| 140 | SW394697 | 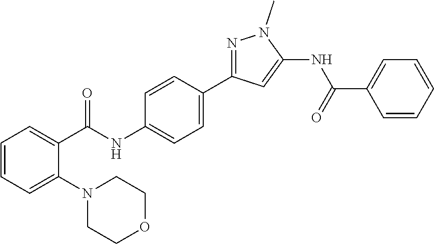 [SMILES: Cn1nc(-c2ccc(NC(=O)c3ccccc3N3CCOCC3)cc2)cc1NC(=O)c1ccccc1] | (600 MHz, Chloroform-d) δ 11.97 (s, 1H), 8.85 (s, 1H), 8.08 (dd, J = 7.8, 1.7 Hz, 1H), 7.86 (d, J = 7.6 Hz, 2H), 7.63-7.57 (m, 4H), 7.46-7.40 (m, 2H), 7.31 (t, J = 7.8 Hz, 2H), 7.22- 7.16 (m, 2H), 6.37 (s, 1H), 3.89- 3.76 (m, 4H), 3.65 (s, 3H), 2.98- 2.88 (m, 4H). | (151 MHz, CDCl₃) δ 166.6, 164.4, 150.6, 149.3, 137.6, 136.8, 133.0, 132.9, 132.5, 131.8, 129.7, 128.8, 127.8, 126.2, 125.7, 120.8, 120.1, 98.21, 67.3, 53.7, 35.9. |
| 141 | SW394698 | 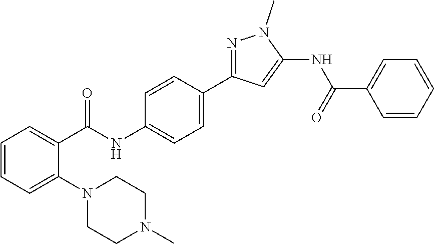 [SMILES: CN1CCN(c2ccccc2C(=O)Nc2ccc(-c3cc(NC(=O)c4ccccc4)n(C)n3)cc2)CC1] | (600 MHz, Methanol-d₄) δ 8.06 (dd, J = 7.8, 1.7 Hz, 1H), 7.98-7.95 (m, 2H), 7.76 (s, 4H), 7.59-7.55 (m, 1H), 7.52-7.45 (m, 3H), 7.33-7.30 (m, 1H), 7.25 (td, J = 7.6, 1.1 Hz, 1H), 6.59 (s, 1H), 3.80 (s, 3H), 3.09 (t, J = 4.8 Hz, 4H), 2.69 (s, 4H), 2.38 (s, 3H). | (151 MHz, MeOD) δ 168.3, 165.8, 151.3, 150.3, 138.5, 138.2, 133.7, 133.4, 133.1, 131.8, 129.9, 129.2, 128.4, 128.3, 126.7, 125.7, 121.3, 120.7, 98.70, 78.5, 78.2, 78.0, 55.9, 53.4, 46.2, 36.0. |
| 142 | SW394699 | 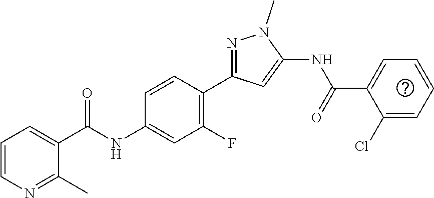 [SMILES: Cc1ncccc1C(=O)Nc1ccc(-c2cc(NC(=O)c3ccccc3Cl)n(C)n2)c(F)c1] | (600 MHz, MeOD) δ 8.51 (d, J = 5.2 Hz, 1H), 7.91 (d, J = 7.7 Hz, 1H), 7.85 (t, J = 8.5 Hz, 1H), 7.76 (d, J = 13.1 Hz, 1H), 7.62 (d, J = 7.5 Hz, 1H), 7.51-7.31 (m, 5H), 6.78 (d, J = 3.3 Hz, 1H), 3.88 (d, J = 3.0 Hz, 3H), 2.70 (s, 3H). | (151 MHz, DMSO) δ 166.87, 164.93, 159.81, 158.18, 155.17, 150.02, 142.80, 139.75, 139.68, 136.88, 135.79, 135.32, 131.89, 131.61, 130.10, 129.80, 129.28, 127.79, 127.76, 127.35, 121.01, 116.23, 116.15, 115.65, 107.07, 106.89, 99.30, 99.24, 36.12, 22.67. |
| 143 | SW394738 | 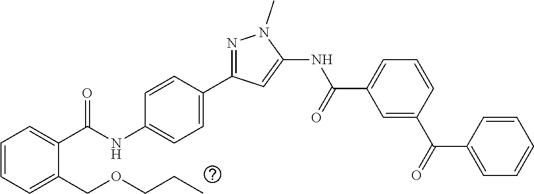 [SMILES: CCCOCc1ccccc1C(=O)Nc1ccc(-c2cc(NC(=O)c3cccc(C(=O)c4ccccc4)c3)n(C)n2)cc1] | (600 MHz, CDCl₃) δ 8.93 (s, 1H), 8.75 (s, 1H), 8.29 (s, 1H), 8.09 (d, J = 7.7 Hz, 1H), 7.84 (dt, J = 7.7, 1.4 Hz, 1H), 7.66 (ddd, J = 9.5, 7.8, 1.4 Hz, 3H), 7.64-7.60 (m, 2H), 7.58-7.51 (m, 3H), 7.44 (td, J = 7.8, 3.4 Hz, 1H), 7.42-7.36 (m, 3H), 7.35-7.30 (m, 2H), 6.41 (s, 1H), 4.62 (s, 2H), 3.69 (d, J = 2.3 Hz, 3H), 3.62 (dd, J = 5.6, 4.4 Hz, 2H), 3.38 (dd, J = 5.5, 4.4 Hz, 2H). | (151 MHz, CDCl₃) δ 195.98, 166.96, 166.69, 165.39, 149.46, 149.43, 137.98, 137.69, 137.43, 137.05, 136.77, 136.57, 136.30, 133.97, 133.70, 133.50, 133.37, 133.07, 132.91, 131.71, 131.46, 131.36, 130.90, 130.87, 130.77, 130.26, 130.07, 129.93, 129.76, 129.43, 129.20, 128.96, 128.89, 128.67, 128.58, 127.56, 127.26, 126.06, 120.39, 120.13, 98.18, 73.73, 71.90, 71.24, 69.52, 68.84, 58.28, 50.89, 50.81, 35.99, 29.73, 28.35. |
| 144 | SW394739 | 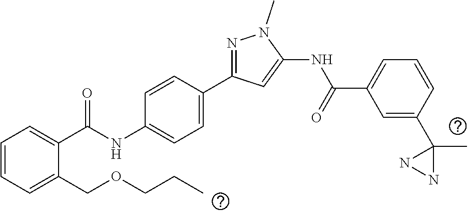 [SMILES: CCCOCc1ccccc1C(=O)Nc1ccc(-c2cc(NC(=O)c3cccc(C4(C)NN4)c3)n(C)n2)cc1] | (600 MHz, CDCl₃) δ 8.93 (s, 1H), 8.75 (s, 1H), 8.29 (s, 1H), 8.09 (d, J = 7.7 Hz, 1H), 7.84 (dt, J = 7.7, 1.4 Hz, 1H), 7.66 (ddd, J = 9.5, 7.8, 1.4 Hz, 3H), 7.64-7.60 (m, 2H), 7.58-7.51 (m, 3H), 7.44 (td, J = 7.8, 3.4 Hz, 1H), 7.42-7.36 (m, 3H), 7.35-7.30 (m, 2H), 6.41 (s, 1H), 4.62 (s, 2H), 3.69 (d, J = 2.3 Hz, 3H), 3.62 (dd, J = 5.6, 4.4 Hz, 2H), 3.38 (dd, J = 5.5, 4.4 Hz, 2H). | (151 MHz, CDCl₃) δ 167.02, 166.76, 165.07, 149.40, 137.30, 137.08, 136.66, 136.11, 136.09, 133.95, 133.82, 133.31, 131.55, 130.92, 130.35, 130.29, 130.05, 130.03, 129.51, 129.36, 129.23, 129.05, 128.53, 126.05, 125.98, 122.82, 121.00, 120.47, 120.20, 98.22, 73.74, 71.96, 68.82, 60.46, 58.28, 50.81, 36.01, 29.73, 28.45, 28.18, 14.22. |
| 145 | SW394740 | 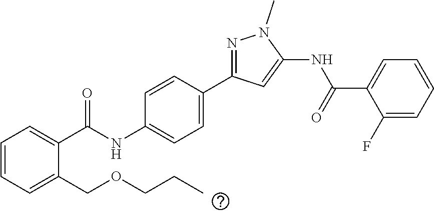 [SMILES: CCCOCc1ccccc1C(=O)Nc1ccc(-c2cc(NC(=O)c3ccccc3F)n(C)n2)cc1] | (600 MHz, MeOD) δ 7.75 (td, J = 7.5, 1.9 Hz, 1H), 7.70 (d, J = 8.5 Hz, 2H), 7.65 (d, J = 8.5 Hz, 2H), 7.56-7.49 (m, 2H), 7.46 (dd, J = 7.7, 1.4 Hz, 1H), 7.43 (td, J = 7.5, 1.4 Hz, 1H), 7.37 (td, J = 7.4, 1.5 Hz, 1H), 7.26 (t, J = 7.5 Hz, 1H), 7.21 (dd, J = 10.9, 8.4 Hz, 1H), 6.64 (s, 1H), 4.69 (s, 2H), 3.75 (s, 3H), 3.61-3.57 (m, 2H), 3.01-2.96 (m, 2H). | (151 MHz, MeOD) δ 169.38, 149.58, 138.20, 137.25, 136.27, 135.54, 133.63 (d, J = 8.8 Hz), 130.38 (d, J = 2.0 Hz), 130.05, 129.27, 128.00, 127.33, 125.60, 124.50 (d, J = 3.6 Hz), 120.28, 116.06 (d, J = 22.8 Hz), 97.30, 70.62, 65.82, 39.16, 34.65. |
| 146 | SW394741 | 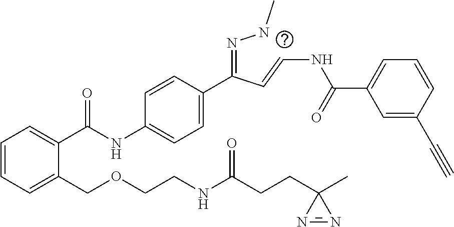 [SMILES: C#Cc1cccc(C(=O)N/C=C/C(=N\NC)c2ccc(NC(=O)c3ccccc3COCCNC(=O)CCC3(C)N=N3)cc2)c1] | (400 MHz, CDCl₃) δ 8.78 (s, 1H), 8.67 (s, 1H), 7.96 (d, J = 1.8 Hz, 1H), 7.81 (d, J = 7.8 Hz, 1H), 7.65-7.54 (m, 4H), 7.49 (d, J = 8.3 Hz, 2H), 7.32 (tdd, J = 18.1, 7.4, 3.7 Hz, 4H), 6.38 (s, 1H), 4.56 (s, 2H), 3.66 (s, 3H), 3.48 (t, J = 5.1 Hz, 2H), 3.29 (q, J = 5.3 Hz, 2H), 3.07 (s, 1H), 1.79 (dd, J = 8.8, 6.7 Hz, 2H), 1.53 (dd, J = 8.7, 6.7 Hz, 2H), 0.85 (s, 3H). | (151 MHz, CDCl₃) δ 171.82, 167.54, 165.52, 149.28, 137.34, 136.50, 136.38, 135.91, 135.80, 135.67, 134.63, 133.27, 131.25, 131.17, 130.68, 130.04, 128.98, 128.87, 128.10, 128.00, 126.21, 126.11, 122.90, 120.49, 120.35, 98.18, 82.42, 78.80, 78.75, 71.65, 69.11, 39.19, 35.95, 35.92, 30.40, 29.93, 25.49, 19.77. |
| 147 | SW394742 | 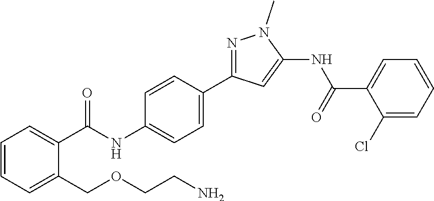 [SMILES: Cn1nc(-c2ccc(NC(=O)c3ccccc3COCCN)cc2)cc1NC(=O)c1ccccc1Cl] | (600 MHz, MeOD) δ 7.70 (d, J = 8.8 Hz, 2H), 7.65 (d, J = 8.7 Hz, 2H), 7.56 (dd, J = 7.6, 1.7 Hz, 1H), 7.52 (dd, J = 7.5, 1.4 Hz, 1H), 7.46 (dt, J = 8.0, 1.5 Hz, 2H), 7.43 (td, J = 7.7, 1.6 Hz, 2H), 7.38 (tt, J = 7.4, 1.8 Hz, 2H), 6.65 (s, 1H), 4.70 (s, 2H), 3.78 (s, 3H), 3.62- 3.57 (m, 2H), 3.01-2.97 (m, 2H). | (151 MHz, MeOD) δ 169.39, 166.94, 149.64, 138.22, 137.09, 136.27, 135.44 (d, J = 30.8 Hz), 131.51, 130.73, 130.06, 129.84, 129.48, 129.28, 128.81, 128.01, 127.33, 126.99, 125.61, 120.29, 97.03, 70.63, 65.89, 48.03, 47.89, 47.75, 47.61, 47.46, 47.32, 47.18, 39.19, 34.88. |
| 148 | SW394743 | 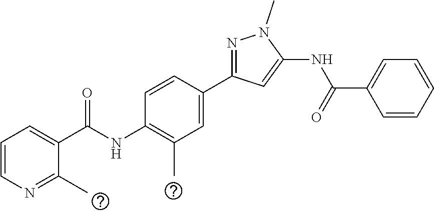 [SMILES: Cc1cc(-c2cc(NC(=O)c3ccccc3)n(C)n2)ccc1NC(=O)c1cccnc1C] | (600 MHz, Methanol-d₄) δ 8.49 (dd, J = 5.1, 1.8 Hz, 1H), 8.03 (t, J = 8.4 Hz, 1H), 7.97-7.94 (m, 2H), 7.89 (dd, J = 7.8, 1.8 Hz, 1H), 7.61-7.52 (m, 3H), 7.49 (t, J = 7.8 Hz, 2H), 7.30 (dd, J = 7.7, 5.0 Hz, 1H), 6.60 (s, 1H), 3.81 (s, 3H), 2.69 (s, 3H). | (151 MHz, Methanol-d₄) δ 168.3 (d, J = 9.3 Hz), 156.5, 156.0, 154.4, 150.1, 149.1 (d, J = 2.6 Hz), 138.3, 136.5, 133.5, 133.0, 132.7, 132.4 (d, J = 7.7 Hz), 129.2, 128.3, 125.3 (d, J = 12.0 Hz), 121.8, 112.8 (d, J = 21.3 Hz), 98.9, 36.1, 22.5. |
| 149 | SW394744 | 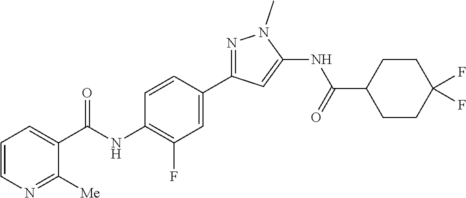 [SMILES: Cc1ncccc1C(=O)Nc1ccc(-c2cc(NC(=O)C3CCC(F)(F)CC3)n(C)n2)cc1F] | (600 MHz, Methanol-d₄) δ 8.49 (dd, J = 5.0, 1.8 Hz, 1H), 8.03 (t, J = 8.1 Hz, 1H), 7.89 (dd, J = 7.7, 1.8 Hz, 1H), 7.55-7.50 (m, 2H), 7.31 (dd, J = 7.7, 5.0 Hz, 1H), 6.57 (s, 1H), 3.75 (s, 3H), 2.69 (s, 3H), 2.56-2.48 (m, 1H), 2.21-2.12 (m, 2H), 2.03-1.95 (m, 2H), 1.94-1.74 (m, 4H). | (151 MHz, Methanol-d₄) δ 175.1 (d, J = 2.1 Hz), 168.2, 156.5, 156.0, 154.4, 150.1, 149.1, 138.2, 136.5, 132.8, 132.4 (d, J = 7.5 Hz), 125.3 (d, J = 11.9 Hz), 125.0, 124.6, 123.0, 121.8, 121.4, 112.8 (d, J = 21.3 Hz), 97.7, 42.7, 35.8, 33.7- 32.5 (m), 26.3 (d, J = 9.2 Hz), 22.5. |
| 150 | SW394745 | 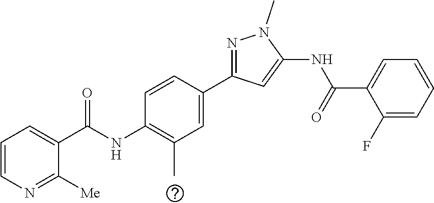 [SMILES: Cc1cc(-c2cc(NC(=O)c3ccccc3F)n(C)n2)ccc1NC(=O)c1cccnc1C] | (600 MHz, Methanol-d₄) δ 8.49 (dd, J = 5.1, 1.7 Hz, 1H), 8.07 (t, J = 8.1 Hz, 1H), 7.89 (td, J = 7.5, 1.8 Hz, 2H), 7.60-7.54 (m, 3H), 7.34-7.27 (m, 2H), 7.24-7.19 (m, 1H), 6.71 (s, 1H), 3.84 (s, 3H), 2.70 (s, 3H). | (151 MHz, Methanol-d₄) δ 168.1, 164.0 (d, J = 2.1 Hz), 161.6, 160.0, 156.5, 155.9, 154.2, 150.1, 149.1, 137.7, 136.5, 134.5 (d, J = 9.0 Hz), 132.7, 132.2 (d, J = 7.7 Hz), 131.6 (d, J = 2.0 Hz), 125.4, 125.3, 124.9, 122.0 (d, J = 12.8 Hz), 121.8 (d, J = 3.2 Hz), 121.7, 116.8 (d, J = 23.3 Hz), 112.8 (d, J = 21.2 Hz), 98.3, 35.9, 22.5. |
| 151 | SW394746 | 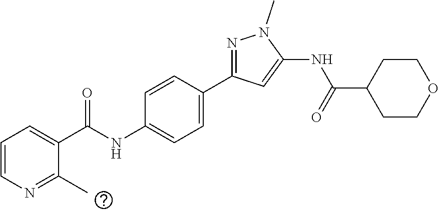 [SMILES: Cc1ncccc1C(=O)Nc1ccc(-c2cc(NC(=O)C3CCOCC3)n(C)n2)cc1] | (600 MHz, Methanol-d₄) δ 8.50 (s, 1H), 7.87 (dd, J = 7.8, 1.6 Hz, 1H), 7.73-7.67 (m, 4H), 7.32 (dd, J = 7.7, 4.9 Hz, 1H), 6.57 (s, 1H), 4.03 (ddd, J = 11.4, 4.4, 2.1 Hz, 2H), 3.75 (s, 3H), 3.49 (td, J = 11.6, 2.3 Hz, 2H), 2.73-2.67 (m, 1H), 1.89 (dtd, J = 13.5, 11.7, 4.4 Hz, 2H), 1.82 (ddt, J = 10.9, 4.2, 2.1 Hz, 2H). | (151 MHz, MeOD) δ 175.2, 168.1, 156.4, 150.4, 149.9, 138.6, 138.2, 136.5, 130.2, 126.5, 121.9, 121.0, 97.6, 67.7, 42.2, 35.7, 29.6, 22.4. |
| 152 | SW394763 | 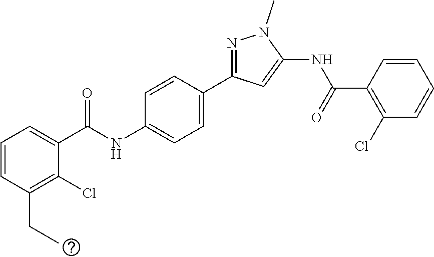 [SMILES: CCc1cccc(C(=O)Nc2ccc(-c3cc(NC(=O)c4ccccc4Cl)n(C)n3)cc2)c1Cl] | (600 MHz, CDCl₃) δ 8.04 (s, 1H), 7.84-7.77 (m, 1H), 7.77-7.71 (m, 2H), 7.65 (s, 1H), 7.63-7.59 (m, 2H), 7.58 (s, 1H), 7.46 (dd, J = 7.6, 1.7 Hz, 1H), 7.45-7.39 (m, 2H), 7.38-7.30 (m, 2H), 6.66 (s, 1H), 4.51 (s, 2H), 3.83 (s, 3H). | (151 MHz, CDCl₃) δ 164.52, 164.05, 149.60, 136.95, 136.91, 135.82, 134.83, 133.18, 132.59, 131.53, 131.29, 130.62, 130.60, 130.19, 130.13, 129.46, 127.64, 127.53, 126.20, 120.17, 97.36, 52.42, 36.05, 29.73, 17.47, 14.22. |
| 153 | SW394764 | 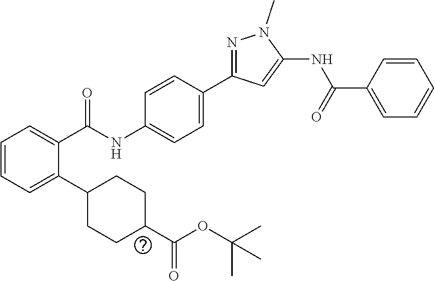 [SMILES: Cn1nc(-c2ccc(NC(=O)c3ccccc3C3CCC(C(=O)OC(C)(C)C)CC3)cc2)cc1NC(=O)c1ccccc1] | (600 MHz, MeOD) δ 7.97 (d, J = 7.7 Hz, 2H), 7.75 (s, 4H), 7.60 (t, J = 7.6 (Hz, 1H), 7.50 (t, J = 7.6 Hz, 2H), 7.43 (dt, J = 7.6, 3.8 Hz, 2H), 7.36 (d, J = 8.0 Hz, 1H), 7.27 (t, J = 7.5 Hz, 1H), 6.59 (s, 1H), 4.18 (d, J = 13.1 Hz, 2H), 3.81 (s, 3H), 3.37 (s, 1H), 3.14 (td, J = 15.4, 13.2, 7.4 Hz, 1H), 2.80 (d, J = 29.7 Hz, 2H), 1.88 (d, J = 12.7 Hz, 2H), 1.65 (qd, J = 12.6, 4.1 Hz, 2H), 1.48 (s, 9H). | (151 MHz, MeOD) δ 169.78, 167.73, 155.21, 149.74, 143.20, 138.17, 137.56, 136.60, 132.95, 132.48, 130.16, 129.41, 128.61, 127.78, 126.94, 126.52, 126.14, 125.90, 120.54, 120.45, 98.17, 79.92, 77.82, 77.61, 77.39, 54.58, 49.30, 43.87, 42.67, 38.78, 35.35, 33.04, 28.11, 12.43. |
| 154 | SW394765 | 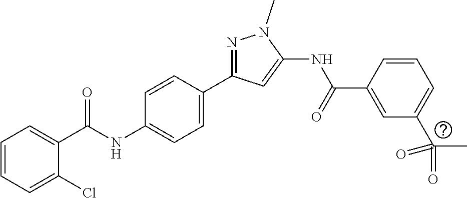 | (600 MHz, Methanol-d₄) δ 11.25 (s, 1H), 10.75 (s, 1H), 9.78 (d, J = 1.9 Hz, 1H), 9.54 (d, J = 7.7 Hz, 1H), 9.35 (dt, J = 7.8, 1.4 Hz, 1H), 9.05-8.94 (m, 5H), 8.79 (dd, J = 7.4, 1.7 Hz, 1H), 8.66-8.60 (m, 2H), 8.57 (td, J = 7.2, 1.8 Hz, 1H), 7.85 (s, 1H), 5.06 (s, 3H), 4.36 (s, 3H). | (151 MHz, MeOD) δ 175.2, 174.4, 158.9, 151.4, 148.1, 146.9, 146.6, 144.7, 143.0, 141.0, 140.7, 140.5, 139.8, 139.7, 139.0, 136.9, 136.6, 135.6, 129.9, 107.5, 53.7, 45.8. |
| 155 | SW394766 | 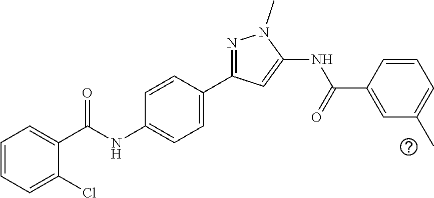 [SMILES: Cc1cccc(C(=O)Nc2cc(-c3ccc(NC(=O)c4ccccc4Cl)cc3)nn2C)c1] | (600 MHz, DMSO-d₆) δ 10.60 (s, 1H), 10.25 (s, 1H), 7.77 (s, 4H), 7.59 (dd, J = 17.5, 7.7 Hz, 2H), 7.51 (t, J = 7.7 Hz, 1H), 7.46 (t, J = 7.5 Hz, 1H), 7.16 (p, J = 7.7 Hz, 3H), 6.80 (d, J = 7.7 Hz, 1H), 6.66 (s, 1H), 5.38 (s, 2H), 3.73 (s, 3H). | (151 MHz, DMSO) δ 166.6, 165.0, 149.0, 147.9, 138.3, 137.8, 137.0, 134.2, 131.2, 130.0, 129.7, 129.2, 129.0, 127.3, 125.2, 119.7, 117.4, 115.0, 113.2, 97.7, 35.8. |
| 156 | SW394767 | 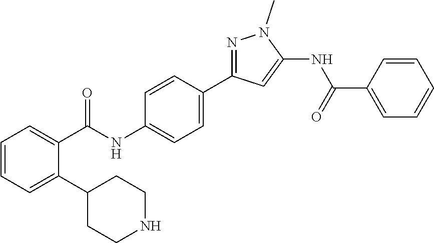 [SMILES: Cn1nc(-c2ccc(NC(=O)c3ccccc3C3CCNCC3)cc2)cc1NC(=O)c1ccccc1] | (600 MHz, MeOD) δ 7.95 (d, J = 7.6 Hz, 2H), 7.76-7.63 (m, 4H), 7.57 (t, J = 7.4 Hz, 1H), 7.48 (q, J = 7.4 Hz, 2H), 7.42 (dt, J = 20.9, 7.6 Hz, 3H), 7.27 (t, J = 7.3 Hz, 1H), 6.59 (s, 1H), 3.33 (s, 3H), 3.18 (dd, J = 40.9, 12.5 Hz, 3H), 2.78 (t, J = 12.4 Hz, 2H), 1.87 (dd, J = 79.9, 13.1 Hz, 4H). | (151 MHz, MeOD) δ 132.46, 128.66, 127.72, 126.00, 120.21, 97.97, 77.37, 77.15, 76.94, 45.80, 32.37. |
| 157 | SW394768 | 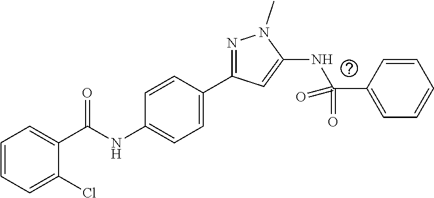 | (600 MHz, Methanol-d₄) δ 7.81-7.77 (m, 2H), 7.70-7.65 (m, 2H), 7.66- 7.60 (m, 1H), 7.60-7.56 (m, 2H), 7.56-7.49 (m, 3H), 7.44 (dd, J = 8.1, 1.3 Hz, 1H), 7.39 (td, J = 7.7, 1.8 Hz, 1H), 7.34 (td, J = 7.4, 1.3 Hz, 1H), 5.94 (s, 1H), 3.71 (s, 3H). | (151 MHz, MeOD) δ 167.4, 150.3, 139.8, 138.7, 137.0, 136.8, 134.0, 131.8, 131.6, 130.6, 129.8, 129.8, 129.4, 127.9, 127.5, 126.4, 121.1, 99.8, 35.5. |
| 158 | SW394770 | 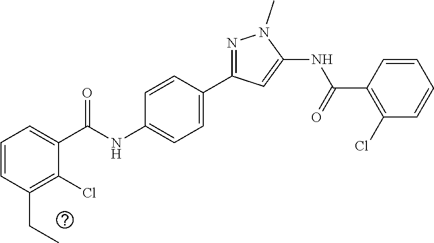 [SMILES: CCc1cccc(C(=O)Nc2ccc(-c3cc(NC(=O)c4ccccc4Cl)n(C)n3)cc2)c1Cl] | (600 MHz, CDCl₃) δ 8.62 (dd, J = 4.4, 1.4 Hz, 1H), 8.29 (dd, J = 8.4, 1.4 Hz, 1H), 8.01 (s, 1H), 7.86-7.81 (m, 2H), 7.76 (d, J = 8.4 Hz, 2H), 7.66- 7.59 (m, 3H), 7.54 (dd, J = 7.6, 1.7 Hz, 1H), 7.45-7.39 (m, 2H), 7.37 (td, J = 7.2, 2.0 Hz, 1H), 7.35-7.31 (m, 1H), 7.27 (t, J = 7.6 Hz, 1H), 6.67 (s, 1H), 5.76 (s, 2H), 3.84 (s, 3H). | (151 MHz, CDCl₃) δ 151.39, 132.62, 132.30, 131.34, 131.08, 130.63, 129.36, 127.66, 127.42, 126.22, 120.77, 120.11, 97.35, 79.46, 36.06. |
| 159 | SW394771 | 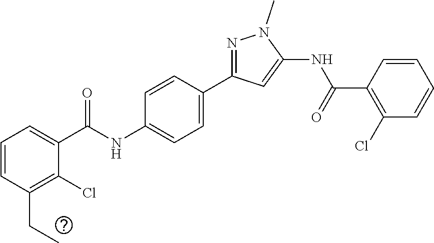 [SMILES: CCc1cccc(C(=O)Nc2ccc(-c3cc(NC(=O)c4ccccc4Cl)n(C)n3)cc2)c1Cl] | (600 MHz, MeOD) δ 7.72-7.66 (m, 2H), 7.66-7.62 (m, 2H), 7.58-7.54 (m, 1H), 7.54-7.50 (m, 1H), 7.49- 7.41 (m, 3H), 7.37 (tdd, J = 7.4, 5.5, 4.2 Hz, 2H), 6.65 (d, J = 3.0 Hz, 1H), 4.02 (d, J = 15.5 Hz, 2H), 3.78 (d, J = 1.8 Hz, 3H). | |
| 160 | SW394800 | 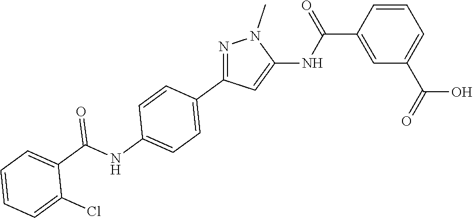 [SMILES: Cn1nc(-c2ccc(NC(=O)c3ccccc3Cl)cc2)cc1NC(=O)c1cccc(C(=O)O)c1] | (600 MHz, MeOD) δ 8.58 (t, J = 1.7 Hz, 1H), 8.20 (dd, J = 7.8, 1.5 Hz, 1H), 8.15-8.10 (m, 1H), 7.56 (t, J = 7.8 Hz, 1H), 7.49 (d, J = 8.3 Hz, 2H), 7.40 (dd, J = 8.1, 1.3 Hz, 1H), 7.35 (td, J = 7.7, 1.7 Hz, 1H), 7.31 (td, J = 7.4, 1.4 Hz, 1H), 6.64 (s, 1H), 3.81 (s, 3H). | |
| 161 | SW394868- 1 | 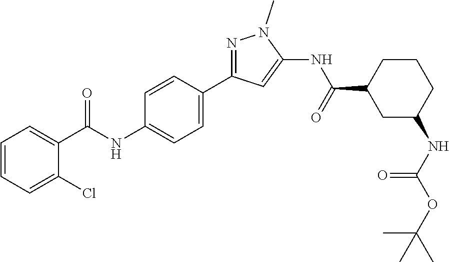 | (600 MHz, Methanol-d₄) δ 7.60 (s, 4H), 7.45 (d, J = 7.5 Hz, 1H), 7.34 (d, J = 8.3 Hz, 1H), 7.28 (t, J = 7.6 Hz, 1H), 7.24 (t, J = 7.5 Hz, 1H), 6.45 (s, 1H), 3.64 (s, 3H), 3.30 (t, J = 11.6 Hz, 1H), 2.43-2.34 (m, 1H), 2.03 (d, J = 12.3 Hz, 1H), 1.86-1.71 (m, 4H), 1.35 (s, 9H), 1.28-1.14 (m, 2H), 1.13-0.95 (m, 1H). | (151 MHz, MeOD) δ 175.3, 166.6, 156.4, 149.9, 137.97, 137.8, 136.4, 131.5, 131.2, 130.3, 129.8, 129.2, 127.2, 126.2, 120.7, 97.1, 79.8, 49.3, 44.0, 36.3, 35.5, 32.4, 28.5, 24.5. |
| 162 | SW394868- 2 | 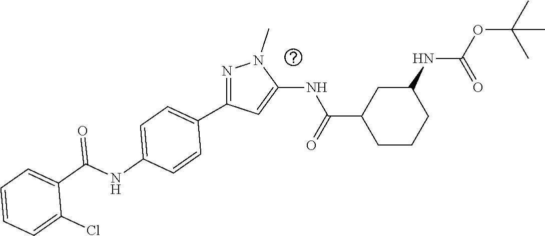 | (600 MHz, Methanol-d₄) δ 7.68 (s, 4H), 7.51 (d, J = 7.5 Hz, 1H), 7.42 (d, J = 8.0 Hz, 1H), 7.37 (t, J = 7.6 Hz, 1H), 7.32 (t, J = 7.5 Hz, 1H), 6.51 (s, 1H), 3.89 (s, 1H), 3.70 (s, 3H), 2.65 (tt, J = 9.4, 4.4 Hz, 1H), 1.91-1.75 (m, 3H), 1.72-1.49 (m, 7H), 1.44 (s, 9H); | (151 MHz, MeOD) δ 196.2, 187.3, 176.9, 176.9, 170.4, 158.5, 158.3, 157.0, 151.9, 151.7, 150.8, 150.3, 149.6, 147.7, 146.7, 141.3, 117.8, 100.1, 66.4, 60.3, 55.9, 54.2, 50.8, 49.2, 49.0, 49.0, 40.9; |
| 163 | SW394869- 1 | 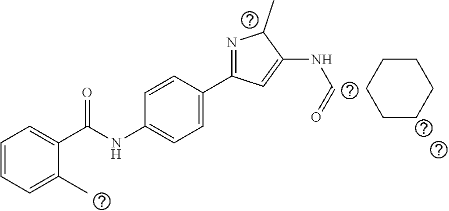 | (600 MHz, Methanol-d₄) δ 7.71 (s, 4H), 7.54 (dd, J = 7.5, 1.8 Hz, 1H), 7.46 (dd, J = 8.0, 1.3 Hz, 1H), 7.42 (td, J = 7.7, 1.8 Hz, 1H), 7.37 (td, J = 7.4, 1.3 Hz, 1H), 6.57 (s, 1H), 3.77 (s, 3H), 3.12 (tt, J = 11.7, 3.9 Hz, 1H), 2.65 (tt, J = 11.7, 3.1 Hz, 1H), 2.19 (d, J = 12.1 Hz, 1H), 2.08-1.88 (m, 4H), 1.53-1.43 (m, 2H), 1.33 (tt, J = 12.4, 5.6 Hz, 1H). | (151 MHz, MeOD) δ 175.1, 167.5, 150.5, 138.7, 138.2, 137.2, 131.8, 131.7, 130.6, 130.2, 129.5, 127.6, 126.6, 121.2, 97.6, 50.1, 43.6, 35.8, 34.1, 31.3, 29.1, 24.1. |
| 164 | SW394869- 2 | 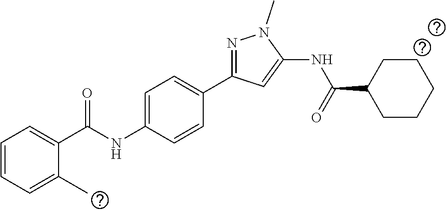 | (600 MHz, Methanol-d₄) δ 7.70 (s, 4H), 7.53 (dd, J = 7.5, 1.8 Hz, 1H), 7.44 (dd, J = 7.9, 1.3 Hz, 1H), 7.39 (td, J = 7.7, 1.8 Hz, 1H), 7.35 (td, J = 7.4, 1.3 Hz, 1H), 6.53 (s, 1H), 3.74 (s, 3H), 3.27 (tt, J = 6.6, 3.3 Hz, 1H), 2.79 (tt, J = 8.5, 4.1 Hz, 1H), 1.93 (ddd, J = 13.1, 8.9, 3.6 Hz, 1H), 1.67 (dddt, J = 40.1, 35.6, 26.4, 4.7 Hz, 6H), 1.43 (dt, J = 15.4, 5.7 Hz, 1H); | (151 MHz, MeOD) δ 176.4, 167.3, 150.3, 138.5, 138.3, 137.0, 131.7, 131.5, 130.5, 130.2, 129.4, 127.5, 126.5, 121.1, 97.6, 62.4, 46.1, 39.6, 36.2, 35.7, 33.2, 29.6, 29.1, 20.4 |
| 165 | SW394870 | 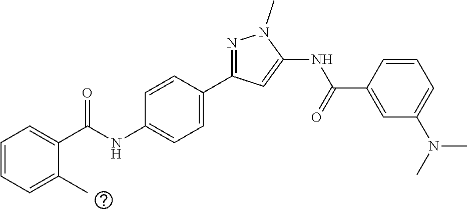 [SMILES: Cc1ccccc1C(=O)Nc1ccc(-c2cc(NC(=O)c3cccc(N(C)C)c3)n(C)n2)cc1] | (600 MHz, DMSO-d₆) δ 10.59 (s, 1H), 10.33 (s, 1H), 7.79 (d, J = 1.6 Hz, 4H), 7.61 (dd, J = 7.5, 1.8 Hz, 1H), 7.59-7.56 (m, 1H), 7.52 (td, J = 7.7, 1.8 Hz, 1H), 7.47 (td, J = 7.5, 1.3 Hz, 1H), 7.35 (t, J = 7.8 Hz, 1H), 7.32- 7.27 (m, 2H), 6.97 (dd, J = 8.4, 2.6 Hz, 1H), 6.69 (s, 1H), 3.75 (s, 3H), 2.97 (s, 6H). | (151 MHz, DMSO) δ 166.4, 164.9, 150.4, 147.94, 138.3, 137.7, 137.0, 134.0, 131.1, 130.0, 129.7, 129.1, 129.0, 127.3, 125.2, 119.7, 115.8, 115.4, 111.4, 97.7, 40.1, 35.9. |
| 166 | SW394871 | 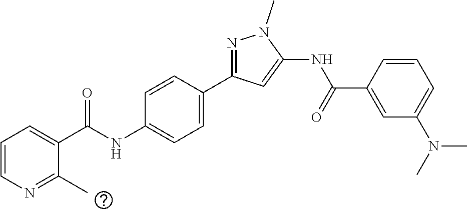 [SMILES: Cc1ncccc1C(=O)Nc1ccc(-c2cc(NC(=O)c3cccc(N(C)C)c3)n(C)n2)cc1] | (600 MHz, Methanol-d₄) δ 8.52 (dd, J = 5.0, 1.7 Hz, 1H), 7.93 (dd, J = 7.6, 1.7 Hz, 1H), 7.80-7.77 (m, 2H), 7.76- 7.72 (m, 2H), 7.39-7.35 (m, 1H), 7.35-7.32 (m, 2H), 7.30-7.26 (m, 1H), 7.00 (ddd, J = 8.2, 2.8, 0.9 Hz, 1H), 6.63 (s, 1H), 3.80 (s, 3H), 3.01 (s, 6H), 2.66 (s, 3H). | (151 MHz, MeOD) δ 170.6, 169.4, 157.3, 152.8, 151.4, 151.1, 139.9, 139.7, 137.6, 135.5, 134.7, 131.5, 130.9, 127.5, 123.1, 122.1, 118.1, 117.1, 113.2, 99.9, 41.2, 36.6, 22.9. |
| 167 | SW394872 | 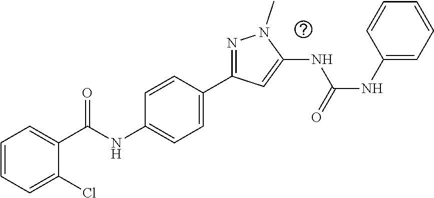 [SMILES: Cn1nc(-c2ccc(NC(=O)c3ccccc3Cl)cc2)cc1NC(=O)Nc1ccccc1] | (600 MHz, DMSO-d₆) δ 10.57 (s, 1H), 8.94 (s, 1H), 8.67 (s, 1H), 7.75 (s, 4H), 7.59 (dd, J = 15.5, 7.7 Hz, 2H), 7.49 (tt, J = 16.1, 7.4 Hz, 4H), 7.30 (t, J = 7.7 Hz, 2H), 7.00 (t, J = 7.3 Hz, 1H), 6.62 (s, 1H), 3.73 (s, 3H). | (151 MHz, DMSO) δ 164.9, 151.9, 147.8, 139.4, 138.5, 138.2, 137.0, 131.1, 130.0, 129.7, 129.3, 129.0, 128.9, 127.3, 125.2, 122.2, 119.7, 118.3, 94.4, 35.4. |
| 168 | SW394873 | 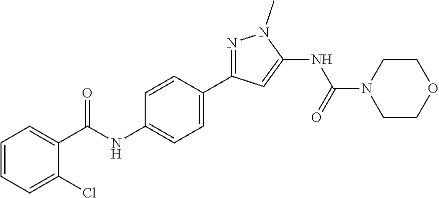 [SMILES: Cn1nc(-c2ccc(NC(=O)c3ccccc3Cl)cc2)cc1NC(=O)N1CCOCC1] | (600 MHz, DMSO-d₆) δ 10.56 (s, 1H), 8.64 (s, 1H), 7.74 (d, J = 2.7 Hz, 4H), 7.62-7.55 (m, 2H), 7.51 (td, J = 7.8, 1.8 Hz, 1H), 7.46 (t, J = 7.4 Hz, 1H), 6.46 (s, 1H), 3.64 (s, 3H), 3.64-3.60 (m, 4H), 3.44 (t, J = 4.8 Hz, 4H). | (151 MHz, DMSO) δ 164.9, 155.0, 147.6, 138.9, 138.1, 137.0, 131.1, 130.0, 129.7, 129.3, 129.0, 127.3, 125.1, 119.7, 97.5, 66.0, 43.8, 35.5. |
| 169 | SW394874 | 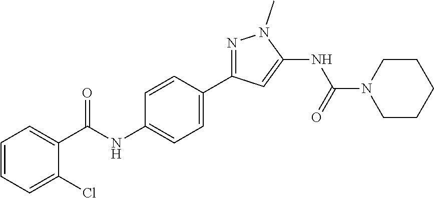 [SMILES: Cn1nc(-c2ccc(NC(=O)c3ccccc3Cl)cc2)cc1NC(=O)N1CCCCC1] | (600 MHz, DMSO-d₆) δ 10.56 (s, 1H), 8.52 (s, 1H), 7.75 (d, J = 10.6 Hz, 4H), 7.59 (dd, J = 15.2, 7.7 Hz, 2H), 7.51 (t, J = 7.6 Hz, 1H), 7.47 (d, J = 7.5 Hz, 1H), 6.43 (s, 1H), 3.63 (s, 3H), 3.42 (t, J = 5.3 Hz, 4H), 1.59 (d, J = 7.6 Hz, 2H), 1.56-1.38 (m, 4H). | (151 MHz, DMSO) δ 164.9, 154.7, 147.5, 139.3, 138.1, 137.0, 131.1, 130.0, 129.7, 129.4, 129.0, 127.3, 125.1, 119.6, 97.4, 55.0, 44.8, 35.4, 25.5, 24.1. |
| 170 | SW394875 | 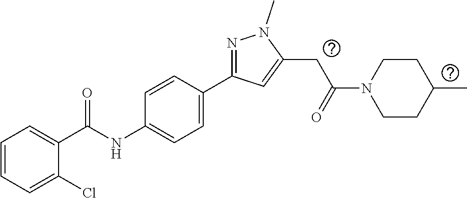 [SMILES: CC1CCN(C(=O)Cc2cc(-c3ccc(NC(=O)c4ccccc4Cl)cc3)nn2C)CC1] | (600 MHz, DMSO-d₆) δ 10.56 (s, 1H), 8.60 (s, 1H), 7.77-7.71 (m, 4H), 7.60 (dd, J = 7.5, 1.8 Hz, 1H), 7.58 (dd, J = 8.1, 1.2 Hz, 1H), 7.51 (td, J = 7.7, 1.8 Hz, 1H), 7.46 (td, J = 7.4, 1.3 Hz, 1H), 6.44 (s, 1H), 3.63 (s, 3H), 3.45 (dd, J = 6.0, 4.0 Hz, 4H), 2.32 (t, J = 5.0 Hz, 4H), 2.20 (s, 3H). | (151 MHz, DMSO) δ 164.9, 154.8, 147.5, 139.1, 138.1, 137.0, 131.1, 130.0, 129.7, 129.3, 129.0, 127.3, 125.1, 119.7, 97.5, 54.4, 45.8, 43.7, 35.4. |
| 171 | SW394876 | 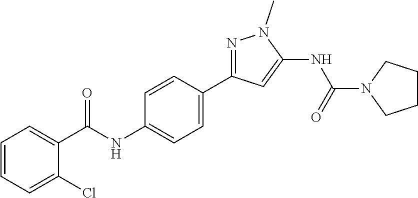 [SMILES: Cn1nc(-c2ccc(NC(=O)c3ccccc3Cl)cc2)cc1NC(=O)N1CCCC1] | (600 MHz, DMSO-d₆) δ 10.56 (s, 1H), 8.23 (s, 1H), 7.75 (t, J = 6.8 Hz, 4H), 7.59 (dd, J = 15.9, 7.7 Hz, 2H), 7.51 (t, J = 7.7 Hz, 1H), 7.46 (t, J = 7.4 Hz, 1H), 6.43 (s, 1H), 3.66 (s, 3H), 3.39- 3.34 (m, 6H), 1.86 (s, 4H). | (151 MHz, DMSO) δ 164.9, 153.8, 147.5, 139.1, 138.1, 137.0, 131.1, 130.0, 129.7, 129.4, 129.0, 127.3, 125.1, 119.7, 97.5, 55.0, 45.8, 35.5. |
| 172 | SW394877 | 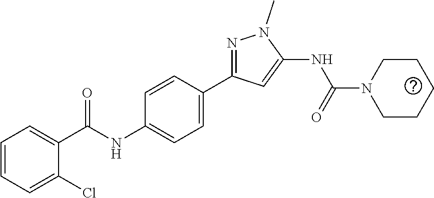 [SMILES: Cn1nc(-c2ccc(NC(=O)c3ccccc3Cl)cc2)cc1NC(=O)N1CCCCC1] | (600 MHz, DMSO-d₆) δ 10.55 (s, 1H), 8.70 (s, 1H), 7.76-7.71 (m, 4H), 7.60 (dd, J = 7.5, 1.8 Hz, 1H), 7.57 (dd, J = 8.0, 1.2 Hz, 1H), 7.51 (td, J = 7.7, 1.8 Hz, 1H), 7.46 (td, J = 7.4, 1.3 Hz, 1H), 6.45 (s, 1H), 3.64 (s, 3H), 3.49 (d, J = 5.1 Hz, 6H), 3.43 (dd, J = 6.7, 3.7 Hz, 2H), 2.04 (s, 3H). | (151 MHz, DMSO) δ 168.6, 164.9, 154.9, 147.6, 139.2, 138.1, 137.0, 131.1, 130.0, 129.7, 129.3, 129.0, 127.3, 125.1, 119.6, 97.3, 45.5, 43.9, 43.6, 40.7, 35.4, 21.4. |
| 173 | SW394878 | 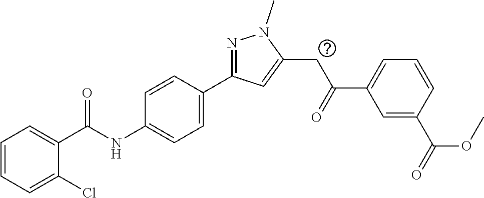 [SMILES: COC(=O)c1cccc(C(=O)Cc2cc(-c3ccc(NC(=O)c4ccccc4Cl)cc3)nn2C)c1] | (600 MHz, Methanol-d₄) δ 8.58 (s, 1H), 8.17 (d, J = 7.8 Hz, 1H), 8.12 (d, J = 7.8 Hz, 1H), 7.67 (s, 4H), 7.53 (t, J = 7.8 Hz, 1H), 7.48-7.45 (m, 1H), 7.37 (d, J = 8.0 Hz, 1H), 7.34-7.30 (m, 1H), 7.28 (t, J = 7.4 Hz, 1H), 6.53 (s, 1H), 3.89 (s, 3H), 3.76 (s, 3H). | (151 MHz, MeOD) δ 167.1, 167.1, 150.3, 138.4, 137.9, 136.8, 134.1, 133.7, 132.9, 131.6, 131.4, 131.0, 130.4, 130.0, 129.5, 129.4, 129.3, 127.4, 126.4, 121.0, 98.7, 52.8, 49.9, 36.0. |
| 174 | SW394880- 1 | 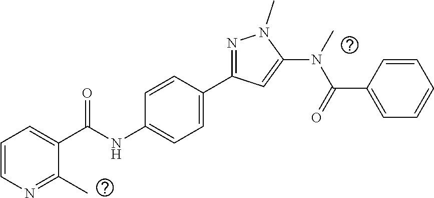 [SMILES: Cc1ncccc1C(=O)Nc1ccc(-c2cc(N(C)C(=O)c3ccccc3)n(C)n2)cc1] | (600 MHz, CDCl₃) δ 8.56-8.50 (m, 1H), 7.75 (d, J = 7.6 Hz, 1H), 7.64 (d, J = 8.2 Hz, 2H), 7.61-7.55 (m, 2H), 7.37-7.24 (m, 3H), 7.24-7.15 (m, 3H), 6.27 (s, 1H), 3.37 (s, 3H), 2.68 (s, 3H). | (151 MHz, CDCl₃) δ 166.34, 156.33, 150.30, 149.65, 137.31, 134.94, 134.40, 130.87, 128.26, 127.92, 126.83, 125.99, 121.03, 120.06, 60.44, 35.58, 29.73, 22.97, 22.72, 21.10, 14.22. |
| 175 | SW394891- 1 | 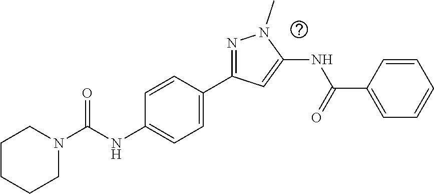 [SMILES: Cn1nc(-c2ccc(NC(=O)N3CCCCC3)cc2)cc1NC(=O)c1ccccc1] | (600 MHz, DMSO-d₆) δ 10.42 (s, 1H), 8.51 (s, 1H), 8.03-8.00 (m, 2H), 7.67-7.62 (m, 3H), 7.57 (t, J = 7.7 Hz, 2H), 7.54-7.49 (m, 2H), 6.64 (s, 1H), 3.74 (s, 3H), 3.43 (dd, J = 6.5, 4.3 Hz, 4H), 1.62-1.56 (m, 2H), 1.50 (td, J = 6.8, 6.2, 4.2 Hz, 4H). | (151 MHz, DMSO) δ 165.7, 154.8, 148.3, 140.2, 137.4, 133.4, 132.2, 128.6, 127.9, 126.9, 124.8, 119.5, 97.3, 44.7, 35.8, 25.6, 24.2. |
| 176 | SW394892- 1 | 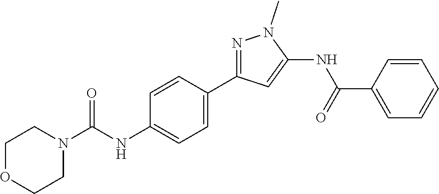 [SMILES: Cn1nc(-c2ccc(NC(=O)N3CCOCC3)cc2)cc1NC(=O)c1ccccc1] | (600 MHz, DMSO-d₆) δ 10.41 (s, 1H), 8.61 (s, 1H), 8.01 (s, 2H), 7.60 (dd, J = 77.6, 20.8 Hz, 7H), 6.64 (s, 1H), 3.73 (s, 3H), 3.61 (s, 4H), 3.44 (s, 4H). | (151 MHz, DMSO) δ 165.7, 155.1, 148.3, 139.8, 137.4, 133.4, 132.2, 128.6, 127.9, 127.2, 124.9, 119.6, 97.3, 66.0, 44.2, 35.8; |
| 177 | SW394893- 1 | 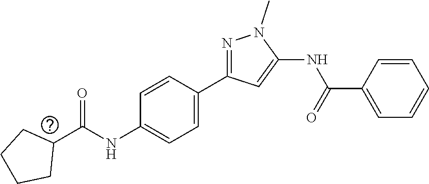 [SMILES: Cn1nc(-c2ccc(NC(=O)C3CCCC3)cc2)cc1NC(=O)c1ccccc1] | (600 MHz, DMSO-d₆) δ 10.41 (s, 1H), 8.17 (s, 1H), 8.01 (d, J = 7.6 Hz, 2H), 7.65 (d, J = 8.6 Hz, 3H), 7.56 (d, J = 8.1 Hz, 4H), 6.63 (s, 1H), 3.73 (s, 3H), 3.37 (d, J = 8.2 Hz, 4H), 1.85 (s, 4H). | (151 MHz, DMSO) δ 165.7, 153.9, 148.3, 140.1, 137.4, 133.4, 132.2, 128.6, 127.9, 126.8, 124.8, 119.4, 97.3, 45.7, 35.8, 25.1. |
| 178 | SW394894- 1 | 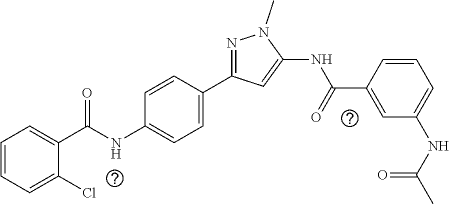 [SMILES: CC(=O)Nc1cccc(C(=O)Nc2cc(-c3ccc(NC(=O)c4ccccc4Cl)cc3)nn2C)c1] | (600 MHz, DMSO-d₆) δ 10.58 (s, 1H), 10.43 (s, 1H), 10.19 (s, 1H), 8.16 (s, 1H), 7.86 (d, J = 8.2 Hz, 1H), 7.81- 7.75 (m, 4H), 7.68 (d, J = 7.7 Hz, 1H), 7.59 (dd, J = 17.3, 7.7 Hz, 2H), 7.50 (dt, J = 21.4, 8.0 Hz, 3H), 6.69 (s, 1H), 3.75 (s, 3H), 2.08 (s, 3H). | (151 MHz, DMSO) δ 168.6, 165.8, 164.9, 148.0, 139.7, 138.3, 137.6, 137.0, 134.0, 131.1, 130.0, 129.7, 129.1, 129.0, 129.0, 127.3, 125.2, 122.5, 122.2, 119.7, 118.7, 97.6, 48.6, 35.8, 24.1. |
| 179 | SW394895- 1 | 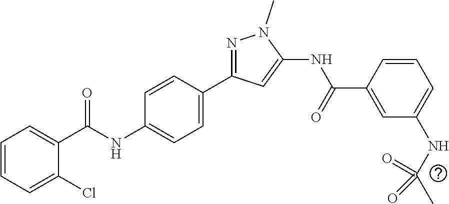 | (600 MHz, DMSO-d₆) δ 10.58 (s, 1H), 10.47 (s, 1H), 10.04 (s, 1H), 7.77 (q, J = 10.7, 8.0 Hz, 6H), 7.59 (dd, J = 17.2, 7.7 Hz, 2H), 7.52 (q, J = 8.3 Hz, 2H), 7.46 (d, J = 7.8 Hz, 2H), 6.70 (s, 1H), 3.75 (s, 3H), 3.06 (s, 3H). | (151 MHz, DMSO) δ 165.5, 164.9, 148.0, 138.9, 138.3, 137.4, 137.0, 134.6, 131.1, 130.0, 129.7, 129.6, 129.0, 129.0, 127.3, 125.2, 123.1, 122.9, 119.7, 119.1, 97.7, 35.9. |
| 180 | SW394896- 1 | 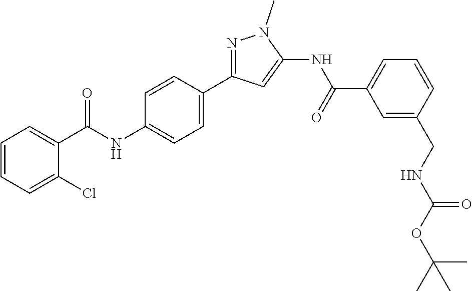 [SMILES: Cn1nc(-c2ccc(NC(=O)c3ccccc3Cl)cc2)cc1NC(=O)c1cccc(CNC(=O)OC(C)(C)C)c1] | (600 MHz, Methanol-d₄) δ 7.86-7.76 (m, 2H), 7.68 (s, 4H), 7.45 (t, J = 6.0 Hz, 2H), 7.41-7.35 (m, 2H), 7.33 (d, J = 7.4 Hz, 1H), 7.28 (d, J = 7.6 Hz, 1H), 6.74 (t, J = 6.3 Hz, 1H), 6.53 (s, 1H), 4.25 (d, J = 5.7 Hz, 2H), 3.74 (s, 3H), 1.41 (s, 9H). | (151 MHz, MeOD) δ 168.2, 167.2, 157.7, 140.7, 138.5, 138.1, 136.9, 133.8, 131.8, 131.7, 131.5, 130.5, 130.1, 129.4, 129.3, 127.5, 127.2, 127.1, 126.5, 121.1, 98.7, 80.1, 49.9, 44.5, 44.4, 35.9, 28.7; |
| 181 | SW394897- 1 | 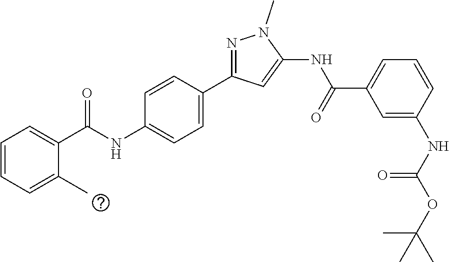 [SMILES: Cc1ccccc1C(=O)Nc1ccc(-c2cc(NC(=O)c3cccc(NC(=O)OC(C)(C)C)c3)n(C)n2)cc1] | (600 MHz, DMSO-d₆) δ 10.59 (s, 1H), 10.40 (s, 1H), 9.61 (s, 1H), 8.13 (s, 1H), 7.81-7.75 (m, 4H), 7.68-7.64 (m, 1H), 7.61 (dd, J = 7.5, 1.8 Hz, 2H), 7.58 (dd, J = 8.1, 1.3 Hz, 1H), 7.51 (td, J = 7.7, 1.8 Hz, 1H), 7.48- 7.41 (m, 2H), 6.69 (s, 1H), 3.75 (s, 3H), 1.49 (s, 9H). | (151 MHz, DMSO) δ 165.9, 164.9, 152.8, 148.0, 140.0, 138.3, 137.6, 137.0, 134.1, 131.1, 130.0, 129.7, 129.1, 129.0, 128.8, 127.3, 125.2, 121.3, 119.8, 117.8, 97.6, 79.4, 35.9, 28.1. |
| 182 | SW394966 | 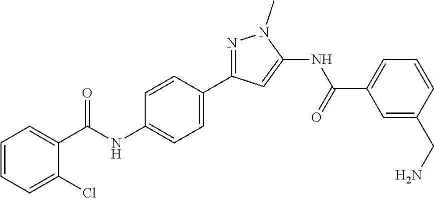 [SMILES: Cn1nc(-c2ccc(NC(=O)c3ccccc3Cl)cc2)cc1NC(=O)c1cccc(CN)c1] | (600 MHz, DMSO-d₆) δ 10.60 (s, 1H), 8.00 (s, 1H), 7.87 (d, J = 7.7 Hz, 1H), 7.82-7.74 (m, 4H), 7.65-7.56 (m, 3H), 7.55-7.44 (m, 3H), 6.69 (s, 1H), 3.84 (s, 2H), 3.76 (s, 3H). | (151 MHz, DMSO) δ 166.0, 164.9, 148.0, 144.1, 138.3, 137.7, 137.0, 133.2, 131.2, 131.0, 130.0, 129.7, 129.1, 129.0, 128.4, 127.3, 126.8, 125.9, 125.2, 119.7, 97.7, 45.2, 35.9 |
| 183 | SW394967 | 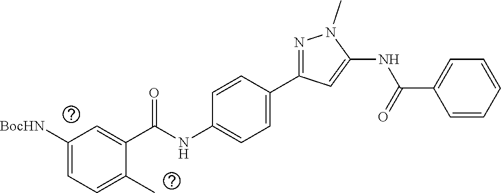 [SMILES: Cc1ccc(NC(=O)OC(C)(C)C)cc1C(=O)Nc1ccc(-c2cc(NC(=O)c3ccccc3)n(C)n2)cc1] | (600 MHz, DMSO-d₆) δ 10.56 (s, 1H), 10.44 (s, 1H), 8.04-7.99 (m, 2H), 7.77 (q, J = 8.9 Hz, 4H), 7.70 (d, J = 2.5 Hz, 1H), 7.66-7.62 (m, 1H), 7.60- 7.51 (m, 4H), 7.45 (d, J = 8.8 Hz, 1H), 6.70 (s, 1H), 3.76 (s, 3H), 1.48 (s, 9H). | (151 MHz, DMSO) δ 165.7, 164.8, 152.7, 148.0, 138.7, 138.2, 137.6, 137.1, 133.3, 132.2, 130.0, 129.1, 128.6, 128.0, 125.3, 122.3, 119.7, 117.8, 97.6, 79.7, 35.9, 28.1; |
| 184 | SW394968 | 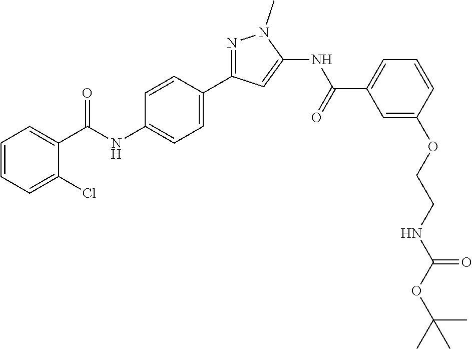 [SMILES: Cn1nc(-c2ccc(NC(=O)c3ccccc3Cl)cc2)cc1NC(=O)c1cccc(OCCNC(=O)OC(C)(C)C)c1] | (600 MHz, DMSO-d₆) δ 10.59 (s, 1H), 10.43 (s, 1H), 7.81-7.76 (m, 4H), 7.63-7.55 (m, 4H), 7.51 (td, J = 7.7, 1.8 Hz, 1H), 7.47 (tq, J = 6.2, 2.9, 2.4 Hz, 2H), 7.21 (dd, J = 8.5, 2.6 Hz, 1H), 7.08 (t, J = 5.7 Hz, 1H), 6.70 (s, 1H), 4.06 (t, J = 5.8 Hz, 2H), 3.76 (s, 3H), 3.35 (q, J = 5.8 Hz, 2H), 1.39 (s, 9H). | (151 MHz, DMSO) δ 165.5, 164.9, 158.5, 155.8, 148.0, 138.3, 137.5, 137.0, 134.7, 131.1, 130.0, 129.8, 129.7, 129.1, 129.0, 127.3, 125.2, 120.3, 119.7, 118.5, 113.7, 97.7, 77.9, 66.7, 35.9, 28.3. |
| 185 | SW394969 | 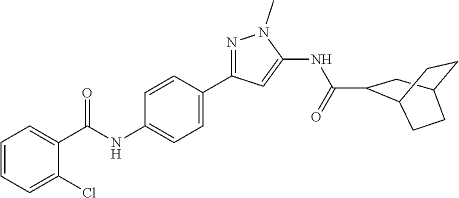 [SMILES: Cn1nc(-c2ccc(NC(=O)c3ccccc3Cl)cc2)cc1NC(=O)C1CC2CCC1CC2] | (600 MHz, DMSO-d₆) δ 10.56 (s, 1H), 9.84 (s, 1H), 7.74 (s, 4H), 7.62-7.55 (m, 2H), 7.51 (td, J = 7.7, 1.8 Hz, 1H), 7.46 (t, J = 7.4 Hz, 1H), 6.60 (s, 1H), 3.68 (s, 3H), 2.76-2.70 (m, 1H), 2.04-1.96 (m, 1H), 1.89 (q, J = 2.9 Hz, 1H), 1.71-1.46 (m, 8H), 1.46- 1.32 (m, 2H). | (151 MHz, DMSO) δ 174.0, 164.9, 147.8, 138.2, 138.1, 137.0, 131.1, 130.0, 129.7, 129.2, 129.0, 127.3, 125.2, 119.7, 96.0, 41.9, 35.7, 28.1, 27.0, 25.9, 24.9, 24.8, 23.2, 21.2. |
| 186 | SW394970 | 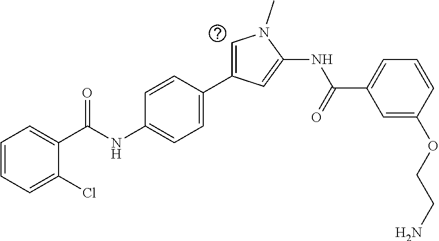 [SMILES: Cn1cc(-c2ccc(NC(=O)c3ccccc3Cl)cc2)cc1NC(=O)c1cccc(OCCN)c1] | (600 MHz, DMSO-d₆) δ 10.59 (s, 1H), 7.81-7.73 (m, 4H), 7.63-7.54 (m, 4H), 7.52 (td, J = 7.7, 1.8 Hz, 1H), 7.49-7.44 (m, 2H), 7.21 (ddd, J = 8.2, 2.6, 0.9 Hz, 1H), 6.69 (s, 1H), 4.01 (t, J = 5.8 Hz, 2H), 3.75 (s, 3H), 2.92 (t, J = 5.8 Hz, 2H). | (151 MHz, DMSO) δ 165.5, 164.9, 158.7, 148.0, 138.3, 137.6, 137.0, 134.7, 131.1, 130.0, 129.8, 129.7, 129.1, 129.0, 127.3, 125.2, 120.1, 119.7, 118.5, 113.7, 97.7, 70.5, 40.9, 35.9. |
| 187 | SW394971 | 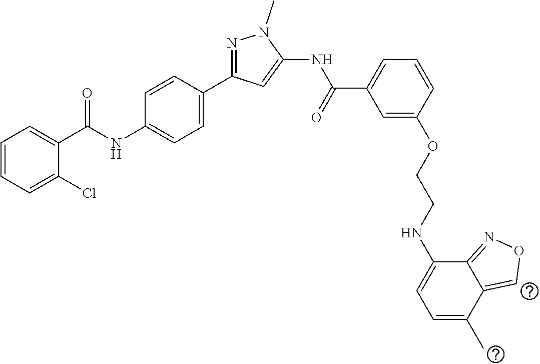 [SMILES: Cc1ccc(NCCOc2cccc(C(=O)Nc3cc(-c4ccc(NC(=O)c5ccccc5Cl)cc4)nn3C)c2)c2nocc12] | (600 MHz, DMSO-d₆) δ 10.58 (s, 1H), 10.40 (s, 1H), 9.65 (s, 1H), 8.56 (d, J = 8.8 Hz, 1H), 7.77 (s, 4H), 7.62-7.54 (m, 4H), 7.51 (td, J = 7.7, 1.8 Hz, 1H), 7.49-7.45 (m, 2H), 7.22 (dd, J = 8.3, 2.5 Hz, 1H), 6.67 (s, 1H), 6.57 (d, J = 9.0 Hz, 1H), 4.40 (t, J = 5.2 Hz, 2H), 3.93 (s, 2H), 3.73 (s, 3H). | (151 MHz, DMSO) δ 165.3, 164.9, 158.3, 148.0, 145.4, 144.5, 144.1, 138.3, 137.9, 137.4, 137.0, 134.6, 131.1, 139.0, 129.8, 129.7, 129.0, 129.0, 127.3, 125.2, 121.2, 120.5, 119.7, 118.6, 113.7, 99.8, 97.7, 65.7, 43.0, 35.9. |
| 188 | SW394972 | 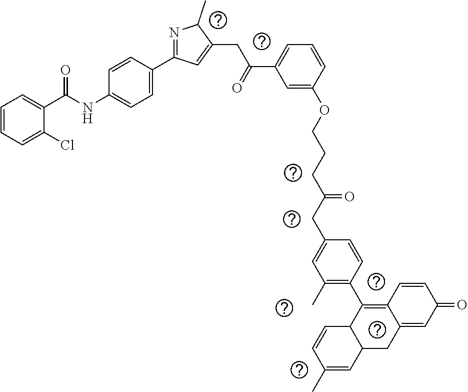 [SMILES: CC1=CC2CC3=CC(=O)C=CC3=C(c3ccc(CC(=O)CCCOc4cccc(C(=O)CC5=CC(c6ccc(NC(=O)c7ccccc7Cl)cc6)=NC5C)c4)cc3C)C2C=C1] | (600 MHz, Methanol-d₄) δ 8.11 (s, 1H), 7.76 (d, J = 8.3 Hz, 1H), 7.70 (s, 4H), 7.60 (s, 1H), 7.55 (dd, J = 13.5, 7.5 Hz, 2H), 7.45 (d, J = 8.0 Hz, 1H), 7.41 (q, J = 7.1, 6.4 Hz, 2H), 7.36 (t, J = 7.4 Hz, 1H), 7.19 (dd, J = 8.3, 2.5 Hz, 1H), 7.12 (d, J = 8.2 Hz, 1H), 6.72 (d, J = 8.8 Hz, 2H), 6.67-6.64 (m, 2H), 6.56 (s, 1H), 6.54-6.48 (m, 2H), 4.30 (t, J = 5.2 Hz, 2H), 4.08 (s, 2H), 3.78 (s, 3H). | (151 MHz, MeOD) δ 182.1, 168.1, 167.4, 159.5, 154.3, 150.4, 141.2, 138.54, 138.2, 137.1, 135.1, 131.7, 131.6, 130.6, 130.5, 130.2, 130.1, 129.4, 127.5, 126.5, 121.1, 121.1, 119.3, 114.6, 111.6, 103.3, 98.8, 66.9, 44.4, 36.0. |

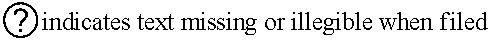

### II. Pharmaceutical Compositions and Methods of Use

A further aspect of the present disclosure provides pharmaceutical compositions comprising one or more of the compounds provided herein. In some aspects, the one or more compounds are those listed in Table 1 as 1-188. The one or more compounds may be pharmaceutically active or are prodrugs.

In some particular aspects, the compounds provided herein can be used for treatment or a wide range of cancer and tumors. As such, provided is a method of treating a cancer and/or a tumor by administering to a subject in need thereof, one or more of the compounds disclosed herein. The one or more compounds may be pharmaceutically active or are prodrugs. The one or more compounds may be a pharmaceutically acceptable salt. The one or more compounds may be one or more of the compounds listed in Table 1 as 1-188. The one or more compounds may be administered to the subject in a therapeutically effective amount. The subject may be an animal or human. The one or more compounds may be administered as a pharmaceutical composition.

The terms “cancer” and “cancerous” refer to or describe the physiological condition in mammals that is typically characterized by unregulated cell growth. The term “tumor” as used herein refers to a solid mass of tissue resulting from unregulated cell growth and proliferation. Cancers and tumors relevant herein may be malignant or benign, having cells exhibiting unregulated or dysregulated growth. Examples of cancer include, but are not limited to the following: cancers of the blood, breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia. In some aspects, the cancer is a colorectal cancer. In some aspects, the cancer is a leukemia. As described herein, the compounds and pharmaceutical compositions disclosed herein can be used for treating both cancer and tumors.

The term “treating,” as used herein refers to reversing, alleviating, inhibiting the progress of, or preventing the disorder or condition to which such term applies, or ameliorating one or more symptoms of such condition or disorder. The term “treatment,” or “therapy” of a subject refers to any type of intervention, or the administration of a compound as disclosed herein, to a subject with the objective of reversing, alleviating, ameliorating, inhibiting, slowing down or preventing the onset, progression, development, severity or recurrence of a symptom, complication, condition or biochemical indicia associated with a disease. In some aspects, the disease is a cancer and/or a tumor as provided herein.

In some aspects, the method of treating a cancer and/or a tumor by administering to a subject in need thereof, one or more of the compounds disclosed herein, comprises administering to the subject one or more of the compounds disclosed in Table 1, derivatives, and analogs, and pharmaceutically acceptable salts thereof. In some aspects, the treatment may comprise administering to a subject in need thereof, the one or more compounds as disclosed herein exhibiting an IC₅₀ in the micromolar to nanomolar range against one or more cancer cell lines, for example colorectal, lymphoma, leukemia or cervical cancer cell lines. IC₅₀ values for some of the compounds against the colorectal cell line HCT116 are provided in Table 2. In some aspects, the IC₅₀ for the one or more compounds may range from 1 pM to about 50 μM, against a cancer cell line, for example HCT116. In some aspects, the IC₅₀ for the compound may range from 0.001 μM to 0.01 μM, or about 0.01 μM to about 0.05 μM, or about 0.05 μM to about 0.1 μM, or about 0.1 μM to about 0.5 μM, or about 0.5 μM to about 1 μM, or about 1 μM to about 5 μM, or about 5 μM to about 10 μM, or about 10 μM to about 20 μM, or about 20 μM to about 30 μM, or about 30 μM to about 40 μM, or about 40 μM to about 50 μM against a cancer cell line, for example HCT116. In some aspects, the treatment may comprise administering to a subject in need thereof, the one or more compounds, wherein the one or more compounds can induce integrated stress response. In some aspects, the treatment may comprise administering to a subject in need thereof an effective amount of the one or more compounds, wherein the one or more compounds can bind RPS23. In some aspects, the treatment may comprise administering to a subject in need thereof, an effective amount of a composition comprising one or more of SW393071, SW388710, SW394672, SW394677, SW394740, SW394742, SW394766 or a derivative, an analog or a pharmaceutically acceptable salt thereof, or any combination thereof. In some aspects, the method of treating a cancer and/or a tumor, comprises administering any one or more of the compounds disclosed herein, except SW388717, SW388715, SW393061, SW394597, SW394800, SW394875, and/or SW394877. The one or more compounds may be administered to the subject in a pharmaceutical composition.

In some aspects, disclosed is a method of activating the integrated stress response in a subject. As used herein, the “Integrated Stress Response” or “ISR” is an intracellular signaling network that is activated in response to stress signals associated with a range of physiological and pathological conditions. The signals may include hypoxia, amino acid deprivation, glucose deprivation, oncogene activation, symptoms often associated with tumors and cancerous growth, and helps to animal cope with such adverse conditions. The common point of convergence for all the stress stimuli that activate ISR is phosphorylation of the alpha subunit of eukaryotic translation initiation factor 2 (elF2α). In an aspect, integrated stress response may be activated to treat cancer a subject in need thereof. As such, methods of treating a cancer and/or a tumor in a subject in need thereof by administering one or more of the compounds disclosed herein, may comprise administering the one or more compounds to the subject, wherein the one or more compounds activate the integrated stress response in the subject. In some aspects, the compounds disclosed herein may enhance phosphorylation of eukaryotic translation initiation factor 2 (elF2α), as disclosed in the examples herein. In some aspects, the method of treating a cancer and/or a tumor by administering to a subject in need thereof, a compound as disclosed herein, that effectively activates ISR. In some aspects, the compound is any one of the compounds provided in Table 1, or derivatives, analogs or combinations thereof, that activate ISR. In some aspects, the one or more compound may comprise SW393071, SW388710, SW394672, SW394677, or a derivative, an analog or a pharmaceutically acceptable salt thereof, or any combination thereof. The one or more compounds disclosed herein may be administered to the subject in a pharmaceutical composition.

In some aspects, the method of treating a cancer and/or a tumor by administering to a subject in need thereof, one or more of the compounds disclosed herein, wherein the one or more compounds bind RPS23. RPS23 is a 40S ribosomal protein S23, encoded by the RPS23 gene. In some aspects, the method of treating a cancer and/or a tumor by administering to a subject in need thereof, a compound as disclosed herein, that effectively binds RPS23. In some aspects, the compound is any one of the compounds provided in Table 1, or derivatives, analogs or combinations thereof, that bind RPS23. In some aspects, the one or more compound may comprise SW393071, SW388710, SW394672, SW394677, or a derivative, an analog or a pharmaceutically acceptable salt thereof, or any combination thereof. The one or more compounds disclosed herein may be administered to the subject in a pharmaceutical composition.

In some aspects, disclosed is a pharmaceutical composition that comprises one or more of the compounds disclosed herein and at least one pharmaceutically acceptable excipient. In some aspects, the pharmaceutical compositions disclosed herein are used in the methods of treating a cancer and/or tumor as disclosed herein. The pharmaceutical compositions disclosed herein may comprise a therapeutically effective amount of one or more of the compounds disclosed herein. The compounds disclosed herein may be administered to a subject in need thereof in a therapeutically effective amount.

The phrase “pharmaceutically acceptable” refers to molecular entities and compositions that are physiologically tolerable and do not typically produce a toxic, allergic, or similar untoward reaction, such as gastric upset, dizziness and the like, when administered to a human. Preferably, as used herein, the term “pharmaceutically acceptable” means approved by a regulatory agency of the Federal or a state government or listed in the U.S. or European Pharmacopeia or other generally recognized pharmacopeia for use in animals, and more particularly in humans.

The language “effective amount” of the compound is that amount necessary or sufficient to treat or prevent cancer or any other disease or disorder that is linked to ISR. In an example, an effective amount of the compound described herein is the amount sufficient to treat leukemia. In an example, an effective amount of the compound described herein is the amount sufficient to treat a colorectal cancer. The effective amount can vary depending on such factors as the size and weight of the subject, the type of illness, or the particular compound described herein. For example, the choice of the compound described herein can affect what constitutes an “effective amount.” One of ordinary skill in the art would be able to study the factors contained herein and make the determination regarding the effective amount of the compounds described herein without undue experimentation.

The regimen of administration can affect what constitutes an effective amount. Further, several divided dosages as well as staggered dosages, can be administered daily or sequentially, or the dose can be continuously infused, or can be a bolus injection. Further, the dosages of the compound(s) described herein can be proportionally increased or decreased as indicated by the exigencies of the therapeutic or prophylactic situation.

Compounds described herein may be used in the treatment of states, disorders or diseases as described herein, or for the manufacture of pharmaceutical compositions for use in the treatment of these diseases for example cancer. Methods of use of the compounds described herein in the treatment of these diseases, or pharmaceutical preparations having the compounds described herein for the treatment of these diseases.

The language “pharmaceutical composition” includes preparations suitable for administration to mammals, e.g., humans. When the compounds described herein are administered as pharmaceuticals to mammals, e.g., humans, they can be given per se or as a pharmaceutical composition containing, for example, 0.1% to 99.5% (preferably, 1% to 90%) of active ingredient in combination with a pharmaceutically acceptable excipient.

The phrase “pharmaceutically acceptable excipient” includes any pharmaceutically acceptable material, composition, or vehicle, suitable for administering the compounds described herein to mammals. The excipient includes liquid or solid filler, a diluent, a binder, a buffering agent, a pH modifying agent, a disintegrant, a dispersant, a preservative, a lubricant or wetting agent, taste-masking agent, an antioxidant, carrier, adjuvant, stabilizing agent, emulsifying agent, solution promoter, salt, solubilizer, antifoaming agent, surfactant, a flavoring agent, a coloring agent, solvent or encapsulating material or any combination thereof. Each excipient must be “acceptable” in the sense of being compatible with the other ingredients of the formulation and not injurious to the patient. The amount and types of excipients utilized to form pharmaceutical compositions may be selected according to known principles of pharmaceutical science. In each of the aspects described herein, a composition of the disclosure may optionally comprise one or more additional drug or therapeutically active agent in addition to the at least one factor disclosed herein. Thus, in addition to the therapies described herein, one may also provide to the subject other therapies known to be efficacious for treatment of the disease, disorder, or condition.

In some aspects, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, row the corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate

In some aspects, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In some aspects, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In some aspects, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In some aspects, the excipient may be a pH modifier. By way of non-limiting example, the pH modifying agent may be sodium carbonate, sodium bicarbonate, sodium citrate, citric acid, or phosphoric acid.

In some aspects, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. Non-limiting examples of suitable effervescent disintegrants include sodium bicarbonate in combination with citric acid and sodium bicarbonate in combination with tartaric acid.

In some aspects, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, iso-amorphous silicate, and microcrystalline cellulose.

In some aspects, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In some aspects, the excipient may be a lubricant. Non limiting examples of suitable lubricants include minerals such as talc or silica; and fats such as vegetable stearin, magnesium stearate, or stearic acid.

Some additional examples of materials which can serve as pharmaceutically acceptable excipients include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers, or lubricants, such as sodium lauryl sulfate and magnesium stearate, as well as coloring agents, release agents, coating agents, sweetening, flavoring and per-fuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active factor described herein, which can be in purified form, together with a suitable amount of carrier so as to provide the form for proper administration to the subject.

Formulations described herein include those suitable for parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal route. The formulations may conveniently be presented in unit dosage form and may be prepared by any methods well known in the art of pharmacy. The amount of active ingredient that can be combined with an excipient material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound as described herein with the excipient and, optionally, one or more accessory ingredients. In general, the formulations are prepared by uniformly and intimately bringing into association a compound described herein with liquid excipients, or finely divided solid excipients, or both, and then, if necessary, shaping the product.

Formulations described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. A compound described herein may also be administered as a bolus, electuary, or paste.

In solid dosage forms described herein for oral administration (capsules, tablets, pills, dragees, powders, granules and the like), the active ingredient is mixed with one or more pharmaceutically acceptable excipients, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described here-in, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or micro-spheres. They may be sterilized by, for example, filtration through a bacteria-retaining filter, or by incorporating sterilizing agents in the form of sterile solid compositions that can be dis-solved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms for oral administration of the compounds described herein include pharmaceutically acceptable emulsions, microemulsions, solutions, suspensions, syrups, or elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying agents, suspending agents, sweetening, flavoring, coloring, perfuming, preservative agents, or combinations thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound described herein include powders, sprays, ointments, pastes, creams, lotions, gels, solutions, patches and inhalants. The active compound may be mixed under sterile conditions with a pharmaceutically acceptable excipient, and with any preservatives, buffers, or propellants that may be required.

The ointments, pastes, creams, and gels may contain, in addition to an active compound de-scribed herein, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and pro-pane.

Transdermal patches have the added advantage of providing controlled delivery of a compound described herein to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions described herein suitable for parenteral administration comprise one or more compounds described herein in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous excipients that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, dispersing agents, or combinations thereof. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may de-pend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations described herein may be given orally, parenterally, topically, or rectally. They are of course given by forms suitable for each administration route. For example, they are ad-ministered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection or infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The subject treated is typically a human subject, although it is to be understood the methods described herein are effective with respect to other animals, such as mammals and vertebrate species.

As used herein, the term “subject” may include an animal, human or non-human, to whom treatment according to the methods of the present disclosure is provided. More particularly, the term subject can include animals used in assays such as those used in preclinical testing including but not limited to mice, rats, monkeys, dogs, pigs and rabbits; as well as domesticated swine (pigs and hogs), ruminants, equine, poultry, felines, bovines, murines, canines, and the like. Human and veterinary applications are anticipated by the present disclosure. The term includes but is not limited to birds, reptiles, amphibians, and mammals, e.g., humans, other primates, pigs, rodents, such as mice and rats, rabbits, guinea pigs, hamsters, horses, cows, cats, dogs, sheep, chickens and goats. In some aspects, the subjects are humans, chickens, or mice. In some aspects, the subject is a human. Both pediatric and adult subjects are included. For example, in any of the methods described herein, the subject can be at least 6 months old (e.g., 6 months or older, 12 months or older, 18 months or older, 2 years or older, 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years or older, 75 years or older, 80 years or older, 85 years or older, 90 years or older, or 1, 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, 104, or more years old).

As described above, in some particular aspects the term “subject” includes individuals that have been diagnosed with leukemia, colorectal cancer or a form of cancer that is susceptible to disruption of ISR.

Thus, in some aspects the current disclosure also encompasses use of disclosed compounds of treatment of a leukemia. In some aspects the current disclosure encompasses, use of disclosed compounds of treatment of a colorectal cancer. In some aspects, the current disclosure encompasses use of the disclosed compositions for activation of the integrated stress response, in a subject in need thereof.

As provided herein, these compounds may be administered to humans and other animals for therapy by any suitable route of administration, including parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal route.

Regardless of the route of administration selected, the compounds described herein, which may be used in a suitable hydrated form, and/or the pharmaceutical compositions described herein, are formulated into pharmaceutically acceptable dosage forms by conventional methods known to those of skill in the art.

Actual dosage levels of the active ingredients in the pharmaceutical compositions described herein may be varied to obtain an amount of the active ingredient that is effective to achieve the desired therapeutic response for a particular patient, composition, and mode of administration, without being toxic to the patient.

The selected dosage level will depend upon a variety of factors including the activity of the particular compound described herein employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In general, a suitable daily dose of a compound described herein will be that amount of the compound that is the lowest dose effective to produce a therapeutic effect. Such an effective dose will generally depend upon the factors described herein.

If desired, the effective daily dose of the active compound may be administered as two, three, four, five, six or more sub-doses administered separately at appropriate intervals throughout the day, optionally, in unit dosage forms.

While it is possible for a compound described herein to be administered alone, it may be administered in combination with other active composition.

By the term “combination” is meant either a fixed combination in one dosage unit form, or a kit of parts for the combined administration where a compound described herein and a combination partner may be administered independently at the same time or separately within time intervals that especially allow that the combination partners show a cooperative, e.g., synergistic, effect, or any combination thereof. The compounds described herein may be administered, simultaneously or sequentially, with an anti-inflammatory, antiproliferative, antibiotics, NSAIDs, painkillers, chemotherapeutic agent, immunosuppressant, other anti-cancer drugs, cytotoxic agent or salt thereof.

### III. Methods of Making the Compounds

A further aspect of the present disclosure provides methods of synthesizing compounds disclosed herein. In some aspects the compounds comprise those listed in Table 1 as 1-188. Another aspect of the present disclosure provides methods of synthesizing the intermediates disclosed herein (see, for example, Example 5) and/or compositions comprising the same.

The compounds described herein are prepared from commonly available compounds using a combination of procedures known to those skilled in the art. Details of the method of preparation of the disclosed compounds are provided in Example 5 herein. Some common methods are described for example in reference works, such as e.g., Science of Synthesis: Houben-Weyl Methods of Molecular Transformation. Georg Thieme Verlag, Stuttgart, Germany (2005); McOmie, “Protective Groups in Organic Chemistry,” Plenum Press, London and New York (1973). Analytical techniques including but not limited to ¹H and ¹³C NMR, thin layer chromatography, and LC/MS may be used to monitor the reactions and to characterize the reaction intermediates and desired final products.

In one or more embodiments, methods of making the compounds include the following reactions: 1) acid-amine coupling using acid chlorides; 2) preparation of amino-pyrazole derivatives; 3) acid-amine coupling using HATU or Ghosez's reagent; 4) deprotection of the Boc-group; and/or preparation of urea derivatives. One or more embodiments of the present disclosure provide a method for acid-amine coupling using acid chlorides. Another aspect of the present disclosure provides a method for preparation of amino-pyrazole derivatives. In another aspect, the present disclosure provides a method for acid-amine coupling using HATU or Ghosez's reagent. In some aspects, one or more embodiments of the present disclosure provide a method for the deprotection of the Boc-group. In certain aspects, the present disclosure provides a method for the preparation of urea derivatives.

Salts of the compounds described herein having at least one salt-forming group may be prepared in a manner known per se. For example, salts of the compounds described herein having acid groups may be formed, for example, by treating the compounds with metal compounds, such as alkali metal salts of suitable organic carboxylic acids, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of the compounds described herein are obtained in customary manner, e.g., by treating the compounds with an acid or a suitable anion exchange reagent. Internal salts of the compounds described herein containing acid and basic salt-forming groups, e.g., a free carboxy group and a free amino group, may be formed, e.g., by the neutralization of salts, such as acid addition salts, to the isoelectric point, e.g., with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable as described herein can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtain-able, for example by means of fractional crystallization, or by chromatography over optically active column materials. Intermediates and final products can be worked up and/or purified according to standard methods, e.g., using chromatographic methods, distribution methods, (re-) crystallization, and the like.

### Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Pharmaceutical composition” means a mixture of substances suitable for administering to an individual that includes a pharmaceutical agent. As used herein a pharmaceutical composition comprises one or more of the compounds as disclosed herein compounded with suitable pharmaceutical excipients.

As used herein, the term “patient”, “subject”, including “test subject” refers to any organism to which provided compound or compounds described herein are administered in accordance with the present disclosure e.g., for experimental, diagnostic, prophylactic, and/or therapeutic purposes. Typical subjects include animals (e.g., mammals such as mice, rats, rabbits, non-human primates, humans, insects, worms etc.). In an aspect, a subject is a human. In some aspects, a subject may be suffering from a tumor and/or cancer as disclosed herein. In some aspects, the cancer is a leukemia. In some aspects, the cancer is colorectal cancer.

The term “effective amount” as used herein is defined as the amount of the molecules of the present disclosure that are necessary to result in the desired physiological change in the cell or tissue to which it is administered. The term “therapeutically effective amount” as used herein is defined as the amount of the molecules or compositions of the present disclosure that achieves a desired effect with respect to cancer. In this context, a “desired effect” is synonymous with “an antitumor activity” or “an anti-cancer activity”. A skilled artisan readily recognizes that in many cases the molecules may not provide a cure but may provide a partial benefit, such as alleviation or improvement of at least one symptom or parameter. In some aspects, a physiological change having some benefit is also considered therapeutically beneficial. Thus, in some aspects, an amount of molecules that provides a physiological change is considered an “effective amount” or a “therapeutically effective amount.”

As used herein, the IC₅₀ refers to an amount, concentration or dosage of a particular test compound that achieves a 50% inhibition of a maximal response in an assay that measures such response.

As used herein, the K_{d} refers to the measured equilibrium dissociation constant between a compound (or ligand) and a protein (or binding domain of a protein).

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. High Throughput Screen for Compounds

An initial compound was identified using a high throughput screen for compounds that arrested the growth of HCT116 human colorectal cancer cells. This screen was performed at the UTSW high throughput screening core using commercially available screening libraries. The initial compound identified in this screen is provided in Table 1, compound name: SW106593.

### Example 2. Synthesis and Testing of Initial Compounds

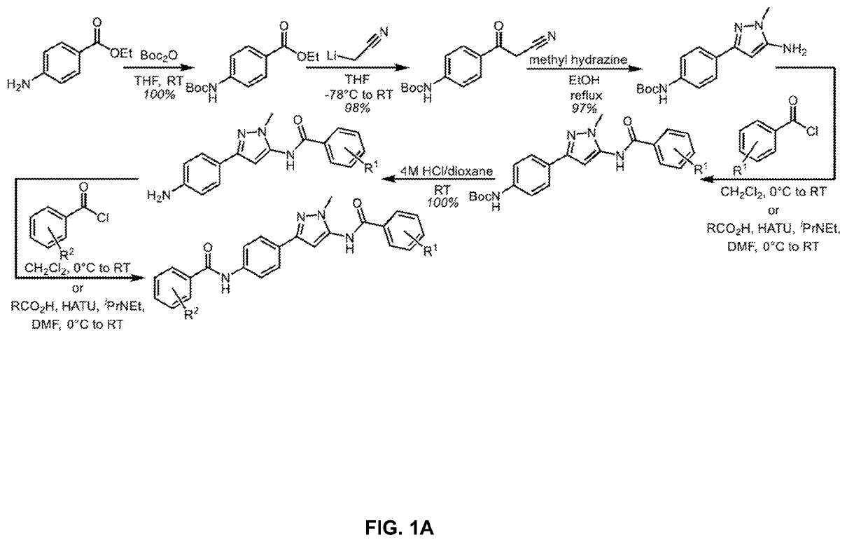

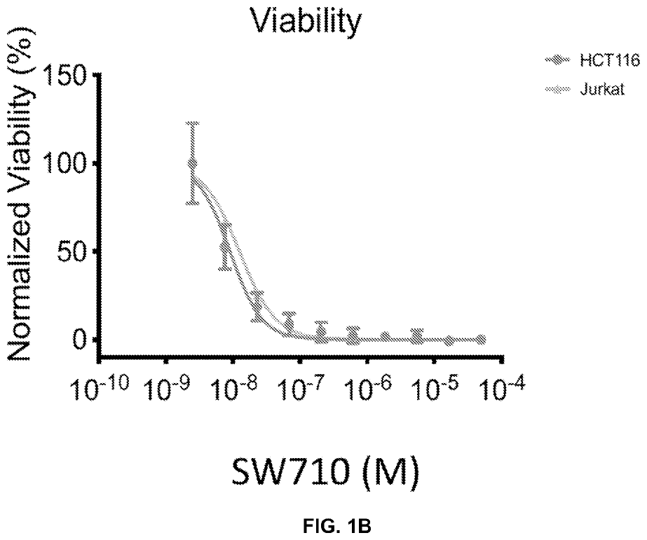

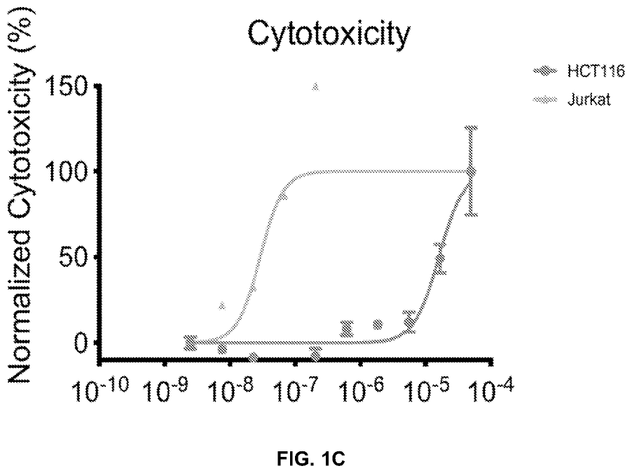

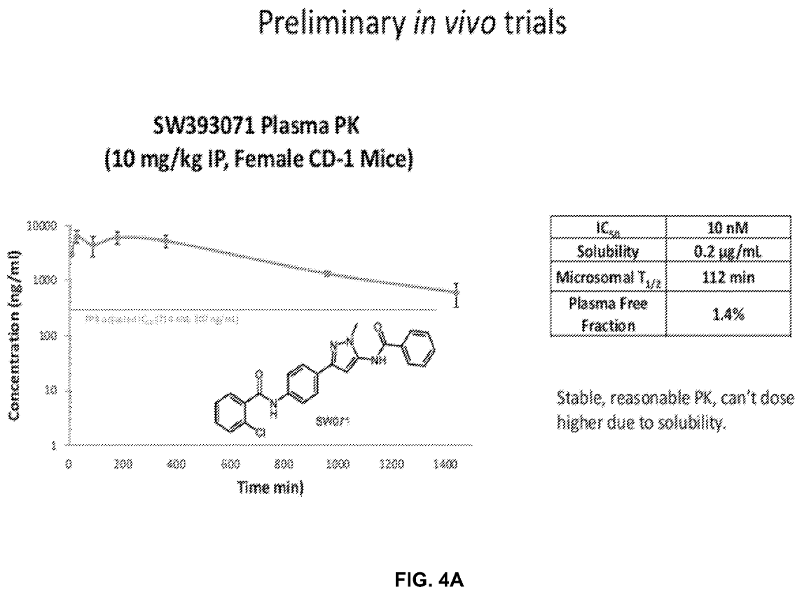

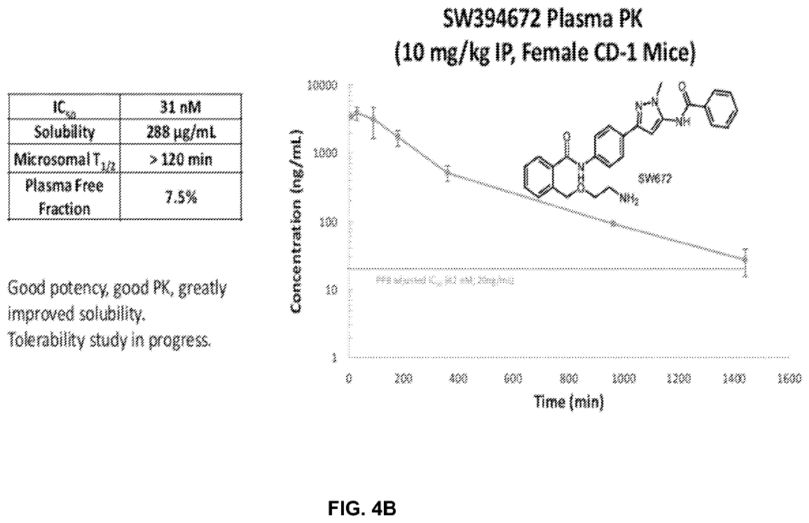

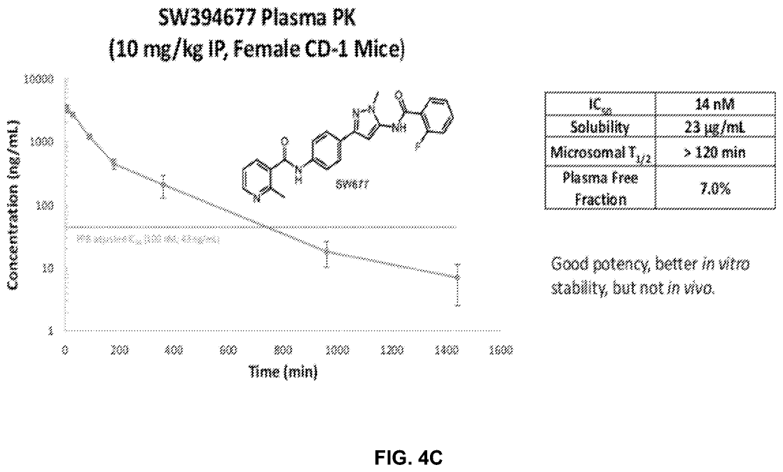

Once a lead compound was identified multiple first and second generation ortho and meta substituents were designed (see FIG. 1A). Activity is maintained with an ortho substituent on the left-hand aromatic ring, and a meta substituent on the right-hand aromatic ring. Multiple ortho substituents showed improved activity in vitro. No obvious correlation was noted between sterics or electronics and IC₅₀ activity of these analogs. Modular synthesis protocols and streamlined proliferation and cytotoxicity assays designed to allow for quick and easy screening of analogs and derivatives (for example as shown in FIG. 1B and FIG. 1C). These first- and second-generation compounds were also tested in vivo to develop effective assay and protocols as provided in FIG. 4A-4C.

### Example 3. Mechanistic Determinations

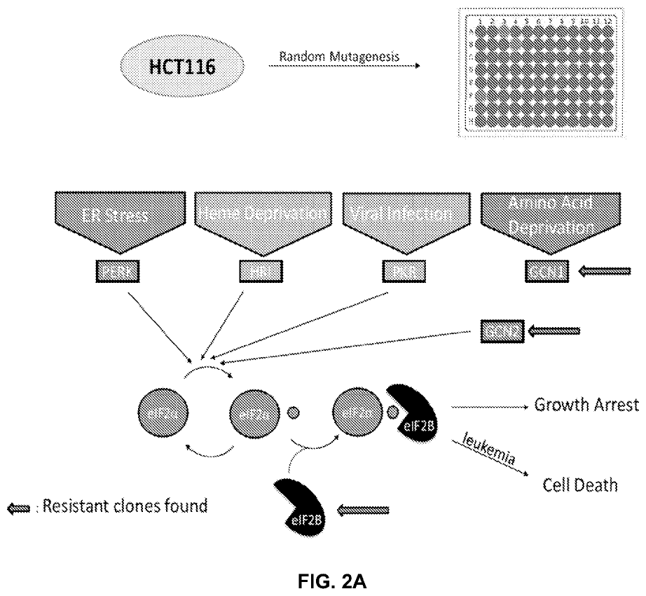

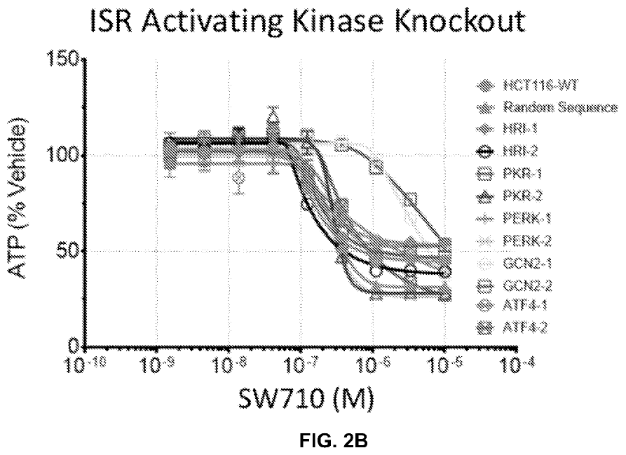

A forward genetics approach was taken to determine the mechanism of action of these initial compounds. Briefly HCT116 was randomly mutagenized as shown in FIG. 2A. Clones of HCT116, that were resistant to otherwise effective analog SW388710, revealed mutations in GN1, GCN2 and eIF2B, all of (which are involved in the ISR pathway. Knockout of GCN2, but not other activating kinases, confers resistance to SW388710 thus confirming the importance of the ISR (see FIG. 21B). Incomplete resistance however may suggest that GCN2 is not the direct binding partner.

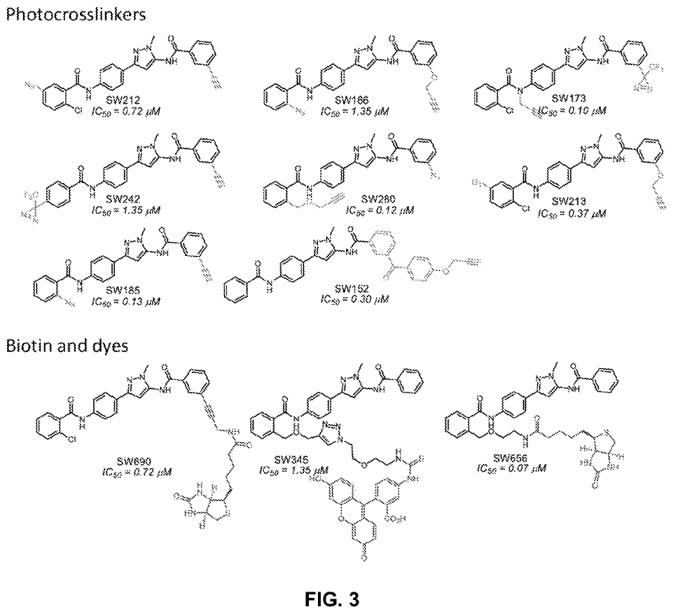

Analogs were also conjugated with probes (as provided in FIG. 3) to determine the binding site.

### Example 4. Synthesis and Testing of Next Generation Analogs and Key Findings

Next generation of novel compounds were synthesized using methodologies developed herein and known in the art. More than >180 analogs (1-188 compounds provided in Table 1) have been synthesized and characterized (see Tables 1) tested for anti-cancer activity (see Table 2).

TABLE 2

| | | HCT116 IC50 (uM) | | | |
|---|---|---|---|---|---|
| | | | HCT116 WT | HCT116 − | HCT116 WT + |
| # | NAME | HCT116 WT | (repeat) | GCN2KO | ISRIB |
| 1 | SW106593 | 0.22 | | | |
| 2 | SW388709 | 16 | | | |
| 3 | SW388710 | 0.23 | 0.21 | 1.95 | 1.89 |
| 4 | SW388711 | 1.09 | | | |
| 5 | SW388712 | 0.69 | | | |
| 6 | SW388713 | 1.1 | | | |
| 7 | SW388714 | 16 | | | |
| 8 | SW388715 | inactive | | | |
| 9 | SW388716 | 19 | | | |
| 10 | SW388717 | inactive | | | |
| 11 | SW388718 | 2.03 | | | |
| 12 | SW389001 | 0.31 | 0.84 | 6.44 | 7.00 |
| 13 | SW389002 | 7.1 | 26.36 | 31.91 | 46.34 |
| 14 | SW389003 | poor | 50.00 | 50.00 | 50.00 |
| | | solubility | | | |
| 15 | SW389004 | 5.7 | 7.47 | 7.75 | 7.22 |
| 16 | SW389005 | 0.313 | 0.33 | 2.97 | 2.96 |
| 17 | SW389006 | 1.225 | 0.82 | 2.34 | 2.40 |
| 18 | SW389007 | 2 | 4.80 | 5.22 | 4.42 |
| 19 | SW389008 | >50 | 50.00 | 50.00 | 50.00 |
| 20 | SW389009 | 0.96 | 1.77 | 50.00 | 50.00 |
| 21 | SW389010 | 20 | 43.06 | 41.11 | 50.00 |
| 22 | SW389012 | >50 | 7.01 | 19.17 | 50.00 |
| 23 | SW389013 | 2.79 | 4.21 | 15.82 | 19.80 |
| 24 | SW389014 | Not | | | |
| | | tested | | | |
| 25 | SW389152 | 0.3047 | 0.31 | 0.65 | 0.71 |
| 26 | SW389153 | 0.2085 | 0.20 | 1.31 | 1.73 |
| 27 | SW389154 | 0.1947 | 0.18 | 0.53 | 0.77 |
| 28 | SW389155 | 0.4156 | 0.46 | 2.20 | 2.97 |
| 29 | SW389156 | 0.2976 | 0.28 | 2.22 | 2.45 |
| 30 | SW389157 | 0.4048 | 0.21 | 2.77 | 2.50 |
| 31 | SW389158 | 0.5607 | 0.39 | 2.96 | 3.01 |
| 32 | SW389159 | 0.788 | 0.38 | 2.54 | 2.59 |
| 33 | SW389160 | 0.2263 | 0.29 | 1.96 | 2.48 |
| 34 | SW389161 | 1.266 | 1.06 | 11.71 | 8.26 |
| 35 | SW389162 | 0.3346 | 0.32 | 3.14 | 2.60 |
| 36 | SW393061 | inactive | 50.00 | 50.00 | 50.00 |
| 37 | SW393062 | 2.1 | 2.37 | 10.46 | 8.72 |
| 38 | SW393063 | 3.2 | 2.04 | 3.69 | 3.10 |
| 39 | SW393065 | 3 | 3.73 | 3.23 | 3.26 |
| 40 | SW393066 | 9.4 | 7.18 | 50.00 | 50.00 |
| 41 | SW393068 | 8.3 | 13.55 | 46.96 | 50.00 |
| 42 | SW393069 | 17 | 7.43 | 15.16 | 12.71 |
| 43 | SW393070 | 16 | 9.06 | 22.00 | 23.31 |
| 44 | SW393071 | 0.025 | 0.03 | 0.29 | 0.23 |
| 45 | SW393072 | 3.3 | 2.83 | 11.06 | 13.83 |
| 46 | SW393073 | 14 | 8.00 | 50.00 | 50.00 |
| 47 | SW393124 | 0.008 | 0.02 | 0.08 | 0.09 |
| 48 | SW393125 | 0.012 | 0.03 | 0.07 | 0.07 |
| 49 | SW393126 | 3.32 | 7.17 | 32.87 | 25.76 |
| 50 | SW393127 | 0.5 | 1.46 | 6.64 | 7.50 |
| 51 | SW393128 | 0.2 | 0.48 | 4.05 | 3.73 |
| 52 | SW393129 | 0.95 | 2.11 | 4.72 | 4.03 |
| 53 | SW393130 | 0.92 | 1.87 | 50.00 | 50.00 |
| 54 | SW393132 | >50 | 50.00 | 50.00 | 50.00 |
| 55 | SW393133 | 0.01 | 0.03 | 0.21 | 0.30 |
| 56 | SW393134 | 0.012 | 0.02 | 0.17 | 0.19 |
| 57 | SW393141 | 0.12 | 0.30 | 0.49 | 0.41 |
| 58 | SW393142 | 3.6 | 1.59 | 3.22 | 3.41 |
| 59 | SW393143 | 0.01 | 0.02 | 0.12 | 0.14 |
| 60 | SW393144 | 0.13 | 0.43 | 2.31 | 2.55 |
| 61 | SW393168 | 0.34 | 1.02 | 2.95 | 3.23 |
| 62 | SW393169 | 0.01 | 0.06 | 0.41 | 0.49 |
| 63 | SW393170 | 0.03 | 0.12 | 0.93 | 0.84 |
| 64 | SW393171 | 0.19 | 0.49 | 2.78 | 2.33 |
| 65 | SW393172 | 3.6 | 5.08 | 4.37 | 5.25 |
| 66 | SW393173 | 2.4 | 4.00 | 2.90 | 3.11 |
| 67 | SW393174 | 6.1 | 7.18 | 6.37 | 6.52 |
| 68 | SW393185 | 0.13 | | | |
| 69 | SW393186 | 0.1 | | | |
| 70 | SW393187 | 0.14 | | | |
| 71 | SW393188 | 0.6 | | | |
| 72 | SW393189 | 0.06 | | | |
| 73 | SW393212 | 0.72 | | | |
| 74 | SW393213 | 0.37 | | | |
| 75 | SW393214 | 0.57 | | | |
| 76 | SW393215 | 0.62 | | | |
| 77 | SW393216 | 0.07 | | | |
| 78 | SW393217 | 0.03 | | | |
| 79 | SW393218 | 0.02 | | | |
| 80 | SW393241 | 0.04 | | | |
| 81 | SW393242 | 1.35 | | | |
| 82 | SW393243 | 17 | | | |
| 83 | SW393244 | ~0.01 | | | |
| 84 | SW393270 | 0.13 | | | |
| 85 | SW393279 | >10 | | | |
| 86 | SW393280 | 0.12 | | | |
| 87 | SW393293 | 0.19 | | | |
| 88 | SW393294 | 0.28 | | | |
| 89 | SW393296 | 1.9 | | | |
| 90 | SW393298 | 0.15 | | | |
| 91 | SW393345 | 11.4 | | | |
| 92 | SW394442 | 0.024 | | | |
| 93 | SW394443 | 0.02 | | | |
| 94 | SW394444 | >50 | | | |
| 95 | SW394445 | 0.18 | | | |
| 96 | SW394446 | 0.05 | | | |
| 97 | SW394447 | 0.58 | | | |
| 98 | SW394484 | 3.94 | | | |
| 99 | SW394485 | 0.107 | | | |
| 100 | SW394486 | 0.025 | | | |
| 101 | SW394487 | 0.023 | | | |
| 102 | SW394488 | 0.32 | | | |
| 103 | SW394489 | 0.61 | | | |
| 104 | SW394490 | 3.84 | | | |
| 105 | SW394491 | 12 | | | |
| 106 | SW394515 | 0.044 | | | |
| 107 | SW394516 | inactive | | | |
| | (isomer 1) | | | | |
| 108 | SW394516 | 0.77 | | | |
| | (isomer 2) | | | | |
| 109 | SW394517 | 0.32 | | | |
| 110 | SW394518 (HCl | 0.016 | | | |
| | Salt: SW394676) | | | | |
| 111 | SW394519 | 0.25 | | | |
| 112 | SW394532 | 0.121 | | | |
| 113 | SW394534 (HCl | 0.046, 0.017; | | | |
| | salt: SW394672) | 30 | | | |
| 114 | SW394535 | inactive | | | |
| 115 | SW394544 | 1.2 | | | |
| 116 | SW394545 | 12.9 | | | |
| 117 | SW394546 | 0.033 | | | |
| 118 | SW394547 | 0.05 | | | |
| 119 | SW394548 | 1.13 | | | |
| 120 | SW394549 | 4.1 | | | |
| 121 | SW394572 | 5.8 | | | |
| 122 | SW394597 | inactive | | | |
| 123 | SW394598 | 0.55 | | | |
| 124 | SW394599 | 0.05 | | | |
| 125 | SW394600 | 0.051 | | | |
| 126 | SW394601 | 7.4 | | | |
| 127 | SW394602 | 0.049 | | | |
| 128 | SW394603 | 0.02 | | | |
| 129 | SW394628 (HCl | 0.007, 0.006, | | | |
| | salt: SW394677) | 0.014 | | | |
| 130 | SW394629 | 0.017 | | | |
| 131 | SW394630 | 0.014 | | | |
| 132 | SW394631 | 0.022 | | | |
| 133 | SW394656 | 0.071 | | | |
| 134 | SW394657 | 0.044 | | | |
| 135 | SW394692 | 0.5 | | | |
| 136 | SW394693 | 0.17 | | | |
| 137 | SW394694 | 0.03 | | | |
| 138 | SW394695 | 0.03 | | | |
| 139 | SW394696 | 0.09 | | | |
| 140 | SW394697 | 0.77 | | | |
| 141 | SW394698 | 10 | | | |
| 142 | SW394699 | 0.02 | | | |
| 143 | SW394738 | 0.07 | | | |
| 144 | SW394739 | 0.4 | | | |
| 145 | SW394740 | 0.009, 0.005, | | | |
| | | 0.018 | | | |
| 146 | SW394741 | 0.17 | | | |
| 147 | SW394742 | 0.015, 0.011, | | | |
| | | 0.031 | | | |
| 148 | SW394743 | 15 | | | |
| 149 | SW394744 | >50 | | | |
| 150 | SW394745 | 21.7 | | | |
| 151 | SW394746 | 0.13 | | | |
| 152 | SW394763 | 0.02 | | | |
| 153 | SW394764 | 0.3 | | | |
| 154 | SW394765 | 0.01 | | | |
| 155 | SW394766 | <0.007, 0.083 | | | |
| 156 | SW394767 | 0.27 | | | |
| 157 | SW394768 | inactive | | | |
| 158 | SW394770 | 0.033 | | | |
| 159 | SW394771 | 0.086 | | | |
| 160 | SW394800 | inactive | | | |
| 161 | SW394868- | 0.1 | | | |
| | isomer 1 | | | | |
| 162 | SW394868- | 0.12 | | | |
| | isomer 2 | | | | |
| 163 | SW394869- | 0.04 | | | |
| | isomer 1 | | | | |
| 164 | SW394869- | 0.08 | | | |
| | isomer 2 | | | | |
| 165 | SW394870 | 0.02 | | | |
| 166 | SW394871 | 0.01 | | | |
| 167 | SW394872 | 0.11 | | | |
| 168 | SW394873 | 0.29 | | | |
| 169 | SW394874 | 0.067 | | | |
| 170 | SW394875 | inactive | | | |
| 171 | SW394876 | 0.19 | | | |
| 172 | SW394877 | inactive | | | |
| 173 | SW394878 | 0.01 | | | |
| 174 | SW394880-1 | 1.04 | | | |
| 175 | SW394891-1 | 0.24 | | | |
| 176 | SW394892-1 | >5 | | | |
| 177 | SW394893-1 | >5 | | | |
| 178 | SW394894-1 | 0.01 | | | |
| 179 | SW394895-1 | 0.03 | | | |
| 180 | SW394896-1 | 0.05 | | | |
| 181 | SW394897-1 | 0.02 | | | |
| 182 | SW394966 | 0.055 | | | |
| 183 | SW394967 | 0.541 | | | |
| 184 | SW394968 | 0.062 | | | |
| 185 | SW394969 | 0.03 | | | |
| 186 | SW394970 | 0.05 | | | |
| 187 | SW394971 | 0.02 | | | |
| 188 | SW394972 | 1.3 | | | |

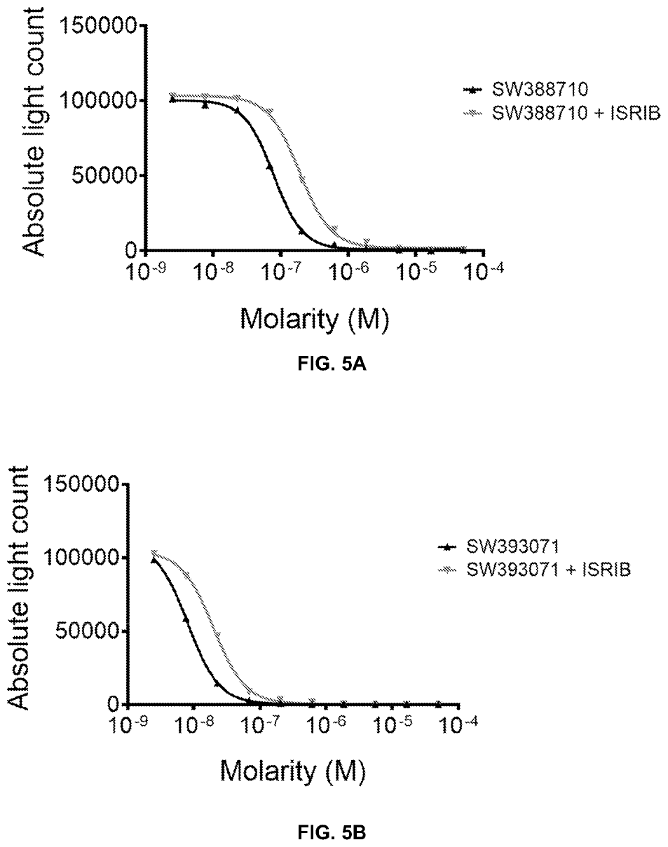

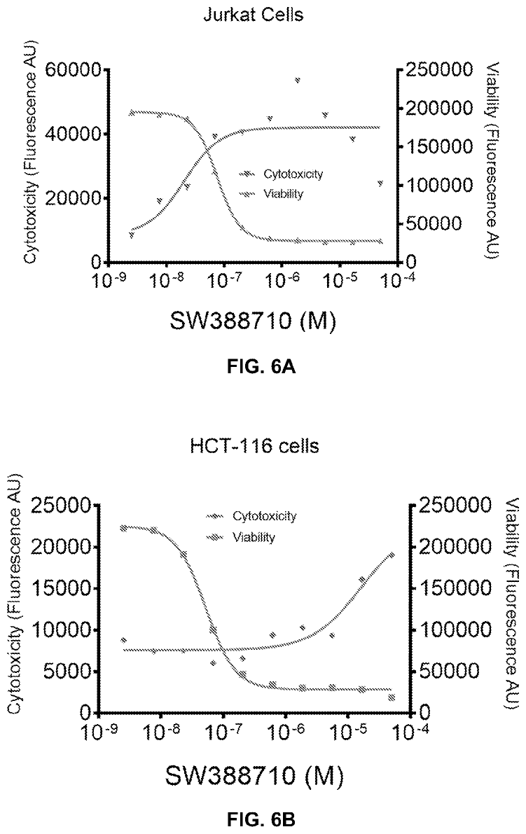

Briefly, compounds were tested for their effect on proliferation of HCT116 colorectal cell lines using Cell TiterGlo (Promega). Each compound was tested in a 10-point dose response experiment in triplicate. Data for these experiments are provided in Table 2. Multiple values in the column titled HCT116 WT reflect separate independent experiments. The three columns titled “HCT116 WT (repeat)”, “HCT116-CGN2KO” and “HCT116 WT+ISRIB” provide data for selected compounds that were tested simultaneously under three conditions. The HCT116 WT column lists IC₅₀ against the wild type HCT116 colorectal cancer cell line. The HCT116-CGN2KO lists IC₅₀ data for cells in which one component of the integrated stress response, GCN2, has been knocked out using CRISPR technology. The column titled “HCT116 WT+ISRIB” shows IC₅₀ data for cells treated with the indicated compound plus 10 nM of ISRIB, a compound known to inhibit the integrated stress response. The approximately 2-10-fold shift observed in the GCN2 KO cells or in the presence of ISRIB indicates that activation of the integrated stress response is essential to the anti-proliferative phenotype. Cell proliferation assay with exemplary compounds SW388710 and SW393071 are provided in FIGS. 5A and 51B. Cytotoxicity assays with SW388710 are provided in FIGS. 6A and 61B.

Genetic screens, biochemical tools and cryoEM studies were done to further determine the mechanism of action of these novel compounds. Key findings from this generation of novel analogs include:
1) Lead compounds have IC₅₀ values <20 nM against several cancer cell lines.
2) Active compounds arrest the growth of most cell lines but kill leukemia cell lines.
3) Active compounds bind to the ribosome at a unique binding site adjacent to the ribosomal protein RPS23. No other compound has been observed to bind at this site. Binding partially inhibits protein translation. 3-dimensional structure of multiple compounds bound to the ribosome by cyroEM with atomic resolution have bene determined as part of the current disclosure.
4) Binding to RPS23 was shown to activate the integrated stress response (ISR), a conserved cellular response to a variety of cellular insults. The activated ISR, in conjunction with RPS23 binding, uniquely results in cytotoxicity towards certain cancer types, including but not restricted to leukemia. Other inducers of the ISR and other inhibitors of translation do not demonstrate this activity thus making these compounds and the underlying activity truly novel. The combination of partial translation inhibition and ISR activation may lead to enhanced cancer cell killing and improved therapeutic index.
5) Several advanced analogs as provided in Table 1 have been developed that have solubility, in vitro stability, and in vivo pharmacokinetic properties sufficient for efficacy testing.
6) These lead compounds appear tolerated in mice at doses that should exceed the IC₅₀ for full dosing periods.

### Example 5. Detailed Methods of Synthesis

Details of the protocols for synthesis are provided herein.

All NMR experiments were recorded on Bruker Ascend-600 spectrometer, Varian Inova-400 spectrometer, and BrukerAscend-400 spectrometer. Data for ¹H and ¹³C NMR spectra are reported as follows: chemical shift (δ, ppm), integration, multiplicity (s=singlet, d=doublet, t=triplet, q=quartet, dd=doublet of doublets, ddd=doublet of doublets of doublets, dt=doublet of triplets and m=multiplet) and coupling constant (Hz). The 7.26, 3.31 and 2.50 resonance residuals of CDCl3, MeOD and DMSO for proton spectra and the 77.23, 49.00 and 39.52 resonance of CDCl3, MeOD and DMSO respectively for carbon spectra were used as internal references. Silica gel flash chromatography purifications were performed using 40-63 micron flash silica gel purchased from Sorbtech Technologies. Thin layer chromatography (TLC) was performed on silica gel 60 F254 pre-coated glass plates (0.25 mm) purchased from E. Merck. Visualization of TLC plates was performed using ultraviolet light (254 nm), ceric ammonium molybdate (CAM) or KMnO₄ stain and heat as developing agents. Mass spectra were acquired on an Agilent technologies 1200 series LC/MS using indicated ionization methods.

All moisture sensitive reactions were run in a flame-dried flask under N₂. Solvents were dried using a J. C. Meyer Solvent Dispensing System (SDS) and dispensed under N₂. Deuterated chloroform (CDCl₃) was dried over 4 Å molecular sieves. All starting materials and reagents were purchased from commercial sources and used as received. Unless otherwise stated, reaction progress was monitored by thin layer chromatography (TLC) performed on glass plates coated with silica gel UV254 or using Agilent technologies 1200 series LC/MS.

### Experimental Procedure

### General Procedure A: Acid-Amine Coupling Using Acid Chlorides

Oxalyl chloride (10 equiv) or thionyl chloride (10 equiv) was added to a solution of the carboxylic acid derivative (1.5 equiv) in DCM (0.4 M) and the solution cooled to 0° C. while stirring. 3 Drops of DMF were added and the reaction mixture was allowed to warm to room temperature and stirred for 1-15 hrs. The formed acid chloride was concentrated and azeotroped three times with toluene to remove excess oxalyl chloride or thionyl chloride and then utilized in the subsequent step without purification. Acid chloride derivative (1.5 equiv) was added to a mixture of the amine (1.0 equiv) and N,N-diisopropylethylamine (2.0 equiv) in DCM (0.2 M) at 0° C. The reaction mixture was stirred at this temperature for 10 min then allowed to warm to room temperature. The reaction mixture was stirred at room temperature for 3-15 hr. The completion of reaction was monitored by LCMS. The reaction mixture was then concentrated, and H₂O and EtOAc were added. The organic layer was separated and aqueous layer extracted with EtOAc (3×). The organic layers were combined, washed with 1 M HCl, 1 M NaOH, brine, dried (Na₂SO₄) and concentrated under reduced pressure. The crude products were purified by either flash column chromatography on silica gel (hexanes/EtOAc or DCM/MeOH) or via recrystallization to give desired product.

### General Procedure B. Preparation of Amino-Pyrazoles Derivatives

n-BuLi (3.3 equiv) was added slowly to a solution of DIPA (3.3 equiv) in THE (1.07 M) at −78° C. The reaction mixture was stirred at this temperature for 10 mins then a solution of alkyl- or phenyl nitrile (3.0 equiv) in THF (7.0 M) was added dropwise. The resulting slurry was stirred for 15 mins at −78° C. then a solution of ethyl benzoate derivative (1.0 equiv) in THF (0.57 M) was added. The reaction mixture was stirred at −78° C. for 90 min then allowed to warm to rt for 3 h. The reaction was quenched by addition of 1 M HCl and extracted with EtOAc. The organic layers were combined, washed with brine, dried (Na₂SO₄) and evaporated under reduced pressure to give keto-nitrile derivatives. The formed keto-nitrile was dried under strong vacuum for 30 min and utilized in the subsequent step without further purification. Hydrazine derivative (7.0 equiv) was added to a solution of keto-nitrile (1.0 equiv) in EtOH (0.38 M) at rt and then stirred at reflux for 24 h. The reaction mixture was cooled to room temperature and solvent was removed under vacuo. The resulting aminopyrazole derivative was used in the subsequent step without further purification unless otherwise noted.

### General Procedure C: Acid-Amine Coupling Using HATU

The carboxylic acid derivative (1.0 equiv) was dissolved in dry DCM or DMF (0.2 M). The solution was stirred for 10 min at room temperature and then cooled to 0° C. HATU (1.5 equiv) was added followed by N,N-diisopropylethylamine (2.0 eq). The reaction mixture was stirred for 1 hr at 0° C., and then it was allowed to warm to room temperature. The amine (1.0 equiv) was added to the mixture and stirring was continued for 12 h at room temperature. The completion of the reaction was monitored by LCMS. The reaction mixture was diluted with EtOAc and washed with H₂O and brine, dried (Na₂SO₄) and concentrated under reduced pressure. The crude product was purified by column chromatography on silica gel (hexanes/EtOAc or DCM/MeOH) to give the desired product.

### General Procedure D: Acid-Amine Coupling Using Ghosez's Reagent

The carboxylic acid derivative (1.2 equiv) was suspended in dry DCM (0.2 M) under nitrogen atmosphere and 1-chloro-N,N,2-trimethylpropenylamine (Ghosez's reagent) (3.0 equiv) was added. The reaction mixture was stirred at rt for 2 h resulting in a homogeneous solution. To this solution was added the amine (1.0 equiv) and pyridine (10 equiv) and the reaction mixture was stirred at room temperature overnight, then the solvent was evaporated. The crude products were either purified by flash column chromatography on silica gel (hexanes/EtOAc or DCM/MeOH) or via recrystallization to give the desired product.

### General Procedure E: Deprotection of the Boc-Group

4 M HCl in dioxane was added to the respective tert-butyl carboxylate derivative. After stirring at room temperature until starting material was consumed (monitored via LCMS), the solution was concentrated in vacuo. The desired amine product was utilized in the subsequent step without further purification unless otherwise noted.

### General Procedure F: Preparation of Urea Derivatives

DIPEA (3.0 equiv) was slowly added to a mixture of carbamate (1.0 equiv) and the amine derivative (1.2 equiv) in DMF (0.5 M) at room temperature. The reaction mixture was then stirred at 50° C. for 12 h. The reaction mixture was diluted with EtOAc and then poured into H₂O. The organic layer was separated, washed with brine, dried (Na₂SO₄) and concentrated under vacuo. The crude product was purified by flash column chromatography on silica gel (DCM/MeOH) to give the desired product.

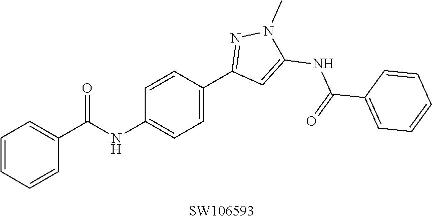 [SMILES: Cn1nc(-c2ccc(NC(=O)c3ccccc3)cc2)cc1NC(=O)c1ccccc1]

N-(4-(5-Benzamido-1-methyl-1H-pyrazol-3-yl)phenyl)benzamide (SW106593): The general procedure A was followed using N-(3-(4-aminophenyl)-1-methyl-1H-pyrazol-5-yl)benzamide (0.050 g, 0.17 mmol), benzoyl chloride (0.030 mL, 0.26 mmol), DIPEA (0.06 mL, 0.34 mmol) and DCM (1.0 mL). The crude product was purified by flash column chromatography on silica gel (DCM/MeOH, 49:1) to give SW106593 as a white solid (0.051 g, 75%). ESI MS for C₂₄H₂₀N₄O₂ m/z [M+H]⁺: calculated: 396.2, found: 397.1.

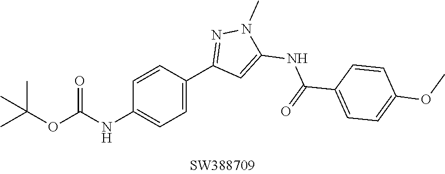 [SMILES: COc1ccc(C(=O)Nc2cc(-c3ccc(NC(=O)OC(C)(C)C)cc3)nn2C)cc1]

tert-Butyl-(4-(5-(4-methoxybenzamido)-1-methyl-1H-pyrazol-3-yl)phenyl)carbamate (SW388709): The general procedure A was followed using tert-butyl (4-(5-amino-1-methyl-1H-pyrazol-3-yl)phenyl)carbamate (0.50 g, 1.73 mmol), 4-methoxy benzoyl chloride (0.44 g, 2.60 mmol), DIPEA (0.61 mL, 3.47 mmol) and DCM (10.0 mL). The crude product was purified by flash column chromatography on silica gel (DCM/MeOH, 49:1) to give SW388709 as a white solid (0.60 g, 82%). ESI MS for C₃₁H₂₄N₄O₃ m/z [M+H]⁺: calculated: 422.2, found: 423.2.

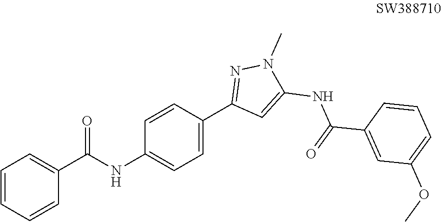 [SMILES: COc1cccc(C(=O)Nc2cc(-c3ccc(NC(=O)c4ccccc4)cc3)nn2C)c1]

N-(3-(4-Benzamidophenyl)-1-methyl-1H-pyrazol-5-yl)-3-methoxybenzamide (SW388710): The general procedure C was followed using N-(4-(5-amino-1-methyl-1H-pyrazol-3-yl)phenyl)benzamide (0.25 g, 0.86 mmol), m-anisic acid (0.156 g, 1.03 mmol), HATU (0.49 g, 1.28 mmol), DIPEA (0.3 mL, 1.71 mmol) and DMF (6.0 mL). The crude product was purified by flash column chromatography on silica gel (DCM/MeOH, 49:1) to give SW388710 as a white solid (0.28 g, 77%). ESI MS for C₂₅H₂₂N₄O₃ m/z [M+H]⁺: calculated: 426.2, found: 427.1.

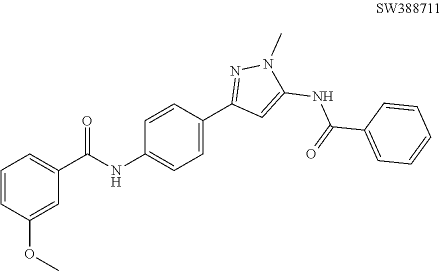 [SMILES: COc1cccc(C(=O)Nc2ccc(-c3cc(NC(=O)c4ccccc4)n(C)n3)cc2)c1]

N-(4-(5-Benzamido-1-methyl-1H-pyrazol-3-yl)phenyl)-3-methoxybenzamide (SW388711): The general procedure A was followed using N-(3-(4-aminophenyl)-1-methyl-1H-pyrazol-5-yl)benzamide (0.050 g, 0.30 mmol), 3-methoxy benzoyl chloride (0.035 mL, 0.26), DIPEA (0.06 mL, 0.34 mmol) and DCM (1.0 mL). The crude product was purified by flash column chromatography on silica gel (DCM/MeOH, 49:1) to give SW388711 as a white solid (0.034 g, 47%). ESI MS for C₂₅H₂₂N₄O₃ m/z [M+H]⁺: calculated: 426.2, found: 427.1.

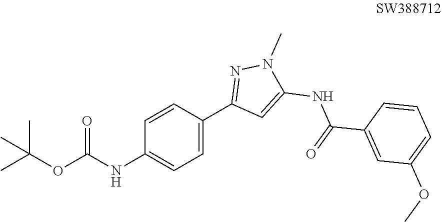 [SMILES: COc1cccc(C(=O)Nc2cc(-c3ccc(NC(=O)OC(C)(C)C)cc3)nn2C)c1]

tert-Butyl (4-(5-(3-methoxybenzamido)-1-methyl-1H-pyrazol-3-yl)phenyl)carbamate (SW388712): The general procedure A was followed using tert-butyl (4-(5-amino-1-methyl-1H-pyrazol-3-yl)phenyl)carbamate (0.50 g, 1.73 mmol), 3-methoxybenzoyl chloride (0.37 mL, 2.60 mmol), DIPEA (0.61 mL, 3.47 mmol) and DCM (10.0 mL). The crude product was purified by flash column chromatography on silica gel (DCM/MeOH, 49:1) to give SW388712 as a white solid (0.454 g, 62%). ESI MS for C₂₃H₂₆N₄O₄ m/z [M+H]⁺: calculated: 422.2, found: 423.2.

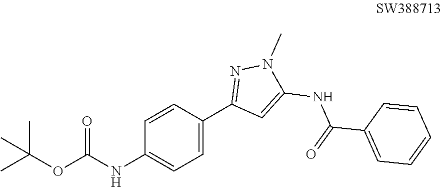 [SMILES: Cn1nc(-c2ccc(NC(=O)OC(C)(C)C)cc2)cc1NC(=O)c1ccccc1]

tert-Butyl (4-(5-benzamido-1-methyl-1H-pyrazol-3-yl)phenyl)carbamate (SW388713): The general procedure A was followed using tert-butyl (4-(5-amino-1-methyl-1H-pyrazol-3-yl)phenyl)carbamate (0.50 g, 1.73 mmol), benzoyl chloride (0.302 mL g, 2.60 mmol), DIPEA (0.61 mL, 3.47 mmol) and DCM (10.0 mL). The crude product was purified by flash column chromatography on silica gel (DCM/MeOH, 49:1) to give SW388713 as a white solid (0.54 g, 79%). ESI MS for C₂₂H₂₄N₄O₃ m/z [M+H]⁺: calculated: 392.2, found: 393.1.

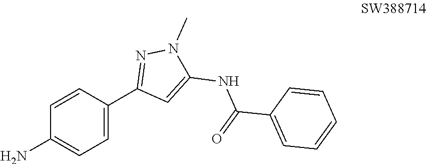 [SMILES: Cn1nc(-c2ccc(N)cc2)cc1NC(=O)c1ccccc1]

N-(3-(4-Aminophenyl)-1-methyl-1H-pyrazol-5-yl)benzamide (SW388714): The general procedure E was followed using tert-butyl (4-(5-benzamido-1-methyl-1H-pyrazol-3-yl)phenyl)carbamate (0.299 g, 1.02 mmol) and 4 M HCl in dioxane (3.0 mL) to give SW388714 as a white solid (0.189 g, 85%). The crude product was utilized in the subsequent step without purification. ESI MS for C₁₇H₁₆N₄O m/z [M+H]⁺: calculated: 292.1, found: 293.1.

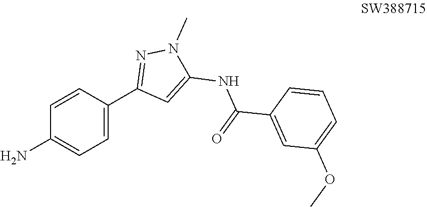 [SMILES: COc1cccc(C(=O)Nc2cc(-c3ccc(N)cc3)nn2C)c1]

N-(3-(4-Aminophenyl)-1-methyl-1H-pyrazol-5-yl)-3-methoxybenzamide (SW388715): The general procedure E was followed using tert-butyl (4-(5-(3-methoxybenzamido)-1-methyl-1H-pyrazol-3-yl)phenyl)carbamate (0.47 g, 1.11 mmol) and 4 M HCl in dioxane (3.3 mL) to give SW388715 as a brown solid (0.326 g, 91%). ESI MS for C₁₈H₁₃N₄O₂ m/z [M+H]⁺: calculated: 322.1, found: 323.1.

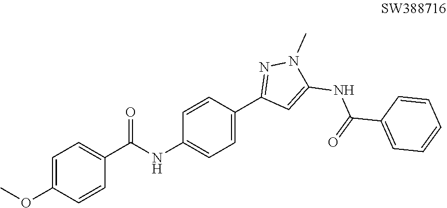 [SMILES: COc1ccc(C(=O)Nc2ccc(-c3cc(NC(=O)c4ccccc4)n(C)n3)cc2)cc1]

N-(4-(5-Benzamido-1-methyl-1H-pyrazol-3-yl)phenyl)-4-methoxybenzamide (SW388716): The general procedure A was followed using N-(3-(4-aminophenyl)-1-methyl-1H-pyrazol-5-yl)benzamide (0.050 g, 0.17 mmol), 4-methoxybenzoyl chloride (0.035 mL, 0.26 mmol), DIPEA (0.06 mL, 0.34 mmol) and DCM (1.0 mL). The crude product was purified by flash column chromatography on silica gel (DCM/MeOH, 49:1) to give SW388716 as a white solid (0.047 g, 64%). ESI MS for C₂₅H₂₂N₄O₃ m/z [M+H]⁺: calculated: 426.2, found: 427.1.

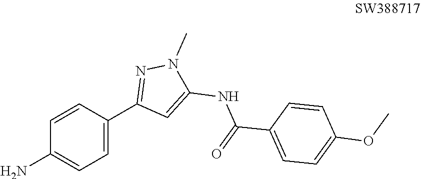 [SMILES: COc1ccc(C(=O)Nc2cc(-c3ccc(N)cc3)nn2C)cc1]

N-(3-(4-Aminophenyl)-1-methyl-1H-pyrazol-5-yl)-4-methoxybenzamide (SW388717): The general procedure E was followed using tert-butyl (4-(5-(4-methoxybenzamido)-1-methyl-1H-pyrazol-3-yl)phenyl)carbamate (0.25 g, 0.58 mmol) and 4 M HCl in dioxane (3.0 mL) to give SW388717 as a white solid (0.18 g, 94%). ESI MS for C₃₁H₂₄N₄O₃ m/z [M+H]⁺: calculated: 322.1, found: 323.2.

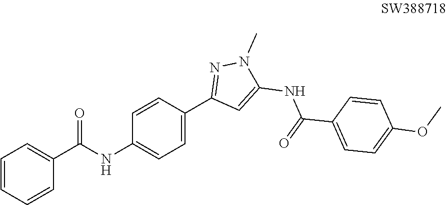 [SMILES: COc1ccc(C(=O)Nc2cc(-c3ccc(NC(=O)c4ccccc4)cc3)nn2C)cc1]

N-(3-(4-Benzamidophenyl)-1-methyl-1H-pyrazol-5-yl)-4-methoxybenzamide (SW388718): The general procedure A was followed using N-(3-(4-aminophenyl)-1-methyl-1H-pyrazol-5-yl)-4-methoxybenzamide (0.063 g, 0.19 mmol), benzoyl chloride (0.034 mL, 0.29 mmol), DIPEA (0.07 mL, 0.39 mmol) and DCM (1.0 mL). The crude product was purified by flash column chromatography on silica gel (DCM/MeOH, 97:3) to give SW388718 as a white solid (0.62 g, 76%). ESI MS for C₂₅H₂₂N₄O₃ m/z [M+H]⁺: calculated: 426.2, found: 427.2.

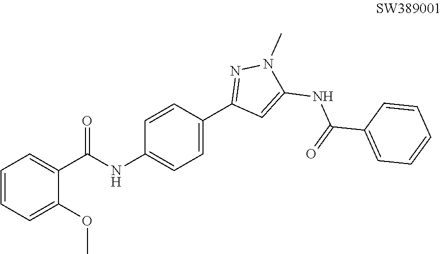 [SMILES: COc1ccccc1C(=O)Nc1ccc(-c2cc(NC(=O)c3ccccc3)n(C)n2)cc1]

N-(4-(5-Benzamido-1-methyl-1H-pyrazol-3-yl)phenyl)-2-methoxybenzamide (SW389001): The general procedure C was followed using N-(3-(4-aminophenyl)-1-methyl-1H-pyrazol-5-yl)benzamide (0.050 g, 0.17 mmol), 2-methoxy benzoic acid (0.031 g, 0.21 mmol), HATU (0.098 g, 0.26 mmol), DIPEA (0.06 mL, 0.34 mmol) and DMF (2.0 mL). The crude product was purified by flash column chromatography on silica gel (DCM/MeOH, 49:1) to give SW389001 as a white solid (0.052 g, 72%). ESI MS for C₂₅H₂₂N₄O₃ m/z [M+H]⁺: calculated: 426.2, found: 427.0.

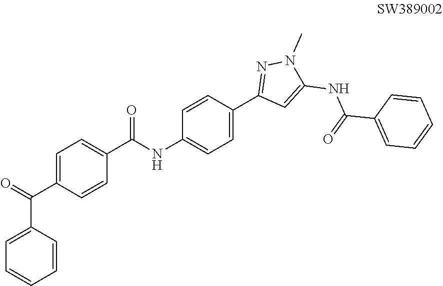 [SMILES: Cn1nc(-c2ccc(NC(=O)c3ccc(C(=O)c4ccccc4)cc3)cc2)cc1NC(=O)c1ccccc1]

N-(4-(5-Benzamido-1-methyl-1H-pyrazol-3-yl)phenyl)-4-benzoylbenzamide (SW389002): The general procedure C was followed using N-(3-(4-aminophenyl)-1-methyl-1H-pyrazol-5-yl)benzamide (0.05 g, 0.17 mmol), 4-benzoyl benzoic acid (0.046 g, 0.21 mmol), HATU (0.098 g, 0.26 mmol), DIPEA (0.06 mL, 0.34 mmol) and DMF (2 mL). The crude product was purified by flash column chromatography on silica gel (DCM/MeOH, 49:1) to give SW389002 as a white solid (0.072 g, 84%). ESI MS for C₃₁H₂₄N₄O₃ m/z [M+H]⁺: calculated: 500.2, found: 501.0.

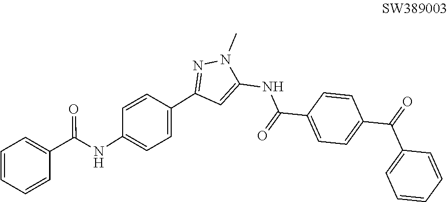 [SMILES: Cn1nc(-c2ccc(NC(=O)c3ccccc3)cc2)cc1NC(=O)c1ccc(C(=O)c2ccccc2)cc1]

N-(3-(4-Benzamidophenyl)-1-methyl-1H-pyrazol-5-yl)-4-benzoylbenzamide (SW389003): The general procedure A was followed using N-(3-(4-aminophenyl)-1-methyl-1H-pyrazol-5-yl)-4-benzoylbenzamide (0.100 g, 0.25 mmol), benzoyl chloride (0.044 mL, 0.38 mmol), DIPEA (0.09 mL, 0.50 mmol) and DCM (1.5 mL). The crude product was purified by flash column chromatography on silica gel (DCM/MeOH, 49:1) to give SW389003 as a white solid (0.102 g, 81%). ESI MS for C₃₁H₂₄N₄O₃ m/z [M+H]⁺: calculated: 500.2, found: 501.0.

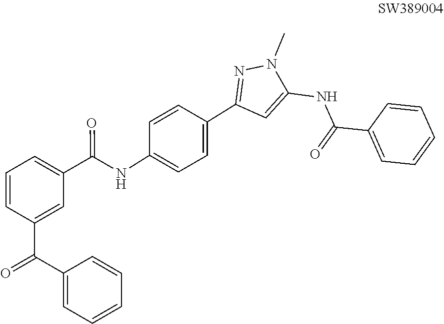 [SMILES: Cn1nc(-c2ccc(NC(=O)c3cccc(C(=O)c4ccccc4)c3)cc2)cc1NC(=O)c1ccccc1]

N-(4-(5-Benzamido-1-methyl-1H-pyrazol-3-yl)phenyl)-3-benzoylbenzamide (SW389004): The general procedure C was followed using N-(3-(4-aminophenyl)-1-methyl-1H-pyrazol-5-yl)benzamide (0.050 g, 0.17 mmol), 3-benzoyl benzoic acid (0.046 g, 0.21 mmol), HATU (0.098 g, 0.26 mmol), DIPEA (0.06 mL, 0.34 mmol) and DMF (2 mL). The crude product was purified by flash column chromatography on silica gel (DCM/MeOH, 49:1) to give SW389004 as a white solid (0.067 g, 78%). ESI MS for C₃₁H₂₄N₄O₃ m/z [M+H]⁺: calculated: 500.2, found: 501.1.

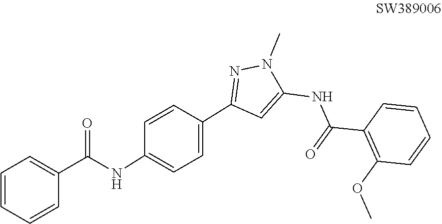 [SMILES: COc1ccccc1C(=O)Nc1cc(-c2ccc(NC(=O)c3ccccc3)cc2)nn1C]

N-(3-(4-Benzamidophenyl)-1-methyl-1H-pyrazol-5-yl)-3-ethynylbenzamide (SW389005): The general procedure C was followed using N-(4-(5-amino-1-methyl-1H-pyrazol-3-yl)phenyl)benzamide (0.100 g, 0.32 mmol), 3-ethynylbenzoyl chloride (0.06 mL, 0.47 mmol), DIPEA (0.11 mL, 0.63 mmol) and DCM (2.0 mL). The crude product was purified by flash column chromatography on silica gel (DCM/MeOH, 49:1) to give SW389005 as a white solid (0.076 g, 57%). ESI MS for C₂₆H₂₀N₄O₂ m/z [M+H]⁺: calculated: 420.2, found: 421.1.

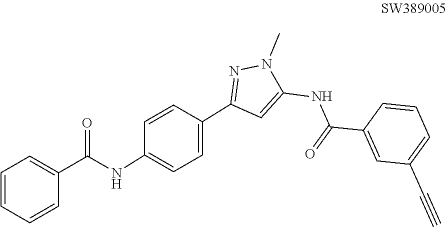 [SMILES: C#Cc1cccc(C(=O)Nc2cc(-c3ccc(NC(=O)c4ccccc4)cc3)nn2C)c1]

N-(3-(4-Benzamidophenyl)-1-methyl-1H-pyrazol-5-yl)-2-methoxybenzamide (SW389006): The general procedure A was followed using N-(3-(4-aminophenyl)-1-methyl-1H-pyrazol-5-yl)-2-methoxybenzamide (0.10 g, 0.31 mmol), benzoyl chloride (0.06 mL, 0.47 mmol), DIPEA (0.11 mL, 0.62 mmol) and DCM (2.0 mL). The crude product was purified by flash column chromatography on silica gel (DCM/MeOH, 49:1) to give SW389006 as a white solid (0.074 g, 56%). ESI MS for C₂₅H₂₂N₄O₃ m/z [M+H]⁺: calculated: 426.2, found: 427.1.

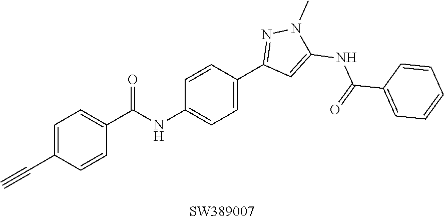 [SMILES: C#Cc1ccc(C(=O)Nc2ccc(-c3cc(NC(=O)c4ccccc4)n(C)n3)cc2)cc1]

N-(4-(5-Benzamido-1-methyl-1H-pyrazol-3-yl)phenyl)-4-ethynylbenzamide (SW389007): The general procedure A was followed using N-(3-(4-aminophenyl)-1-methyl-1H-pyrazol-5-yl)benzamide (0.050 g, 0.17 mmol), 4-ethynylbenzoyl chloride (0.042 g, 0.26 mmol), DIPEA (0.06 mL, 0.34 mmol) and DCM (2.0 mL). The crude product was purified by flash column chromatography on silica gel (DCM/MeOH, 49:1) to give SW389007 as a white solid (0.048 g, 67%). ESI MS for C₂₃H₂₀N₄O₂ m/z [M+H]⁺: calculated: 420.2, found: 421.1.

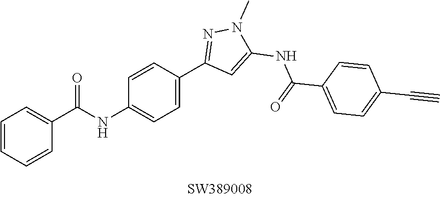 [SMILES: C#Cc1ccc(C(=O)Nc2cc(-c3ccc(NC(=O)c4ccccc4)cc3)nn2C)cc1]

N-(3-(4-Benzamidophenyl)-1-methyl-1H-pyrazol-5-yl)-4-ethynylbenzamide (SW389008): The general procedure A was followed using N-(3-(4-aminophenyl)-1-methyl-1H-pyrazol-5-yl)-4-ethynylbenzamide (0.19 g, 0.59 mmol), benzoyl chloride (0.1 mL, 0.89 mmol), DIPEA (0.21 mL, 1.19 mmol) and DCM (5.0 mL). The crude product was purified by flash column chromatography on silica gel (DCM/MeOH, 49:1) to give SW389008 as a white solid (0.14 g, 57%). ESI MS for C₂₆H₂₀N₄O₂ m/z [M+H]⁺: calculated: 420.2, found 421.1.

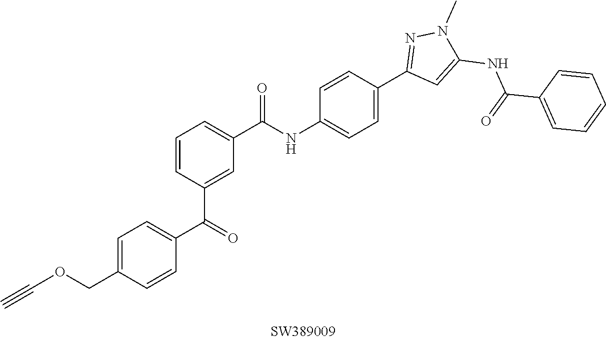 [SMILES: C#COCc1ccc(C(=O)c2cccc(C(=O)Nc3ccc(-c4cc(NC(=O)c5ccccc5)n(C)n4)cc3)c2)cc1]

4-(5-Benzamido-1-methyl-1H-pyrazol-3-yl)phenyl3-(4-((ethynyloxy)methyl)benzoyl)benzoate (SW389009): The general procedure A was followed using N-(3-(4-aminophenyl)-1-methyl-1H-pyrazol-5-yl)benzamide (0.100 g, 0.34 mmol), 3-(4-(prop-2-yn-1-yloxy)benzoyl)benzoyl chloride (0.150 g, 0.51 mmol), DIPEA (0.12 mL, 0.68 mmol) and DCM (5.0 mL). The crude product was purified by flash column chromatography on silica gel (DCM/MeOH, 97:3) to give SW389009 as a white solid (0.1 g, 53%). ESI MS for C₃₄H₂₆N₄O₄ m/z [M+H]⁺: calculated: 554.2, found: 555.0.

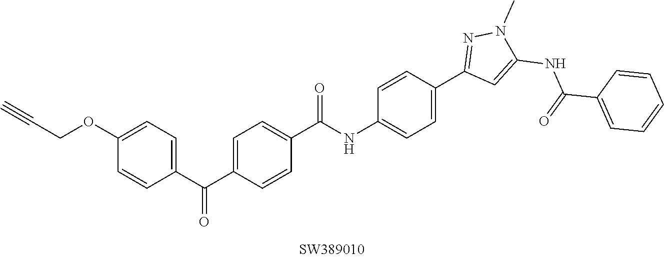 [SMILES: C#CCOc1ccc(C(=O)c2ccc(C(=O)Nc3ccc(-c4cc(NC(=O)c5ccccc5)n(C)n4)cc3)cc2)cc1]

N-(4-(5-Benzamido-1-methyl-1H-pyrazol-3-yl)phenyl)-4-(4-(prop-2-yn-1-yloxy)benzoyl)benzamide (SW389010): The general procedure A was followed using N-(3-(4-aminophenyl)-1-methyl-1H-pyrazol-5-yl)benzamide (0.100 g, 0.34 mmol), 4-(4-(prop-2-yn-1-yloxy)benzoyl)benzoyl chloride (0.150 g, 0.51 mmol), DIPEA (0.12 mL, 0.68 mmol) and DCM (5.0 mL). The crude product was purified by flash column chromatography on silica gel (DCM/MeOH, 97:3) to give SW389010 as a white solid (0.130 g, 70%). ESI MS for C₃₄H₂₆N₄O₄ m/z [M+H]⁺: calculated: 554.2, found: 555.1.

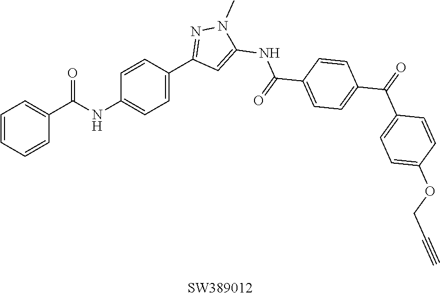 [SMILES: C#CCOc1ccc(C(=O)c2ccc(C(=O)Nc3cc(-c4ccc(NC(=O)c5ccccc5)cc4)nn3C)cc2)cc1]

N-(3-(4-Benzamidophenyl)-1-methyl-1H-pyrazol-5-yl)-4-(4-(prop-2-yn-1-yloxy)benzoyl)benzamide (SW389012): The general procedure A was followed using N-(3-(4-aminophenyl)-1-methyl-1H-pyrazol-5-yl)-4-(4-(prop-2-yn-1-yloxy)benzoyl)benzamide (0.100 g, 0.22 mmol), benzoyl chloride (0.040 mL, 0.33 mmol), DIPEA (0.08 mL, 0.44 mmol) and DCM (5.0 mL). The crude product was purified by flash column chromatography on silica gel (DCM/MeOH, 97:3) to give SW389012 as a white solid (0.100 g, 81%). ESI MS for C₃₄H₂₆N₄O₄ m/z [M+H]⁺: calculated: 554.2, found: 555.0.

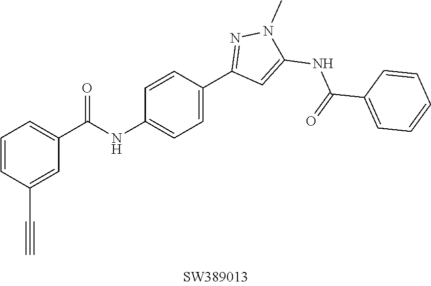 [SMILES: C#Cc1cccc(C(=O)Nc2ccc(-c3cc(NC(=O)c4ccccc4)n(C)n3)cc2)c1]

N-(4-(5-Benzamido-1-methyl-1H-pyrazol-3-yl)phenyl)-3-ethynylbenzamide (SW389013): The general procedure C was followed using N-(3-(4-aminophenyl)-1-methyl-1H-pyrazol-5-yl)benzamide (0.050 g, 0.17 mmol), 3-ethynyl benzoic acid (0.030 g, 0.21 mmol), HATU (0.097 g, 0.26 mmol), DIPEA (0.06 mL, 0.34 mmol) and DMF (2.0 mL). The crude product was purified by flash column chromatography on silica gel (DCM/MeOH, 49:1) to give SW389013 as a white solid (0.051 g, 71%). ESI MS for C₂₈H₂₀N₄O₂ m/z [M+H]⁺: calculated: 420.2, found: 421.1.

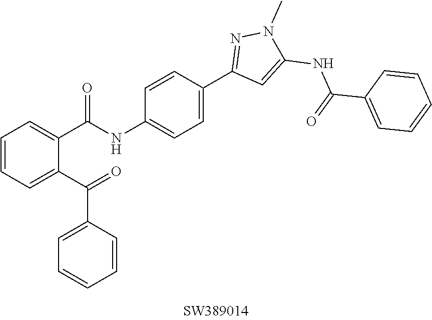 [SMILES: Cn1nc(-c2ccc(NC(=O)c3ccccc3C(=O)c3ccccc3)cc2)cc1NC(=O)c1ccccc1]

N-(4-(5-Benzamido-1-methyl-1H-pyrazol-3-yl)phenyl)-2-benzoylbenzamide (SW389014): The general procedure C was followed using N-(3-(4-aminophenyl)-1-methyl-1H-pyrazol-5-yl)benzamide (0.05 g, 0.17 mmol), 2-benzoyl benzoic acid (0.046 g, 0.21 mmol), HATU (0.098 g, 0.26 mmol), DIPEA (0.06 mL, 0.34 mmol) and DMF (2 mL). The crude product was purified by flash column chromatography on silica gel (DCM/MeOH, 49:1) to give SW389014 as a white solid (0.052 g, 61%). ESI MS for C₃₁H₂₄N₄O₃ m/z [M+H]⁺: calculated: 500.2, found: 501.1.

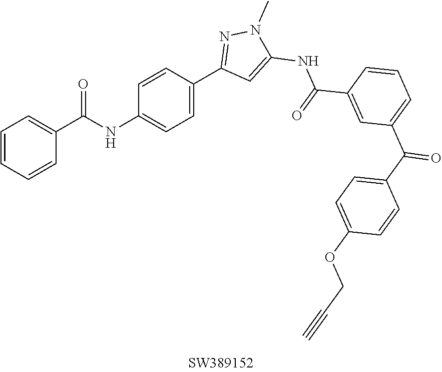 [SMILES: C#CCOc1ccc(C(=O)c2cccc(C(=O)Nc3cc(-c4ccc(NC(=O)c5ccccc5)cc4)nn3C)c2)cc1]

N-(3-(4-Benzamidophenyl)-1-methyl-1H-pyrazol-5-yl)-3-(4-(prop-2-yn-1-yloxy)benzoyl)benzamide (SW389152): The general procedure A was followed using N-(3-(4-aminophenyl)-1-methyl-1H-pyrazol-5-yl)-3-(4-(prop-2-yn-1-yloxy)benzoyl)benzamide (0.322 g, 0.71 mmol), benzoyl chloride (0.130 mL, 1.07 mmol), DIPEA (0.25 mL, 1.43 mmol) and DCM (7.0 mL). The crude product was purified by flash column chromatography on silica gel (DCM/MeOH, 97:3) to give SW389152 as a white solid (0.29 g, 75%). ESI MS for C₃₄H₂₆N₄O₄ m/z [M+H]⁺: calculated: 554.2, found: 555.2.

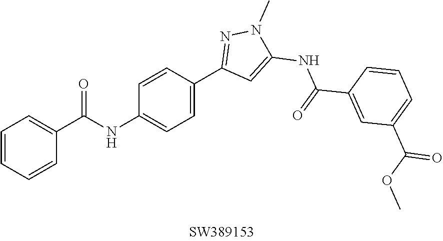 [SMILES: COC(=O)c1cccc(C(=O)Nc2cc(-c3ccc(NC(=O)c4ccccc4)cc3)nn2C)c1]

Methyl 3-((3-(4-benzamidophenyl)-1-methyl-1H-pyrazol-5-yl)carbamoyl)benzoate (SW389153): The general procedure C was followed using N-(4-(5-amino-1-methyl-1H-pyrazol-3-yl)phenyl)benzamide (0.08 g, 0.27 mmol), 3-(methoxycarbonyl)benzoic acid (0.059 g, 0.34 mmol), HATU (0.156 g, 0.41 mmol), DIPEA (0.1 mL, 0.55 mmol) and DMF (5.0 mL). The crude product was purified by flash column chromatography on silica gel (DCM/MeOH, 49:1) to give SW389153 as a white solid (0.083 g, 67%). ESI MS for C₂₆H₂₂N₄O₄ m/z [M+H]⁺: calculated: 454.2, found: 455.1.

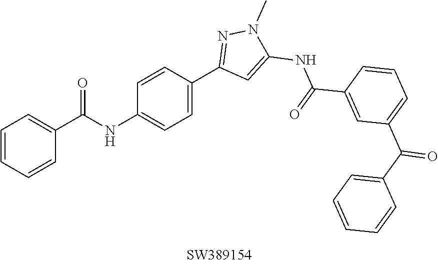 [SMILES: Cn1nc(-c2ccc(NC(=O)c3ccccc3)cc2)cc1NC(=O)c1cccc(C(=O)c2ccccc2)c1]

N-(3-(4-Benzamidophenyl)-1-methyl-1H-pyrazol-5-yl)-3-benzoylbenzamide (SW389154): The general procedure C was followed using N-(4-(5-amino-1-methyl-1H-pyrazol-3-yl)phenyl)benzamide (0.080 g, 0.27 mmol), 3-benzoylbenzoic acid (0.074 g, 0.33 mmol), HATU (0.156 g, 0.41 mmol), DIPEA (0.1 mL, 0.55 mmol) and DMF (5.0 mL). The crude product was purified by flash column chromatography on silica gel (DCM/MeOH, 49:1) to give SW389154 as a white solid (0.100 g, 74%). ESI MS for C₃₁H₂₄N₄O₃ m/z [M+H]⁺: calculated: 500.2, found: 501.0.

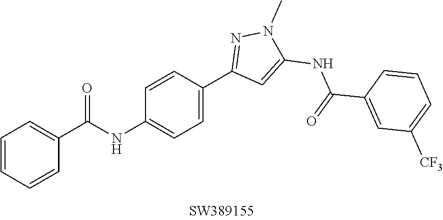 [SMILES: Cn1nc(-c2ccc(NC(=O)c3ccccc3)cc2)cc1NC(=O)c1cccc(C(F)(F)F)c1]

N-(3-(4-Benzamidophenyl)-1-methyl-1H-pyrazol-5-yl)-3-(trifluoromethyl)benzamide (SW389155): The general procedure C was followed using N-(4-(5-amino-1-methyl-1H-pyrazol-3-yl)phenyl)benzamide (0.050 g, 0.17 mmol), 3-(trifluoromethyl)benzoic acid (0.039 g, 0.21 mmol), HATU (0.098 g, 0.26 mmol), DIPEA (0.06 mL, 0.34 mmol) and DMF (3.0 mL). The crude product was purified by flash column chromatography on silica gel (DCM/MeOH, 49:1) to give SW389155 as a white solid (0.047 g, 60%). ESI MS for C₂₅H₁₉F₃N₄O₂ m/z [M+H]⁺: calculated: 464.2, found: 465.0.

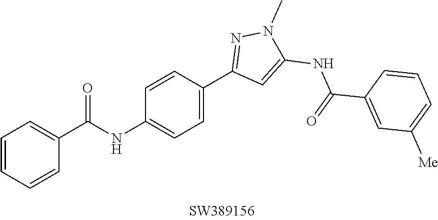 [SMILES: Cc1cccc(C(=O)Nc2cc(-c3ccc(NC(=O)c4ccccc4)cc3)nn2C)c1]

N-(3-(4-Benzamidophenyl)-1-methyl-1H-pyrazol-5-yl)-3-methylbenzamide (SW389156): The general procedure C was followed using N-(4-(5-amino-1-methyl-1H-pyrazol-3-yl)phenyl)benzamide (0.050 g, 0.17 mmol), m-toluic acid (0.028 g, 0.21 mmol), HATU (0.098 g, 0.26 mmol), DIPEA (0.06 mL, 0.34 mmol) and DMF (3.0 mL). The crude product was purified by flash column chromatography on silica gel (DCM/MeOH, 49:1) to give SW389156 as a white solid (0.060 g, 78%). ESI MS for C₂₅H₂₂N₄O₂ m/z [M+H]⁺: calculated: 410.2, found: 411.0.

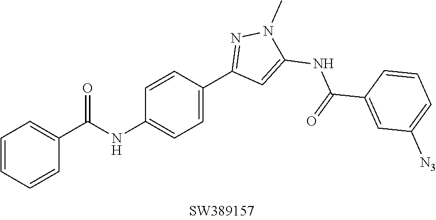 [SMILES: Cn1nc(-c2ccc(NC(=O)c3ccccc3)cc2)cc1NC(=O)c1cccc(N=[N+]=[N-])c1]

3-Azido-N-(3-(4-benzamidophenyl)-1-methyl-1H-pyrazol-5-yl)benzamide (SW389157): The general procedure C was followed using N-(4-(5-amino-1-methyl-1H-pyrazol-3-yl)phenyl)benzamide (0.080 g, 0.27 mmol), 3-azido benzoic acid (0.054 g, 0.34 mmol), HATU (0.156 g, 0.26 mmol), DIPEA (0.1 mL, 0.56 mmol) and DMF (4.0 mL). The crude product was purified by flash column chromatography on silica gel (DCM/MeOH, 97:3) to give SW389157 as a white solid (0.060 g, 49%). ESI MS for C₂₄H₁₉N₇O₂ m/z [M+H]⁺: calculated: 437.2, found: 438.0.

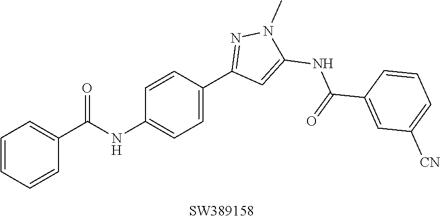 [SMILES: Cn1nc(-c2ccc(NC(=O)c3ccccc3)cc2)cc1NC(=O)c1cccc(C#N)c1]

N-(3-(4-Benzamidophenyl)-1-methyl-1H-pyrazol-5-yl)-3-cyanobenzamide (SW389158): The general procedure C was followed using N-(4-(5-amino-1-methyl-1H-pyrazol-3-yl)phenyl)benzamide (0.080 g, 0.27 mmol), 3-cyanobenzoic acid (0.048 g, 0.33 mmol), HATU (0.156 g, 0.26 mmol), DIPEA (0.1 mL, 0.56 mmol) and DMF (4.0 mL). The crude product was purified by flash column chromatography on silica gel (DCM/MeOH, 24:1) to give SW389158 as a white solid (0.060 g, 51%). ESI MS for C₂₅H₁₉N₅O₂ m/z [M+H]⁺: calculated: 421.2, found: 422.1.

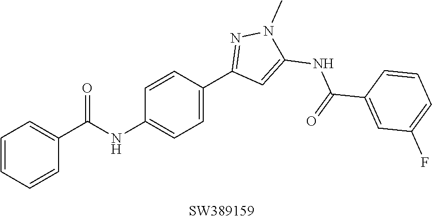 [SMILES: Cn1nc(-c2ccc(NC(=O)c3ccccc3)cc2)cc1NC(=O)c1cccc(F)c1]

N-(3-(4-Benzamidophenyl)-1-methyl-1H-pyrazol-5-yl)-3-fluorobenzamide (SW389159): The general procedure C was followed using N-(4-(5-amino-1-methyl-1H-pyrazol-3-yl)phenyl)benzamide (0.080 g, 0.27 mmol), 3-fluoro benzoic acid (0.046 g, 0.33 mmol), HATU (0.156 g, 0.26 mmol), DIPEA (0.1 mL, 0.56 mmol) and DMF (3.0 mL). The crude product was purified by flash column chromatography on silica gel (DCM/MeOH, 97:3) to give SW389159 as a white solid (0.080 g, 71%). ESI MS for C₂₄H₁₉FN₄O₂ m/z [M+H]⁺: calculated: 414.1, found: 415.0.

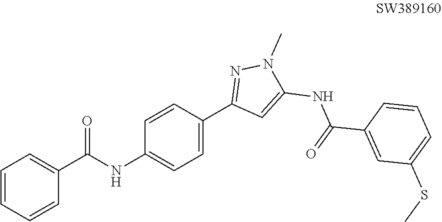 [SMILES: CSc1cccc(C(=O)Nc2cc(-c3ccc(NC(=O)c4ccccc4)cc3)nn2C)c1]

N-(3-(4-Benzamidophenyl)-1-methyl-1H-pyrazol-5-yl)-3-(methylthio)benzamide (SW389160): The general procedure C was followed using N-(4-(5-amino-1-methyl-1H-pyrazol-3-yl)phenyl)benzamide (0.100 g, 0.34 mmol), 3-(methylthio)-benzoic acid (0.069 g, 0.41 mmol), HATU (0.195 g, 0.51 mmol), DIPEA (0.12 mL, 0.68 mmol) and DMF (4.0 mL). The crude product was purified by flash column chromatography on silica gel (DCM/MeOH, 97:3) to give SW389160 as a white solid (0.100 g, 68%). ESI MS for C₂₅H₂₂N₄O₂S m/z [M+H]⁺: calculated: 442.1, found: 443.0.

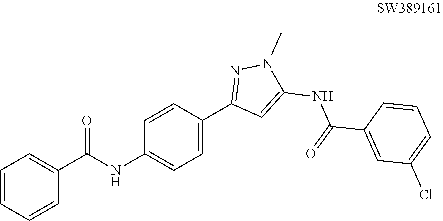 [SMILES: Cn1nc(-c2ccc(NC(=O)c3ccccc3)cc2)cc1NC(=O)c1cccc(Cl)c1]

N-(3-(4-benzamidophenyl)-1-methyl-1H-pyrazol-5-yl)-3-chlorobenzamide (SW389161): The general procedure C was followed using N-(4-(5-amino-1-methyl-1H-pyrazol-3-yl)phenyl)benzamide (0.050 g, 0.12 mmol), 3-chlorobenzoic acid (0.032 g, 0.21 mmol), HATU (0.096 g, 0.257 mmol), DIPEA (0.06 mL, 0.34 mmol) and DMF (3.0 mL). The crude product was purified by flash column chromatography on silica gel (DCM/MeOH, 97:3) to give SW389161 as a white solid (0.038 g, 73%). ¹³C NMR ESI MS for C₂₄H₁₉ClN₄O₂S m/z [M+H]⁺: calculated: 430.1, found: 431.0.

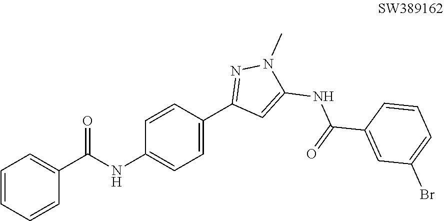 [SMILES: Cn1nc(-c2ccc(NC(=O)c3ccccc3)cc2)cc1NC(=O)c1cccc(Br)c1]

N-(3-(4-benzamidophenyl)-1-methyl-1H-pyrazol-5-yl)-3-bromobenzamide (SW389162): The general procedure C was followed using N-(4-(5-amino-1-methyl-1H-pyrazol-3-yl)phenyl)benzamide (0.050 g, 0.12 mmol), 3-bromobenzoic acid (0.032 g, 0.21 mmol), HATU (0.096 g, 0.257 mmol), DIPEA (0.06 mL, 0.34 mmol) and DMF (3.0 mL). The crude product was purified by flash column chromatography on silica gel (DCM/MeOH, 97:3) to give SW389162 as a white solid (0.038 g, 73%). ESI MS for C₂₄H₁₉BrN₄O₂S m/z [M+H]⁺: calculated: 474.1, found: 475.0.

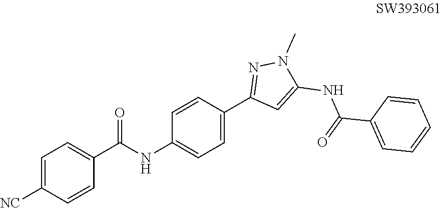 [SMILES: Cn1nc(-c2ccc(NC(=O)c3ccc(C#N)cc3)cc2)cc1NC(=O)c1ccccc1]

N-(4-(5-benzamido-1-methyl-1H-pyrazol-3-yl)phenyl)-4-cyanobenzamide (SW393061): The general procedure C was followed using N-(3-(4-aminophenyl)-1-methyl-1H-pyrazol-5-yl)benzamide (0.05 g, 0.17 mmol) 4-cyano benzoic acid (36.6 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393061 as a yellow solid (0.061 g, 85%). ESI MS for C₂₅H₁₉N₅O₂ m/z [M+H]⁺: calculated: 421.5, found: 423.1.

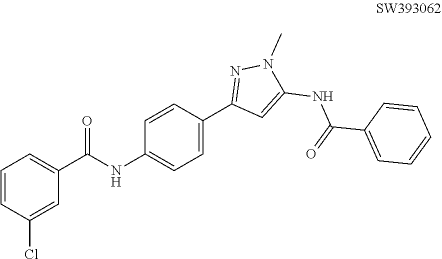 [SMILES: Cn1nc(-c2ccc(NC(=O)c3cccc(Cl)c3)cc2)cc1NC(=O)c1ccccc1]

N-(4-(5-benzamido-1-methyl-1H-pyrazol-3-yl)phenyl)-3-chlorobenzamide SW393062: The general procedure C was followed using N-(3-(4-aminophenyl)-1-methyl-1H-pyrazol-5-yl)benzamide (0.05 g, 0.17 mmol) 3-chloro benzoic acid (31.8 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393062 as a tan solid (0.056 g, 76%). ESI MS for C₂₄H₁₉ClN₄O₂ m/z [M+H]⁺: calculated: 430.9, found: 431.1.

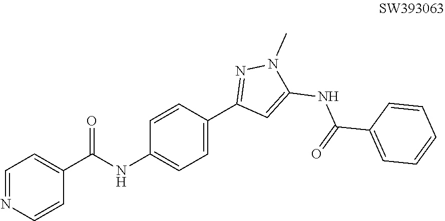 [SMILES: Cn1nc(-c2ccc(NC(=O)c3ccncc3)cc2)cc1NC(=O)c1ccccc1]

N-(4-(5-benzamido-1-methyl-1H-pyrazol-3-yl)phenyl)isonicotinamide (SW393063): The general procedure C was followed using N-(3-(4-aminophenyl)-1-methyl-1H-pyrazol-5-yl)benzamide (0.10 g, 0.34 mmol), isonicotinic acid (49.2 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393063 as a tan solid (0.078 g, 58%). ESI MS for C₂₃H₁₉N₅O₂ m/z [M+H]⁺: calculated: 397.4, found: 398.1.

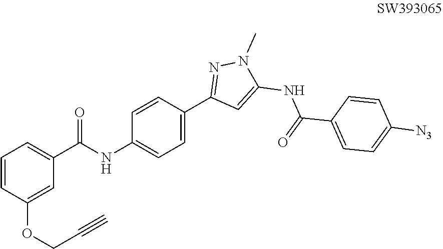 [SMILES: C#CCOc1cccc(C(=O)Nc2ccc(-c3cc(NC(=O)c4ccc(N=[N+]=[N-])cc4)n(C)n3)cc2)c1]

N-(4-(5-(4-Azidobenzamido)-1-methyl-1H-pyrazol-3-yl)phenyl)-3-(prop-2-yn-1-yloxy)benza-mide (SW393065): The general procedure A was followed using N-(3-(4-aminophenyl)-1-methyl-1H-pyrazol-5-yl)-4-azidobenzamide (0.1 g, 0.30 mmol), 3-(prop-2-yn-1-yloxy)benzoic acid (0.079 g, 0.45 mmol), SOCl₂ (0.54 mL, 4.50 mmol), DMF (3 drops), DIPEA (0.11 mL, 0.60 mmol) and DCM (7.0 mL). The crude product was purified by flash column chromatography on silica gel (DCM/MeOH, 97:3) to give SW393065 as a white solid (0.08 g, 54%). ESI MS for C₂₇H₂₁N₇O₃ m/z [M+H]⁺: calculated: 491.2, found: 492.1.

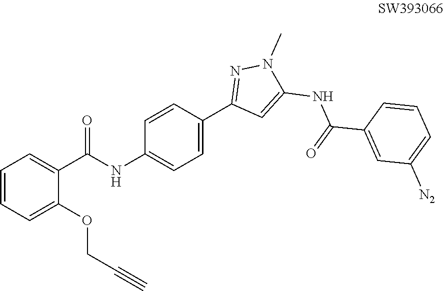

N-(4-(5-(3-Azidobenzamido)-1-methyl-1H-pyrazol-3-yl)phenyl)-2-(prop-2-yn-1-yloxy)benza-mide (SW393066): The general procedure A was followed using N-(3-(4-aminophenyl)-1-methyl-1H-pyrazol-5-yl)-3-azidobenzamide (0.1 g, 0.30 mmol), 2-(prop-2-yn-1-yloxy)benzoic acid (0.079 g, 0.45 mmol), SOCl₂ (0.54 mL, 4.50 mmol), DMF (3 drops), DIPEA (0.11 mL, 0.60 mmol) and DCM (8.0 mL). The crude product was purified by flash column chromatography on silica gel (DCM/MeOH, 97:3) to give SW393066 as a white solid (0.06 g, 42%). ESI MS for C₂₇H₂₁N₇O₃ m/z [M+H]⁺: calculated: 491.2, found: 492.2.

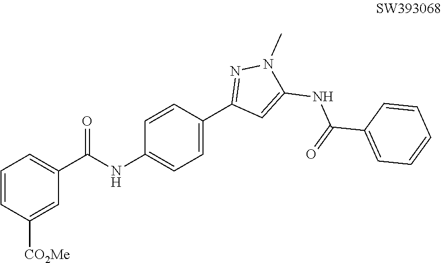 [SMILES: COC(=O)c1cccc(C(=O)Nc2ccc(-c3cc(NC(=O)c4ccccc4)n(C)n3)cc2)c1]

methyl 3-((4-(5-benzamido-1-methyl-1H-pyrazol-3-yl)phenyl)carbamoyl)benzoate (SW393068): The general procedure C was followed using N-(3-(4-aminophenyl)-1-methyl-1H-pyrazol-5-yl)benzamide (0.05 g, 0.17 mmol), mono-Methyl isophthalate (36.0 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 95:5) to give SW393068 as a tan solid (0.030 g, 39%). ESI MS for C₂₆H₂₂N₄O₄ m/z [M+H]⁺: calculated: 454.5, found: 455.1.

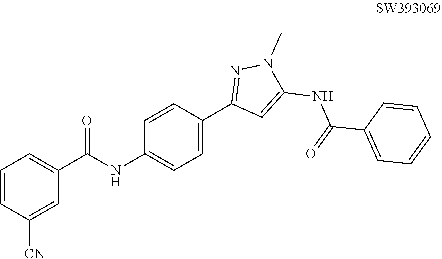 [SMILES: Cn1nc(-c2ccc(NC(=O)c3cccc(C#N)c3)cc2)cc1NC(=O)c1ccccc1]

N-(4-(5-benzamido-1-methyl-1H-pyrazol-3-yl)phenyl)-3-cyanobenzamide (SW393069): The general procedure C was followed using N-(3-(4-aminophenyl)-1-methyl-1H-pyrazol-5-yl)benzamide (0.05 g, 0.17 mmol) 3-cyano benzoic acid (36.6 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393069 as a tan solid (0.064 g, 89%). ESI MS for C₂₅H₁₉N₅O₂ m/z [M+H]⁺: calculated: 421.5, found: 423.1.

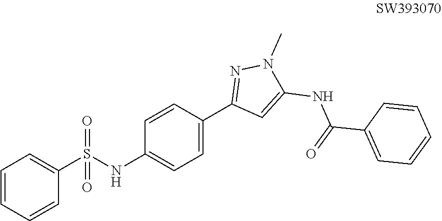 [SMILES: Cn1nc(-c2ccc(NS(=O)(=O)c3ccccc3)cc2)cc1NC(=O)c1ccccc1]

N-(1-methyl-3-(4-(phenylsulfonamido)phenyl)-1H-pyrazol-5-yl)benzamide (SW393070): A solution of benzenesulfonyl chloride (0.06 mL, 0.48 mmol) in DCM (0.5 mL) was cooled to 0° C. To the solution was slowly added a solution of using N-(3-(4-aminophenyl)-1-methyl-1H-pyrazol-5-yl)benzamide (0.10 g, 0.34 mmol) in pyridine (3 mL). The solution was raised to room temperature and stirred overnight. The completion of the reaction was monitored by LCMS. The reaction mixture was diluted with EtOAc and washed with H₂O, brine, dried (Na₂SO₄) and concentrated under reduced pressure. The crude product was purified by column chromatography on silica gel (DCM/MeOH, 98:2) to give SW393070 as a yellow solid (0.055 g, 37%). ESI MS for C₂₃H₂₀N₄O₃S m/z [M+H]⁺: calculated: 432.5, found: 434.1.

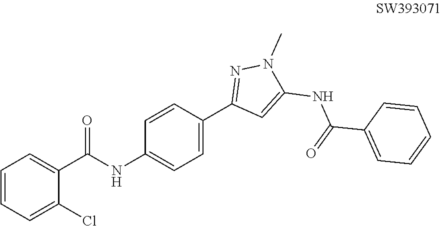 [SMILES: Cn1nc(-c2ccc(NC(=O)c3ccccc3Cl)cc2)cc1NC(=O)c1ccccc1]

N-(4-(5-benzamido-1-methyl-1H-pyrazol-3-yl)phenyl)-2-chlorobenzamide (SW393071): The general procedure C was followed using N-(3-(4-aminophenyl)-1-methyl-1H-pyrazol-5-yl)benzamide (0.05 g, 0.17 mmol) 2-chloro benzoic acid (31.8 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393071 as a tan solid (0.055 g, 75%). ESI MS for C₂₄H₁₉ClN₄O₂ m/z [M+H]⁺: calculated: 430.9, found: 431.1.

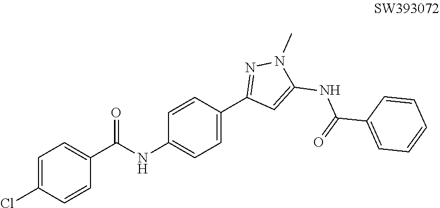 [SMILES: Cn1nc(-c2ccc(NC(=O)c3ccc(Cl)cc3)cc2)cc1NC(=O)c1ccccc1]

N-(4-(5-benzamido-1-methyl-1H-pyrazol-3-yl)phenyl)-4-chlorobenzamide (SW393072): The general procedure C was followed using N-(3-(4-aminophenyl)-1-methyl-1H-pyrazol-5-yl)benzamide (0.05 g, 0.17 mmol) 4-chloro benzoic acid (31.8 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393072 as a tan solid (0.061 g, 83%). ESI MS for C₂₄H₁₉ClN₄O₂ m/z [M+H]⁺: calculated: 430.9, found: 431.1.

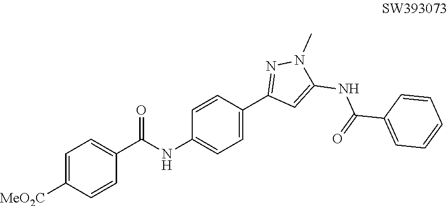 [SMILES: COC(=O)c1ccc(C(=O)Nc2ccc(-c3cc(NC(=O)c4ccccc4)n(C)n3)cc2)cc1]

methyl 4-((4-(5-benzamido-1-methyl-1H-pyrazol-3-yl)phenyl)carbamoyl)benzoate (SW393073): The general procedure C was followed using N-(3-(4-aminophenyl)-1-methyl-1H-pyrazol-5-yl)benzamide (0.05 g, 0.17 mmol), mono-Methyl terephthalate (36.6 mg, 0.2 mmol), HATU (0.097 mg, 0.26 mmol), DIPEA (0.06 mL, 0.34 mmol) and DMF (3 mL). The crude product was purified by flash column chromatography on silica gel (DCM/MeOH, 99:1) to give SW393073 as a tan solid (0.036 g, 47%). ESI MS for C₂₆H₂₂N₄O₄ m/z [M+H]⁺: calculated: 454.5, found: 455.1.

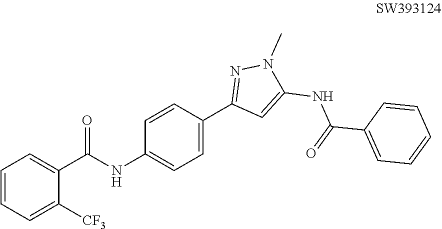 [SMILES: Cn1nc(-c2ccc(NC(=O)c3ccccc3C(F)(F)F)cc2)cc1NC(=O)c1ccccc1]

N-(4-(5-benzamido-1-methyl-1H-pyrazol-3-yl)phenyl)-2-(trifluoromethyl)benzamide (SW393124): The general procedure C was followed using N-(3-(4-aminophenyl)-1-methyl-1H-pyrazol-5-yl)benzamide (0.10 g, 0.34 mmol), 2-(trifluoromethyl)benzoic acid (76 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393124 as a tan solid (0.112 g, 71%). ESI MS for C₂₅H₁₉F₃N₄O₂ m/z [M+H]⁺: calculated: 464.5, found: 465.1.

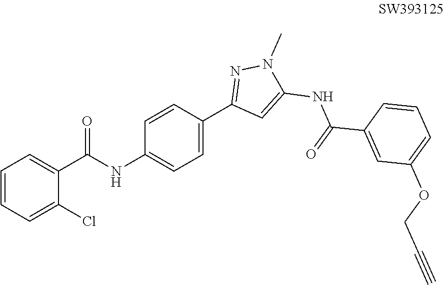 [SMILES: C#CCOc1cccc(C(=O)Nc2cc(-c3ccc(NC(=O)c4ccccc4Cl)cc3)nn2C)c1]

2-Chloro-N-(4-(1-methyl-5-(3-(prop-2-yn-1-yloxy)benzamido)-1H-pyrazol-3-yl)phenyl)benz-amide (SW393125): The general procedure A was followed using N-(4-(5-amino-1-methyl-1H-pyrazol-3-yl)phenyl)-2-chlorobenzamide (0.100 g, 0.31 mmol), 3-(prop-2-yn-1-yloxy)benzoic acid (0.081 g, 0.46 mmol), SOCl₂ (0.55 mL, 4.59 mmol), DMF (3 drops), DIPEA (0.12 mL, 0.61 mmol) and DCM (7.0 mL). The crude product was purified by flash column chromatography on silica gel (DCM/MeOH, 97:3) to give SW393125 as a white solid (0.09 g, 61%). ESI MS for C₂₇H₂₁ClN₄O₃ m/z [M+H]⁺: calculated: 484.1, found: 485.1.

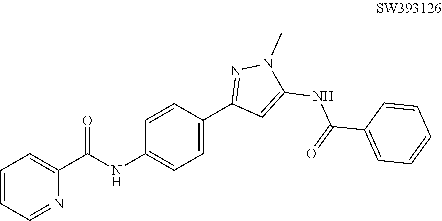 [SMILES: Cn1nc(-c2ccc(NC(=O)c3ccccn3)cc2)cc1NC(=O)c1ccccc1]

N-(4-(5-benzamido-1-methyl-1H-pyrazol-3-yl)phenyl)picolinamide (SW393126): The general procedure C was followed using N-(3-(4-aminophenyl)-1-methyl-1H-pyrazol-5-yl)benzamide (0.10 g, 0.34 mmol) 2-picolinic acid (49.2 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393126 as a tan solid (0.089 g, 66%). ESI MS for C₂₃H₉N₅O₂ m/z [M+H]⁺: calculated: 397.4, found: 398.1.

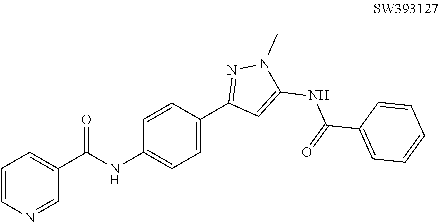 [SMILES: Cn1nc(-c2ccc(NC(=O)c3cccnc3)cc2)cc1NC(=O)c1ccccc1]

N-(4-(5-benzamido-1-methyl-1H-pyrazol-3-yl)phenyl)nicotinamide (SW393127): The general procedure C was followed using N-(3-(4-aminophenyl)-1-methyl-1H-pyrazol-5-yl)benzamide (0.10 g, 0.34 mmol) nicotinic acid (49.2 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393127 as a tan solid (0.093 g, 69%). ESI MS for C₂₃H₁₉N₅O₂ m/z [M+H]⁺: calculated: 397.4, found: 398.1.

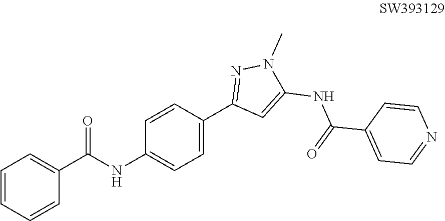 [SMILES: Cn1nc(-c2ccc(NC(=O)c3ccccc3)cc2)cc1NC(=O)c1ccncc1]

N-(3-(4-benzamidophenyl)-1-methyl-1H-pyrazol-5-yl)isonicotinamide (SW393129): The general procedure C was followed using N-(4-(5-amino-1-methyl-1H-pyrazol-3-yl)phenyl)benzamide (0.10 g, 0.34 mmol) isonicotinic acid (49.2 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393129 as a tan solid (0.057 g, 42%). ESI MS for C₂₃H₁₉N₅O₂ m/z [M+H]⁺: calculated: 397.4, found: 398.1.

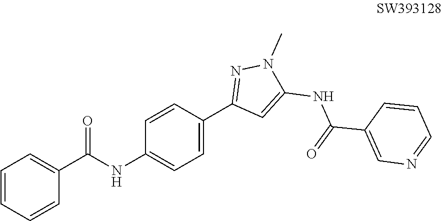 [SMILES: Cn1nc(-c2ccc(NC(=O)c3ccccc3)cc2)cc1NC(=O)c1cccnc1]

N-(3-(4-benzamidophenyl)-1-methyl-1H-pyrazol-5-yl)nicotinamide (SW393128): The general procedure C was followed using N-(4-(5-amino-1-methyl-1H-pyrazol-3-yl)phenyl)benzamide (0.10 g, 0.34 mmol) isonicotinic acid (49.2 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393128 as a tan solid (0.052 g, 39%). ESI MS for C₂₃H₁₉N₅O₂ m/z [M+H]⁺: calculated: 397.4, found: 398.1.

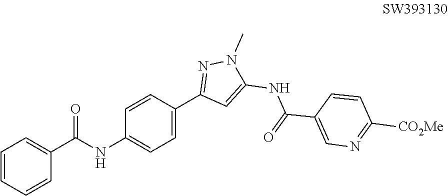 [SMILES: COC(=O)c1ccc(C(=O)Nc2cc(-c3ccc(NC(=O)c4ccccc4)cc3)nn2C)cn1]

methyl 4-((3-(4-benzamidophenyl)-1-methyl-1H-pyrazol-5-yl)carbamoyl)benzoate (SW393130): The general procedure C was followed using N-(4-(5-amino-1-methyl-1H-pyrazol-3-yl)phenyl)benzamide (0.10 g, 0.34 mmol), mono-Methyl terephthalate (72 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 94:6) to give SW393130 as a tan solid (0.034 g, 24%). ESI MS for C₂₆H₂₂N₄O₄ m/z [M+H]⁺: calculated: 454.5, found: 455.1.

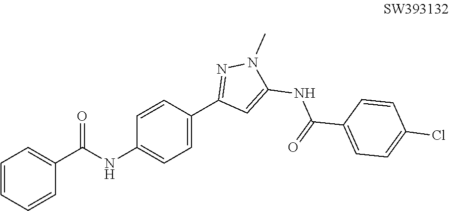 [SMILES: Cn1nc(-c2ccc(NC(=O)c3ccccc3)cc2)cc1NC(=O)c1ccc(Cl)cc1]

N-(3-(4-benzamidophenyl)-1-methyl-1H-pyrazol-5-yl)-4-chlorobenzamide (SW393132): The general procedure C was followed using N-(4-(5-amino-1-methyl-1H-pyrazol-3-yl)phenyl)benzamide (0.10 g, 0.34 mmol), 4-chloro benzoic acid (64 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 99:1) to give SW393132 as a white solid (0.048 g, 33%). ESI MS for C₂₄H₁₉ClN₄O₂ m/z [M+H]⁺: calculated: 430.9, found: 432.1.

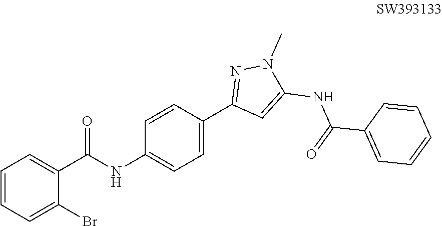 [SMILES: Cn1nc(-c2ccc(NC(=O)c3ccccc3Br)cc2)cc1NC(=O)c1ccccc1]

N-(4-(5-benzamido-1-methyl-1H-pyrazol-3-yl)phenyl)-2-bromobenzamide (SW393133): The general procedure C was followed using N-(3-(4-aminophenyl)-1-methyl-1H-pyrazol-5-yl)benzamide (0.10 g, 0.34 mmol), 2-bromo benzoic acid (80.4 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393133 as a tan solid (0.114 g, 70%). ESI MS for C₂₄H₁₉BrN₄O₂ m/z [M+H]⁺: calculated: 475.4, found: 475.1.

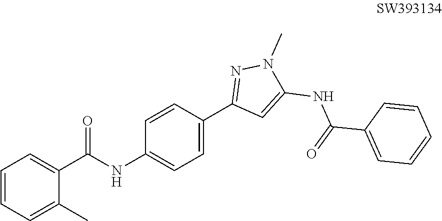 [SMILES: Cc1ccccc1C(=O)Nc1ccc(-c2cc(NC(=O)c3ccccc3)n(C)n2)cc1]

N-(4-(5-benzamido-1-methyl-1H-pyrazol-3-yl)phenyl)-2-methylbenzamide (SW393134): The general procedure C was followed using N-(3-(4-aminophenyl)-1-methyl-1H-pyrazol-5-yl)benzamide (0.10 g, 0.34 mmol), o-toluic acid (54.5 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393134 as a tan solid (0.077 g, 55%). ESI MS for C₂₅H₂₂N₄O₂ m/z [M+H]⁺: calculated: 410.5, found: 411.1.

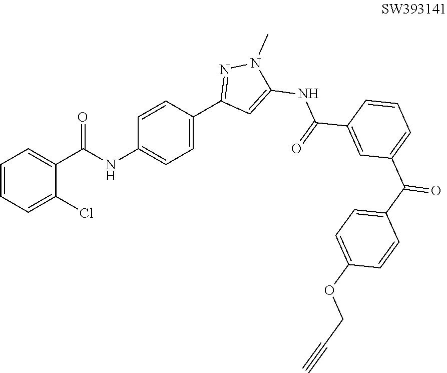 [SMILES: C#CCOc1ccc(C(=O)c2cccc(C(=O)Nc3cc(-c4ccc(NC(=O)c5ccccc5Cl)cc4)nn3C)c2)cc1]

2-Chloro-N-(4-(1-methyl-5-(3-(4-(prop-2-yn-1-yloxy)benzoyl)benzamido)-1H-pyrazol-3-yl)phenyl)benzamide (SW393141): The general procedure A was followed using N-(4-(5-amino-1-methyl-1H-pyrazol-3-yl)phenyl)-2-chlorobenzamide (0.1 g, 0.31 mmol), 3-(4-(prop-2-yn-1-yloxy)benzoyl)benzoic acid (0.129 g, 0.46 mmol), SOCl₂ (0.55 mL, 4.59 mmol), DMF (3 drops), DIPEA (0.12 mL, 0.61 mmol) and DCM (7.0 mL). The crude product was purified by flash column chromatography on silica gel (DCM/MeOH, 97:3) to give SW393141 as a white solid (0.10 g, 54%). ESI MS for C₃₄H₂₅ClN₄O₄ m/z [M+H]⁺: calculated: 588.2, found: 589.1.

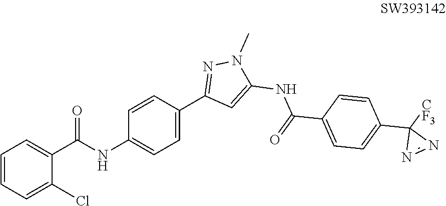 [SMILES: Cn1nc(-c2ccc(NC(=O)c3ccccc3Cl)cc2)cc1NC(=O)c1ccc(C2(C(F)(F)F)N=N2)cc1]

2-Chloro-N-(4-(1-methyl-5-(4-(3-(trifluoromethyl)-3H-diazirin-3-yl)benzamido)-1H-pyrazol-3-yl)phenyl)benzamide (SW393142): The general procedure A was followed using N-(4-(5-amino-1-methyl-1H-pyrazol-3-yl)phenyl)-2-chlorobenzamide (0.027 g, 0.084 mmol), 4-(3-(trifluoromethyl)-3H-diazirin-3-yl)benzoic acid (0.025 g, 0.109 mmol), SOCl₂ (0.13 mL, 1.07 mmol), DMF (2 drops), DIPEA (0.03 mL, 0.17 mmol) and DCM (2.5 mL). The crude product was purified by flash column chromatography on silica gel (DCM/MeOH, 24:1) to give SW393142 as a white solid (0.023 g, 52%). ESI MS for C₂₆H₁₈ClF₃N₄O₂ m/z [M+H]⁺: calculated: 538.1, found: 539.1.

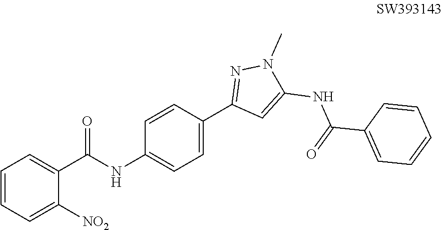 [SMILES: Cn1nc(-c2ccc(NC(=O)c3ccccc3[N+](=O)[O-])cc2)cc1NC(=O)c1ccccc1]

N-(4-(5-benzamido-1-methyl-1H-pyrazol-3-yl)phenyl)-2-nitrobenzamide (SW393143): The general procedure C was followed using N-(3-(4-aminophenyl)-1-methyl-1H-pyrazol-5-yl)benzamide (0.10 g, 0.34 mmol), 2-nitro benzoic acid (66.8 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393143 as a tan solid (0.007 g, 5%). ESI MS for C₂₄H₁₉N₅O₄ m/z [M+H]⁺: calculated: 441.5, found: 442.1.

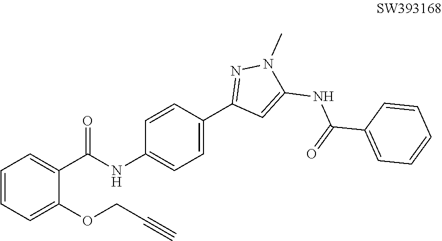 [SMILES: C#CCOc1ccccc1C(=O)Nc1ccc(-c2cc(NC(=O)c3ccccc3)n(C)n2)cc1]

N-(4-(5-benzamido-1-methyl-1H-pyrazol-3-yl)phenyl)-2-(prop-2-yn-1-yloxy)benzamide (SW393168): The general procedure C was followed using N-(3-(4-aminophenyl)-1-methyl-1H-pyrazol-5-yl)benzamide (0.10 g, 0.34 mmol), 5-chloro-2-prop-2-ynyloxy benzoic acid (70.5 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393168 as a tan solid (0.063 g, 41%). ESI MS for C₂₇H₂₂N₄O₃ m/z [M+H]⁺: calculated: 450.5, found: 451.1.

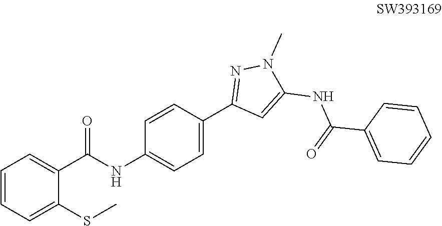 [SMILES: CSc1ccccc1C(=O)Nc1ccc(-c2cc(NC(=O)c3ccccc3)n(C)n2)cc1]

N-(4-(5-benzamido-1-methyl-1H-pyrazol-3-yl)phenyl)-2-(methylthio)benzamide (SW393169): The general procedure C was followed using N-(3-(4-aminophenyl)-1-methyl-1H-pyrazol-5-yl)benzamide (0.10 g, 0.34 mmol), 2-methylthio benzoic acid (68.3 mg, 0.4 mmol), HATU (194 mg, 0.52 mmol), DIPEA (0.12 mL, 0.68 mmol) and DMF (5 mL). The crude product was purified by flash column chromatography on silica gel (DCM/MeOH, 98:2) to give SW393169 as a tan solid (0.080 g, 53%). ESI MS for C₂₅H₂₂N₄O₂S m/z [M+H]⁺: calculated: 442.5, found: 443.1.

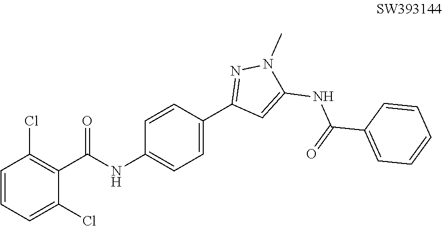 [SMILES: Cn1nc(-c2ccc(NC(=O)c3c(Cl)cccc3Cl)cc2)cc1NC(=O)c1ccccc1]

N-(4-(5-benzamido-1-methyl-1H-pyrazol-3-yl)phenyl)-2,6-dichlorobenzamide (SW393144): The general procedure A was followed using N-(3-(4-aminophenyl)-1-methyl-1H-pyrazol-5-yl)benzamide (0.10 g, 0.34 mmol), 2,6-dichloro benzoic acid (98 mg, 0.51 mmol), oxalyl chloride (0.29 mL, 3.4 mmol), DIPEA (0.3 mL, 1.7 mmol), DMF (3 drops) and DCM (5 mL). The crude product was purified by flash column chromatography on silica gel (DCM/MeOH, 98:2) to give SW393144 as a tan solid (0.036 g, 23%). ESI MS for C₂₄H₁₈Cl₂N₄O₂ m/z [M+H]⁺: calculated: 465.3, found: 467.0.

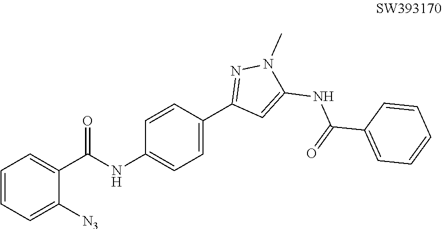 [SMILES: Cn1nc(-c2ccc(NC(=O)c3ccccc3N=[N+]=[N-])cc2)cc1NC(=O)c1ccccc1]

N-(4-(5-benzamido-1-methyl-1H-pyrazol-3-yl)phenyl)-2-azidobenzamide (SW393170): The general procedure A was followed using N-(3-(4-aminophenyl)-1-methyl-1H-pyrazol-5-yl)benzamide (0.10 g, 0.34 mmol), 2-azido benzoic acid (83.1 mg, 0.51 mmol), oxalyl chloride (0.29 mL, 3.4 mmol), DIPEA (0.3 mL, 1.7 mmol), DMF (3 drops) and DCM (5 mL). The crude product was purified by flash column chromatography on silica gel (DCM/MeOH, 98:2) to give SW393170 as a tan solid (0.046 g, 31%). ESI MS for C₂₄H₁₉N₇O₂ m/z [M+H]⁺: calculated: 437.5, found: 438.1.

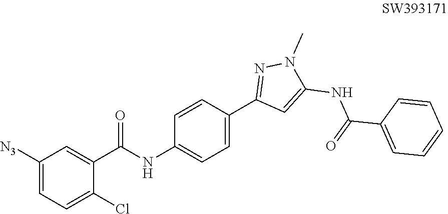 [SMILES: Cn1nc(-c2ccc(NC(=O)c3cc(N=[N+]=[N-])ccc3Cl)cc2)cc1NC(=O)c1ccccc1]

5-azido-N-(4-(5-benzamido-1-methyl-1H-pyrazol-3-yl)phenyl)-2-chlorobenzamide (SW393171): The general procedure A was followed using N-(3-(4-aminophenyl)-1-methyl-1H-pyrazol-5-yl)benzamide (0.10 g, 0.34 mmol), 2-chloro-5-azido benzoic acid (101 mg, 0.51 mmol), oxalyl chloride (0.29 mL, 3.4 mmol), DIPEA (0.3 mL, 1.7 mmol), DMF (3 drops) and DCM (5 mL). The crude product was purified by flash column chromatography on silica gel (DCM/MeOH, 98:2) to give SW393171 as a tan solid (0.046 g, 29%). ESI MS for C₂₄H₁₈ClN₇O₂ m/z [M+H]⁺: calculated: 471.9, found: 472.1.

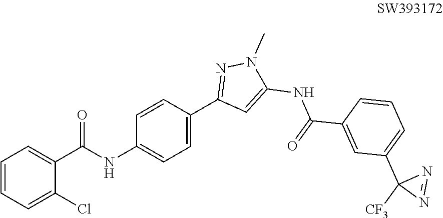 [SMILES: Cn1nc(-c2ccc(NC(=O)c3ccccc3Cl)cc2)cc1NC(=O)c1cccc(C2(C(F)(F)F)N=N2)c1]

2-Chloro-N-(4-(1-methyl-5-(3-(3-(trifluoromethyl)-3H-diazirin-3-yl)benzamido)-1H-pyrazol-3-yl)phenyl)benzamide (SW393172): The general procedure A was followed using N-(4-(5-amino-1-methyl-1H-pyrazol-3-yl)phenyl)-2-chlorobenzamide (0.027 g, 0.084 mmol), 3-(3-(trifluoromethyl)-3H-diazirin-3-yl)benzoic acid (0.025 g, 0.109 mmol), SOCl₂ (0.09 mL, 1.08 mmol), DMF (2 drops), DIPEA (0.03 mL, 0.17 mmol) and DCM (2.5 mL). The crude product was purified by flash column chromatography on silica gel (DCM/MeOH, 24:1) to give SW393172 as a white solid (0.026 g, 58%). ESI MS for C₂₆H₁₈ClF₃N₄O₂ m/z [M+H]⁺: calculated: 538.1, found: 539.1.

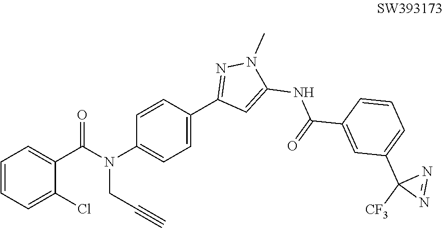 [SMILES: C#CCN(C(=O)c1ccccc1Cl)c1ccc(-c2cc(NC(=O)c3cccc(C4(C(F)(F)F)N=N4)c3)n(C)n2)cc1]

2-Chloro-N-(4-(1-methyl-5-(3-(3-(trifluoromethyl)-3H-diazirin-3-yl)benzamido)-1H-pyrazol-3-yl)phenyl)-N-(prop-2-yn-1-yl)benzamide (SW393173): The general procedure A was followed using N-(4-(5-amino-1-methyl-1H-pyrazol-3-yl)phenyl)-2-chloro-N-(prop-2-yn-1-yl)benz-amide (0.031 g, 0.084 mmol), 3-(3-(trifluoromethyl)-3H-diazirin-3-yl)benzoic acid (0.025 g, 0.109 mmol), (COCl)₂ (0.09 mL, 1.08 mmol), DMF (2 drops), DIPEA (0.03 mL, 0.17 mmol) and DCM (2.5 mL). The crude product was purified by flash column chromatography on silica gel (DCM/MeOH, 97:3) to give SW393173 as a white solid (0.027 g, 56%). ESI MS for C₂₉H₂₀ClF₃N₆O₂ m/z [M+H]⁺: calculated: 576.1, found: 577.1.

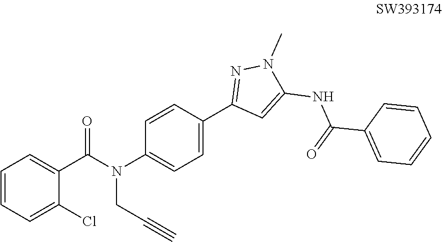 [SMILES: C#CCN(C(=O)c1ccccc1Cl)c1ccc(-c2cc(NC(=O)c3ccccc3)n(C)n2)cc1]

N-(4-(5-Benzamido-1-methyl-1H-pyrazol-3-yl)phenyl)-2-chloro-N-(prop-2-yn-1-yl)benzamil-de (SW393174): The general procedure A was followed using N-(4-(5-amino-1-methyl-1H-pyrazol-3-yl)phenyl)-2-chloro-N-(prop-2-yn-1-yl)benz-amide (0.06 g, 0.16 mmol), benzoyl chloride (0.03 mL, 0.25 mmol), DIPEA (0.06 mL, 0.33 mmol) and DCM (2.0 mL). The crude product was purified by flash column chromatography on silica gel (DCM/MeOH, 49:1) to give SW393174 as a white solid (0.046 g, 55%). ESI MS for C₂₉H₂₀ClN₄O₂ m/z [M+H]⁺: calculated: 468.1, found: 469.1.

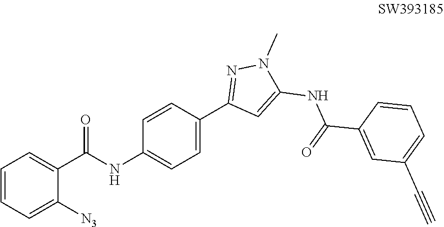 [SMILES: C#Cc1cccc(C(=O)Nc2cc(-c3ccc(NC(=O)c4ccccc4N=[N+]=[N-])cc3)nn2C)c1]

2-Azido-N-(4-(5-(3-ethynylbenzamido)-1-methyl-1H-pyrazol-3-yl)phenyl)benzamide (SW393185): The general procedure A was followed using N-(3-(4-aminophenyl)-1-methyl-1H-pyrazol-5-yl)-3-ethynylbenzamide (0.065 g, 0.21 mmol), 2-azidobenzoic acid (0.050 g, 0.31 mmol), (COCl)₂ (0.26 mL, 3.08 mmol), DMF (2 drops), DIPEA (0.072 mL, 0.41 mmol) and DCM (5.0 mL). The crude product was purified by flash column chromatography on silica gel (DCM/MeOH, 97:3) to give SW393185 as a brown solid (0.061 g, 65%). ESI MS for C₂₆H₁₉N₇O₂ m/z [M+H]⁺: calculated: 461.2, found: 462.3.

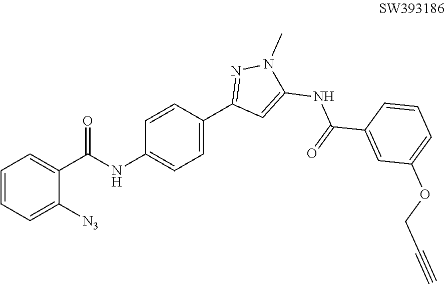 [SMILES: C#CCOc1cccc(C(=O)Nc2cc(-c3ccc(NC(=O)c4ccccc4N=[N+]=[N-])cc3)nn2C)c1]

2-Azido-N-(4-(1-methyl-5-(3-(prop-2-yn-1-yloxy)benzamido)-1H-pyrazol-3-yl)phenyl)benza-mide (SW393186): The general procedure A was followed using N-(3-(4-aminophenyl)-1-methyl-1H-pyrazol-5-yl)-3-(prop-2-yn-1-yloxy)benzamide (0.079 g, 0.23 mmol), 2-azidobenzoic acid (0.056 g, 0.34 mmol), (COCl)₂ (0.30 mL, 3.42 mmol), DMF (2 drops), DIPEA (0.080 mL, 0.46 mmol) and DCM (5.0 mL). The crude product was purified by flash column chromatography on silica gel (DCM/MeOH, 97:3) to give SW393186 as a white solid (0.056 g, 50%). 130.3, 129.9, 126.0, 125.5, 125.0, 121.0, 120.5, 119.3, 118.6, 114.1, 98.0, 78.2, 76.2, 56.1, 35.9; ESI MS for C₂₇H₂₁N₇O₃ m/z [M+H]⁺: calculated: 491.2, found: 492.1.

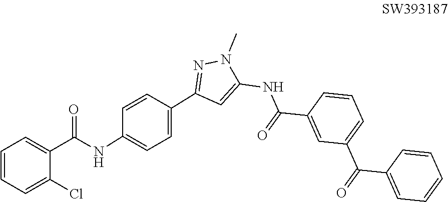 [SMILES: Cn1nc(-c2ccc(NC(=O)c3ccccc3Cl)cc2)cc1NC(=O)c1cccc(C(=O)c2ccccc2)c1]

N-(4-(5-(3-benzoylbenzamido)-1-methyl-1H-pyrazol-3-yl)phenyl)-2-chlorobenzamide (SW393187): The general procedure A was followed using N-(4-(5-amino-1-methyl-1H-pyrazol-3-yl)phenyl)-2-chlorobenzamide (0.10 g, 0.30 mmol), 3-benzoyl benzoic acid (101.8 mg, 0.45 mmol), oxalyl chloride (0.26 mL, 3.0 mmol), DIPEA (0.16 mL, 0.9 mmol), DMF (3 drops) and DCM (5 mL). The crude product was purified by flash column chromatography on silica gel (DCM/MeOH, 98:2) to give SW393187 as a tan solid (0.087 g, 54%). ESI MS for C₃₁H₂₃ClN₄O₃ m/z [M+H]⁺: calculated: 535.0, found: 537.1.

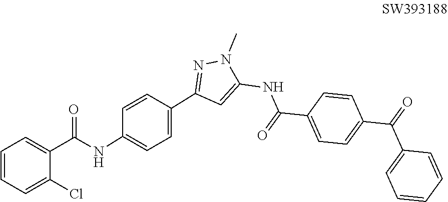 [SMILES: Cn1nc(-c2ccc(NC(=O)c3ccccc3Cl)cc2)cc1NC(=O)c1ccc(C(=O)c2ccccc2)cc1]

N-(4-(5-(4-benzoylbenzamido)-1-methyl-1H-pyrazol-3-yl)phenyl)-2-chlorobenzamide (SW393188): The general procedure A was followed using N-(4-(5-amino-1-methyl-1H-pyrazol-3-yl)phenyl)-2-chlorobenzamide (0.10 g, 0.30 mmol), 4-benzoyl benzoic acid (101.8 mg, 0.45 mmol), oxalyl chloride (0.26 mL, 3.0 mmol), DIPEA (0.16 mL, 0.9 mmol), DMF (3 drops) and DCM (5 mL). The crude product was purified by flash column chromatography on silica gel (DCM/MeOH, 98:2) to give SW393188 as a tan solid (0.094 g, 59%). ESI MS for C₃₁H₂₃ClN₄O₃ m/z [M+H]⁺: calculated: 535.0, found: 536.1.

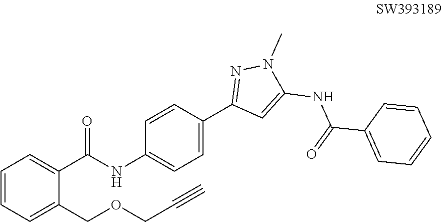 [SMILES: C#CCOCc1ccccc1C(=O)Nc1ccc(-c2cc(NC(=O)c3ccccc3)n(C)n2)cc1]

N-(4-(5-benzamido-1-methyl-1H-pyrazol-3-yl)phenyl)-2-((prop-2-yn-1-yloxy)methyl)benzamide (SW393189): The general procedure A was followed using N-(4-(5-amino-1-methyl-1H-pyrazol-3-yl)phenyl)benzamide (0.10 g, 0.34 mmol), 2-((prop-2-yn-1-yloxy)methyl)benzoic acid (97 mg, 0.51 mmol), oxalyl chloride (0.29 mL, 3.4 mmol), DIPEA (0.18 mL, 1.02 mmol), DMF (3 drops) and DCM (5 mL). The crude product was purified by flash column chromatography on silica gel (DCM/MeOH, 98:2) to give SW393189 as a tan solid (0.028 g, 18%). ESI MS for C₂₈H₂₄N₄O₃ m/z [M+H]⁺: calculated: 464.5, found: 465.1.

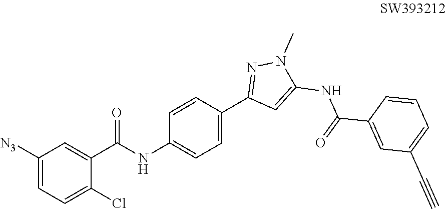 [SMILES: C#Cc1cccc(C(=O)Nc2cc(-c3ccc(NC(=O)c4cc(N=[N+]=[N-])ccc4Cl)cc3)nn2C)c1]

5-Azido-2-chloro-N-(4-(5-(3-ethynylbenzamido)-1-methyl-1H-pyrazol-3-yl)phenyl)benza-mide (SW393212): The general procedure A was followed using N-(3-(4-aminophenyl)-1-methyl-1H-pyrazol-5-yl)-3-ethynylbenzamide (0.10 g, 0.32 mmol), 5-azido-2-chlorobenzoic acid (0.094 g, 0.47 mmol), (COCl)₂ (0.43 mL, 4.74 mmol), DMF (2 drops), DIPEA (0.11 mL, 0.63 mmol) and DCM (5.0 mL). The crude product was purified by flash column chromatography on silica gel (DCM/MeOH, 97:3) to give SW393212 as a brown solid (0.083 g, 53%). ESI MS for C₂₆H₁₈ClN₇O₂ m/z [M+H]⁺: calculated: 495.1, found: 496.3.

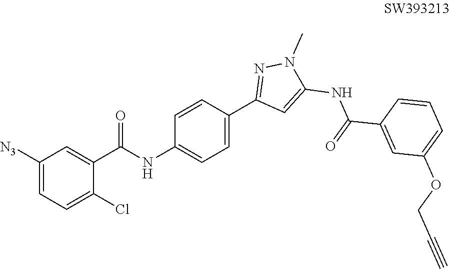 [SMILES: C#CCOc1cccc(C(=O)Nc2cc(-c3ccc(NC(=O)c4cc(N=[N+]=[N-])ccc4Cl)cc3)nn2C)c1]

5-Azido-2-chloro-N-(4-(1-methyl-5-(3-(prop-2-yn-1-yloxy)benzamido)-1H-pyrazol-3-yl)phe-nyl)benzamide (SW393213): The general procedure A was followed using N-(3-(4-aminophenyl)-1-methyl-1H-pyrazol-5-yl)-3-(prop-2-yn-1-yloxy)benzamide (0.100 g, 0.29 mmol), 5-azido-2-chlorobenzoic acid (0.086 g, 0.43 mmol), (COCl)₂ (0.43 mL, 4.74 mmol), DMF (2 drops), DIPEA (0.10 mL, 0.58 mmol) and DCM (5.0 mL). The crude product was purified by flash column chromatography on silica gel (DCM/MeOH, 97:3) to give SW393213 as a brown solid (0.085 g, 56%). ESI MS for C₂₇H₂₀ClN₇O₃ m/z [M+H]⁺: calculated: 525.1, found: 526.1.

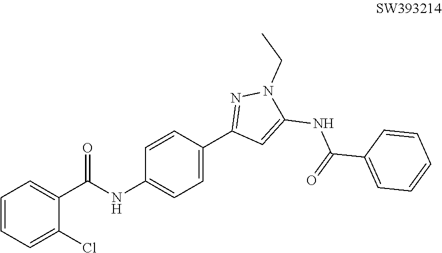 [SMILES: CCn1nc(-c2ccc(NC(=O)c3ccccc3Cl)cc2)cc1NC(=O)c1ccccc1]

N-(4-(5-Benzamido-1-ethyl-1H-pyrazol-3-yl)phenyl)-2-chlorobenzamide (SW393214): The general procedure A was followed using N-(4-(5-amino-1-ethyl-1H-pyrazol-3-yl)phenyl)-2-chlorobenzamide (0.10 g, 0.29 mmol), benzoyl chloride (0.051 mL, 0.44 mmol), DIPEA (0.10 mL, 0.58 mmol) and DCM (3.0 mL). The crude product was purified by flash column chromatography on silica gel (DCM/MeOH, 49:1) to give SW393214 as a white solid (0.068 g, 52%). ESI MS for C₂₅H₂₁ClN₄O₂ m/z [M+H]⁺: calculated: 444.1, found: 445.1.

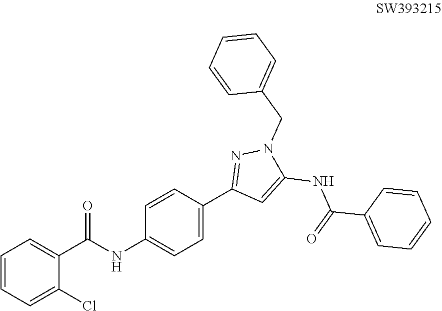 [SMILES: O=C(Nc1cc(-c2ccc(NC(=O)c3ccccc3Cl)cc2)nn1Cc1ccccc1)c1ccccc1]

N-(4-(5-Benzamido-1-benzyl-1H-pyrazol-3-yl)phenyl)-2-chlorobenzamide (SW393215): The general procedure A was followed using N-(4-(5-amino-1-benzyl-1H-pyrazol-3-yl)phenyl)-2-chlorobenzamide (0.100 g, 0.25 mmol), benzoyl chloride (0.043 mL, 0.37 mmol), DIPEA (0.09 mL, 0.50 mmol) and DCM (2.0 mL). The crude product was purified by flash column chromatography on silica gel (DCM/MeOH, 49:1) to give SW393215 as a white solid (0.077 g, 61%). ESI MS for C₃₀H₂₃ClN₄O₂ m/z [M+H]⁺: calculated: 506.2, found: 507.1.

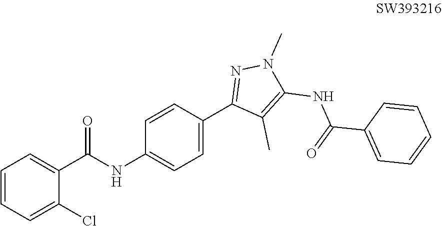 [SMILES: Cc1c(-c2ccc(NC(=O)c3ccccc3Cl)cc2)nn(C)c1NC(=O)c1ccccc1]

N-(4-(5-Benzamido-1,4-dimethyl-1H-pyrazol-3-yl)phenyl)-2-chlorobenzamide (SW393216): The general procedure A was followed using N-(4-(5-amino-1,4-dimethyl-1H-pyrazol-3-yl)phenyl)-2-chlorobenzamide (0.5 g, 1.46 mmol), benzoyl chloride (0.31 mL, 2.20 mmol), DIPEA (0.51 mL, 2.93 mmol) and DCM (10.0 mL). The crude product was purified by flash column chromatography on silica gel (DCM/MeOH, 49:1) to give SW393216 as a white solid (0.351 g, 53%). ESI MS for C₂₅H₂₁ClN₄O₂ m/z [M+H]⁺: calculated: 444.1, found: 445.1.

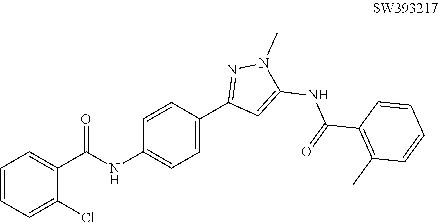 [SMILES: Cc1ccccc1C(=O)Nc1cc(-c2ccc(NC(=O)c3ccccc3Cl)cc2)nn1C]

2-chloro-N-(4-(1-methyl-5-(2-methylbenzamido)-1H-pyrazol-3-yl)phenyl)benzamide (SW393217): The general procedure A was followed using N-(4-(5-amino-1-methyl-1H-pyrazol-3-yl)phenyl)-2-chlorobenzamide (0.10 g, 0.30 mmol), o-toluic benzoic acid (61.3 mg, 0.45 mmol), oxalyl chloride (0.26 mL, 3.0 mmol), DIPEA (0.16 mL, 0.9 mmol), DMF (3 drops) and DCM (5 mL). The crude product was purified by flash column chromatography on silica gel (DCM/MeOH, 98:2) to give SW393217 as a tan solid (0.039 g, 26%). ESI MS for C₂₅H₂₁ClN₄O₂ m/z [M+H]⁺: calculated: 444.9, found: 445.1.

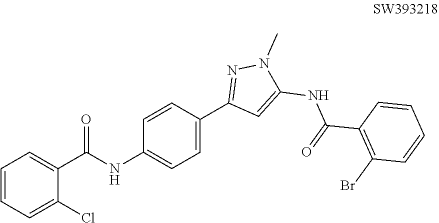 [SMILES: Cn1nc(-c2ccc(NC(=O)c3ccccc3Cl)cc2)cc1NC(=O)c1ccccc1Br]

2-bromo-N-(3-(4-(2-chlorobenzamido)phenyl)-1-methyl-1H-pyrazol-5-yl)benzamide (SW393218): The general procedure A was followed using N-(4-(5-amino-1-methyl-1H-pyrazol-3-yl)phenyl)-2-chlorobenzamide (0.10 g, 0.30 mmol), 2-bromo benzoic acid (90.5 mg, 0.45 mmol), oxalyl chloride (0.26 mL, 3.0 mmol), DIPEA (0.16 mL, 0.9 mmol), DMF (3 drops) and DCM (5 mL). The crude product was purified by flash column chromatography on silica gel (DCM/MeOH, 99:1) to give SW393218 as a yellow solid (0.076 g, 50%). ESI MS for C₂₄H₁₈BrClN₄O₂ m/z [M+H]⁺: calculated: 509.8, found: 511.0.

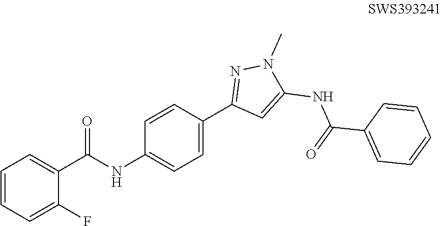 [SMILES: Cn1nc(-c2ccc(NC(=O)c3ccccc3F)cc2)cc1NC(=O)c1ccccc1]

N-(4-(5-Benzamido-1-methyl-1H-pyrazol-3-yl)phenyl)-2-fluorobenzamide (SW393241): The general procedure A was followed using N-(3-(4-aminophenyl)-1-methyl-1H-pyrazol-5-yl)benzamide (0.090 g, 0.32 mmol), 2-fluorobenzoic acid (0.068 g, 0.49 mmol), (COCl)₂ (0.43 mL, 4.85 mmol), DMF (2 drops), DIPEA (0.23 mL, 1.29 mmol) and DCM (7.0 mL). The crude product was purified by flash column chromatography on silica gel (DCM/MeOH, 97:3) to give SW393241 as a white solid (0.086 g, 64%). ESI MS for C₂₄H₁₉FN₄O₂ m/z [M+H]⁺: calculated: 414.1, found: 415.1.

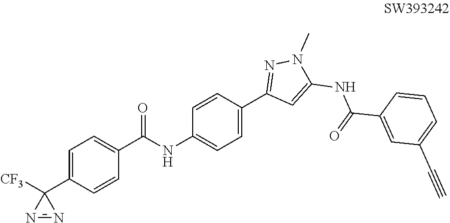 [SMILES: C#Cc1cccc(C(=O)Nc2cc(-c3ccc(NC(=O)c4ccc(C5(C(F)(F)F)N=N5)cc4)cc3)nn2C)c1]

3-Ethynyl-N-(1-methyl-3-(4-(4-(3-(trifluoromethyl)-3H-diazirin-3-yl)benzamido)phenyl)-1H-pyrazol-5-yl)benzamide (SW393242): The general procedure A was followed using N-(3-(4-aminophenyl)-1-methyl-1H-pyrazol-5-yl)-3-ethynylbenzamide (0.026 g, 0.084 mmol), 4-(3-(trifluoromethyl)-3H-diazirin-3-yl)benzoic acid (0.025 g, 0.109 mmol), (COCl)₂ (0.093 mL, 1.09 mmol), DMF (2 drops), DIPEA (0.03 mL, 0.17 mmol) and DCM (2.5 mL). The crude product was purified by flash column chromatography on silica gel (DCM/MeOH, 97:3) to give SW393242 as a white solid (0.027 g, 61%). ESI MS for C₂₈H₁₉F₃N₆O₂ m/z [M+H]⁺: calculated: 528.2, found: 529.1.

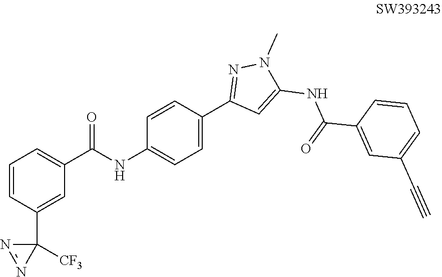 [SMILES: C#Cc1cccc(C(=O)Nc2cc(-c3ccc(NC(=O)c4cccc(C5(C(F)(F)F)N=N5)c4)cc3)nn2C)c1]

3-Ethynyl-N-(1-methyl-3-(4-(3-(3-(trifluoromethyl)-3H-diazirin-3-yl)benzamido)phenyl)-1H-pyrazol-5-yl)benzamide (SW393243): The general procedure A was followed using N-(3-(4-aminophenyl)-1-methyl-1H-pyrazol-5-yl)-3-ethynylbenzamide (0.026 g, 0.084 mmol), 3-(3-(trifluoromethyl)-3H-diazirin-3-yl)benzoic acid (0.025 g, 0.109 mmol), (COCl)₂ (0.093 mL, 1.09 mmol), DMF (2 drops), DIPEA (0.03 mL, 0.17 mmol) and DCM (2.5 mL). The crude product was purified by flash column chromatography on silica gel (DCM/MeOH, 97:3) to give SW393243 as a white solid (0.018 g, 42%). ESI MS for C₂₆H₁₉F₃N_{G}O₂ m/z [M+H]⁺: calculated: 528.2, found: 529.1.

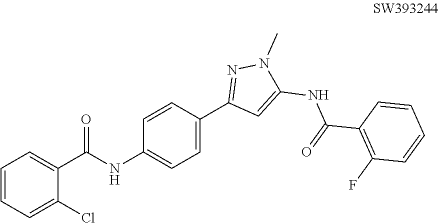 [SMILES: Cn1nc(-c2ccc(NC(=O)c3ccccc3Cl)cc2)cc1NC(=O)c1ccccc1F]

2-chloro-N-(4-(5-(2-fluorobenzamido)-1-methyl-1H-pyrazol-3-yl)phenyl)benzamide (SW393244): The general procedure A was followed using N-(4-(5-amino-1-methyl-1H-pyrazol-3-yl)phenyl)-2-chlorobenzamide (0.10 g, 0.30 mmol), 2-fluoro benzoic acid (63.0 mg, 0.45 mmol), oxalyl chloride (0.26 mL, 3.0 mmol), DIPEA (0.16 mL, 0.9 mmol), DMF (3 drops) and DCM (5 mL). The crude product was purified by flash column chromatography on silica gel (DCM/MeOH, 99:1) to give SW393244 as a tan solid (0.065 g, 48%). ESI MS for C₂₄H₁₈ClFN₄O₂ m/z [M+H]⁺: calculated: 448.9, found: 450.1.

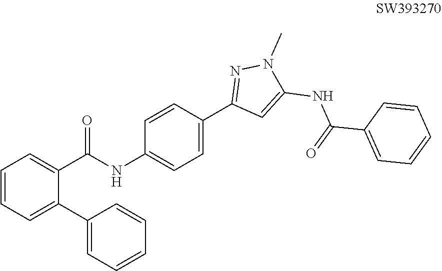 [SMILES: Cn1nc(-c2ccc(NC(=O)c3ccccc3-c3ccccc3)cc2)cc1NC(=O)c1ccccc1]

N-(4-(5-benzamido-1-methyl-1H-pyrazol-3-yl)phenyl)-[1,1′-biphenyl]-2-carboxamide (SW393270): The general procedure A was followed using N-(3-(4-aminophenyl)1-methyl-1H-pyrazol-5-yl)benzamide (0.1 g, 0.34 mmol), [1,1′-biphenyl]-2-carboxylic acid (0.102 g, 0.51 mmol), (COCl)₂ (0.44 mL, 5.13 mmol), DMF (2 drops), DIPEA (0.12 mL, 0.68 mmol) and DCM (5.0 mL). The crude product was purified by flash column chromatography on silica gel (DCM/MeOH, 97:3) to give SW393270 as a white solid (0.094 g, 58%). ESI MS for C₃₀H₂₄N₄O₂ m/z [M+H]⁺: calculated: 472.2, found: 473.2.

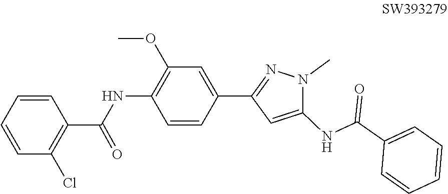 [SMILES: COc1cc(-c2cc(NC(=O)c3ccccc3)n(C)n2)ccc1NC(=O)c1ccccc1Cl]

N-(4-(5-benzamido-1-methyl-1H-pyrazol-3-yl)-2-methoxyphenyl)-2-chlorobenzamide (SW393279) was synthesized from methyl-4-nitro-3-methoxy-benzoate in analogy to SW393071. 1H NMR (400 MHz, cdcl3) δ 8.57 (s, 1H), 8.44-8.37 (m, 1H), 7.95-7.89 (m, 2H), 7.73-7.67 (m, 1H), 7.52 (tt, J=7.6, 1.7 Hz, 1H), 7.48-7.32 (m, 6H), 7.32-7.27 (m, 1H), 6.58-6.45 (m, 1H), 3.92 (d, J=1.6 Hz, 3H), 3.88-3.72 (m, 3H).

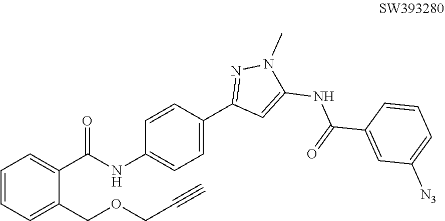 [SMILES: C#CCOCc1ccccc1C(=O)Nc1ccc(-c2cc(NC(=O)c3cccc(N=[N+]=[N-])c3)n(C)n2)cc1]

N-(4-(5-(3-azidobenzamido)-1-methyl-1H-pyrazol-3-yl)phenyl)-2-((prop-2-yn-1-yloxy)methyl)benzamide (SW393280): The general procedure A was followed using N-(3-(4-aminophenyl)-1-methyl-1H-pyrazol-5-yl)-3-azidobenzamide (0.115 g, 0.35 mmol), 2-((prop-2-yn-1-yloxy)methyl)benzoic acid (98.8 mg, 0.52 mmol), oxalyl chloride (0.3 mL, 3.5 mmol), DIPEA (0.25 mL, 1.4 mmol), DMF (3 drops) and DCM (5 mL). The crude product was purified by flash column chromatography on silica gel (DCM/MeOH, 98:2) to give SW393280 as a white solid (0.020 g, 12%). ESI MS for C₃₇H₄₂N₈O₆ m/z [M+H]⁺: calculated: 505.4, found: 506.1.

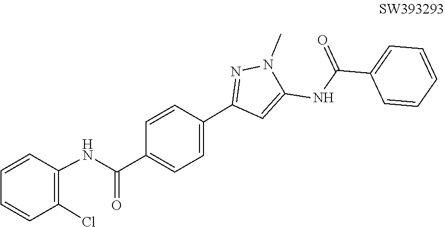 [SMILES: Cn1nc(-c2ccc(C(=O)Nc3ccccc3Cl)cc2)cc1NC(=O)c1ccccc1]

4-(5-benzamido-1-methyl-1H-pyrazol-3-yl)-N-(2-chlorophenyl)benzamide (SW393293) was synthesized analogously to SW393071 starting from 4-(methoxycarbonyl)benzoic acid. 1H NMR (400 MHz, MeOD) δ 8.12 (dd, J=8.2, 1.6 Hz, 1H), 8.02-7.90 (m, 7H), 7.62-7.58 (m, 1H), 7.57-7.49 (m, 4H), 7.49-7.44 (m, 2H), 7.34 (td, J=7.7, 1.6 Hz, 2H), 7.17 (td, J=7.7, 1.7 Hz, 1H), 6.72 (s, 1H), 3.87 (d, J=3.6 Hz, 3H).

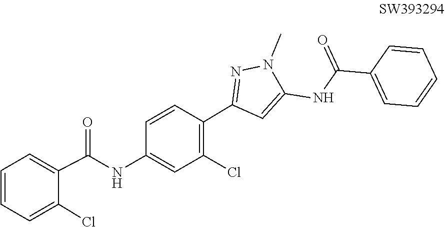 [SMILES: Cn1nc(-c2ccc(NC(=O)c3ccccc3Cl)cc2Cl)cc1NC(=O)c1ccccc1]

N-(4-(5-benzamido-1-methyl-1H-pyrazol-3-yl)-3-chlorophenyl)-2-chlorobenzamide (SW393294): The general procedure A was followed using N-(4-(5-amino-1-methyl-1H-pyrazol-3-yl)-3-chlorophenyl)-2-chlorobenzamide (0.20 g, 0.55 mmol), benzoyl chloride (0.09 mL, 0.83 mmol), DIPEA (0.19 mL, 1.11 mmol) and DCM (5.0 mL). The crude product was purified by recrystallization in DCM/hexanes mixtures to give SW393294 as a white solid (0.18 g, 71%). ESI MS for C₂₄H₁₈Cl₂N₄O₂ m/z [M+H]⁺: calculated:464.1, found 465.1.

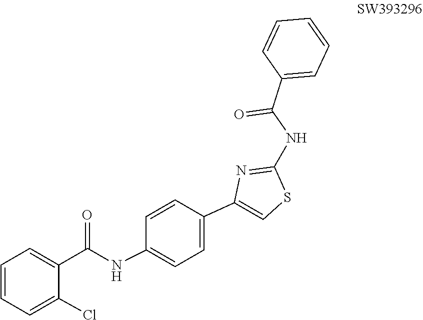 [SMILES: O=C(Nc1nc(-c2ccc(NC(=O)c3ccccc3Cl)cc2)cs1)c1ccccc1]

N-(4-(2-Benzamidothiazol-4-yl)phenyl)-2-chlorobenzamide (SW393296): The general procedure A was followed using N-(4-(4-aminophenyl)thiazol-2-yl)benzamide (0.1 g, 0.34 mmol), 2-chlorobenzoic acid (0.080 g, 0.51 mmol), (COCl)₂ (0.44 mL, 5.13 mmol), DMF (2 drops), DIPEA (0.12 mL, 0.68 mmol) and DCM (5.0 mL). The crude product was purified by flash column chromatography on silica gel (DCM/MeOH, 97:3) to give SW393296 as a white solid (0.104 g, 71%). ESI MS for C₂₃H₁₆ClN₃O₂S m/z [M+H]⁺: calculated: 433.1, found: 434.0.

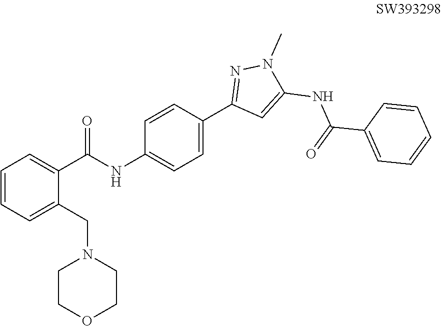 [SMILES: Cn1nc(-c2ccc(NC(=O)c3ccccc3CN3CCOCC3)cc2)cc1NC(=O)c1ccccc1]

N-(4-(5-Benzamido-1-methyl-1H-pyrazol-3-yl)phenyl)-2-(morpholinomethyl)benzamide (SW393298): The general procedure A was followed using N-(3-(4-aminophenyl)-1-methyl-1H-pyrazol-5-yl)benzamide (0.10 g, 0.34 mmol), 2-(morpholinomethyl)benzoic acid (0.113 g, 0.51 mmol), SOCl₂ (0.37 mL, 5.10 mmol), DMF (3 drops), Et₃N (0.10 mL, 0.68 mmol) and DMF (5.0 mL). The crude product was purified by flash column chromatography on silica gel (DCM/MeOH, 97:3) to give SW393298 as a white solid (0.098 g, 58%). ESI MS for C₂₉H₂₉N₅O₃ m/z [M+H]⁺: calculated: 495.2, found: 496.2.

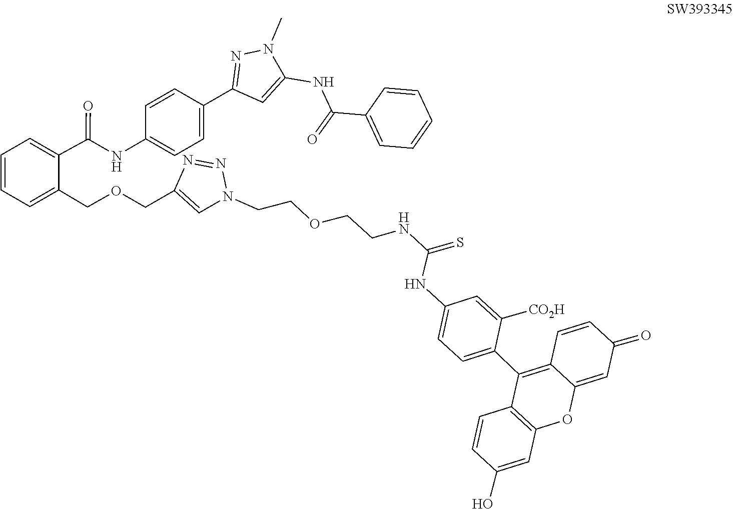 [SMILES: Cn1nc(-c2ccc(NC(=O)c3ccccc3COCc3cn(CCOCCNC(=S)Nc4ccc(-c5c6ccc(=O)cc-6oc6cc(O)ccc56)c(C(=O)O)c4)nn3)cc2)cc1NC(=O)c1ccccc1]

5-(3-(2-(2-(4-(((2-((4-(5-benzamido-1-methyl-1H-pyrazol-3-yl)phenyl)carbamoyl)benzyl)oxy)methyl)-1H-1,2,3-triazol-1-yl)ethoxy)ethyl)thioureido)-2-(6-hydroxy-3-oxo-3H-xanthen-9-yl)benzoic acid (SW393345): A solution of FITC (58.6 mg, 0.15 mmol) and NEt₃ (0.02 mL, 0.13 mL) in DMF (1 mL) was cooled to 0° C. 2-(((1-(2-(2-aminoethoxy)ethyl)-1H-1,2,3-triazol-4-yl)methoxy)methyl)-N-(4-(5-benzamido-1-methyl-1H-pyrazol-3-yl)phenyl)benzamide (72.8 mg, 0.13 mL) in DMF (1 mL) was added, raised to room temperature and stirred under N₂ for 2 hours. The mixture was diluted with EtOAc and washed with H₂O, brine, dried (Na₂SO₄) and concentrated under reduced pressure. The crude product was purified by column chromatography on silica gel (DCM/MeOH 90:10) to give SW393345 as a white solid (0.079 g, 62%). ESI MS for C₅₃H₄₅N₉O₉S m/z [M+H]⁺: calculated: 983.3, found: 984.1.

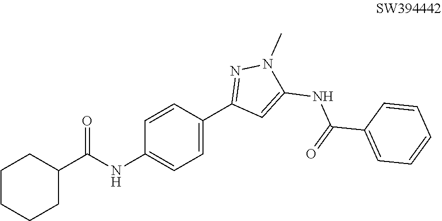 [SMILES: Cn1nc(-c2ccc(NC(=O)C3CCCCC3)cc2)cc1NC(=O)c1ccccc1]

N-(3-(4-(Cyclohexanecarboxamido)phenyl)-1-methyl-1H-pyrazol-5-yl)benzamide (SW394442): The general procedure C was followed using N-(3-(4-aminophenyl)1-methyl-1H-pyrazol-5-yl)benzamide (0.10 g, 0.34 mmol), cyclohexanecarboxylic acid (0.053 g, 0.41 mmol), HATU (0.144 g, 0.51 mmol), DIPEA (0.12 mL, 0.68 mmol) and DCM (7.0 mL). The crude product was purified by flash column chromatography on silica gel (DCM/MeOH, 49:1) to give SW394442 as a white solid (0.085 g, 62%). ESI MS for C₂₄H₂₆N₄O₂ m/z [M+H]⁺: calculated: 402.2, found: 403.2

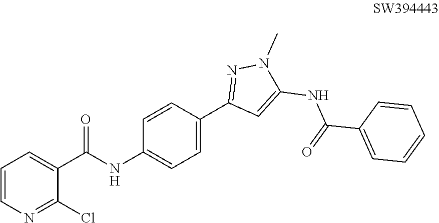 [SMILES: Cn1nc(-c2ccc(NC(=O)c3cccnc3Cl)cc2)cc1NC(=O)c1ccccc1]

N-(4-(5-Benzamido-1-methyl-1H-pyrazol-3-yl)phenyl)-2-chloronicotinamide (SW394443): The general procedure C was followed using N-(3-(4-aminophenyl)-1-methyl-1H-pyrazol-5-yl)benzamide (0.10 g, 0.34 mmol), 2-chloronicotinic acid (0.065 g, 0.41 mmol), HATU (0.144 g, 0.51 mmol), DIPEA (0.12 mL, 0.68 mmol) and DCM (3.5 mL). The crude product was purified by flash column chromatography on silica gel (DCM/MeOH, 19:1) to give SW394443 as a white solid (0.103 g, 70%). ESI MS for C₂₃H₁₈ClN₅O₂ m/z [M+H]⁺: calculated: 431.1, found: 432.1.

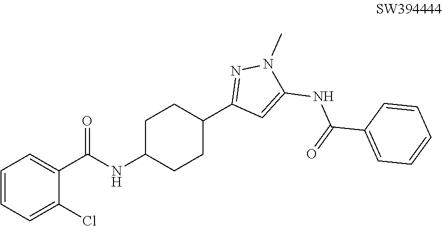 [SMILES: Cn1nc(C2CCC(NC(=O)c3ccccc3Cl)CC2)cc1NC(=O)c1ccccc1]

N-(4-(5-benzamido-1-methyl-1H-pyrazol-3-yl)cyclohexyl)-2-chlorobenzamide (SW394444): The general procedure A was followed using N-(3-(4-aminohexyl)-1-methyl-1H-pyrazol-5-yl)benzamide (0.20 g, 0.67 mmol), 2-chlorobenzoic acid (0.16 g, 1.0 mmol), oxalyl chloride (0.86 mL, 10 mmol), DIPEA (0.24 mL, 1.3 mmol), DMF (3 drops) and DCM (6 mL). The crude product was purified by flash column chromatography on silica gel (DCM/MeOH, 98:2) to give SW394444 as a white solid (0.14 g, 48%). ESI MS for C₂₄H₂₅ClN₄O₂ m/z [M+H]⁺: calculated: 436.2, found: 437.2.

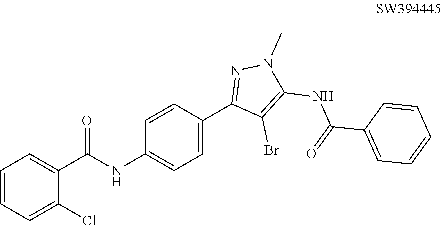 [SMILES: Cn1nc(-c2ccc(NC(=O)c3ccccc3Cl)cc2)c(Br)c1NC(=O)c1ccccc1]

N-(4-(5-benzamido-4-bromo-1-methyl-1H-pyrazol-3-yl)phenyl)-2-(trifluoromethyl)benzamide (SW394445): The general procedure A was followed using N-(3-(4-aminophenyl)4-bromo-1-methyl-1H-pyrazol-5-yl)benzamide (0.18 g, 0.48 mmol), 2-trifluoromethylbenzoic acid (0.14 g, 0.72 mmol), oxalyl chloride (0.12 mL, 0.72 mmol), DIPEA (0.17 mL, 0.96 mmol), DMF (3 drops) and DCM (8 mL). The crude product was purified by flash column chromatography on silica gel (DCM/MeOH, 98:2) to give SW394445 as a white solid (0.14 g, 48%). ESI MS for C₂₅H₁₈BrF₃N₄O₂ m/z [M+H]⁺: calculated: 542.1, found: 543.0.

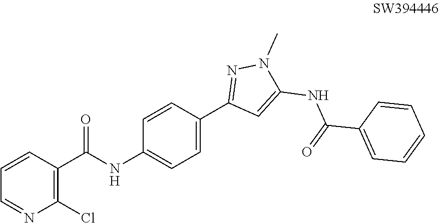 [SMILES: Cn1nc(-c2ccc(NC(=O)c3cccnc3Cl)cc2)cc1NC(=O)c1ccccc1]

N-(4-(5-Benzamido-1-methyl-1H-pyrazol-3-yl)phenyl)-3-chloroisonicotinamide (SW394446): The general procedure C was followed using N-(3-(4-aminophenyl)-1-methyl-1H-pyrazol-5-yl)benzamide (0.10 g, 0.34 mmol), 3-chloroisonicotinic acid (0.065 g, 0.41 mmol), HATU (0.144 g, 0.51 mmol), DIPEA (0.12 mL, 0.68 mmol) and DCM (3.5 mL). The crude product was purified by flash column chromatography on silica gel (DCM/MeOH, 19:1) to give SW394446 as a white solid (0.072 g, 49%). ESI MS for C₂₃H₁₈ClN₅O₂ m/z [M+H]⁺: calculated: 431.1, found: 432.3.

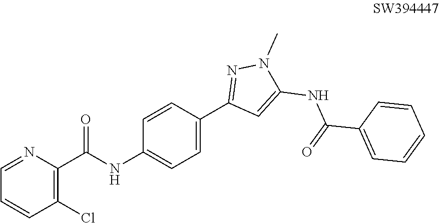 [SMILES: Cn1nc(-c2ccc(NC(=O)c3ncccc3Cl)cc2)cc1NC(=O)c1ccccc1]

N-(4-(5-Benzamido-1-methyl-1H-pyrazol-3-yl)phenyl)-3-chloropicolinamide (SW394447): The general procedure C was followed using N-(3-(4-aminophenyl)-1-methyl-1H-pyrazol-5-yl)benzamide (0.10 g, 0.34 mmol), 3-chloropicolinic acid (0.065 g, 0.41 mmol), HATU (0.144 g, 0.51 mmol), DIPEA (0.12 mL, 0.68 mmol) and DCM (3.5 mL). The crude product was purified by flash column chromatography on silica gel (DCM/MeOH, 19:1) to give SW394447 as a white solid (0.091 g, 62%). ESI MS for C₂₃H₁₈ClN₅O₂ m/z [M+H]⁺: calculated: 431.1, found: 432.1

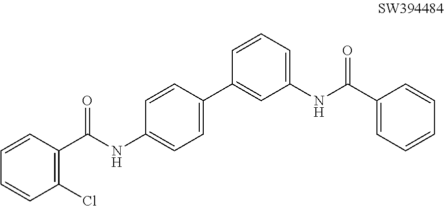 [SMILES: O=C(Nc1cccc(-c2ccc(NC(=O)c3ccccc3Cl)cc2)c1)c1ccccc1]

N-(3′-benzamido-[1,1′-biphenyl]-4-yl)-2-chlorobenzamide (SW394484): The general procedure A was followed using N-(4′-amino-[1,1′-biphenyl]-3-yl)benzamide (0.51 g, 0.18 mmol), 2-chloro benzoic acid (41.3 mg, 0.26 mmol), oxalyl chloride (0.15 mL, 1.8 mmol), DIPEA (0.1 mL, 0.53 mmol), DMF (3 drops) and DCM (2 mL). The crude product was purified by flash column chromatography on silica gel (EtOAc/hex, 60:40) to give SW394484 as a white solid (0.022 g, 30%). ESI MS for C₂₆H₁₉ClN₂O₂ m/z [M+H]⁺: calculated: 427.9, found: 428.1.

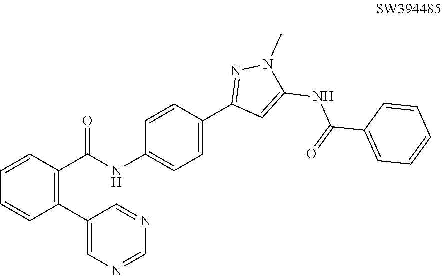 [SMILES: Cn1nc(-c2ccc(NC(=O)c3ccccc3-c3cncnc3)cc2)cc1NC(=O)c1ccccc1]

N-(4-(5-Benzamido-1-methyl-1H-pyrazol-3-yl)phenyl)-2-(pyrimidin-5-yl)benzamide (SW394485): The general procedure C was followed using N-(3-(4-aminophenyl)1-methyl-1H-pyrazol-5-yl)benzamide (0.136 g, 0.47 mmol), 2-(pyrimidin-5-yl)benzoic acid (0.112 g, 0.56 mmol), HATU (0.196 g, 0.70 mmol), DIPEA (0.17 mL, 0.93 mmol) and DCM (6.0 mL). The crude product was purified by flash column chromatography on silica gel (DCM/MeOH, 19:1) to give SW394485 as a white solid (0.147 g, 67%). ESI MS for C₂₈H₂₂N₆O₂ m/z [M+H]⁺: calculated: 474.2, found: 475.2.

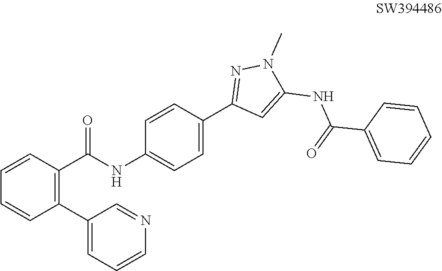 [SMILES: Cn1nc(-c2ccc(NC(=O)c3ccccc3-c3cccnc3)cc2)cc1NC(=O)c1ccccc1]

N-(4-(5-Benzamido-1-methyl-1H-pyrazol-3-yl)phenyl)-2-(pyridin-3-yl)benzamide (SW394486): The general procedure C was followed using N-(3-(4-aminophenyl)1-methyl-1H-pyrazol-5-yl)benzamide (0.160 g, 0.55 mmol), 2-(pyridin-3-yl)benzoic acid (0.131 g, 0.66 mmol), HATU (0.231 g, 0.82 mmol), DIPEA (0.19 mL, 1.09 mmol) and DCM (8.0 mL). The crude product was purified by flash column chromatography on silica gel (DCM/MeOH, 19:1) to give SW394486 as a white solid (0.176 g, 68%). ESI MS for C₂₉H₂₃N₅O₂ m/z [M+H]⁺: calculated: 473.2, found: 474.2.

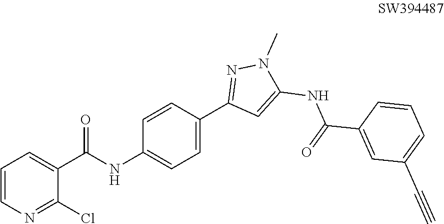 [SMILES: C#Cc1cccc(C(=O)Nc2cc(-c3ccc(NC(=O)c4cccnc4Cl)cc3)nn2C)c1]

2-Chloro-N-(4-(5-(3-ethynylbenzamido)-1-methyl-1H-pyrazol-3-yl)phenyl)nicotinamide (SW394487): The general procedure C was followed using N-(3-(4-aminophenyl)-1-methyl-1H-pyrazol-5-yl)-3-ethynylbenzamide (0.100 g, 0.32 mmol), 2-chloronicotinic acid (0.060 g, 0.34 mmol), HATU (0.133 g, 0.47 mmol), DIPEA (0.11 mL, 0.63 mmol) and DCM (3.5 mL). The crude product was purified by flash column chromatography on silica gel (DCM/MeOH, 19:1) to give SW394487 as a white solid (0.081 g, 57%). ESI MS for C₂₅H₁₈ClN₅O₂ m/z [M+H]⁺: calculated: 455.1, found: 456.1.

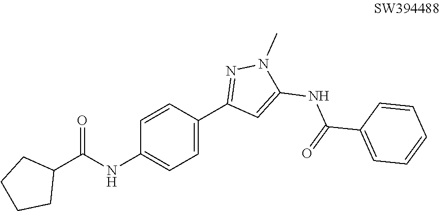 [SMILES: Cn1nc(-c2ccc(NC(=O)C3CCCC3)cc2)cc1NC(=O)c1ccccc1]

N-(3-(4-(Cyclopentanecarboxamido)phenyl)-1-methyl-1H-pyrazol-5-yl)benzamide (SW394488): The general procedure C was followed using N-(3-(4-aminophenyl)1-methyl-1H-pyrazol-5-yl)benzamide (0.150 g, 0.51 mmol), cyclopentanecarboxylic acid (0.070 g, 0.62 mmol), HATU (0.236 g, 0.77 mmol), DIPEA (0.36 mL, 2.05 mmol) and DCM (6.0 mL). The crude product was purified by flash column chromatography on silica gel (DCM/MeOH, 49:1) to give SW394488 as a white solid (0.143 g, 72%). ESI MS for C₂₃H₂₄N₄O₂ m/z [M+H]⁺: calculated: 388.2, found 389.2.

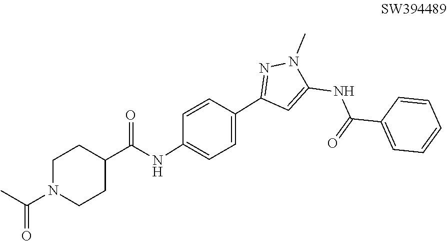 [SMILES: CC(=O)N1CCC(C(=O)Nc2ccc(-c3cc(NC(=O)c4ccccc4)n(C)n3)cc2)CC1]

1-acetyl-N-(4-(5-benzamido-1-methyl-1H-pyrazol-3-yl)phenyl)piperidine-4-carboxamide (SW394489): The general procedure C was followed using N-(3-(4-aminophenyl)1-methyl-1H-pyrazol-5-yl)benzamide (0.20 g, 0.68 mmol), 1-acetylpiperidine-4-carboxylic acid (0.14 g, 0.82 mmol), HATU (0.26 g, 1.02 mmol), DIPEA (0.48 mL, 2.74 mmol) and DCM (8.0 mL). The crude product was purified by flash column chromatography on silica gel (DCM/MeOH, 49:1) to give SW394489 as a white solid (0.153 g, 50%). ESI MS for C₂₅H₂₇N₅O₃ m/z [M+H]⁺: calculated: 445.2, found: 446.1.

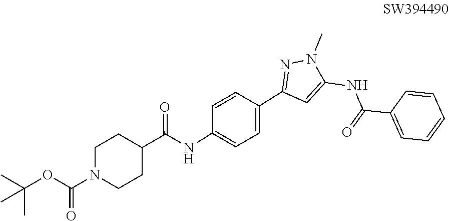 [SMILES: Cn1nc(-c2ccc(NC(=O)C3CCN(C(=O)OC(C)(C)C)CC3)cc2)cc1NC(=O)c1ccccc1]

tert-butyl4-((4-(5-benzamido-1-methyl-1H-pyrazol-3-yl)phenyl)carbamoyl)piperidine-1-carboxylate (SW394490): The general procedure C was followed using N-(3-(4-aminophenyl)-1-methyl-1H-pyrazol-5-yl)benzamide (0.20 g, 0.68 mmol), 1-(tert-butoxycarbonyl)piperidine-4-carboxylic acid (0.19 g, 0.82 mmol), HATU (0.26 g, 1.02 mmol), DIPEA (0.48 mL, 2.74 mmol) and DCM (8.0 mL). The crude product was purified by flash column chromatography on silica gel (DCM/MeOH, 49:1) to give SW394490 as a white solid (0.261 g, 76%). ESI MS for C₂₈H₃₃N₅O₄ m/z [M+H]⁺: calculated: 503.3, found: 504.2.

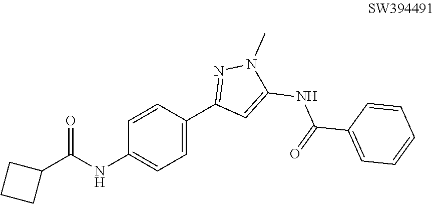 [SMILES: Cn1nc(-c2ccc(NC(=O)C3CCC3)cc2)cc1NC(=O)c1ccccc1]

N-(3-(4-(Cyclobutanecarboxamido)phenyl)-1-methyl-1H-pyrazol-5-yl)benzamide (SW394491) The general procedure C was followed using N-(3-(4-aminophenyl)1-methyl-1H-pyrazol-5-yl)benzamide (0.150 g, 0.51 mmol), cyclobutanecarboxylic acid (0.062 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 49:1) to give SW394491 as a white solid (0.068 g, 53%). ESI MS for C₂₂H₂₂N₄O₂ m/z [M+H]⁺: calculated: 374.2, found: 375.1.

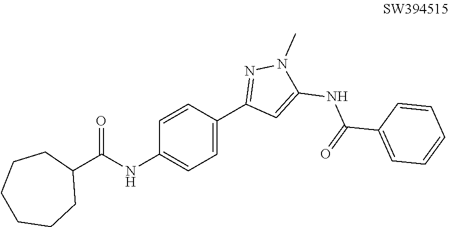 [SMILES: Cn1nc(-c2ccc(NC(=O)C3CCCCCC3)cc2)cc1NC(=O)c1ccccc1]

N-(4-(5-Benzamido-1-methyl-1H-pyrazol-3-yl)phenyl)cycloheptanecarboxamide (SW394515): The general procedure A was followed using N-(3-(4-aminophenyl)-1-methyl-1H-pyrazol-5-yl)benzamide (0.150 g, 0.51 mmol), cycloheptanecarboxylic acid (0.109 g, 0.77 mmol), (COCl)₂ (0.66 mL, 7.70 mmol), DMF (2 drops), DIPEA (0.36 mL, 2.05 mmol) and DCM (6.0 mL). The crude product was purified by flash column chromatography on silica gel (DCM/MeOH, 49:1) to give SW394515 as a white solid (0.093 g, 65%). ESI MS for C₂₅H₂₈N₄O₂ m/z [M+H]⁺: calculated: 416.2, found: 417.2.

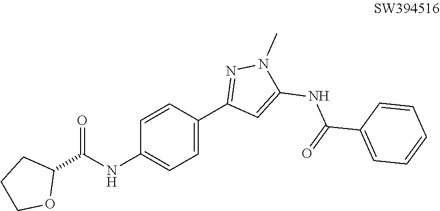 [SMILES: Cn1nc(-c2ccc(NC(=O)[C@H]3CCCO3)cc2)cc1NC(=O)c1ccccc1]

(R)—N-(4-(5-Benzamido-1-methyl-1H-pyrazol-3-yl)phenyl)tetrahydrofuran-2-carboxamide (SW394516): The general procedure A was followed using N-(3-(4-aminophenyl)1-methyl-1H-pyrazol-5-yl)benzamide (0.150 g, 0.51 mmol), (R)-tetrahydrofuran-2-carboxylic acid (0.089 g, 0.77 mmol), (COCl)₂ (0.66 mL, 7.70 mmol), DMF (2 drops), DIPEA (0.36 mL, 2.05 mmol) and DCM (6.0 mL). The crude product was purified by flash column chromatography on silica gel (DCM/MeOH, 49:1) to give SW394516 as a white solid (0.102 g, 51%). ESI MS for C₂₂H₂₂N₄O₃ m/z [M+H]⁺: calculated: 390.2, found: 391.1.

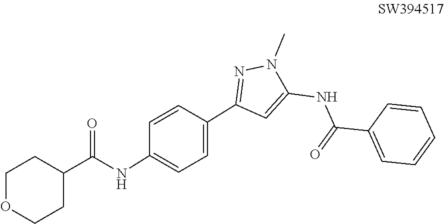 [SMILES: Cn1nc(-c2ccc(NC(=O)C3CCOCC3)cc2)cc1NC(=O)c1ccccc1]

N-(4-(5-Benzamido-1-methyl-1H-pyrazol-3-yl)phenyl)tetrahydro-2H-pyran-4-carboxamide (SW394517): The general procedure A was followed using N-(3-(4-aminophenyl)1-methyl-1H-pyrazol-5-yl)benzamide (0.150 g, 0.51 mmol), tetrahydro-2H-pyran-4-carboxylic acid (0.114 g, 0.77 mmol), (COCl)₂ (0.66 mL, 7.70 mmol), DMF (2 drops), DIPEA (0.36 mL, 2.05 mmol) and DCM (6.0 mL). The crude product was purified by flash column chromatography on silica gel (DCM/MeOH, 49:1) to give SW394517 as a white solid (0.128 g, 62%). ESI MS for C₂₃H₂₄N₄O₃ m/z [M+H]⁺: calculated: 404.2, found: 405.1.

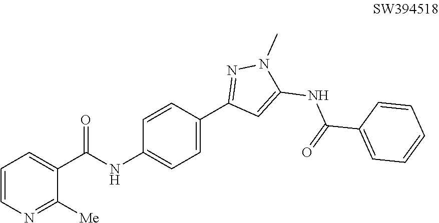 [SMILES: Cc1ncccc1C(=O)Nc1ccc(-c2cc(NC(=O)c3ccccc3)n(C)n2)cc1]

N-(4-(5-Benzamido-1-methyl-1H-pyrazol-3-yl)phenyl)-2-methylnicotinamide (SW394518): The general procedure C was followed using N-(3-(4-aminophenyl)-1-methyl-1H-pyrazol-5-yl)benzamide (0.150 g, 0.51 mmol), 2-methylnicotinic acid (0.084 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 19:1) to give SW394518 as a brown solid (0.154 g, 73%). ESI MS for C₂₄H₂₁N₅O₂ m/z [M+H]⁺: calculated: 411.2, found: 412.1.

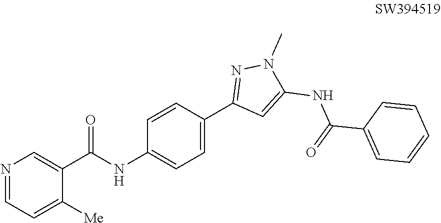 [SMILES: Cc1ccncc1C(=O)Nc1ccc(-c2cc(NC(=O)c3ccccc3)n(C)n2)cc1]

N-(4-(5-Benzamido-1-methyl-1H-pyrazol-3-yl)phenyl)-4-methylnicotinamide (SW394519): The general procedure C was followed using N-(3-(4-aminophenyl)-1-methyl-1H-pyrazol-5-yl)benzamide (0.150 g, 0.51 mmol), 4-methylnicotinic acid (0.084 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 19:1) to give SW394519 as a brown solid (0.114 g, 54%). ESI MS for C₂₄H₂₁N₅O₂ m/z [M+H]⁺: calculated: 411.2, found: 412.1.

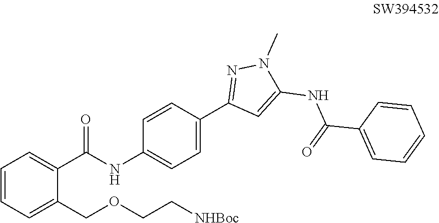 [SMILES: Cn1nc(-c2ccc(NC(=O)c3ccccc3COCCNC(=O)OC(C)(C)C)cc2)cc1NC(=O)c1ccccc1]

tert-butyl (2-((2-((4-(5-benzamido-1-methyl-1H-pyrazol-3-yl)phenyl)carbamoyl)benzyl)oxy)ethyl)carbamate (SW394532): The general procedure D was followed using 2-((2-tert-butoxycarbonyl)amino)ethoxy)methyl)benzoic acid (62 mg, 0.21 mmol), N-(4-(5-amino-1-methyl-1H-pyrazol-3-yl)phenyl)benzamide (0.05 g, 0.17 mmol), Ghosez reagent (0.07 mL, 0.54 mmol), pyridine (0.14 mL, 1.7 mmol) and DCM (2 mL). The crude product was purified by flash chromatography on silica gel (DCM/MeOH 98:1) to give SW394532 as a tan solid (37.4 mg, 39%). ESI MS for C₃₂H₃₅N₅O₅ m/z [M+H]⁺: calculated: 569.7, found: 571.2.

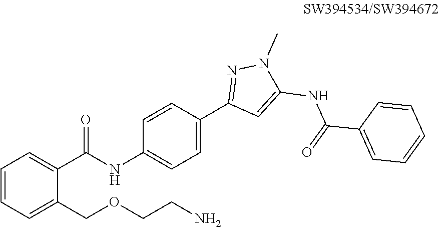 [SMILES: Cn1nc(-c2ccc(NC(=O)c3ccccc3COCCN)cc2)cc1NC(=O)c1ccccc1]

2-((2-aminoethoxy)methyl)-N-(4-(5-benzamido-1-methyl-1H-pyrazol-3-yl)phenyl)benzamide (SW394672): The general procedure E was followed using SW394532 (27.3 mg, 0.048 mmol) in 4 M HCl in dioxane (5 mL) was stirred at room temperature overnight. The solution was concentrated in vacuo to give SW394672 as a white solid (22.5 mg, 100%). ESI MS for C₂₇H₂₇N₅O₃ m/z [M+H]⁺: calculated: 469.6, found: 470.1.

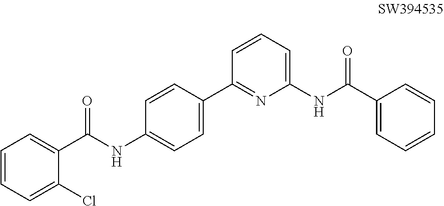 [SMILES: O=C(Nc1cccc(-c2ccc(NC(=O)c3ccccc3Cl)cc2)n1)c1ccccc1]

N-(4-(6-benzamidopyridin-2-yl)phenyl)-2-chlorobenzamide (SW394535): The general procedure C was followed using N-(6-(4-aminophenyl)pyridin-2-yl)benzamide (0.28 g, 0.97 mmol), 2-chloro benzoic acid (183 mg, 1.17 mmol), HATU (554 mg, 1.46 mmol), DIPEA (0.85 mL, 4.85 mmol) and DMF (10 mL). The crude product was purified by flash column chromatography on silica gel (EtOAc/hex, 25:75) to give SW394535 as a yellow solid (0.195 g, 47%). ESI MS for C₂₅H₁ClN₃O₂ m/z [M+H]⁺: calculated: 427.9, found: 429.1.

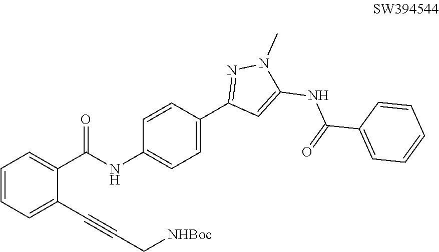 [SMILES: Cn1nc(-c2ccc(NC(=O)c3ccccc3C#CCNC(=O)OC(C)(C)C)cc2)cc1NC(=O)c1ccccc1]

tert-butyl(3-(2-((4-(5-benzamido-1-methyl-1H-pyrazol-3-yl)phenyl)carbamoyl)phenyl)prop-2-yn-1-yl)carbamate (SW394544): The general procedure C was followed using N-(3-(4-aminophenyl)-1-methyl-1H-pyrazol-5-yl)benzamide (0.05 g, 0.17 mmol), 2-(3-((tert-butoxycarbonyl)amino)prop-1-yn-1-yl)benzoic acid (58 mg, 0.2 mmol), HATU (99 mg, 0.26 mmol), DIPEA (0.15 mL, 0.85 mmol) and DMF (5 mL). The crude product was purified by flash column chromatography on silica gel (DCM/MeOH, 97:3) to give SW394544 as a white solid (0.039 g, 42%). ESI MS for C₃₂H₃₁N₅O₄ m/z [M+H]⁺: calculated: 549.6, found: 550.2.

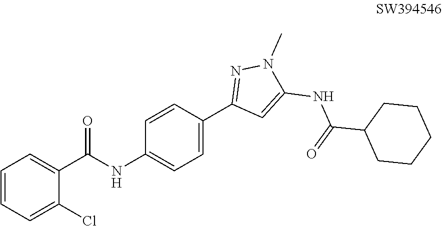 [SMILES: Cn1nc(-c2ccc(NC(=O)c3ccccc3Cl)cc2)cc1NC(=O)C1CCCCC1]

2-Chloro-N-(4-(5-(Cyclohexanecarboxamido)-1-methyl-1H-pyrazol-3-yl)phenyl)benzamide (SW394546): The general procedure A was followed using N-(4-(5-amino-1-methyl-1H-pyrazol-3-yl)phenyl)-2-chlorobenzamide (0.150 g, 0.46 mmol), cyclohexanecarboxylic acid (0.088 g, 0.69 mmol), (COCl)₂ (0.59 mL, 6.89 mmol), DMF (2 drops), DIPEA (0.32 mL, 1.84 mmol) and DCM (6.0 mL). The crude product was purified by flash column chromatography on silica gel (DCM/MeOH, 49:1) to give SW394546 as a white solid (0.126 g, 63%). ESI MS for C₂₄H₂₅ClN₄O₂ m/z [M+H]⁺: calculated: 436.2, found: 437.1.

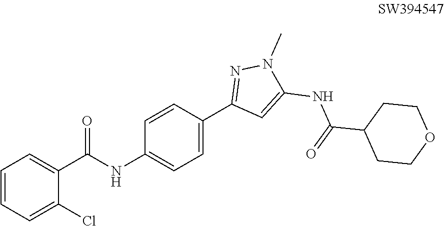 [SMILES: Cn1nc(-c2ccc(NC(=O)c3ccccc3Cl)cc2)cc1NC(=O)C1CCOCC1]

N-(3-(4-(2-Chlorobenzamido)phenyl)-1-methyl-1H-pyrazol-5-yl)tetrahydro-2H-pyran-4-car-boxamide (SW394547): The general procedure A was followed using N-(4-(5-amino-1-methyl-1H-pyrazol-3-yl)phenyl)-2-chlorobenzamide (0.150 g, 0.46 mmol), tetrahydro-2H-pyran-4-carboxylic acid (0.102 g, 0.69 mmol), (COCl)₂ (0.52 mL, 5.99 mmol), DMF (2 drops), DIPEA (0.32 mL, 1.84 mmol) and DCM (6.0 mL). The crude product was purified by crystallization using DCM/Hexanes mixture to give SW394547 as a white solid (0.175 g, 87%). ESI MS for C₂₃H₂₃ClN₄O₃ m/z [M+H]⁺: calculated: 438.2, found: 439.1.

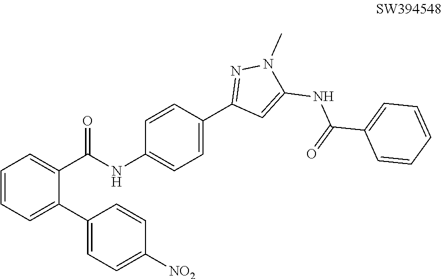 [SMILES: Cn1nc(-c2ccc(NC(=O)c3ccccc3-c3ccc([N+](=O)[O-])cc3)cc2)cc1NC(=O)c1ccccc1]

N-(4-(5-Benzamido-1-methyl-1H-pyrazol-3-yl)phenyl)-4′-nitro-[1,1′-biphenyl]-2-carboxam-ide (SW394548): The general procedure A was followed using N-(3-(4-aminophenyl)-1-methyl-1H-pyrazol-5-yl)benzamide (0.200 g, 0.68 mmol), 4′-nitro-[1,1′-biphenyl]-2-carboxylic acid (0.216 g, 0.89 mmol), (COCl)₂ (0.76 mL, 8.89 mmol), DMF (2 drops), DIPEA (0.48 mL, 2.74 mmol) and DCM (10.0 mL). The crude product was purified by flash column chromatography on silica gel (DCM/MeOH, 47:3) to give SW394548 as a brown solid (0.180 g, 51%). ESI MS for C₃₀H₂₃N₅O₄ m/z [M+H]⁺: calculated: 517.2, found: 518.1.

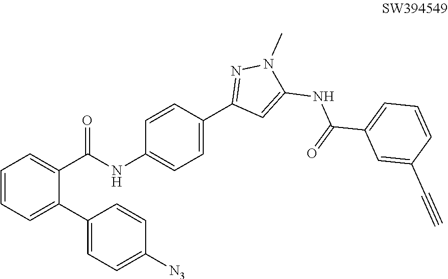 [SMILES: C#Cc1cccc(C(=O)Nc2cc(-c3ccc(NC(=O)c4ccccc4-c4ccc(N=[N+]=[N-])cc4)cc3)nn2C)c1]

4′-Azido-N-(4-(5-(3-ethynylbenzamido)-1-methyl-1H-pyrazol-3-yl)phe-nyl)[1,1′-biphenyl]-2-carboxamide (SW394549): The general procedure A was followed using N-(3-(4-aminophenyl)-1-methyl-1H-pyrazol-5-yl)-3-ethynylbenzamide (0.100 g, 0.32 mmol), 4′-azido-[1,1′-biphenyl]-2-carboxylic acid (0.113 g, 0.47 mmol), (COCl)₂ (0.41 mL, 4.74 mmol), DMF (2 drops), DIPEA (0.11 mL, 0.63 mmol) and DCM (5.0 mL). The crude product was purified by flash column chromatography on silica gel (DCM/MeOH, 47:3) to give SW394549 as a white solid (0.097 g, 57%). ESI MS for C₃₂H₂₃N₇O₂ m/z [M+H]⁺: calculated: 537.2, found: 465.3.

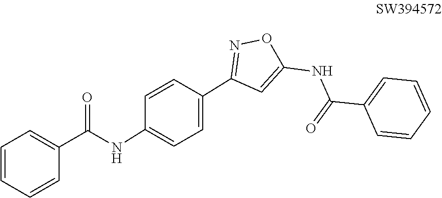 [SMILES: O=C(Nc1ccc(-c2cc(NC(=O)c3ccccc3)on2)cc1)c1ccccc1]

N-(4-(5-benzamidoisoxazol-3-yl)phenyl)benzamide (SW394572): The general procedure A was followed using tert-butyl (4-(5-aminoisoxazol-3-yl)phenyl)carbamate (20.2 mg, 0.07 mmol) and DIPEA (0.025 mL, 0.14 mmol) in DCM (2 mL) was lowered to 0° C. Benzoyl chloride (0.013 mL, 0.11 mmol) was added and raised to room temperature and stirred overnight. The mixture was diluted with EtOAc and washed with H₂O, brine, dried (Na₂SO₄) and concentrated under reduced pressure. The crude product was purified by column chromatography on silica gel (DCM/MeOH 95:5) SW394572 as a tan solid (0.003 g, 11%). ESI MS for C₂₃H₁₇N₃O₃ m/z [M+H]⁺: calculated: 383.4, found: 384.1.

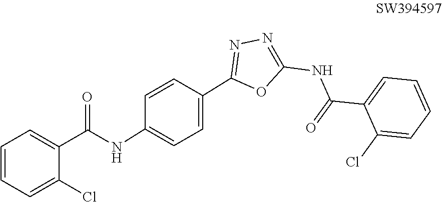 [SMILES: O=C(Nc1ccc(-c2nnc(NC(=O)c3ccccc3Cl)o2)cc1)c1ccccc1Cl]

2-chloro-N-(4-(5-(2-chlorobenzamido)-1,3,4-oxadiazol-2-yl)phenyl)benzamide (SW394597): The general procedure C was followed using 5-(4-aminophenyl)-1,3,4-oxadiazol-2-amine (0.043 g, 0.24 mmol), 2-chloro benzoic acid (91.7 mg, 0.59 mmol), HATU (0.278 mg, 0.73 mmol), DIPEA (0.9 mL, 0.49 mmol) and DMF (2 mL). The crude product was purified by flash column chromatography on silica gel (DCM/MeOH, 99:1) to give SW394597 as a tan solid (0.002 g, 2%). ESI MS for C₂₂H₁₄Cl₂N₄O₃ m/z [M+H]⁺: calculated: 453.3, found: 454.0.

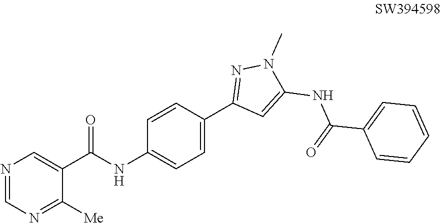 [SMILES: Cc1ncncc1C(=O)Nc1ccc(-c2cc(NC(=O)c3ccccc3)n(C)n2)cc1]

N-(4-(5-Benzamido-1-methyl-1H-pyrazol-3-yl)phenyl)-4-methylpyrimidine-5-carboxamide (SW394598): The general procedure C was followed using N-(3-(4-aminophenyl)1-methyl-1H-pyrazol-5-yl)benzamide (0.150 g, 0.51 mmol), 4-methylpyrimidine-5-carboxylic acid (0.085 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 24:1) to give SW394598 as a white solid (0.150 g, 71%). ESI MS for C₂₃H₂₀N₆O₂ m/z [M+H]⁺: calculated: 412.2, found: 413.1.

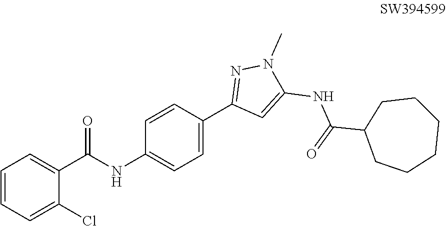 [SMILES: Cn1nc(-c2ccc(NC(=O)c3ccccc3Cl)cc2)cc1NC(=O)C1CCCCCC1]

N-(3-(4-(2-Chlorobenzamido)phenyl)-1-methyl-1H-pyrazol-5-yl)cycloheptanecarboxamide (SW394599): The general procedure A was followed using N-(4-(5-amino-1-methyl-1H-pyrazol-3-yl)phenyl)-2-chlorobenzamide (0.150 g, 0.46 mmol), cycloheptanecarboxylic acid (0.098 g, 0.69 mmol), (COCl)₂ (0.60 mL, 6.89 mmol), DMF (2 drops), DIPEA (0.32 mL, 1.84 mmol) and DCM (6.0 mL). The crude product was purified by flash column chromatography on silica gel (DCM/MeOH, 49:1) to give SW394599 as a white solid (0.117 g, 57%). ESI MS for C₂₅H₂₇ClN₄O₂ m/z [M+H]⁺: calculated: 450.2, found: 451.1.

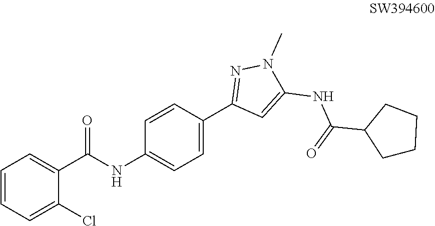 [SMILES: Cn1nc(-c2ccc(NC(=O)c3ccccc3Cl)cc2)cc1NC(=O)C1CCCC1]

2-Chloro-N-(4-(5-(cyclopentanecarboxamido)-1-methyl-1H-pyrazol-3-yl)phenyl)benzamide (SW394600): The general procedure A was followed using N-(4-(5-amino-1-methyl-1H-pyrazol-3-yl)phenyl)-2-chlorobenzamide (0.150 g, 0.46 mmol), cyclopentanecarboxylic acid (0.079 g, 0.69 mmol), (COCl)₂ (0.60 mL, 6.89 mmol), DMF (2 drops), DIPEA (0.32 mL, 1.84 mmol) and DCM (6.0 mL). The crude product was purified by flash column chromatography on silica gel (DCM/MeOH, 49:1) to give SW394600 as a white solid (0.118 g, 61%). ESI MS for C₂₃H₂₃ClN₄O₂ m/z [M+H]⁺: calculated: 422.2, found: 423.1.

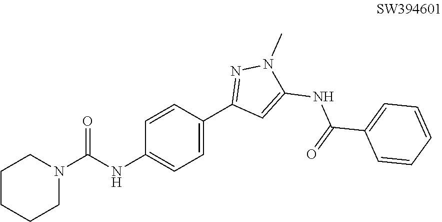 [SMILES: Cn1nc(-c2ccc(NC(=O)N3CCCCC3)cc2)cc1NC(=O)c1ccccc1]

N-(4-(5-benzamido-1-methyl-1H-pyrazol-3-yl)phenyl)piperidine-1-carboxamide (SW394601): The general procedure C was followed using N-(4-(5-amino-1-methyl-1H-pyrazol-3-yl)phenyl)-2-chlorobenzamide (0.150 g, 0.51 mmol), piperidine-1-carboxylic acid (0.102 g, 0.62 mmol), HATU (0.216 mg, 0.77 mmol), DIPEA (0.45 mL, 2.56 mmol) and DMF (6 mL). The crude product was purified by flash column chromatography on silica gel (DCM/MeOH, 99:1) to give SW394601 as a white solid (0.93 g, 45%). ESI MS for C₂₃H₂₅N₅O₂ m/z [M+H]⁺: calculated: 417.2, found: 418.3.

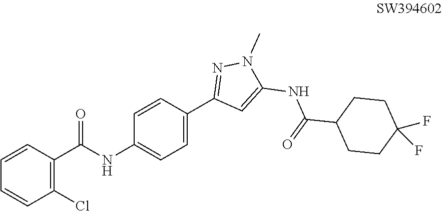 [SMILES: Cn1nc(-c2ccc(NC(=O)c3ccccc3Cl)cc2)cc1NC(=O)C1CCC(F)(F)CC1]

2-Chloro-N-(4-(5-(4,4-difluorocyclohexane-1-carboxamido)-1-methyl-1H-pyrazol-3-yl)phen-yl)benzamide (SW394602): The general procedure A was followed using N-(4-(5-amino-1-methyl-1H-pyrazol-3-yl)phenyl)-2-chlorobenzamide (0.150 g, 0.46 mmol), 4,4-difluorocyclohexa-ne-1-carboxylic acid (0.113 g, 0.69 mmol), (COCl)₂ (0.60 mL, 6.89 mmol), DMF (2 drops), DIPEA (0.32 mL, 1.84 mmol) and DCM (6.0 mL). The crude product was purified by flash column chromatography on silica gel (DCM/MeOH, 49:1) to give SW394602 as a white solid (0.126 g, 58%). ESI MS for C₂₄H₂₃ClF₂N₄O₂ m/z [M+H]⁺: calculated: 472.1, found: 473.1.

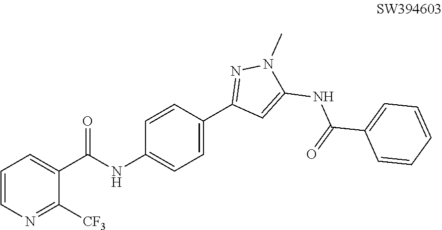 [SMILES: Cn1nc(-c2ccc(NC(=O)c3cccnc3C(F)(F)F)cc2)cc1NC(=O)c1ccccc1]

N-(4-(5-Benzamido-1-methyl-1H-pyrazol-3-yl)phenyl)-2-(trifluoromethyl)nicotinamide (SW394603): The general procedure C was followed using N-(3-(4-aminophenyl)-1-methyl-1H-pyrazol-5-yl)benzamide (0.150 g, 0.51 mmol), 2-(trifluoromethyl)nicotinic acid (0.118 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 19:1) to give SW394603 as a white solid (0.160 g, 67%). ESI MS for C₂₄H₁₈F₃N₅O₂ m/z [M+H]⁺: calculated: 465.1, found: 466.1.

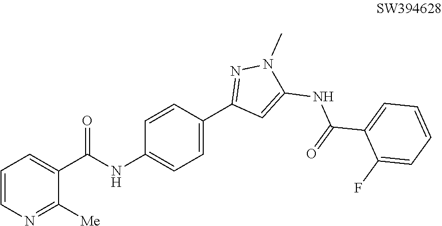 [SMILES: Cc1ncccc1C(=O)Nc1ccc(-c2cc(NC(=O)c3ccccc3F)n(C)n2)cc1]

N-(4-(5-(2-Fluorobenzamido)-1-methyl-1H-pyrazol-3-yl)phenyl)-2-methylnicotinamide (SW394628): The general procedure C was followed using N-(3-(4-aminophenyl)-1-methyl-1H-pyrazol-5-yl)-2-fluorobenzamide (0.125 g, 0.40 mmol), 2-methylnicotinic acid (0.066 g, 0.48 mmol), HATU (0.169 g, 0.60 mmol), DIPEA (0.30 mL, 1.61 mmol) and DCM (6.0 mL). The crude product was purified by flash column chromatography on silica gel (DCM/MeOH, 19:1) to give SW39428 as a white solid (0.124 g, 72%). ESI MS for C₂₄H₂₀FN₅O₂ m/z [M+H]⁺: calculated: 429.2, found: 430.1.

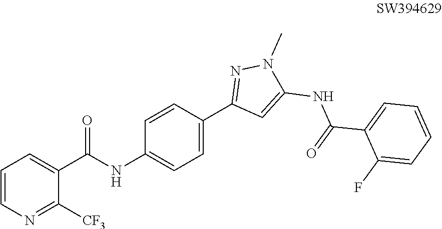 [SMILES: Cn1nc(-c2ccc(NC(=O)c3cccnc3C(F)(F)F)cc2)cc1NC(=O)c1ccccc1F]

N-(4-(5-(2-Fluorobenzamido)-1-methyl-1H-pyrazol-3-yl)phenyl)-2-(trifluoromethyl)nicotinamide (SW394629): The general procedure C was followed using N-(3-(4-aminophenyl)-1-methyl-1H-pyrazol-5-yl)-2-fluorobenzamide (0.125 g, 0.40 mmol), 2-(trifluoromethyl)nicotinic acid (0.092 g, 0.48 mmol), HATU (0.169 g, 0.60 mmol), DIPEA (0.30 mL, 1.61 mmol) and DCM (6.0 mL). The crude product was purified by flash column chromatography on silica gel (DCM/MeOH, 19:1) to give SW394629 as a white solid (0.092 g, 47%). ESI MS for C₂₄H₁₇F₄N₅O₂ m/z [M+H]⁺: calculated: 483.1, found: 484.1.

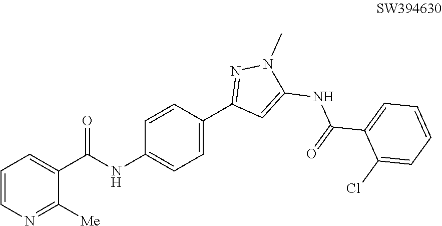 [SMILES: Cc1ncccc1C(=O)Nc1ccc(-c2cc(NC(=O)c3ccccc3Cl)n(C)n2)cc1]

N-(4-(5-(2-Chlorobenzamido)-1-methyl-1H-pyrazol-3-yl)phenyl)-2-methylnicotinamide (SW394630): The general procedure C was followed using N-(3-(4-aminophenyl)-1-methyl-1H-pyrazol-5-yl)-2-chlorobenzamide (0.150 g, 0.46 mmol), 2-methylnicotinic acid (0.076 g, 0.55 mmol), HATU (0.193 g, 0.69 mmol), DIPEA (0.32 mL, 1.84 mmol) and DCM (6.0 mL). The crude product was purified by flash column chromatography on silica gel (DCM/MeOH, 19:1) to give SW394630 as a white solid (0.139 g, 68%). ESI MS for C₂₄H₂₀ClN₅O₂ m/z [M+H]⁺: calculated: 445.1, found: 446.1.

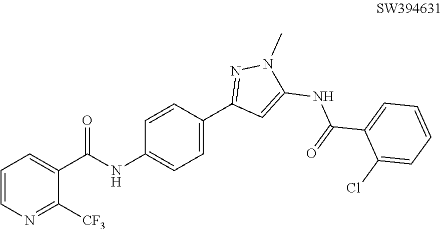 [SMILES: Cn1nc(-c2ccc(NC(=O)c3cccnc3C(F)(F)F)cc2)cc1NC(=O)c1ccccc1Cl]

N-(4-(5-(2-Chlorobenzamido)-1-methyl-1H-pyrazol-3-yl)phenyl)-2-(trifluoromethyl)nicotinamide (SW394631): The general procedure C was followed using N-(3-(4-aminophenyl)-1-methyl-1H-pyrazol-5-yl)-2-chlorobenzamide (0.150 g, 0.46 mmol), 2-(trifluoromethyl)nicotinic acid (0.105 g, 0.55 mmol), HATU (0.193 g, 0.69 mmol), DIPEA (0.32 mL, 1.84 mmol) and DCM (6.0 mL). The crude product was purified by flash column chromatography on silica gel (DCM/MeOH, 19:1) to give SW394631 as a white solid (0.117 g, 51%). ESI MS for C₂₄H₁₇ClF₃N₅O₂ m/z [M+H]⁺: calculated: 499.1, found: 500.3.

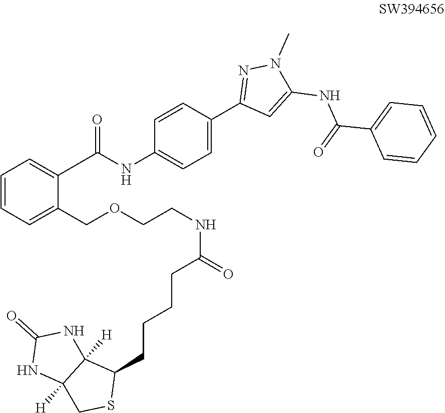 [SMILES: Cn1nc(-c2ccc(NC(=O)c3ccccc3COCCNC(=O)CCCC[C@H]3SC[C@H]4NC(=O)N[C@H]43)cc2)cc1NC(=O)c1ccccc1]

N-(4-(5-benzamido-1-methyl-1H-pyrazol-3-yl)phenyl)-2-((2-(5-((3aR,4R,6aS)-2-oxohexahydro-1H-thieno[3,4-d]imidazol-4-yl)pentanamido)ethoxy)methyl)benzamide (SW394656): The general procedure C was followed using 2-((2-aminoethoxy)methyl)-N-(4-(5-benzamido-1-methyl-1H-pyrazol-3-yl)phenyl)benzamide (11.2 mg, 0.024 mmol), D-Biotin (7 mg, 0.029 mmol), HATU (14 mg, 0.036 mmol), DIPEA (0.013 mL, 0.072 mmol) and DMF (0.25 mL). The crude product was purified by flash column chromatography on silica gel (DCM/MeOH, 90:10) to give SW394656 as a tan solid (0.010 g, 57%). ESI MS for C₃₇H₄₁N₇O₅S m/z [M+H]⁺: calculated: 695.8, found: 696.3.

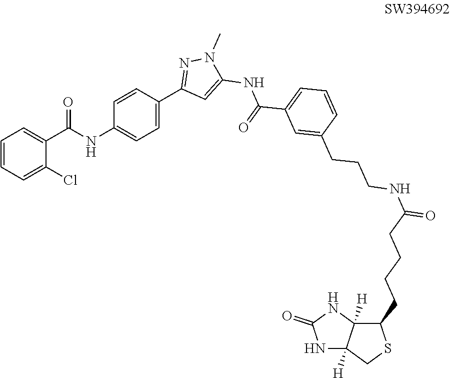 [SMILES: Cn1nc(-c2ccc(NC(=O)c3ccccc3Cl)cc2)cc1NC(=O)c1cccc(CCCNC(=O)CCCC[C@H]2SC[C@H]3NC(=O)N[C@H]32)c1]

2-chloro-N-(4-(1-methyl-5-(3-(3-(5-((3aR,4R,6aS)-2-oxohexahydro-1H-thieno[3,4-d]142 imidazole-4-yl)pentanamido)propyl)benzamido)-1H-pyrazol-3-yl)phenyl)benzamide (SW394692): The general procedure C was followed using N-(4-(5-(3-(3-aminopropyl)benzamido)-1-methyl-1H-pyrazol-3-yl)phenyl)-2-chlorobenzamide (8.3 mg, 0.017 mmol), D-Biotin (5 mg, 0.02 mmol), HATU (10 mg, 0.025 mmol), DIPEA (0.009 mL, 0.051 mmol) and DMF (0.2 mL). The crude product was purified by flash column chromatography on silica gel (DCM/MeOH, 90:10) to give SW394692 as a tan solid (0.003 g, 23%). ESI MS for C₃₇H₄₀ClN₇O₄S m/z [M+H]⁺: calculated: 714.3, found: 715.2.

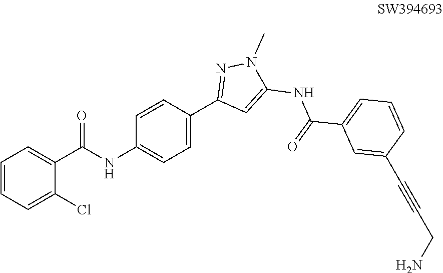 [SMILES: Cn1nc(-c2ccc(NC(=O)c3ccccc3Cl)cc2)cc1NC(=O)c1cccc(C#CCN)c1]

N-(4-(5-(3-(3-aminoprop-1-yn-1-yl)benzamido)-1-methyl-1H-pyrazol-3-yl)phenyl)-2-chlorobenzamide (SW394693): The general procedure E was followed tert-butyl (3-(3-((3-(4-(2-chlorobenzamido)phenyl)-1-methyl-1H-pyrazol-5-yl)carbamoyl)phenyl)prop-2-yn-1-yl)carbamate (13 mg, 0.02 mmol) in 4 M HCl in dioxane (3 mL) was stirred at room temperature overnight. The solution was concentrated in vacuo to give SW394693 as a white solid (10 mg, 100%). ESI MS for C₂₇H₂₂ClN₅O₂ m/z [M+H]⁺: calculated: 483.9, found: 484.2.

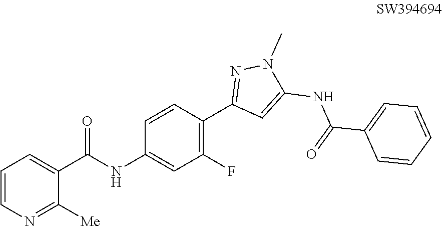 [SMILES: Cc1ncccc1C(=O)Nc1ccc(-c2cc(NC(=O)c3ccccc3)n(C)n2)c(F)c1]

N-(4-(5-Benzamido-1-methyl-1H-pyrazol-3-yl)-3-fluorophenyl)-2-methylnicotinamide (SW394694): The general procedure C was followed using N-(3-(4-amino-2-fluorophenyl)-1-methyl-1H-pyrazol-5-yl)benzamide (0.150 g, 0.48 mmol), 2-methylnicotinic acid (0.079 g, 0.58 mmol), HATU (0.203 g, 0.73 mmol), DIPEA (0.34 mL, 1.93 mmol) and DCM (6.0 mL). The crude product was purified by flash column chromatography on silica gel (DCM/MeOH, 19:1) to give SW394694 as a brown solid (0.107 g, 52%). ESI MS for C₂₄H₂₀FN₅O₂ m/z [M+H]⁺: calculated: 429.2, found: 430.1.

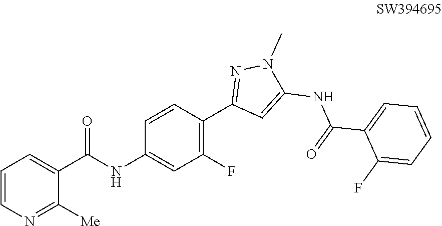 [SMILES: Cc1ncccc1C(=O)Nc1ccc(-c2cc(NC(=O)c3ccccc3F)n(C)n2)c(F)c1]

N-(3-Fluoro-4-(5-(2-fluorobenzamido)-1-methyl-1H-pyrazol-3-yl)phenyl)-2-methylnicotinamide (SW394695): The general procedure C was followed using N-(3-(4-amino-2-fluorophenyl)-1-methyl-1H-pyrazol-5-yl)-2-fluorobenzamide (0.150 g, 0.46 mmol), 2-methylnicotinic acid (0.075 g, 0.55 mmol), HATU (0.192 g, 0.69 mmol), DIPEA (0.32 mL, 1.83 mmol) and DCM (6.0 mL). The crude product was purified by flash column chromatography on silica gel (DCM/MeOH, 19:1) to give SW394695 as a white solid (0.096 g, 47%). ESI MS for C₂₄H₁₉F₂N₅O₂ m/z [M+H]⁺: calculated: 447.2, found: 448.1.

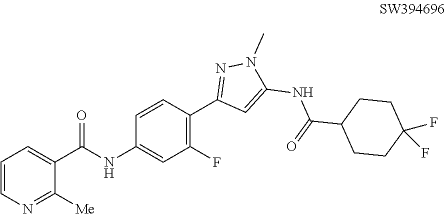 [SMILES: Cc1ncccc1C(=O)Nc1ccc(-c2cc(NC(=O)C3CCC(F)(F)CC3)n(C)n2)c(F)c1]

N-(4-(5-(4,4-Difluorocyclohexane-1-carboxamido)-1-methyl-1H-pyrazol-3-yl)-3-fluorophen-yl)-2-methylnicotinamide (SW394696): The general procedure A was followed using N-(3-(4-amino-2-fluorophenyl)-1-methyl-1H-pyrazol-5-yl)-4,4-difluorocyclohexane-1-carboxamide (0.15 g, 0.44 mmol), 2-methylnicotinic acid (0.091 g, 0.69 mmol), (COCl)₂ (0.60 mL, 6.63 mmol), DMF (2 drops), Et₃N (0.25 mL, 1.77 mmol, DCM (5.0 mL) and CH₃CN (5.0 mL). The crude product was purified by flash column chromatography on silica gel (DCM/MeOH, 19:1) to give SW394696 as a white solid (0.083 g, 40%). ESI MS for C₂₄H₂₄F₃N₅O₂ m/z [M+H]⁺: calculated: 471.2, found: 472.2.

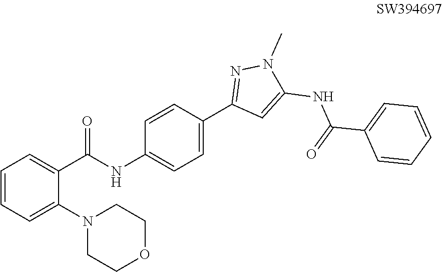 [SMILES: Cn1nc(-c2ccc(NC(=O)c3ccccc3N3CCOCC3)cc2)cc1NC(=O)c1ccccc1]

N-(4-(5-Benzamido-1-methyl-1H-pyrazol-3-yl)phenyl)-2-morpholinobenzamide (SW394697): The general procedure C was followed using N-(3-(4-aminophenyl)-1-methyl-1H-pyrazol-5-yl)benzamide (0.150 g, 0.51 mmol), 2-morpholinobenzoic acid (0.127 g, 0.62 mmol), HATU (0.217 g, 0.78 mmol), DIPEA (0.36 mL, 2.05 mmol) and DCM (6.0 mL). The crude product was purified by flash column chromatography on silica gel (DCM/MeOH, 19:1) to give SW394597 as a white solid (0.106 g, 43%). ESI MS for C₂₈H₂₇N₅O₃ m/z [M+H]⁺: calculated: 481.2, found: 482.2.

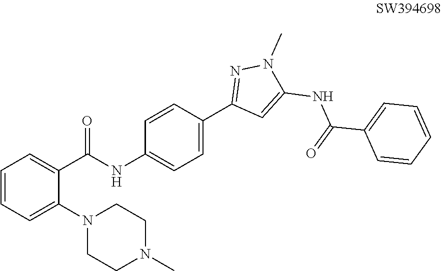 [SMILES: CN1CCN(c2ccccc2C(=O)Nc2ccc(-c3cc(NC(=O)c4ccccc4)n(C)n3)cc2)CC1]

N-(4-(5-Benzamido-1-methyl-1H-pyrazol-3-yl)phenyl)-2-(4-methylpiperazin-1-yl)benzamide (SW394698): The general procedure C was followed using N-(3-(4-aminophenyl)1-methyl-1H-pyrazol-5-yl)benzamide (0.150 g, 0.51 mmol), 2-(4-methylpiperazin-1-yl)benzoic acid (0.136 g, 0.62 mmol), HATU (0.216 g, 0.77 mmol), DIPEA (0.36 mL, 2.05 mmol) and DCM (6.0 mL). The crude product was purified by flash column chromatography on silica gel (DCM/MeOH, 19:1) to give SW394598 as a white solid (0.098 g, 39%). ESI MS for C₂₉H₃₀NO₂ m/z [M+H]⁺: calculated: 494.2, found: 495.2.

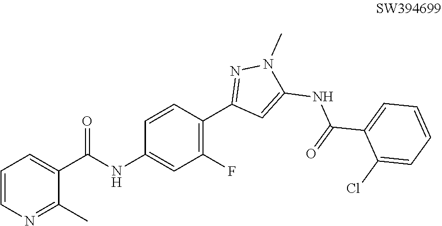 [SMILES: Cc1ncccc1C(=O)Nc1ccc(-c2cc(NC(=O)c3ccccc3Cl)n(C)n2)c(F)c1]

N-(4-(5-(2-chlorobenzamido)-1-methyl-1H-pyrazol-3-yl)-3-fluorophenyl)-2-methylnicotinamide (SW394699): The general procedure C was followed using N-(3-(4-amino-2-fluorophenyl)-1-methyl-1H-pyrazol-5-yl)-2-chlorobenzamide (0.200 g, 0.60 mmol), 2-(4-methylpiperazin-1-yl)benzoic acid (0.99 g, 0.72 mmol), HATU (0.253 g, 0.90 mmol), DIPEA (0.42 mL, 2.41 mmol) and ACN (8.0 mL). The crude product was purified by flash column chromatography on silica gel (DCM/MeOH, 19:1) to give SW394599 as a white solid (0.053 g, 19%). ESI MS for C₂₄H₁₉ClFN₅O₂ m/z [M+H]⁺: calculated: 463.1, found: 464.1.

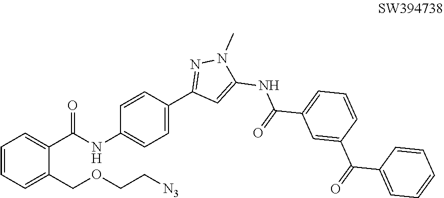 [SMILES: Cn1nc(-c2ccc(NC(=O)c3ccccc3COCCN=[N+]=[N-])cc2)cc1NC(=O)c1cccc(C(=O)c2ccccc2)c1]

2-((2-azidoethoxy)methyl)-N-(4-(5-(3-benzoylbenzamido)-1-methyl-1H-pyrazol-3-yl)phenyl)benzamide (SW394738): The general procedure D was followed using 2-((2-azidoethoxy)methyl)benzoic acid (60 mg, 0.27 mmol), N-(3-(4-aminophenyl)-1-methyl-1H-pyrazol-5-yl)-3-benzoylbenzamide (87 mg, 0.22 mmol), Ghosez reagent (0.095 mL, 0.71 mmol), pyridine (0.18 mL, 2.2 mmol) and DCM (2 mL). The crude product was purified by flash chromatography on silica gel (DCM/MeOH 99:1) to give SW394738 as a white solid (23.6 mg, 18%). ESI MS for C₃₄H₂₉N₇O₄ m/z [M+H]⁺: calculated: 599.7, found: 600.2.

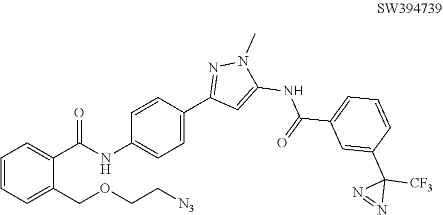 [SMILES: Cn1nc(-c2ccc(NC(=O)c3ccccc3COCCN=[N+]=[N-])cc2)cc1NC(=O)c1cccc(C2(C(F)(F)F)N=N2)c1]

2-((2-azidoethoxy)methyl)-N-(4-(1-methyl-5-(3-(3-(trifluoromethyl)-3H-diazirin-3-yl)benzamido)-1H-pyrazol-3-yl)phenyl)benzamide (SW394739): The general procedure D was followed using 2-((2-azidoethoxy)methyl)benzoic acid (60 mg, 0.27 mmol), N-(3-(4-aminophenyl)-1-methyl-1H-pyrazol-5-yl)-3-(3-(trifluoromethyl)-3H-diazirin-3-yl)benzamide (88 mg, 0.22 mmol), Ghosez reagent (0.095 mL, 0.71 mmol), pyridine (0.18 mL, 2.2 mmol) and DCM (2 mL). The crude product was purified by flash chromatography on silica gel (DCM/MeOH 99:1) to give SW394739 as a white solid (21.2 mg, 16%). ESI MS for C₂₉H₂₄F₃N₉O₃ m/z [M+H]⁺: calculated: 603.6, found: 604.2.

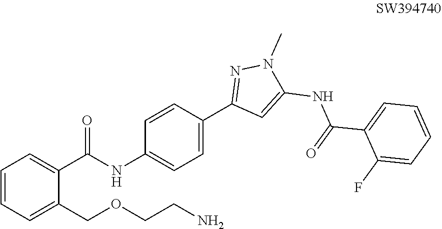 [SMILES: Cn1nc(-c2ccc(NC(=O)c3ccccc3COCCN)cc2)cc1NC(=O)c1ccccc1F]

2-((2-aminoethoxy)methyl)-N-(4-(5-(2-fluorobenzamido)-1-methyl-1H-pyrazol-3-yl)phenyl)benzamide (SW394740): The general procedure E was followed using tert-butyl (2-((2-((4-(5-(2-fluorobenzamido)-1-methyl-1H-pyrazol-3-yl)phenyl)carbamoyl)benzyl)oxy)ethyl)carbamate (39 mg, 0.07 mmol) in 4 M HCl in dioxane (3 mL) was stirred at room temperature overnight. The solution was concentrated in vacuo to give SW394740 as a white solid (10 mg, 29%). ESI MS for C₂₇H₂₆FN₅O₃ m/z [M+H]⁺: calculated: 487.5, found: 488.2.

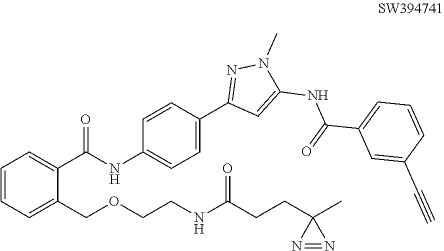 [SMILES: C#Cc1cccc(C(=O)Nc2cc(-c3ccc(NC(=O)c4ccccc4COCCNC(=O)CCC4(C)N=N4)cc3)nn2C)c1]

N-(4-(5-(3-ethynylbenzamido)-1-methyl-1H-pyrazol-3-yl)phenyl)-2-((2-(3-(3-methyl-3H-diazirin-3-yl)propanamido)ethoxy)methyl)benzamide (SW394741): The general procedure C was followed using 2-((2-aminoethoxy)methyl)-N-(4-(5-(3-ethynylbenzamido)-1-methyl-1H-pyrazol-3-yl)phenyl)benzamide (67 mg, 0.163 mmol), 3-(3-methyl-3H-diazirin-3-yl)propanoic acid (25 mg, 0.2 mmol), HATU (93.1 mg, 0.245 mmol), DIPEA (0.09 mL, 0.489 mmol) and DMF (1.5 mL). The crude product was purified by flash column chromatography on silica gel (DCM/MeOH, 95:5) to give SW394741 as a tan solid (21.3 mg, 21%). ESI MS for C₃₄H₃₃N₇O₄ m/z [M+H]⁺: calculated: 603.7, found: 604.3.

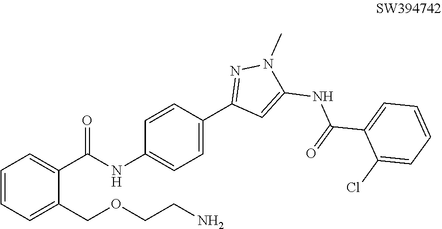 [SMILES: Cn1nc(-c2ccc(NC(=O)c3ccccc3COCCN)cc2)cc1NC(=O)c1ccccc1Cl]

2-((2-aminoethoxy)methyl)-N-(4-(5-(2-chlorobenzamido)-1-methyl-1H-pyrazol-3-yl)phenyl)benzamide (SW394742): The general procedure E was followed using tert-butyl (2-((2-((4-(5-(2-chlorobenzamido)-1-methyl-1H-pyrazol-3-yl)phenyl)carbamoyl)benzyl)oxy)ethyl)carbamate (21 mg, 0.035 mmol) in 4 M HCl in dioxane (5 mL) was stirred at room temperature overnight. The solution was concentrated in vacuo to give SW394742 as a white solid (7.5 mg, 43%). ESI MS for C₂₇H₂ClN₅O₃ m/z [M+H]⁺: calculated: 504.0, found: 505.2.

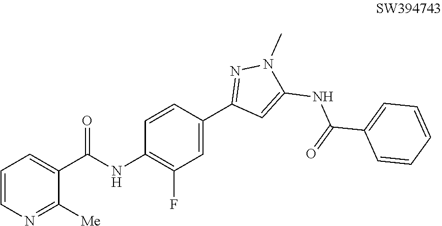 [SMILES: Cc1ncccc1C(=O)Nc1ccc(-c2cc(NC(=O)c3ccccc3)n(C)n2)cc1F]

N-(4-(5-Benzamido-1-methyl-1H-pyrazol-3-yl)-2-fluorophenyl)-2-methylnicotinamide (SW394743): The general procedure C was followed using N-(3-(4-amino-3-fluorophenyl)-1-methyl-1H-pyrazol-5-yl)benzamide (0.100 g, 0.32 mmol), 2-methylnicotinic acid (0.053 g, 0.39 mmol), HATU (0.136 g, 0.48 mmol), DIPEA (0.23 mL, 1.29 mmol) and DCM (5.0 mL). The crude product was purified by flash column chromatography on silica gel (DCM/MeOH, 19:1) to give SW394743 as a brown solid (0.071 g, 51%). ESI MS for C₂₄H₂₀FN₅O₂ m/z [M+H]⁺: calculated: 429.2, found: 430.1.

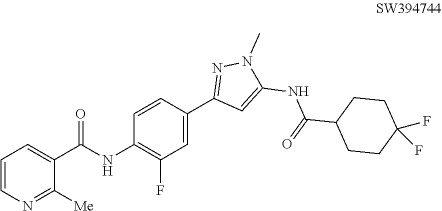 [SMILES: Cc1ncccc1C(=O)Nc1ccc(-c2cc(NC(=O)C3CCC(F)(F)CC3)n(C)n2)cc1F]

N-(4-(5-(4,4-Difluorocyclohexane-1-carboxamido)-1-methyl-1H-pyrazol-3-yl)-2-fluorophen-yl)-2-methylnicotinamide (SW394744): The general procedure C was followed using N-(3-(4-amino-3-fluorophenyl)-1-methyl-1H-pyrazol-5-yl)-4,4-difluorocyclohexane-1-carboxamide (0.100 g, 0.28 mmol), 2-methylnicotinic acid (0.047 g, 0.34 mmol), HATU (0.119 g, 0.43 mmol), DIPEA (0.20 mL, 1.14 mmol) and DCM (5.0 mL). The crude product was purified by flash column chromatography on silica gel (DCM/MeOH, 19:1) to give SW394744 as a brown solid (0.056 g, 42%). ESI MS for C₂₄H₂₄F₃N₅O₂ m/z [M+H]⁺: calculated: 471.2, found: 472.1.

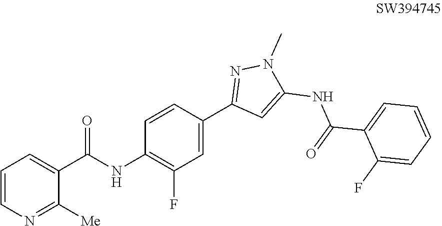 [SMILES: Cc1ncccc1C(=O)Nc1ccc(-c2cc(NC(=O)c3ccccc3F)n(C)n2)cc1F]

N-(2-Fluoro-4-(5-(2-fluorobenzamido)-1-methyl-1H-pyrazol-3-yl)phenyl)-2-methylnicotinamide (SW394745): The general procedure C was followed using N-(3-(4-amino-3-fluorophenyl)-1-methyl-1H-pyrazol-5-yl)-2-fluorobenzamide (0.150 g, 0.46 mmol), 2-methylnicotinic acid (0.075 g, 0.55 mmol), HATU (0.192 g, 0.69 mmol), DIPEA (0.32 mL, 1.83 mmol) and DCM (5.0 mL). The crude product was purified by flash column chromatography on silica gel (DCM/MeOH, 19:1) to give SW394745 as a brown solid (0.080 g, 39%). ESI MS for C₂₄H₂₉F₂N₅O₂ m/z [M+H]⁺: calculated: 447.2, found: 448.1.

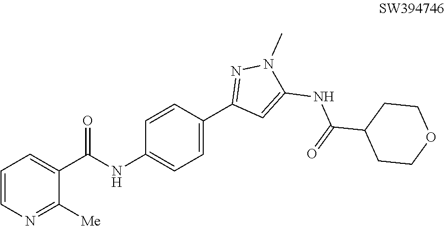 [SMILES: Cc1ncccc1C(=O)Nc1ccc(-c2cc(NC(=O)C3CCOCC3)n(C)n2)cc1]

2-Methyl-N-(4-(1-methyl-5-(tetrahydro-2H-pyran-4-carboxamido)-1H-pyrazol-3-yl)phenyl)nicotinamide (SW394746): The general procedure C was followed using N-(3-(4-aminophenyl)-1-methyl-1H-pyrazol-5-yl)tetrahydro-2H-pyran-4-carboxamide (0.150 g, 0.50 mmol), 2-methylnicotinic acid (0.082 g, 0.60 mmol), HATU (0.210 g, 0.75 mmol), DIPEA (0.35 mL, 2.00 mmol) and DCM (6.0 mL). The crude product was purified by flash column chromatography on silica gel (DCM/MeOH, 19:1) to give SW394746 as a white solid (0.213 g, 47%). ESI MS for C₂₃H₂₅N₅O₃ m/z [M−H]⁺: calculated: 419.2, found: 418.2.

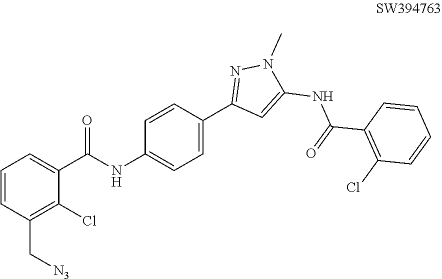 [SMILES: Cn1nc(-c2ccc(NC(=O)c3cccc(CN=[N+]=[N-])c3Cl)cc2)cc1NC(=O)c1ccccc1Cl]

3-(azidomethyl)-2-chloro-N-(4-(5-(2-chlorobenzamido)-1-methyl-1H-pyrazol-3-yl)phenyl)benzamide (SW394763): To a solution of 3-(bromomethyl)-2-chloro-N-(4-(5-(2-chlorobenzamido)-1-methyl-1H-pyrazol-3-yl)phenyl)benzamide (14.8 mg, 0.026 mmol) in DMF (1 mL) was added NaN₃ (3 mg, 0.04 mmol) and heated to 50° C. overnight. The mixture was diluted with EtOAc and washed with H₂O, brine, dried (Na₂SO₄) and concentrated under reduced pressure. The crude product was purified by column chromatography on silica gel (EtOAc/hex 80:20) SW394763 as a white solid (3.3 mg, 24%). ESI MS for C₂₅H₁Cl₂N₇O₂ m/z [M+H]⁺: calculated: 520.4, found: 521.1.

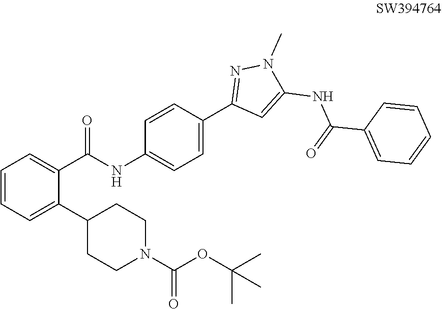 [SMILES: Cn1nc(-c2ccc(NC(=O)c3ccccc3C3CCN(C(=O)OC(C)(C)C)CC3)cc2)cc1NC(=O)c1ccccc1]

tert-butyl4-(2-((4-(5-benzamido-1-methyl-1H-pyrazol-3-yl)phenyl)carbamoyl)phenyl)piperidine-1-carboxylate (SW394764): The general procedure C was followed using N-(3-(4-aminophenyl)-1-methyl-1H-pyrazol-5-yl)benzamide (0.187 g, 0.64 mmol), 2-(1-(tert-butoxycarbonyl)piperidin-4-yl)benzoic acid 0.235 g, 0.77 mmol), HATU (0.270 g, 0.96 mmol), DIPEA (0.70 mL, 3.84 mmol) and DCM (10.0 mL). The crude product was purified by flash column chromatography on silica gel (DCM/MeOH, 19:1) to give SW394764 as a white solid (0.286 g, 77%). ESI MS for C₃₄H₃₇N₅O₄ m/z [M−H]⁺: calculated: 579.3, found: 580.0.

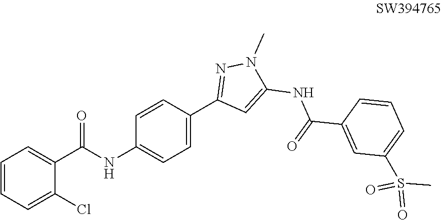 [SMILES: Cn1nc(-c2ccc(NC(=O)c3ccccc3Cl)cc2)cc1NC(=O)c1cccc(S(C)(=O)=O)c1]

2-Chloro-N-(4-(1-methyl-5-(3-(methylsulfonyl)benzamido)-1H-pyrazol-3-yl)phenyl)benza-mide (SW394765): The general procedure A was followed using N-(4-(5-amino-1-methyl-1H-pyrazol-3-yl)phenyl)-2-chlorobenzamide (0.150 g, 0.46 mmol), 3-(methylsulfonyl)benzoic acid (0.138 g, 0.69 mmol), (COCl)₂ (0.52 mL, 5.99 mmol), DMF (2 drops), DIPEA (0.2 mL, mmol) and DCM (6.0 mL). The crude product was purified by flash column chromatography on silica gel (DCM/MeOH, 49:1) to give SW394765 as a white solid (0.102 g, 44%). ESI MS for C₂₅H₂₁ClN₄O₄S m/z [M+H]⁺: calculated: 508.1, found: 509.1.

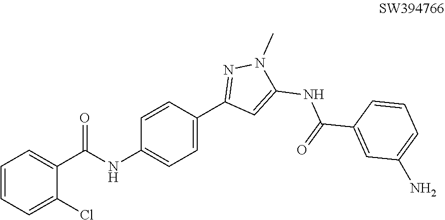 [SMILES: Cn1nc(-c2ccc(NC(=O)c3ccccc3Cl)cc2)cc1NC(=O)c1cccc(N)c1]

N-(4-(5-(3-Aminobenzamido)-1-methyl-1H-pyrazol-3-yl)phenyl)-2-chlorobenzamide (SW394766): The general procedure E was followed using tert-butyl (3-((3-(4-(2-chlorobenzamido)phenyl)-1-methyl-1H-pyrazol-5-yl)carbamoyl)phenyl)carbamate (0.915 g, 1.68 mmol) and 4 M HCl in dioxane (7.0 mL) to give SW394766 as a white solid (0.411 g, 56%). ESI MS for C₂₄H₂₀ClN₅O₂ m/z [M+H]⁺: calculated: 445.1, found: 446.3.

### SW394767

N-(4-(5-benzamido-1-methyl-1H-pyrazol-3-yl)phenyl)-2-(piperidin-4-yl)benzamide (SW394767): The general procedure E was followed using tert-butyl 4-(2-((4-(5-benzamido-1-methyl-1H-pyrazol-3-yl)phenyl)carbamoyl)phenyl)piperidine-1-carboxylate (0.250 g, 0.43 mmol) and 4 M HCl in dioxane (1.8 mL) to give SW394767 as a white solid (0.411 g, 100%). ESI MS for C₂₉H₂₉N₅O₂ m/z [M+H]⁺: calculated: 479.2, found: 480.0.

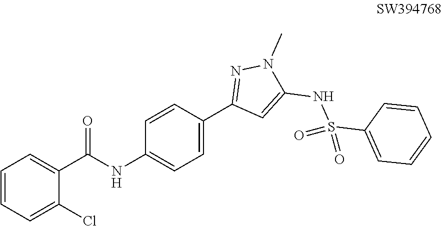 [SMILES: Cn1nc(-c2ccc(NC(=O)c3ccccc3Cl)cc2)cc1NS(=O)(=O)c1ccccc1]

2-Chloro-N-(4-(1-methyl-5-(phenylsulfonamido)-1H-pyrazol-3-yl)phenyl)benzamide (SW394768): Benzenesulfonyl chloride (0.047 mL, 0.37 mmol) in DCM (0.5 mL) was added slowly to a solution of N-(4-(5-amino-1-methyl-1H-pyrazol-3-yl)phenyl)-2-chlorobenzamide (0.100 g, 0.34 mmol) in pyridine (3.0 mL). The resulting reaction mixture was stirred at rt for 18 h. The solvent was evaporated under vacuo. The crude product was purified by flash column chromatography on silica gel (DCM/MeOH, 49:1) to give SW394768 as a colorless oil (0.078 g, 55%). ESI MS for C₂₃H₁₉ClN₄O₃S m/z [M+H]⁺: calculated: 466.1, found: 467.0.

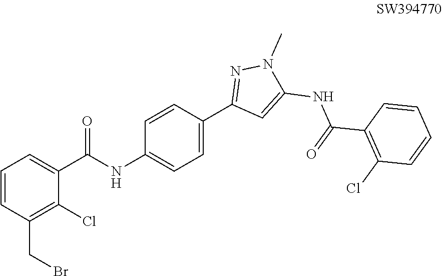 [SMILES: Cn1nc(-c2ccc(NC(=O)c3cccc(CBr)c3Cl)cc2)cc1NC(=O)c1ccccc1Cl]

3-(bromomethyl)-2-chloro-N-(4-(5-(2-chlorobenzamido)-1-methyl-1H-pyrazol-3-yl)phenyl)benzamide (SW394770): The general procedure C was followed using N-(3-(4-aminophenyl)-1-methyl-1H-pyrazol-5-yl)-2-chlorobenzamide (50 mg, 0.15 mmol), 3-(bromomethyl)-2-chlorobenzoic acid (45.8 mg, 0.18 mmol), HATU (87.4 mg, 0.23 mmol), DIPEA (0.08 mL, 0.46 mmol) and DMF (1.5 mL). The crude product was purified by flash column chromatography on silica gel (DCM/MeOH, 98:2) to give SW394770 as a white solid (25.9 mg, 31%). ESI MS for C₂₅H₁₉Cl₂N₇O₂ m/z [M+H]⁺: calculated: 556.0, found: 557.1.

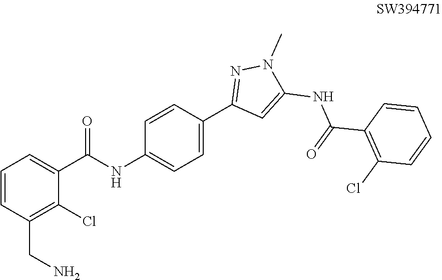 [SMILES: Cn1nc(-c2ccc(NC(=O)c3cccc(CN)c3Cl)cc2)cc1NC(=O)c1ccccc1Cl]

3-(aminomethyl)-2-chloro-N-(4-(5-(2-chlorobenzamido)-1-methyl-1H-pyrazol-3-yl)phenyl)benzamide (SW394771): A flame dried flask was charged with 3-(azidomethyl)-2-chloro-N-(4-(5-(2-chlorobenzamido)-1-methyl-1H-pyrazol-3-yl)phenyl)benzamide (12.5 mg, 0.024 mmol) and Pd/C (10 mg). THE (1 mL) was added under N₂, then the reaction was placed under H₂ and run overnight at room temperature. The crude reaction was filtered through celite and concentrated in vacuo. to give SW394771 as a white solid (2 mg, 17%). ESI MS for C₂₅H₁₉Cl₂N₇O₂ m/z [M+H]⁺: calculated: 494.4, found: 495.1.

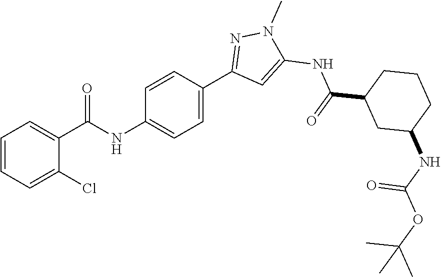

tert-Butyl ((1R,3S)-3-((3-(4-(2-chlorobenzamido)phenyl)-1-methyl-1H-pyrazol-5-yl)carbamoyl)cyclohexyl)carbamate (SW394868-1): The general procedure D was followed using (1S,3R)-3-((tert-butoxycarbonyl)amino)cyclohexane-1-carboxylic acid (0.045 g, 0.18 mmol), 1-chloro-N,N,2-trimethylpropenylamine (Ghosez's reagent) (0.065 mL, 0.49 mmol), N-(4-(5-amino-1-methyl-1H-pyrazol-3-yl)phenyl)-2-chlorobenzamide (0.050 g, 0.15 mmol), pyridine (0.12 mL, 1.53 mmol) and DCM (10.0 mL). The crude product was purified by flash column chromatography on silica gel (DCM/MeOH, 49:1) to give SW394868-1 as a white solid (0.075 g, 89%). ESI MS for C₂₉H₃₄ClN₅O₄ m/z [M+H]⁺: calculated: 551.2, found: 552.2.

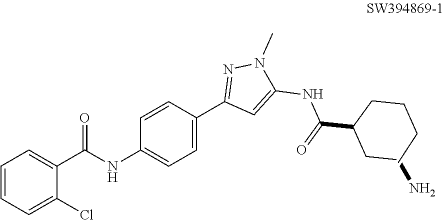

N-(4-(5-((1S,3R)-3-Aminocyclohexane-1-carboxamido)-1-methyl-1H-pyrazol-3-yl)phenyl)-2-chlorobenzamide (SW394869-1): The general procedure E was followed using tert-butyl ((1R,3S)-3-((3-(4-(2-chlorobenzamido)phenyl)-1-methyl-1H-pyrazol-5-yl)carbamoyl)cyclohexyl)-carbamate (0.075 g, 0.14 mmol) and 4 M HCl in dioxane (0.6 mL) to give SW394869-1 as a white solid (0.023 g, 38%). ESI MS for C₂₄H₂₆ClN₅O₂ m/z [M+H]⁺: calculated: 451.2, found: 452.2.

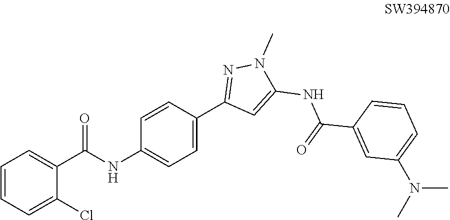 [SMILES: CN(C)c1cccc(C(=O)Nc2cc(-c3ccc(NC(=O)c4ccccc4Cl)cc3)nn2C)c1]

2-Chloro-N-(4-(5-(3-(dimethylamino)benzamido)-1-methyl-1H-pyrazol-3-yl)phenyl)benzamide (SW394870): The general procedure A was followed using N-(3-(4-aminophenyl)-1-methyl-1H-pyrazol-5-yl)-3-(dimethylamino)benzamide (0.150 g, 0.45 mmol), 2-chlorobenzoic acid (0.105 g, 0.67 mmol), (COCl)₂ (0.50 mL, 5.78 mmol), DMF (2 drops), DIPEA (0.32 mL, 1.84 mmol) and DCM (8.0 mL). The crude product was purified by crystallization using DCM/hexanes mixture to give SW394870 as a white solid (0.164 g, 78%). ESI MS for C₂₆H₂₄ClN₅O₂ m/z [M+H]⁺: calculated: 473.2, found: 474.1.

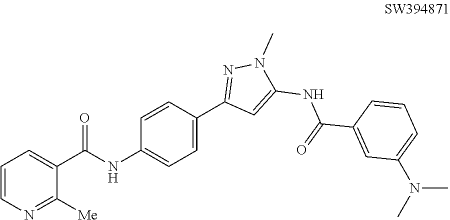 [SMILES: Cc1ncccc1C(=O)Nc1ccc(-c2cc(NC(=O)c3cccc(N(C)C)c3)n(C)n2)cc1]

N-(4-(5-(3-(Dimethylamino)benzamido)-1-methyl-1H-pyrazol-3-yl)phenyl)-2-methylnicotine-amide (SW394871): The general procedure C was followed using N-(3-(4-aminophenyl)-1-methyl-1H-pyrazol-5-yl)-3-(dimethylamino)benzamide (0.150 g, 0.45 mmol), 2-methylnicotinic acid (0.074 g, 0.54 mmol), HATU (0.188 g, 0.67 mmol), DIPEA (0.32 mL, 1.79 mmol) and DCM (8.0 mL). The crude product was purified by flash column chromatography on silica gel (DCM/MeOH, 47:3) to give SW394871 as a brown solid (0.104 g, 51%). ESI MS for C₂₆H₂₆N₆O₂ m/z [M+H]⁺: calculated: 454.2, found: 455.4.

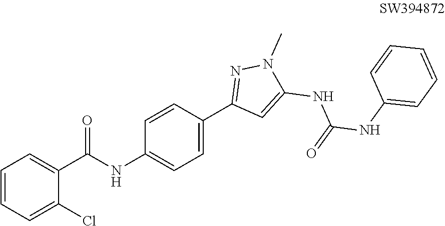 [SMILES: Cn1nc(-c2ccc(NC(=O)c3ccccc3Cl)cc2)cc1NC(=O)Nc1ccccc1]

2-Chloro-N-(4-(1-methyl-5-(3-phenylureido)-1H-pyrazol-3-yl)phenyl)benzamide (SW394872): Phenyl isocyanate (0.033 mL, 0.31 mmol) was added to a solution of N-(4-(5-amino-1-methyl-1H-pyrazol-3-yl)phenyl)-2-chlorobenzamide (0.1 g, 0.31 mmol) in toluene (10 mL). The reaction mixture was refluxed at 110° C. for 24 h and then cooled to rt. H₂O was added and the layers were separated. The aqueous layer was extracted with EtOAc (20 mL×3) and the combined organic layers were washed with brine (50 mL), dried (Na₂SO₄) and concentrated under vacuo. The crude product was purified via crystallization from hot DCM to give SW394872 as a white solid (0.115 g, 85%). ESI MS for C₂₄H₂₀ClN₅O₂ m/z [M+H]⁺: calculated: 445.1, found: 446.1.

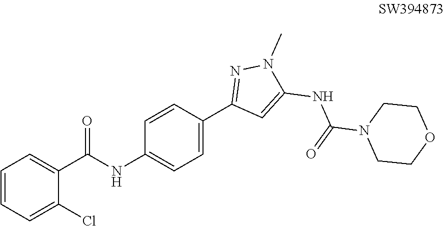 [SMILES: Cn1nc(-c2ccc(NC(=O)c3ccccc3Cl)cc2)cc1NC(=O)N1CCOCC1]

N-(3-(4-(2-Chlorobenzamido)phenyl)-1-methyl-1H-pyrazol-5-yl)morpholine-4-carboxamide (SW394873): General procedure F was followed using DIPEA (0.24 mL, 1.38 mmol), phenyl (3-(4-(2-chlorobenzamido)phenyl)-1-methyl-1H-pyrazol-5-yl)carbamate (0.205 g, 0.46 mmol) and morpholine (0.048 mL, 0.55 mmol) in DMF (5.0 mL). The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394873 as a white solid (0.097 g, 48%). ESI MS for C₂₂H₂₂ClN₅O₃ m/z [M+H]⁺: calculated: 439.1, found: 440.1.

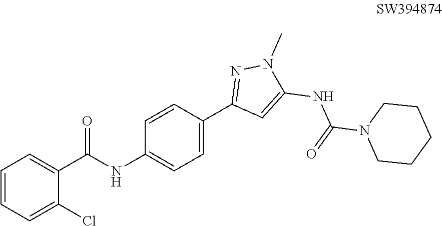 [SMILES: Cn1nc(-c2ccc(NC(=O)c3ccccc3Cl)cc2)cc1NC(=O)N1CCCCC1]

N-(3-(4-(2-Chlorobenzamido)phenyl)-1-methyl-1H-pyrazol-5-yl)piperidine-1-carboxamide (SW394874): General procedure F was followed using DIPEA (0.12 mL, 0.67 mmol), phenyl (3-(4-(2-chlorobenzamido)phenyl)-1-methyl-1H-pyrazol-5-yl)carbamate (0.100 g, 0.22 mmol) and piperidine (0.027 mL, 0.27 mmol) in DMF (5.0 mL). The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394874 as a white solid (0.055 g, 56%). ESI MS for C₂₃H₂₄ClN₅O₂ m/z [M+H]⁺: calculated: 437.2, found: 438.1.

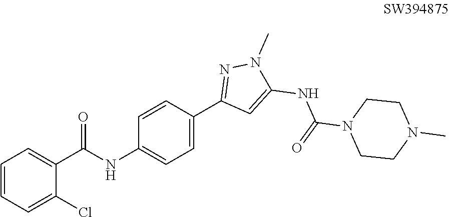 [SMILES: CN1CCN(C(=O)Nc2cc(-c3ccc(NC(=O)c4ccccc4Cl)cc3)nn2C)CC1]

N-(3-(4-(2-Chlorobenzamido)phenyl)-1-methyl-1H-pyrazol-5-yl)-4-methylpiperazine-1-carb-oxamide (SW394875): General procedure F was followed using DIPEA (0.12 mL, 0.67 mmol), phenyl (3-(4-(2-chlorobenzamido)phenyl)-1-methyl-1H-pyrazol-5-yl)carbamate (0.100 g, 0.22 mmol) and 1-methylpiperazine (0.030 mL, 0.27 mmol) in DMF (5.0 mL). The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 47:3) to give SW394875 as a white solid (0.045 g, 44%). ESI MS for C₂₃H₂₅ClN₆O₂ m/z [M+H]⁺: calculated: 452.2, found: 453.2.

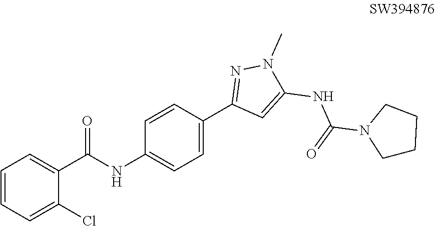 [SMILES: Cn1nc(-c2ccc(NC(=O)c3ccccc3Cl)cc2)cc1NC(=O)N1CCCC1]

N-(3-(4-(2-Chlorobenzamido)phenyl)-1-methyl-1H-pyrazol-5-yl)pyrrolidine-1-carboxamide (SW394876): General procedure F was followed using DIPEA (0.12 mL, 0.67 mmol), phenyl (3-(4-(2-chlorobenzamido)phenyl)-1-methyl-1H-pyrazol-5-yl)carbamate (0.100 g, 0.22 mmol) and pyrrolidine (0.022 mL, 0.27 mmol) in DMF (5.0 mL). The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394876 as a white solid (0.062 g, 65%). ESI MS for C₂₂H₂₂ClN₅O₂ m/z [M+H]⁺: calculated: 423.1, found: 424.1.

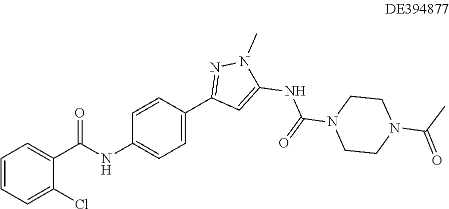 [SMILES: CC(=O)N1CCN(C(=O)Nc2cc(-c3ccc(NC(=O)c4ccccc4Cl)cc3)nn2C)CC1]

4-Acetyl-N-(3-(4-(2-chlorobenzamido)phenyl)-1-methyl-1H-pyrazol-5-yl)piperazine-1-carb-oxamide (SW394877): General procedure F was followed using DIPEA (0.12 mL, 0.67 mmol), phenyl (3-(4-(2-chlorobenzamido)phenyl)-1-methyl-1H-pyrazol-5-yl)carbamate (0.100 g, 0.22 mmol) and 1-acetylpiperazine (0.034 mL, 0.27 mmol) in DMF (5.0 mL). The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394877 as a white solid (0.076 g, 71%). ESI MS for C₂₄H₂₅ClN₆O₃ m/z [M+H]⁺: calculated: 480.2, found: 481.3.

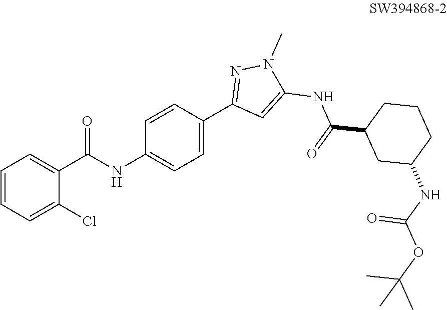

tert-Butyl ((1S,3S)-3-((3-(4-(2-chlorobenzamido)phenyl)-1-methyl-1H-pyrazol-5-yl)carbamoyl)cyclohexyl)carbamate (SW394868-2): The general procedure D was followed using (1S,3S)-3-((tert-butoxycarbonyl)amino)cyclohexane-1-carboxylic acid (0.179 g, 0.73 mmol), 1-chloro-N,N,2-trimethylpropenylamine (Ghosez's reagent) (0.26 mL, 1.96 mmol), N-(4-(5-amino-1-methyl-1H-pyrazol-3-yl)phenyl)-2-chlorobenzamide (0.200 g, 0.61 mmol), pyridine (0.49 mL, 6.12 mmol) and DCM (10.0 mL). The crude product was purified by flash column chromatography on silica gel (DCM/MeOH, 19:1) to give SW394868-2 as a white solid (0.222 g, 66%). ESI MS for C₂₉H₃₄ClN₅O₄ m/z [M+H]⁺: calculated: 551.2, found: 552.2.

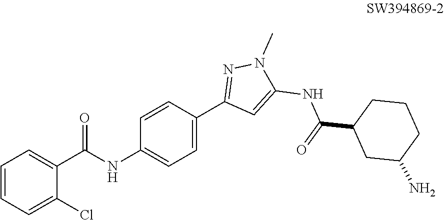

N-(4-(5-((1S,3S)-3-Aminocyclohexane-1-carboxamido)-1-methyl-1H-pyrazol-3-yl)phenyl)-2-chlorobenzamide (SW394869-2): The general procedure E was followed using tert-butyl ((1S,3S)-3-((3-(4-(2-chlorobenzamido)phenyl)-1-methyl-1H-pyrazol-5-yl)carbamoyl)cyclohexyl)-carbamate (0.222 g, 0.40 mmol), and 4 M HCl in dioxane (1.0 mL) to give SW394869-2 as a white solid (0.094 g, 52%). ESI MS for C₂₄H₂ClN₅O₂ m/z [M+H]⁺: calculated: 451.2, found: 452.1.

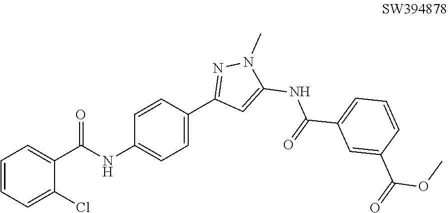 [SMILES: COC(=O)c1cccc(C(=O)Nc2cc(-c3ccc(NC(=O)c4ccccc4Cl)cc3)nn2C)c1]

Methyl 3-((3-(4-(2-chlorobenzamido)phenyl)-1-methyl-1H-pyrazol-5-yl)carbamoyl)benzoate (SW394878): The general procedure A was followed using N-(4-(5-amino-1-methyl-1H-pyrazol-3-yl)phenyl)-2-chlorobenzamide (0.200 g, 0.61 mmol), isophthalic acid monomethyl ester (0.165 g, 0.92 mmol), (COCl)₂ (1.00 mL, 11.7 mmol), DMF (3 drops), DIPEA (0.43 mL, 2.45 mmol) and DCM (6.0 mL). The crude product was purified by flash column chromatography on silica gel (DCM/MeOH, 49:1) to give SW394878 as a white solid (0.212 g, 71%). ESI MS for C₂₆H₂₁ClN₄O₄ m/z [M−H]⁺: calculated: 488.1, found: 487.1.

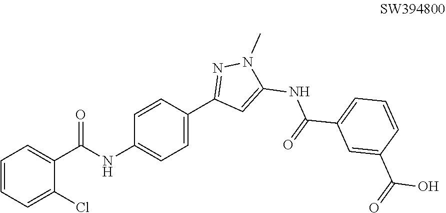 [SMILES: Cn1nc(-c2ccc(NC(=O)c3ccccc3Cl)cc2)cc1NC(=O)c1cccc(C(=O)O)c1]

3-((3-(4-(2-Chlorobenzamido)phenyl)-1-methyl-1H-pyrazol-5-yl)carbamoyl)benzoic acid (SW394800): NaOH (0.019 g, 0.46 mmol) was added to a solution of methyl 3-((3-(4-(2-chlorobenzamido)phenyl)-1-methyl-1H-pyrazol-5-yl)carbamoyl)benzoate (0.113 g, 0.23 mmol) in a 1:1:1 mixture of MeOH/THF/H₂O (6.0 mL). The reaction was stirred at 60° C. and reaction progress was monitored via LCMS. On completion of the reaction, the solution was cool to room temperature and adjusted to pH 5-6 with 2N HCl. The organic layer was extracted with EtOAc (3×10 mL). The combined organic layers were dried (Na₂SO₄) and concentrated. The crude product was purified by reverse phase combiflash chromatography to give SW394800 as a white solid (0.021 g, 19%). 1H NMR (600 MHz, MeOD) δ 8.58 (t, J=1.7 Hz, 1H), 8.20 (dd, J=7.8, 1.5 Hz, 1H), 8.15-8.10 (m, 1H), 7.56 (t, J=7.8 Hz, 1H), 7.49 (d, J=8.3 Hz, 2H), 7.40 (dd, J=8.1, 1.3 Hz, 1H), 7.35 (td, J=7.7, 1.7 Hz, 1H), 7.31 (td, J=7.4, 1.4 Hz, 1H), 6.64 (s, 1H), 3.81 (s, 3H). ESI MS for C₂₅H₁₉ClN₄O₄ m/z [M+H]⁺: calculated: 474.1, found: 475.0.

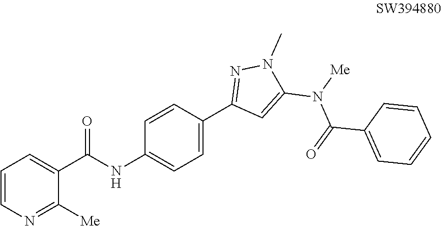 [SMILES: Cc1ncccc1C(=O)Nc1ccc(-c2cc(N(C)C(=O)c3ccccc3)n(C)n2)cc1]

2-methyl-N-(4-(1-methyl-5-(N-methylbenzamido)-1H-pyrazol-3-yl)phenyl)nicotinamide (SW394880): The general procedure C was followed using N-(3-(4-aminophenyl)-1-methyl-1H-pyrazol-5-yl)-N-methylbenzamide (0.113 g, 0.37 mmol), 2-methylnicotinic acid (0.061 g, 0.44 mmol), HATU (0.211 g, 0.55 mmol), DIPEA (0.19 mL, 1.11 mmol) and DCM (4.0 mL). The crude product was purified by flash column chromatography on silica gel (DCM/MeOH, 97:3) to give SW394880 as a brown solid (0.077 g, 49%). ESI MS for C₂₅H₂₃N₅O₂ m/z [M+H]⁺: calculated: 425.5, found: 426.3.

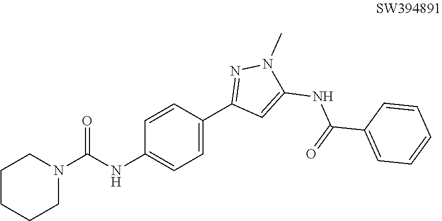 [SMILES: Cn1nc(-c2ccc(NC(=O)N3CCCCC3)cc2)cc1NC(=O)c1ccccc1]

N-(4-(5-benzamido-1-methyl-1H-pyrazol-3-yl)phenyl)piperidine-1-carboxamide (SW394891): General procedure F was followed using DIPEA (0.13 mL, 0.73 mmol), phenyl (4-(5-benzamido-1-methyl-1H-pyrazol-3-yl)phenyl)carbamate (0.100 g, 0.24 mmol) and piperidine (0.029 mL, 0.29 mmol) in 2.5 mL DMF. The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394891 as a yellow solid (0.081 g, 83%). ESI MS for C₂₃H₂₅N₅O₂ m/z [M+H]⁺: calculated: 403.2, found: 404.2.

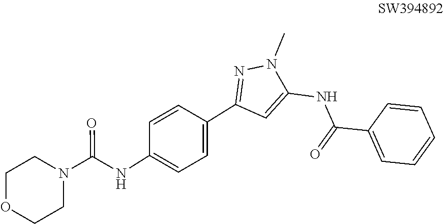 [SMILES: Cn1nc(-c2ccc(NC(=O)N3CCOCC3)cc2)cc1NC(=O)c1ccccc1]

N-(4-(5-benzamido-1-methyl-1H-pyrazol-3-yl)phenyl)morpholine-4-carboxamide (SW394892): General procedure F was followed using DIPEA (0.13 mL, 0.73 mmol), phenyl (4-(5-benzamido-1-methyl-1H-pyrazol-3-yl)phenyl)carbamate (0.100 g, 0.24 mmol) and morpholine (0.025 mL, 0.29 mmol) in 2.5 mL DMF. The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394892 as a white solid (0.075 g, 76%). ESI MS for C₂₂H₂₃N₅O₃ m/z [M+H]⁺: calculated: 405.2, found: 406.1.

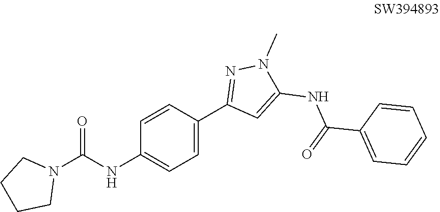 [SMILES: Cn1nc(-c2ccc(NC(=O)N3CCCC3)cc2)cc1NC(=O)c1ccccc1]

N-(4-(5-benzamido-1-methyl-1H-pyrazol-3-yl)phenyl)pyrrolidine-1-carboxamide (SW394893): General procedure F was followed using DIPEA (0.13 mL, 0.73 mmol), phenyl (4-(5-benzamido-1-methyl-1H-pyrazol-3-yl)phenyl)carbamate (0.100 g, 0.24 mmol) and pyrrolidine (0.024 mL, 0.29 mmol) in 2.5 mL. The reaction mixture was diluted with EtOAc (40.0 mL) and then poured into H₂O (40.0 mL). The organic layer was separated, washed with brine (40.0 mL), dried (Na₂SO₄) and concentrated under vacuo. The crude product was purified by flash column chromatography on silica gel (DCM/MeOH, 19:1) to give SW394893 as a white solid (0.066 g, 70%). ESI MS for C₂₂H₂₃N₅O₂ m/z [M+H]⁺: calculated: 389.2, found: 390.1.

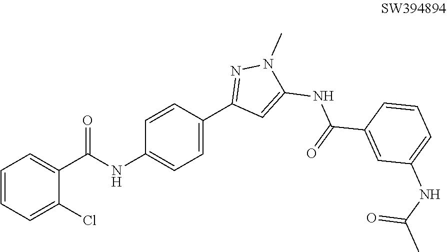 [SMILES: CC(=O)Nc1cccc(C(=O)Nc2cc(-c3ccc(NC(=O)c4ccccc4Cl)cc3)nn2C)c1]

N-(4-(5-(3-acetamidobenzamido)-1-methyl-1H-pyrazol-3-yl)phenyl)-2-chlorobenzamide (SW394894): Et₃N (0.022 mL) was added to a mixture of N-(4-(5-(3-aminobenzamido)-1-methyl-1H-pyrazol-3-yl)phenyl)-2-chlorobenzamide (0.05 g, 0.11 mmol) and acetyl chloride (0.01 mL, 0.13 mmol) in DCM (1.0 mL). The reaction mixture was stirred at room temperature overnight. H₂O (10.0 mL) was added and then extracted with EtOAc (10.0 mL×3). The combined organic layers were washed with brine (30.0 mL), dried (Na₂SO₄) and concentrated under vacuo. The crude product was purified via crystallization in a mixture DCM/hexanes to give SW394894 as a white solid (0.048 g, 87%). ESI MS for C₂₆H₂₂ClN₅O₃ m/z [M+H]⁺: calculated: 487.1, found: 488.1.

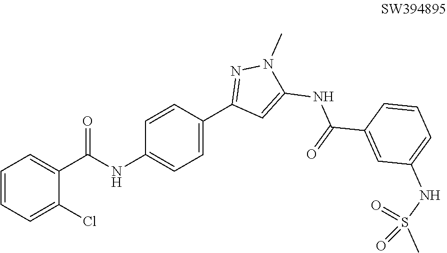 [SMILES: Cn1nc(-c2ccc(NC(=O)c3ccccc3Cl)cc2)cc1NC(=O)c1cccc(NS(C)(=O)=O)c1]

2-chloro-N-(4-(1-methyl-5-(3-(methylsulfonamido)benzamido)-1H-pyrazol-3-yl)phenyl)benzamide (SW394895): Methanesulfonyl chloride (0.019 mL, 0.025 mmol) was added to N-(4-(5-(3-aminobenzamido)-1-methyl-1H-pyrazol-3-yl)phenyl)-2-chlorobenzamide (0.05 g, 0.11 mmol) and in pyridine/DCM mixture (1.0 mL, 1:1). The reaction mixture was stirred at room temperature overnight. The reaction was diluted with saturated NH₄Cl solution (10.0 mL) and extracted with EtOAc (10.0 mL×3) H₂O (10.0 mL). The combined organic layers were washed with 2N HCl (30 mL×2), brine (30.0 mL), dried (Na₂SO₄) and concentrated under vacuo. The crude product was purified by flash column chromatography on silica gel (DCM/MeOH, 24:1) to give SW394895 as a white solid (0.039 g, 67%). ESI MS for C₂₅H₂₂ClN₅O₄S m/z [M+H]⁺: calculated: 523.1, found: 524.1.

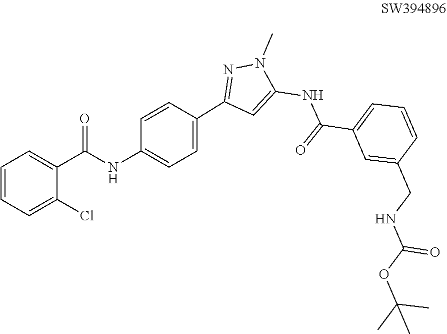 [SMILES: Cn1nc(-c2ccc(NC(=O)c3ccccc3Cl)cc2)cc1NC(=O)c1cccc(CNC(=O)OC(C)(C)C)c1]

tert-butyl (3-((3-(4-(2-chlorobenzamido)phenyl)-1-methyl-1H-pyrazol-5-yl)carbamoyl)benzyl)carbamate (SW394896): The general procedure D was followed using Boc-(3-aminomethyl)benzoic acid (0.231 g, 0.92 mmol), 1-chloro-N,N,2-trimethylpropenylamine (Ghosez's reagent) (0.33 mL, 2.44 mmol), N-(4-(5-amino-1-methyl-1H-pyrazol-3-yl)phenyl)-2-chlorobenzamide (0.250 g, 0.77 mmol), pyridine (0.62 mL, 7.65 mmol) and DCM (10.0 mL). The crude product was purified by flash column chromatography on silica gel (DCM/MeOH, 19:1) to give SW394896 as a white solid (0.325 g, 76%). ESI MS for C₃H₃₀ClN₅O₄ m/z [M+H]⁺: calculated: 559.2, found: 560.2.

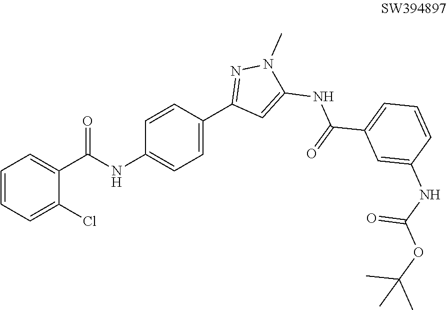 [SMILES: Cn1nc(-c2ccc(NC(=O)c3ccccc3Cl)cc2)cc1NC(=O)c1cccc(NC(=O)OC(C)(C)C)c1]

tert-butyl (3-((3-(4-(2-chlorobenzamido)phenyl)-1-methyl-1H-pyrazol-5-yl)carbamoyl)phenyl)carbamate (SW394897): The general procedure D was followed using 3-(Boc-amino)benzoic acid (0.172 g, 0.73 mmol), 1-chloro-N,N,2-trimethylpropenylamine (Ghosez's reagent) (0.26 mL, 1.96 mmol), N-(4-(5-amino-1-methyl-1H-pyrazol-3-yl)phenyl)-2-chlorobenzamide (0.200 g, 0.61 mmol), pyridine (0.49 mL, 6.12 mmol) and DCM (10.0 mL). The crude product was purified by flash column chromatography on silica gel (DCM/MeOH, 19:1) to give SW394897 as a white solid (0.270 g, 81%). ESI MS for C₂₉H₂ClN₅O₄ m/z [M+H]⁺: calculated: 545.2, found: 546.2.

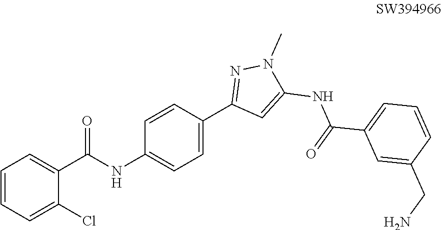 [SMILES: Cn1nc(-c2ccc(NC(=O)c3ccccc3Cl)cc2)cc1NC(=O)c1cccc(CN)c1]

N-(4-(5-(3-(aminomethyl)benzamido)-1-methyl-1H-pyrazol-3-yl)phenyl)-2-chlorobenzamide (SW394966): The general procedure E was followed using tert-butyl (3-((3-(4-(2-chlorobenzamido)phenyl)-1-methyl-1H-pyrazol-5-yl)carbamoyl)benzyl)carbamate (0.37 g, 0.66 mmol) and 4 M HCl in dioxane (2.7 mL) to give SW394966 as a white solid (0.26 g, 85%). ESI MS for C₂₅H₂₂ClN₅O₂ m/z [M+H]⁺: calculated: 459.9, found: 460.2.

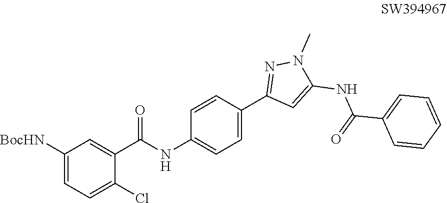 [SMILES: Cn1nc(-c2ccc(NC(=O)c3cc(NC(=O)OC(C)(C)C)ccc3Cl)cc2)cc1NC(=O)c1ccccc1]

tert-butyl (3-((4-(5-benzamido-1-methyl-1H-pyrazol-3-yl)phenyl)carbamoyl)-4-chlorophenyl)carbamate (SW394967): The general procedure D was followed using 5-((tertbutoxycarbonyl)amino)-2-chlorobenzoic acid (0.56 g, 2.05 mmol), 1-chloro-N,N,2-trimethylpropenylamine (Ghosez's reagent) (0.72 mL, 1.38 mmol), N-(3-(4-aminophenyl)-1-methyl-1H-pyrazol-5-yl)benzamide (0.500 g, 1.71), pyridine (1.38 mL, 17.10 mmol) and DCM (15.0 mL). The crude product was purified by flash column chromatography on silica gel (DCM/MeOH, 19:1) to give SW394967 as a white solid (0.408 g, 44%). ESI MS for C₂₉H₂ClN₅O₄ m/z [M+H]⁺: calculated: 545.2, found.

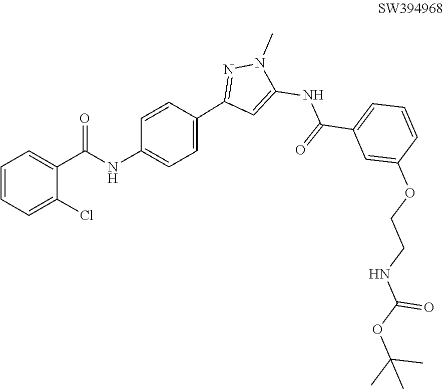 [SMILES: Cn1nc(-c2ccc(NC(=O)c3ccccc3Cl)cc2)cc1NC(=O)c1cccc(OCCNC(=O)OC(C)(C)C)c1]

tert-butyl (2-(3-((3-(4-(2-chlorobenzamido)phenyl)-1-methyl-1H-pyrazol-5-yl)carbamoyl)phenoxy)ethyl)carbamate (SW394968): The general procedure D was followed using 3-(2-((tert-butoxycarbonyl)amino)ethoxy)benzoic acid (0.646 g, 2.30 mmol), 1-chloro-N,N,2-trimethylpropenylamine (Ghosez's reagent) (0.65 mL, 4.90 mmol), N-(4-(5-amino-1-methyl-1H-pyrazol-3-yl)phenyl)-2-chlorobenzamide (0.50 g, 1.53 mmol), pyridine (1.23 mL, 15.3 mmol) and DCM (15.0 mL). The crude product was purified by flash column chromatography on silica gel (DCM/MeOH, 97:3) to give SW394968 as a white solid (0.567 g, 63%). ESI MS for C₃₁H₃₂ClN₅O₅ m/z [M+H]⁺: calculated: 589.2, found: 590.3.

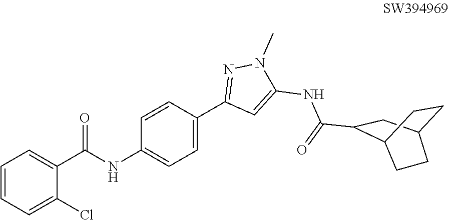 [SMILES: Cn1nc(-c2ccc(NC(=O)c3ccccc3Cl)cc2)cc1NC(=O)C1CC2CCC1CC2]

N-(3-(4-(2-chlorobenzamido)phenyl)-1-methyl-1H-pyrazol-5-yl)bicyclo[2.2.2]octane-2-carboxamide (SW394969): The general procedure A was followed using N-(4-(5-amino-1-methyl-1H-pyrazol-3-yl)phenyl)-2-chlorobenzamide (0.088 g, 0.27 mmol), bicyclo[2.2.2]octane-2-carboxylic acid (0.050 g, 0.32 mmol), (COCl)₂ (0.35 ML, 4.82 mmol), DMF (2 drops), DIPEA (0.1 mL, 0.54 mmol) and DCM (2.0 mL). The crude product was purified by flash column chromatography on silica gel (DCM/MeOH, 49:1) to give SW394969 as a white solid (0.212 g, 61%). ESI MS for C₂H₂₇ClN₄O₂ m/z [M+H]⁺: calculated: 462.2, found: 463.3.

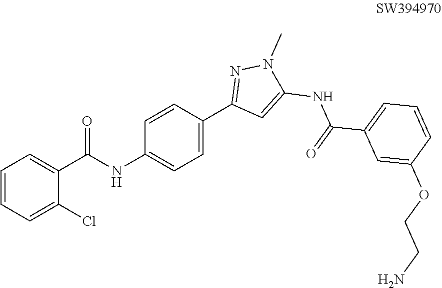 [SMILES: Cn1nc(-c2ccc(NC(=O)c3ccccc3Cl)cc2)cc1NC(=O)c1cccc(OCCN)c1]

N-(4-(5-(3-(2-aminoethoxy)benzamido)-1-methyl-1H-pyrazol-3-yl)phenyl)-2-chlorobenzamide (SW394970): The general procedure E was followed using 2-chloro-N-(4-(1-methyl-5-(3-(2-pivalamidoethoxy)benzamido)-1H-pyrazol-3-yl)phenyl)benzamide (0.5 g, 0.85 mmol), and 4 M HCl in dioxane (5.0 mL) to give SW394970 as a white solid (0.26 g, 85%). ESI MS for C₂₆H₂₄ClN₅O₃ m/z [M+H]⁺: calculated: 489.2, found: 490.2.

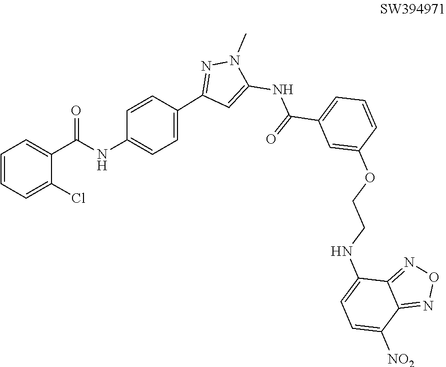 [SMILES: Cn1nc(-c2ccc(NC(=O)c3ccccc3Cl)cc2)cc1NC(=O)c1cccc(OCCNc2ccc([N+](=O)[O-])c3nonc23)c1]

2-Chloro-N-(4-(1-methyl-5-(3-(2-((7-nitrobenzo[c][1,2,5]oxadiazol-4-yl)amino)ethoxy)benz-amido)-1H-pyrazol-3-yl)phenyl)benzamide (SW394971): N-(4-(5-(3-(2-aminoethoxy)benzamido)-1-methyl-1H-pyrazol-3-yl)phenyl)-2-chloro-benzamide (0.010 g, 0.0204 mmol) and triethylamine (0.006 mL, 0.0408 mmol) were added to the solution of NBD-Cl (0.005 g, 0.0224 mmol) in THE (2 mL). The reaction was stirred at reflux under nitrogen atmosphere for 16 h. Solvent was removed under vacuo, the residue was redissolved in EtOAc (10 mL), washed with 1 M HCl (8 mL), H₂O (8 mL), brine, dried (Na₂SO₄) and concentrated. The crude product was purified via flash column chromatography on silica gel (DCM/MeOH, 49:1) to give SW394971 as orange solid (0.010 g, 80%). ESI MS for C₃₂H₂₅ClN₈O₆ m/z [M+H]⁺: calculated: 652.2, found: 653.3.

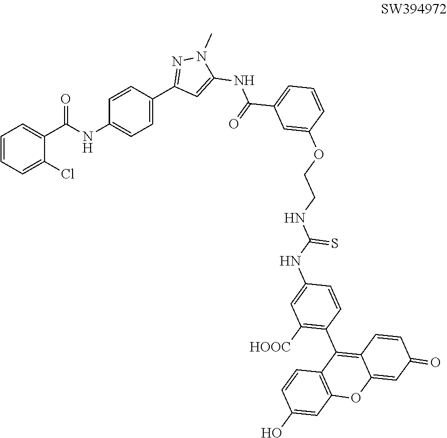 [SMILES: Cn1nc(-c2ccc(NC(=O)c3ccccc3Cl)cc2)cc1NC(=O)c1cccc(OCCNC(=S)Nc2ccc(-c3c4ccc(=O)cc-4oc4cc(O)ccc34)c(C(=O)O)c2)c1]

5-(3-(2-(3-((3-(4-(2-chlorobenzamido)phenyl)-1-methyl-1H-pyrazol-5-yl)carbamoyl)phenoxy)ethyl)thioureido)-2-(6-hydroxy-3-oxo-3H-xanthen-9-yl)benzoic acid (SW394972): Triethylamine (0.23 mL, 1.56 mmol) was added to a solution of fluorescin isothiocyanate isomer (0.0175 g, 0.0449 mmol) in DMF (0.3 mL). A solution of N-(4-(5-(3-(2-aminoethoxy)benzamido)-1-methyl-1H-pyrazol-3-yl)phenyl)-2-chloro-benzamide (0.020 g, 0.0408 mmol) in DMF (0.2 ML) was then added. The reaction was stirred at room temperature for 5 h under nitrogen atmosphere. Solvent was removed under vacuo and the crude product purified by reverse phase combiflash chromatography to give SW394972 as orange solid (0.013 g, 37%). ESI MS for C₄₇H₃₅ClN₆O₈ m/z [M+H]⁺: calculated: 878.2, found: 879.5.

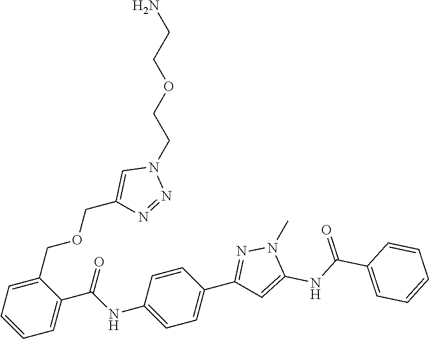 [SMILES: Cn1nc(-c2ccc(NC(=O)c3ccccc3COCc3cn(CCOCCN)nn3)cc2)cc1NC(=O)c1ccccc1]

### Preparation of Intermediates

2-(((1-(2-(2-aminoethoxy)ethyl)-1H-1,2,3-triazol-4-yl)methoxy)methyl)-N-(4-(5-benzamido-1-methyl-1H-pyrazol-3-yl)phenyl)benzamide: The general procedure E was followed using tert-butyl (2-(2-(4-(((2-((4-(5-benzamido-1-methyl-1H-pyrazol-3-yl)phenyl)carbamoyl)benzyl)oxy)methyl)-1H-1,2,3-triazol-1-yl)ethoxy)ethyl)carbamate (187.6 mg, 0.27 mmol) in 4 M HCl in dioxane (5 mL) was stirred at room temperature overnight. The solution was concentrated in vacuo to give 2-(((1-(2-(2-aminoethoxy)ethyl)-1H-1,2,3-triazol-4-yl)methoxy)methyl)-N-(4-(5-benzamido-1-methyl-1H-pyrazol-3-yl)phenyl)benzamide as a white solid (72.8 mg, 45%). ESI MS for C₃₂H₃₄N₈O₄ m/z [M+H]⁺: calculated: 594.7, found: 595.2.

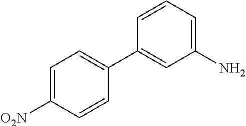 [SMILES: Nc1cccc(-c2ccc([N+](=O)[O-])cc2)c1]

4′-nitro-[1,1′-biphenyl]-3-amine: To a solution of 3-iodoaniline (0.14 mL, 1.2 mmol) and 4-nitrophenyl boronic acid (200 mg, 1.2 mmol) in THF (20 mL) was added Pd(dppf)Cl₂ (17.7 mg, 0.02 mmol) and 5 M NaOH (1 g, 25 mmol), and refluxed overnight. The mixture was diluted with EtOAc and washed with H₂O, brine, dried (Na₂SO₄) and concentrated under reduced pressure. The crude product was purified by column chromatography on silica gel (EtOAc/hex 20:80) 4′-nitro-[1,1′-biphenyl]-3-amine as a yellow solid (0.110 g, 46%). ¹H NMR (400 MHz, CDCl₃) δ 8.32-8.24 (m, 2H), 7.75-7.67 (m, 2H), 7.29 (t, J=7.8 Hz, 1H), 7.02 (dt, J=7.8, 1.3 Hz, 1H), 6.93 (t, J=2.0 Hz, 1H), 6.78 (ddd, J=7.9, 2.4, 1.0 Hz, 1H). ESI MS for C₁₂H₁₀N₂O₂ m/z [M+H]⁺: calculated: 214.2, found: 215.0.

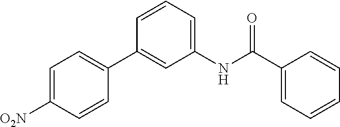 [SMILES: O=C(Nc1cccc(-c2ccc([N+](=O)[O-])cc2)c1)c1ccccc1]

N-(4′-nitro-[1,1′-biphenyl]-3-yl)benzamide: The general procedure A was followed using 4′-nitro-[1,1′-biphenyl]-3-amine and DIPEA (0.15 mL, 0.84 mmol) in DCM (2 mL) was lowered to 0° C. Benzoyl chloride (0.1 mL, 0.84 mmol) was added and raised to room temperature and stirred overnight. The mixture was diluted with EtOAc and washed with H₂O, brine, dried (Na₂SO₄) and concentrated under reduced pressure. The crude product was purified by column chromatography on silica gel (EtOAc/hex 20:80)N-(4′-nitro-[1,1′-biphenyl]-3-yl)benzamide as a tan solid (0.056 g, 63%). ¹H NMR (400 MHz, CDCl₃) δ 8.18 (s, 1H), 8.17-8.12 (m, 2H), 8.04-7.96 (m, 2H), 7.85-7.76 (m, 2H), 7.65-7.60 (m, 2H), 7.56-7.43 (m, 2H), 7.41-7.33 (m, 2H), 7.29 (dt, J=7.8, 1.4 Hz, 1H). ESI MS for C₁₉H₁₄N₂O₃ m/z [M+H]⁺: calculated: 318.3, found: 319.0.

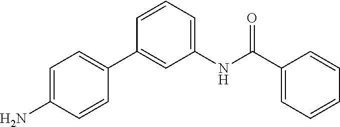 [SMILES: Nc1ccc(-c2cccc(NC(=O)c3ccccc3)c2)cc1]

N-(4′-amino-[1,1′-biphenyl]-3-yl)benzamide: A flame dried flask was charged with N-(4′-nitro-[1,1′-biphenyl]-3-yl)benzamide (56 mg, 0.176 mmol) and Pd/C (33 mg). THE was added under N₂, then the reaction was placed under H₂ and run overnight at room temperature. The crude reaction was filtered through celite and concentrated in vacuo give N-(4′-amino-[1,1′-biphenyl]-3-yl)benzamide as a yellow solid (0.051 g, 100%). ESI MS for C₁₉H₁₆N₂O m/z [M+H]⁺: calculated: 288.6, found: 289.1.

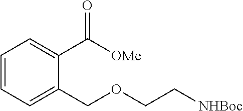 [SMILES: COC(=O)c1ccccc1COCCNC(=O)OC(C)(C)C]

methyl 2-((2-((tert-butoxycarbonyl)amino)ethoxy)methyl)benzoate: A solution of 2-bromomethyl benzoate (1.5 g, 6.55 mmol) in THE (70 mL) was cooled to 0° C. 60% NaH (580 mg, 14.4 mmol) in mineral oil was added and stirred at 0° C. for 10 min. Propargyl alcohol (1.12 mL, 7.2 mmol) in THF (5 mL) was added at 0° C., then raised to room temperature and stirred overnight. The mixture was diluted with EtOAc and washed with H₂O, brine, dried (Na₂SO₄) and concentrated under reduced pressure. The crude product was purified by column chromatography on silica gel (EtOAc/hex 15:85) to give methyl 2-((2-((tertbutoxycarbonyl)amino)ethoxy)methyl)benzoate as a clear oil (0.684 g, 34%) ¹H NMRa: (600 MHz, CDCl₃) δ 7.86 (td, J=8.2, 7.8, 1.4 Hz, 1H), 7.52 (d, J=7.7 Hz, 1H), 7.43 (td, J=7.6, 1.5 Hz, 1H), 7.25 (td, J=7.6, 1.4 Hz, 1H), 5.04 (s, 1H), 4.80 (d, J=6.0 Hz, 2H), 3.53 (t, J=5.1 Hz, 2H), 3.30 (q, J=5.4 Hz, 2H), 1.36 (s, 9H). ESI MS for C₁₆H₂₃NO₅ m/z [M+H]⁺: calculated: 309.4, found: 310.1.

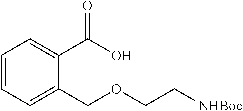 [SMILES: CC(C)(C)OC(=O)NCCOCc1ccccc1C(=O)O]

2-((2-((tert-butoxycarbonyl)amino)ethoxy)methyl)benzoic acid: To a solution of methyl 2-((2-((tert-butoxycarbonyl)amino)ethoxy)methyl)benzoate (813 mg, 2.18 mmol) in MeOH (20 mL) was added 3.5 M NaOH (0.87 g, 21.8 mmol) and heated to 50° C. for 3 hours. The reaction mixture was cooled to room temperature and 3.5 M HCl (6.25 mL) was added. The mixture was diluted with EtOAc and washed with H₂O, brine, dried (Na₂SO₄) and concentrated under reduced pressure to give 2-((2-((tert-butoxycarbonyl)amino)ethoxy)methyl)benzoic acid as a white solid (0.643 g, 100%). ¹H NMR: (400 MHz, CDCl₃) δ 10.31 (s, 1H), 7.98 (d, J=7.8 Hz, 1H), 7.58 (d, J=7.8 Hz, 1H), 7.52-7.43 (m, 1H), 7.27 (t, J=7.7 Hz, 1H), 5.03 (s, 1H), 4.87 (s, 2H), 3.56 (t, J=5.2 Hz, 2H), 3.33 (q, J=5.8 Hz, 2H), 1.37 (s, 9H). ESI MS for C₃₁H₂₃ClN₄O₃ m/z [M+H]⁺: calculated: 295.3, found: 296.2.

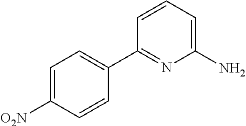 [SMILES: Nc1cccc(-c2ccc([N+](=O)[O-])cc2)n1]

6-(4-nitrophenyl)pyridin-2-amine: To a solution of 2-amino-6-bromopyridine (0.5 g, 2.89 mmol) and 4-nitrophenyl boronic acid (482 mg, 2.89 mmol) in THE (20 mL) was added Pd(dppf)Cl₂ (42.3 mg, 0.06 mmol) and 5 M NaOH (1 g, 25 mmol), and refluxed overnight. The mixture was diluted with EtOAc and washed with H₂O, brine, dried (Na₂SO₄) and concentrated under reduced pressure. The crude product was purified by column chromatography on silica gel (EtOAc/hex 20:80) 6-(4-nitrophenyl)pyridin-2-amine as a brown solid (0.253 g, 41%). ¹H NMR (400 MHz, CDCl₃) δ 8.35-8.27 (m, 2H), 8.18-8.10 (m, 2H), 7.57 (q, J=8.1 Hz, 1H), 7.20 (dd, J=7.5, 0.7 Hz, 1H), 6.58 (dd, J=8.2, 0.7 Hz, 1H). ESI MS for C H₉N₃O₂ m/z [M+H]⁺: calculated: 215.2, found: 216.0.

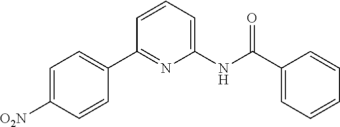 [SMILES: O=C(Nc1cccc(-c2ccc([N+](=O)[O-])cc2)n1)c1ccccc1]

N-(6-(4-nitrophenyl)pyridin-2-yl)benzamide: The general procedure A was followed using 6-(4-nitrophenyl)pyridin-2-amine (253.3 mg, 1.18 mmol) and DIPEA (1.03 mL, 5.9 mmol) in DCM (2 mL) was lowered to 0° C. Benzoyl chloride (0.41 mL, 3.53 mmol) was added and raised to room temperature and stirred overnight. The mixture was diluted with EtOAc and washed with H₂O, brine, dried (Na₂SO₄) and concentrated under reduced pressure. The crude product was purified by column chromatography on silica gel (EtOAc/hex 20:80)N-(6-(4-nitrophenyl)pyridin-2-yl)benzamide as a tan solid (0.313 g, 83%). ¹H NMR (400 MHz, cdcl₃) δ 8.71 (s, 1H), 8.44 (d, J=8.3 Hz, 1H), 8.15 (d, J=8.7 Hz, 2H), 8.00-7.94 (m, 2H), 7.90 (t, J=8.0 Hz, 1H), 7.65-7.57 (m, 2H), 7.53 (t, J=7.7 Hz, 2H).

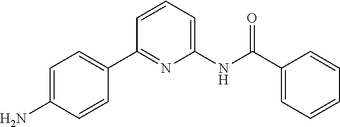 [SMILES: Nc1ccc(-c2cccc(NC(=O)c3ccccc3)n2)cc1]

N-(6-(4-aminophenyl)pyridin-2-yl)benzamide: A flame dried flask was charged with N-(6-(4-nitrophenyl)pyridin-2-yl)benzamide (313 mg, 0.98 mmol) and Pd/C (188 mg). THE (20 mL) was added under N₂, then the reaction was placed under H₂ and run overnight at room temperature. The crude reaction was filtered through celite and concentrated in vacuo give N-(6-(4-aminophenyl)pyridin-2-yl)benzamide as a yellow solid (0.281 g, 99%). ESI MS for C₂₆H₁₉ClN₂O₂ m/z [M+H]⁺: calculated: 426.9, found: 428.1.

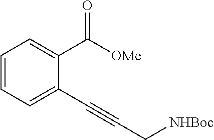 [SMILES: COC(=O)c1ccccc1C#CCNC(=O)OC(C)(C)C]

methyl 2-(3-((tert-butoxycarbonyl)amino)prop-1-yn-1-yl)benzoate: Methyl 2-iodobenzoate (1.516 mL, 10.32 mmol), N-Boc-propargylamine (1 g, 6.45 mmol) and piperidine (3.83 mL, 38.7 mmol) in THF (12 mL) was degassed via purging with N₂ for one hour. PdCl₂(PPh₃)₂ (45 mg, 0.06 mmol) and CuI (25 mg, 0.13 mmol) were added to the solution and was stirred overnight at room temperature. NH₄Cl (10 mL) was added until the white precipitate dissolved. The reaction was quenched with 2 M HCl (20 mL), and the aqueous layer was separated. The aqueous layer was washed with Et₂O (50 mL×3), and the organic layers were combined and dried with Na₂SO₄ and concentrated under reduced pressure. The crude product was purified by flash column chromatography on silica gel (EtOAc/hex, 15:85) to give methyl 2-(3-((tert-butoxycarbonyl)amino)prop-1-yn-1-yl)benzoate as a yellow solid (1.127 g, 60%). ¹H NMR (400 MHz, CDCl₃) δ 7.78 (dd, J=7.9, 1.4 Hz, 1H), 7.38 (dd, J=7.8, 1.4 Hz, 1H), 7.30 (td, J=7.6, 1.5 Hz, 1H), 7.21 (td, J=7.7, 1.4 Hz, 1H), 5.32-5.24 (m, 1H), 4.11 (d, J=5.7 Hz, 2H), 3.78 (s, 3H), 1.35 (s, 9H).

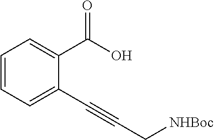 [SMILES: CC(C)(C)OC(=O)NCC#Cc1ccccc1C(=O)O]

2-(3-((tert-butoxycarbonyl)amino)prop-1-yn-1-yl)benzoic acid: To a solution of methyl 2-(3-((tert-butoxycarbonyl)amino)prop-1-yn-1-yl)benzoate (1.127 g, 3.9 mmol) in MeOH (50 mL) was added 3.5 M NaOH (1.56 g, 39 mmol) and heated to 50° C. for 3 hours. The reaction mixture was cooled to room temperature and 3.5 M HCl (12 mL) was added. The mixture was diluted with EtOAc and washed with H₂O, brine, dried (Na₂SO₄) and concentrated under reduced pressure to give 2-(3-((tert-butoxycarbonyl)amino)prop-1-yn-1-yl)benzoic acid as a white solid (1.07 g, 100%). ¹H NMR: (400 MHz, CDCl₃) δ 7.94 (d, J=7.8 Hz, 1H), 7.45 (d, J=7.8 Hz, 1H), 7.40 (q, J=7.7, 7.3 Hz, 1H), 7.28 (t, J=7.6 Hz, 1H), 4.12 (t, J=8.5 Hz, 2H), 1.43-1.35 (m, 16H). ESI MS for Cl₅H₁₇NO₄ m/z [M+H]⁺: calculated: 275.3, found: 276.1.

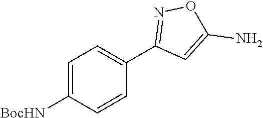 [SMILES: CC(C)(C)OC(=O)Nc1ccc(-c2cc(N)on2)cc1]

tert-butyl (4-(5-aminoisoxazol-3-yl)phenyl)carbamate: To a solution of tert-butyl (4-(5-aminoisoxazol-3-yl)phenyl)carbamate (134.1 mg, 0.52 mmol) and NaOH (41.3 mg, 1.03 mmol) in H₂O (2 mL) was added hydroxylamine hydrochloride (39.4 mg, 0.57 mmol) and was heated to 100° C. for 3 hours. The crude product was purified by flash column chromatography on silica gel (DCM/MeOH, 90:10) to give tert-butyl (4-(5-aminoisoxazol-3-yl)phenyl)carbamate as a yellow solid (0.020 g, 14%). ESI MS for C₂₃H₁₇N₃O₃ m/z [M+H]⁺: calculated: 275.3, found: 276.1.

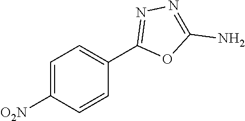 [SMILES: Nc1nnc(-c2ccc([N+](=O)[O-])cc2)o1]

5-(4-nitrophenyl)-1,3,4-oxadiazol-2-amine: EDCl (1.16 g, 7.46 mmol) was added portionwise to a solution of 4-nitrobenzoic acid (1 g, 4.975 mmol) and thiosemicarbazide (453.4 mg, 4.975 mmol) in DMF (20 mL) at 0° C. and stirred for one hour at room temperature. EDCl (1.16 g, 7.46 mmol) was added in one portion and stirred at 60° C. for one hour. The solution was poured into ice water and filtered to give 5-(4-nitrophenyl)-1,3,4-oxadiazol-2-amine as a red solid (0.435 g, 42%). ¹H NMR (400 MHz, DMSO) δ 8.42-8.32 (m, 2H), 8.06-7.99 (m, 2H), 7.55 (s, 2H).

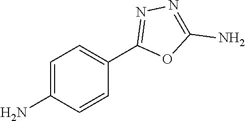 [SMILES: Nc1ccc(-c2nnc(N)o2)cc1]

5-(4-aminophenyl)-1,3,4-oxadiazol-2-amine: A flame dried flask was charged with give 5-(4-nitrophenyl)-1,3,4-oxadiazol-2-amine (210.2 mg, 1.02 mmol) and Pd/C (196 mg). THF (20 mL) was added under N₂, then the reaction was placed under H₂ and run overnight at room temperature. The crude reaction was filtered through celite and concentrated in vacuo. to give 5-(4-aminophenyl)-1,3,4-oxadiazol-2-amine as a yellow solid (179.7 mg, 100%). ¹H NMR ¹¹C NMR ¹³C NMR; ESI MS for C₃H₃N₄O m/z [M+H]⁺: calculated: 176.2, found: 177.1.

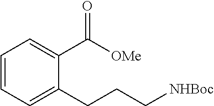 [SMILES: COC(=O)c1ccccc1CCCNC(=O)OC(C)(C)C]

methyl 2-(3-((tert-butoxycarbonyl)amino)propyl)benzoate: A flame dried flask was charged with 2-(3-((tert-butoxycarbonyl)amino)prop-1-yn-1-yl)benzoic acid (374.7 mg, 1.3 mmol) and Pd/C (250 mg). THE (10 mL) was added under N₂, then the reaction was placed under H₂ and run overnight at room temperature. The crude reaction was filtered through celite and concentrated in vacuo. The crude product was purified by flash column chromatography on silica gel (EtOAc/hex, 10:90) to give methyl 2-(3-((tert-butoxycarbonyl)amino)propyl)benzoate as a clear oil (380.9 mg, 100%). ¹H NMR (400 MHz, CDCl₃) δ 7.77 (d, J=7.8 Hz, 1H), 7.31 (td, J=7.5, 1.5 Hz, 1H), 7.19-7.09 (m, 2H), 3.78 (s, 3H), 3.06 (t, J=7.3 Hz, 2H), 2.86 (dd, J=9.0, 6.7 Hz, 2H), 1.35 (s, 9H).

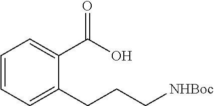 [SMILES: CC(C)(C)OC(=O)NCCCc1ccccc1C(=O)O]

2-(3-((tert-butoxycarbonyl)amino)propyl)benzoic acid: To a solution of methyl 2-(3-((tert-butoxycarbonyl)amino)propyl)benzoate (380.9 mg, 1.3 mmol) in MeOH (10 mL) was added 3.5 M NaOH (520 mg, 13 mmol) and heated to 50° C. for 3 hours. The reaction mixture was cooled to room temperature and 3.5 M HCl (4.5 mL) was added. The mixture was diluted with EtOAc and washed with H₂O, brine, dried (Na₂SO₄) and concentrated under reduced pressure to give 2-(3-((tert-butoxycarbonyl)amino)propyl)benzoic acid as a white solid (293 mg, 81%). ¹H NMR (400 MHz, CDCl₃) δ 7.93 (d, J=7.9 Hz, 1H), 7.35 (t, J=7.4 Hz, 1H), 7.17 (dd, J=8.0, 5.3 Hz, 2H), 3.10 (q, J=7.3, 6.6 Hz, 2H), 2.96 (t, J=7.7 Hz, 2H), 1.74 (p, J=7.3 Hz, 2H), 1.37 (d, J=11.9 Hz, 9H).

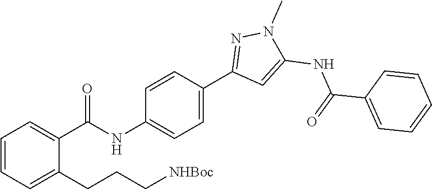 [SMILES: Cn1nc(-c2ccc(NC(=O)c3ccccc3CCCNC(=O)OC(C)(C)C)cc2)cc1NC(=O)c1ccccc1]

tert-butyl (3-(2-((4-(5-benzamido-1-methyl-1H-pyrazol-3-yl)phenyl)carbamoyl)phenyl)propyl)carbamate: The general procedure C was followed using N-(3-(4-aminophenyl)-1-methyl-1H-pyrazol-5-yl)benzamide (100 mg, 0.34 mmol), 2-(3-((tertbutoxycarbonyl)amino)propyl)benzoic acid (114.7 mg, 0.41 mmol), HATU (194 mg, 0.51 mmol), DIPEA (0.18 mL, 1.02 mmol) and DMF (3 mL). The crude product was purified by flash column chromatography on silica gel (DCM/MeOH, 98:2) to give tert-butyl (3-(2-((4-(5-benzamido-1-methyl-1H-pyrazol-3-yl)phenyl)carbamoyl)phenyl)propyl)carbamate as a white solid (32.7 mg, 17%). ¹H NMR (600 MHz, MeOD) δ 7.88 (d, J=7.0 Hz, 2H), 7.85-7.79 (m, 2H), 7.63 (dt, J=12.9, 7.3 Hz, 3H), 7.50 (d, J=7.0 Hz, 1H), 7.40 (dq, J=13.6, 7.1 Hz, 3H), 7.31-7.24 (m, 1H), 7.18 (dt, J=13.0, 6.5 Hz, 1H), 6.47 (dd, J=15.4, 7.9 Hz, 1H), 3.71 (t, J=5.6 Hz, 3H), 3.07-2.95 (m, 2H), 2.74 (dt, J=13.9, 5.7 Hz, 2H), 1.75 (p, J=7.0 Hz, 2H), 1.39-1.27 (m, 9H). ESI MS for C₃₂H₃₅N₅O₄ m/z [M+H]⁺: calculated: 553.7, found: 554.2.

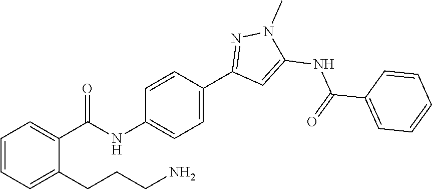 [SMILES: Cn1nc(-c2ccc(NC(=O)c3ccccc3CCCN)cc2)cc1NC(=O)c1ccccc1]

2-(3-aminopropyl)-N-(4-(5-benzamido-1-methyl-1H-pyrazol-3-yl)phenyl)benzamide: The general procedure E was followed using tert-butyl (3-(2-((4-(5-benzamido-1-methyl-1H-pyrazol-3-yl)phenyl)carbamoyl)phenyl)propyl)carbamate (37.2 mg, 0.06 mmol) in 4 M HCl in dioxane (5 mL) was stirred at room temperature overnight. The solution was concentrated in vacuo to give 2-(3-aminopropyl)-N-(4-(5-benzamido-1-methyl-1H-pyrazol-3-yl)phenyl)benzamide as a white solid (20.2 mg, 74%). ESI MS for C₂₇H₂₇N₅O₃ m/z [M+H]⁺: calculated: 453.6, found: 454.1.

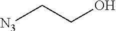

2-azidoethan-1-ol: To a solution of 2-bromoethanol (0.57 mL, 8 mmol) in H₂O (10 mL) was added NaN₃ (1.56 g, 24 mmol), which was stirred overnight at 80° C. The solution was washed with Et₂O (4×50 mL) and concentrated in vacuo to give 2-azidoethan-1-ol as a clear oil (696.6 mg, 100%). ESI MS for C₂₇H₂₂ClN₅O₂ m/z [M+H]⁺: calculated: 87.1, found: 88.1.

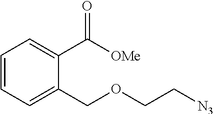 [SMILES: COC(=O)c1ccccc1COCCN=[N+]=[N-]]

methyl 2-((2-azidoethoxy)methyl)benzoate: A solution of 2-bromomethyl benzoate (0.832 g, 3.63 mmol) in THF (50 mL) was cooled to 0° C. 60% NaH (320 mg, 8 mmol) in mineral oil was added and stirred at 0° C. for 10 min. 2-azidoethan-1-ol (348.3 mg, 4 mmol) in THF (10 mL) was added at 0° C., then raised to room temperature and stirred overnight. The mixture was diluted with EtOAc and washed with H₂O, brine, dried (Na₂SO₄) and concentrated under reduced pressure. The crude product was purified by column chromatography on silica gel (EtOAc/hex 5:95) to give methyl 2-((2-azidoethoxy)methyl)benzoate as a tan solid (0.232 g, 28%). ESI MS for C₂₇H₂₂ClN₅O₂ m/z [M+H]⁺: calculated: 235.2, found: 236.1.

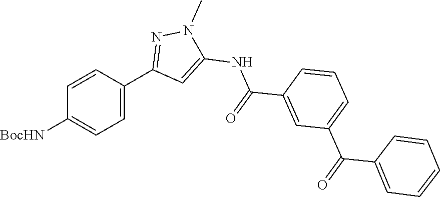 [SMILES: Cn1nc(-c2ccc(NC(=O)OC(C)(C)C)cc2)cc1NC(=O)c1cccc(C(=O)c2ccccc2)c1]

tert-butyl (4-(5-(3-benzoylbenzamido)-1-methyl-1H-pyrazol-3-yl)phenyl)carbamate: The general procedure C was followed using tert-butyl-(4-(5-amino-1-methyl-1H-pyrazol-3-yl)phenyl)carbamate (250 mg, 0.87 mmol), 3-benzoyl benzoic acid (235 mg, 1.04 mmol), HATU (496 mg, 1.31 mmol), DIPEA (0.45 mL, 2.61 mmol) and DMF (8 mL). The crude product was purified by flash column chromatography on silica gel (DCM/MeOH, 99:1) to give tert-butyl (4-(5-(3-benzoylbenzamido)-1-methyl-1H-pyrazol-3-yl)phenyl)carbamate as a tan solid (195 mg, 45%). ¹H NMR (400 MHz, CDCl₃) δ 8.47 (s, 1H), 8.35 (s, 1H), 8.17 (d, J=7.8 Hz, 1H), 7.95 (dt, J=7.8, 1.4 Hz, 1H), 7.82-7.74 (m, 2H), 7.68-7.57 (m, 4H), 7.50 (t, J=7.7 Hz, 2H), 7.36 (d, J=8.4 Hz, 2H), 6.63 (s, 1H), 6.49 (s, 1H), 3.77 (s, 3H), 1.53 (s, 9H). ESI MS for C₂₉H₂₈N₄O₄ m/z [M+H]⁺: calculated: 496.6, found: 497.2.

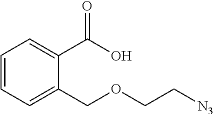 [SMILES: [N-]=[N+]=NCCOCc1ccccc1C(=O)O]

2-((2-azidoethoxy)methyl)benzoic acid: To a solution of methyl 2-((2-azidoethoxy)methyl)benzoate (231.5 mg, 0.4 mmol) in MeOH (9 mL) was added 3.5 M NaOH (392 mg, 9.8 mmol) and heated to 50° C. for 3 hours. The reaction mixture was cooled to room temperature and 3.5 M HCl (2.8 mL) was added. The mixture was diluted with EtOAc and washed with H₂O, brine, dried (Na₂SO₄) and concentrated under reduced pressure to give 2-((2-azidoethoxy)methyl)benzoic acid as a white solid (183 mg, 85%). ¹H NMR (400 MHz, CDCl₃) δ 7.64 (dd, J=30.0, 7.8 Hz, 1H), 7.53 (qd, J=9.0, 8.3, 1.4 Hz, 1H), 7.32 (t, J=7.6 Hz, 1H), 4.95 (s, 1H), 3.72 (t, J=4.9 Hz, 1H), 3.41 (dd, J=9.9, 4.8 Hz, 2H).

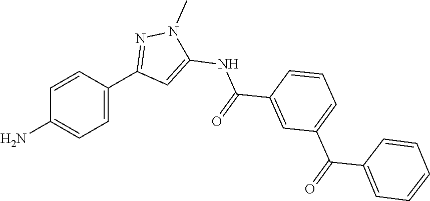 [SMILES: Cn1nc(-c2ccc(N)cc2)cc1NC(=O)c1cccc(C(=O)c2ccccc2)c1]

N-(3-(4-aminophenyl)-1-methyl-1H-pyrazol-5-yl)-3-benzoylbenzamide: The general procedure E was followed using tert-butyl (4-(5-(3-benzoylbenzamido)-1-methyl-1H-pyrazol-3-yl)phenyl)carbamate (195 mg, 0.4 mmol) in 4 M HCl in dioxane (10 mL) was stirred at room temperature overnight. The solution was concentrated in vacuo to give N-(3-(4-aminophenyl)-1-methyl-1H-pyrazol-5-yl)-3-benzoylbenzamide as a tan solid (159 mg, 100%). ESI MS for C₂₄H₂₀N₄O₂ m/z [M+H]⁺: calculated: 396.5, found: 397.1.

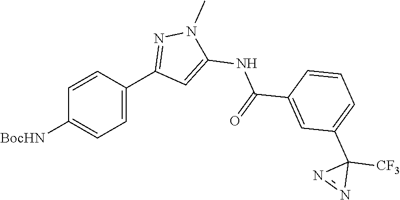 [SMILES: Cn1nc(-c2ccc(NC(=O)OC(C)(C)C)cc2)cc1NC(=O)c1cccc(C2(C(F)(F)F)N=N2)c1]

tert-butyl (4-(1-methyl-5-(3-(3-(trifluoromethyl)-3H-diazirin-3-yl)benzamido)-1H-pyrazol-3-yl)phenyl)carbamate: The general procedure C was followed using tert-butyl-(4-(5-amino-1-methyl-1H-pyrazol-3-yl)phenyl)carbamate (250 mg, 0.87 mmol), 3-(3-(trifluoromethyl)-3H-diazirin-3-yl)benzoic acid (239 mg, 1.04 mmol), HATU (496 mg, 1.31 mmol), DIPEA (0.45 mL, 2.61 mmol) and DMF (8 mL). The crude product was recrystallized to give tert-butyl (4-(1-methyl-5-(3-(3-(trifluoromethyl)-3H-diazirin-3-yl)benzamido)-1H-pyrazol-3-yl)phenyl)carbamate as a yellow solid (226 mg, 52%). ESI MS for C₂₄H₂₃F₃N₆O₃ m/z [M+H]⁺: calculated: 500.5, found: 501.2.

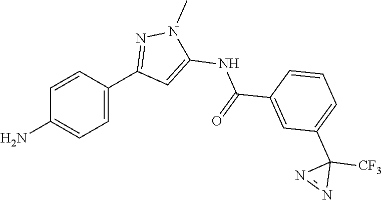 [SMILES: Cn1nc(-c2ccc(N)cc2)cc1NC(=O)c1cccc(C2(C(F)(F)F)N=N2)c1]

N-(3-(4-aminophenyl)-1-methyl-1H-pyrazol-5-yl)-3-(3-(trifluoromethyl)-3H-diazirin-3-yl)benzamide: The general procedure E was followed using tert-butyl (4-(1-methyl-5-(3-(3-(trifluoromethyl)-3H-diazirin-3-yl)benzamido)-1H-pyrazol-3-yl)phenyl)carbamate (226 mg, 0.45 mmol) in 4 M HCl in dioxane (10 mL) was stirred at room temperature overnight. The solution was concentrated in vacuo to give N-(3-(4-aminophenyl)-1-methyl-1H-pyrazol-5-yl)-3-(3-(trifluoromethyl)-3H-diazirin-3-yl)benzamide as a tan solid (180 mg, 100%). ESI MS for C₁₉H₁₅F₃N₆O m/z [M+H]⁺: calculated: 400.4, found: 401.1.

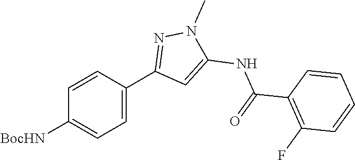 [SMILES: Cn1nc(-c2ccc(NC(=O)OC(C)(C)C)cc2)cc1NC(=O)c1ccccc1F]

tert-butyl (4-(5-(2-fluorobenzamido)-1-methyl-1H-pyrazol-3-yl)phenyl)carbamate: The general procedure C was followed using tert-butyl-(4-(5-amino-1-methyl-1H-pyrazol-3-yl)phenyl)carbamate (250 mg, 0.87 mmol), 2-fluoro benzoic acid (146 mg, 1.04 mmol), HATU (496 mg, 1.31 mmol), DIPEA (0.45 mL, 2.61 mmol) and DMF (8 mL). The crude product was recrystallized to give tert-butyl (4-(5-(2-fluorobenzamido)-1-methyl-1H-pyrazol-3-yl)phenyl)carbamate as a yellow solid (196 mg, 55%). ESI MS for C₂₂H₂₃FN₄O₃ m/z [M+H]⁺: calculated: 410.5, found: 411.1.

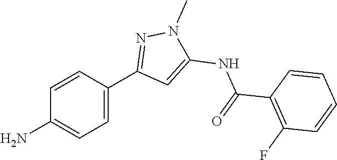 [SMILES: Cn1nc(-c2ccc(N)cc2)cc1NC(=O)c1ccccc1F]

N-(3-(4-aminophenyl)-1-methyl-1H-pyrazol-5-yl)-2-fluorobenzamide: The general procedure E was followed using tert-butyl (4-(5-(2-fluorobenzamido)-1-methyl-1H-pyrazol-3-yl)phenyl)carbamate (196 mg, 0.48 mmol) in 4 M HCl in dioxane (10 mL) was stirred at room temperature overnight. The solution was concentrated in vacuo to give N-(3-(4-aminophenyl)-1-methyl-1H-pyrazol-5-yl)-2-fluorobenzamide as a tan solid (149 mg, 100%). ESI MS for C₁₇H₁₅FN₄O m/z [M+H]⁺: calculated: 310.3, found: 311.1.

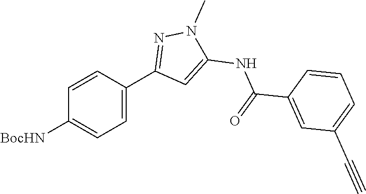 [SMILES: C#Cc1cccc(C(=O)Nc2cc(-c3ccc(NC(=O)OC(C)(C)C)cc3)nn2C)c1]

tert-butyl (4-(5-(3-ethynylbenzamido)-1-methyl-1H-pyrazol-3-yl)phenyl)carbamate: The general procedure A was followed using tert-butyl-(4-(5-amino-1-methyl-1H-pyrazol-3-yl)phenyl)carbamate (250 mg, 0.87 mmol), 3-ethynyl benzoic acid (152 mg, 1.04 mmol), oxalyl chloride (0.75 mL, 8.7 mmol), DIPEA (0.45 mL, 2.61 mmol), DMF (3 drops) and DCM (8 mL). The crude product was recrystallized to give tert-butyl (4-(5-(3-ethynylbenzamido)-1-methyl-1H-pyrazol-3-yl)phenyl)carbamate as a tan solid (206.8 mg, 57%). ESI MS for C₂₄H₂₄N₄O₃ m/z [M+H]⁺: calculated: 416.5, found: 417.1.

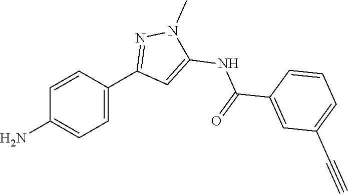 [SMILES: C#Cc1cccc(C(=O)Nc2cc(-c3ccc(N)cc3)nn2C)c1]

N-(3-(4-aminophenyl)-1-methyl-1H-pyrazol-5-yl)-3-ethynylbenzamide: The general procedure E was followed using tert-butyl (4-(5-(3-ethynylbenzamido)-1-methyl-1H-pyrazol-3-yl)phenyl)carbamate (206.8 mg, 0.5 mmol) in 4 M HCl in dioxane (10 mL) was stirred at room temperature overnight. The solution was concentrated in vacuo to give N-(3-(4-aminophenyl)-1-methyl-1H-pyrazol-5-yl)-3-ethynylbenzamide as a tan solid (158 mg, 100%). ESI MS for C₂₇H₂₂ClN₅O₂ m/z [M+H]⁺: calculated: 316.4, found: 317.2.

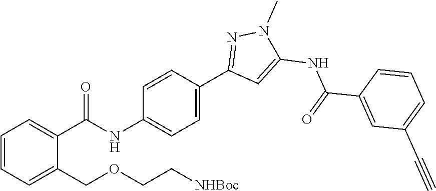 [SMILES: C#Cc1cccc(C(=O)Nc2cc(-c3ccc(NC(=O)c4ccccc4COCCNC(=O)OC(C)(C)C)cc3)nn2C)c1]

tert-butyl (2-((2-((4-(5-(3-ethynylbenzamido)-1-methyl-1H-pyrazol-3-yl)phenyl)carbamoyl)benzyl)oxy)ethyl)carbamate: The general procedure D was followed using 2-((2-((tert-butoxycarbonyl)amino)ethoxy)methyl)benzoic acid (112 mg, 0.38 mmol), N-(3-(4-aminophenyl)-1-methyl-1H-pyrazol-5-yl)-3-ethynylbenzamide (100 mg, 0.32 mmol), Ghosez reagent (0.14 mL, 1.02 mmol), pyridine (0.26 mL, 3.2 mmol) and DCM (3 mL). The crude product was purified by flash chromatography on silica gel (DCM/MeOH 98:2) to give tert-butyl (2-((2-((4-(5-(3-ethynylbenzamido)-1-methyl-1H-pyrazol-3-yl)phenyl)carbamoyl)benzyl)oxy)ethyl)carbamate as a tan solid (76.7 mg, 40%). ESI MS for C₃₄H₃₅N₅O₅ m/z [M+H]⁺: calculated: 593.7, found: 594.3.

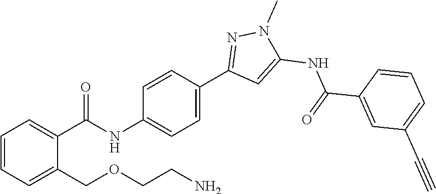 [SMILES: C#Cc1cccc(C(=O)Nc2cc(-c3ccc(NC(=O)c4ccccc4COCCN)cc3)nn2C)c1]

2-((2-aminoethoxy)methyl)-N-(4-(5-(3-ethynylbenzamido)-1-methyl-1H-pyrazol-3-yl)phenyl)benzamide: The general procedure E was followed using tert-butyl (2-((2-((4-(5-(3-ethynylbenzamido)-1-methyl-1H-pyrazol-3-yl)phenyl)carbamoyl)benzyl)oxy)ethyl)carbamate (76.7 mg, 0.13 mmol) in 4 M HCl in dioxane (5 mL) was stirred at room temperature overnight. The solution was concentrated in vacuo to give 2-((2-aminoethoxy)methyl)-N-(4-(5-(3-ethynylbenzamido)-1-methyl-1H-pyrazol-3-yl)phenyl)benzamide as a tan solid (64 mg, 100%). ESI MS for C₂₇H₂₂ClN₅O₂ m/z [M+H]⁺: calculated: 493.6, found: 494.1.

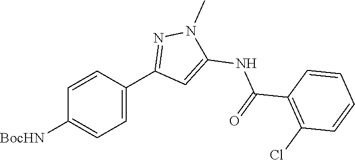 [SMILES: Cn1nc(-c2ccc(NC(=O)OC(C)(C)C)cc2)cc1NC(=O)c1ccccc1Cl]

tert-butyl (4-(5-(2-chlorobenzamido)-1-methyl-1H-pyrazol-3-yl)phenyl)carbamate: The general procedure C was followed using tert-butyl-(4-(5-amino-1-methyl-1H-pyrazol-3-yl)phenyl)carbamate (250 mg, 0.87 mmol), 2-chloro benzoic acid (162.8 mg, 1.04 mmol), HATU (496 mg, 1.31 mmol), DIPEA (0.45 mL, 2.61 mmol) and DMF (8 mL). The crude product was recrystallized to give tert-butyl (4-(5-(2-chlorobenzamido)-1-methyl-1H-pyrazol-3-yl)phenyl)carbamate as a tan solid (197 mg, 53%). ESI MS for C₂₂H₂₃ClN₄O₃ m/z [M+H]⁺: calculated: 426.9, found: 427.1.

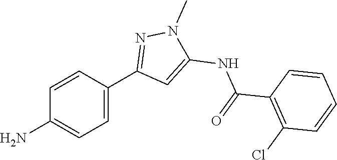 [SMILES: Cn1nc(-c2ccc(N)cc2)cc1NC(=O)c1ccccc1Cl]

N-(3-(4-aminophenyl)-1-methyl-1H-pyrazol-5-yl)-2-chlorobenzamide: The general procedure E was followed using tert-butyl (4-(5-(2-chlorobenzamido)-1-methyl-1H-pyrazol-3-yl)phenyl)carbamate (197 mg, 0.46 mmol) in 4 M HCl in dioxane (10 mL) was stirred at room temperature overnight. The solution was concentrated in vacuo to give N-(3-(4-aminophenyl)-1-methyl-1H-pyrazol-5-yl)-2-chlorobenzamide as a tan solid (150 mg, 100%). ESI MS for C₁₇H₁₅ClN₄O m/z [M+H]⁺: calculated: 326.8, found: 327.1.

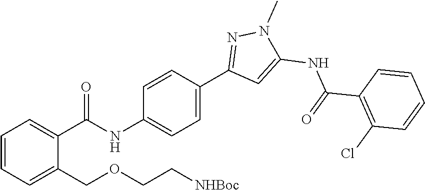 [SMILES: Cn1nc(-c2ccc(NC(=O)c3ccccc3COCCNC(=O)OC(C)(C)C)cc2)cc1NC(=O)c1ccccc1Cl]

tert-butyl (2-((2-((4-(5-(2-chlorobenzamido)-1-methyl-1H-pyrazol-3-yl)phenyl)carbamoyl)benzyl)oxy)ethyl)carbamate: The general procedure D was followed using 2-((2-((tert-butoxycarbonyl)amino)ethoxy)methyl)benzoic acid (54.3 mg, 0.18 mmol), N-(3-(4-aminophenyl)-1-methyl-1H-pyrazol-5-yl)-2-chlorobenzamide (50 mg, 0.15 mmol), Ghosez reagent (0.064 mL, 0.48 mmol), pyridine (0.12 mL, 1.5 mmol) and DCM (1.5 mL). The crude product was purified by flash chromatography on silica gel (DCM/MeOH 98:2) to give tert-butyl (2-((2-((4-(5-(2-chlorobenzamido)-1-methyl-1H-pyrazol-3-yl)phenyl)carbamoyl)benzyl)oxy)ethyl)carbamate as a tan solid (21 mg, 23%). ESI MS for C₃₂H₃₄ClN₅O₅ m/z [M+H]⁺: calculated: 604.1, found: 604.2.

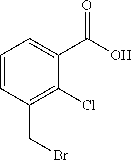 [SMILES: O=C(O)c1cccc(CBr)c1Cl]

3-(bromomethyl)-2-chlorobenzoic acid: Benzoyl peroxide was added to a suspension of 2-chloro-3-methyl benzoic acid and NBS in chlorobenzene. The solution was heated to 85° C. for 4 hours. The mixture was diluted with EtOAc and washed with H₂O, brine, dried (Na₂SO₄) and concentrated under reduced pressure to give 3-(bromomethyl)-2-chlorobenzoic acid as a light pink solid (291 mg, 100%). ESI MS for C₈H₆BrClO₂ m/z [M+H]⁺: calculated: 249.5, found: 248.9, 251.0.

### Example 6. SW393071 Inhibits Translation In Vitro

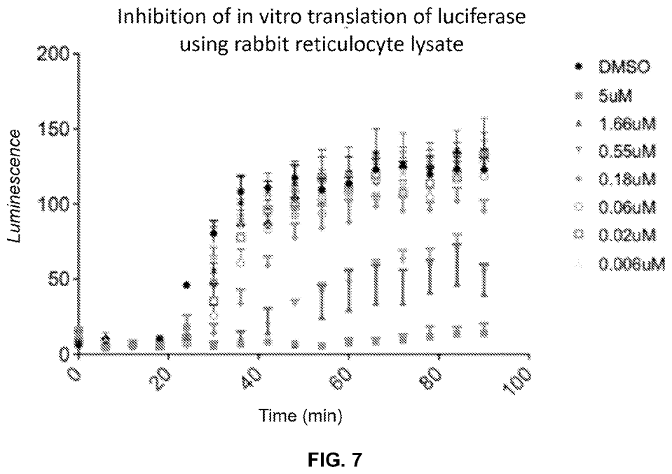

To test the impact of select compounds on translation, an in vitro translation system was set-up using full-length luciferase mRNA. Rabbit reticulocyte lysate was incubated in the presence of luciferase mRNA, NTPs, and various concentrations of SW393071. Transcription of full-length luciferase was monitored by luminescence. The data as shown in FIG. 7 suggests a dose-dependent inhibition of translation by SW393071.

### Example 7. SW388710 Probes Bind to RPS23 in Cells

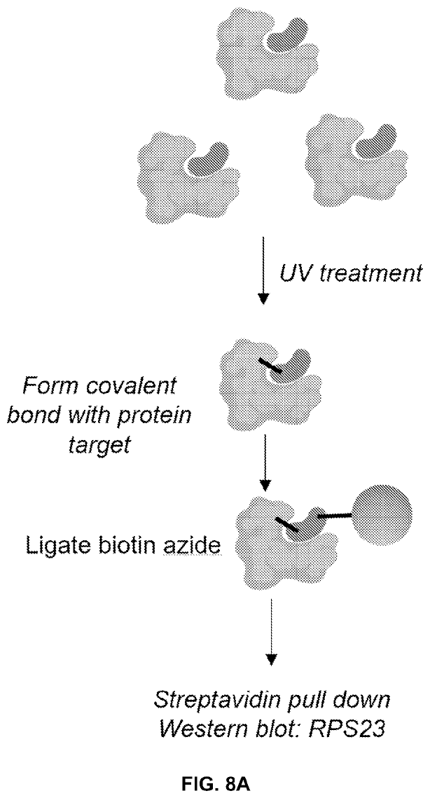

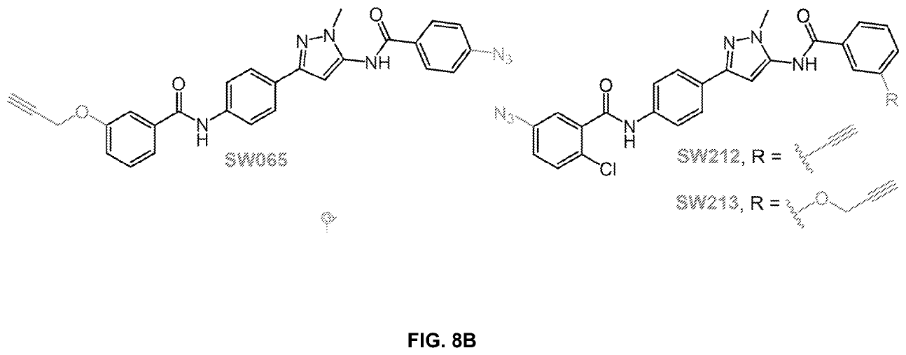

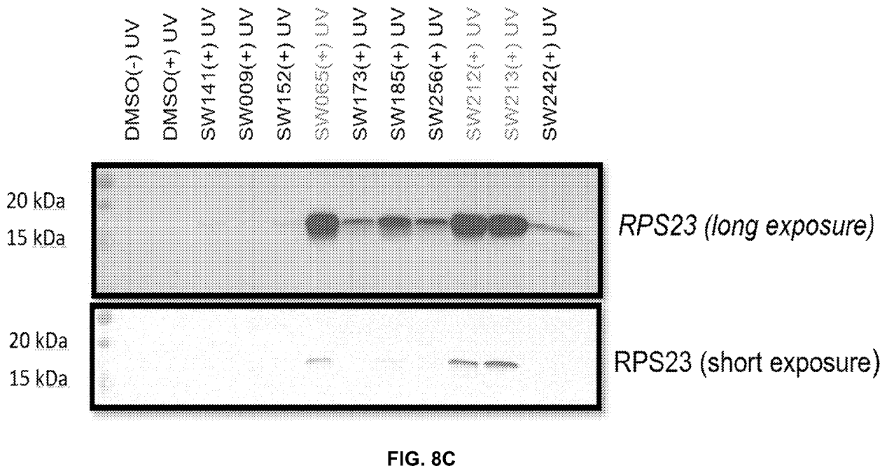

Next HCT116 cells were incubated with SW393071 analogs conjugated to a photo-crosslinker (analogs: SW393065, SW393212 and SW393213), and irradiated with UV light. The cells were lysed and click-conjugated to biotin-azide. Drug-bound proteins were pulled down with streptavidin coated beads and resolved on an SDS-PAGE gel. A schematic of the experiment is provided in FIG. 8A and the analogs are provided in FIG. 8B. Western blot analysis for RPS23 was conducted and shows bands with several probes, confirming binding with RPS23 in cells (FIG. 8C).

### Example 8. RPS23 Mutant Populations are Strongly Resistant to SW393071

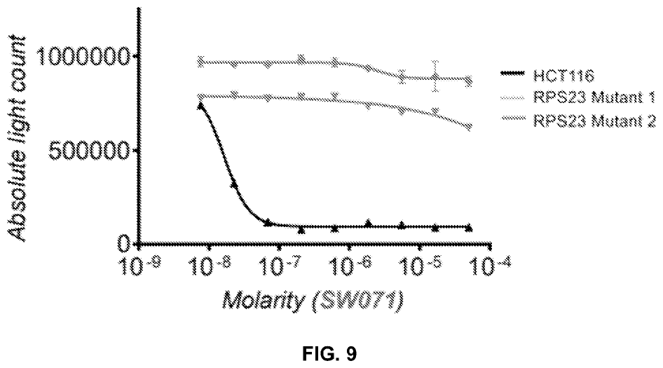

To further confirm the mechanism of action, wild-type or mutant RPS23 del R107-G109 knock-in mutant cells were incubated with SW393071. As seen in FIG. 9, RPS23 mutant lines were completely resistant to SW393071, confirming RPS23 as the functional target. This was further confirmed testing binding of SW393071 analog SW393212 to mutant RPS23.

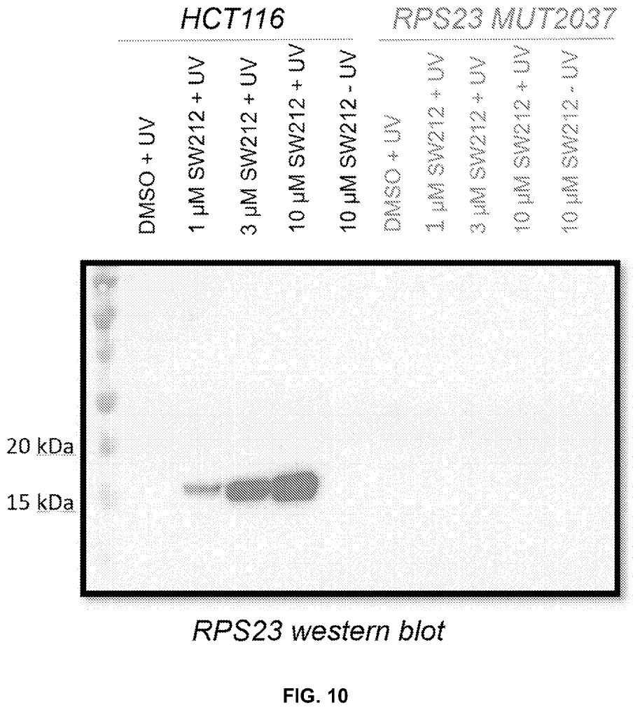

Wild-type or RPS23 del R107-G109 knock-in mutant HCT116 cells were incubated with photo-crosslinker analog SW393212 and irradiated with UV light. The cells were lysed, and click-conjugated to biotin-azide essentially as shown in Example 7. Drug-bound proteins were pulled down with streptavidin coated beads and resolved on an SDS-PAGE gel. Western blotting for RPS23 shows bands from WT but not RPS23 mutant cells, confirming that mutant cells do not bind SW393212 (see FIG. 10).

### Example 9. SW393071 Activates ISR, Leads to elF2α Phosphorylation

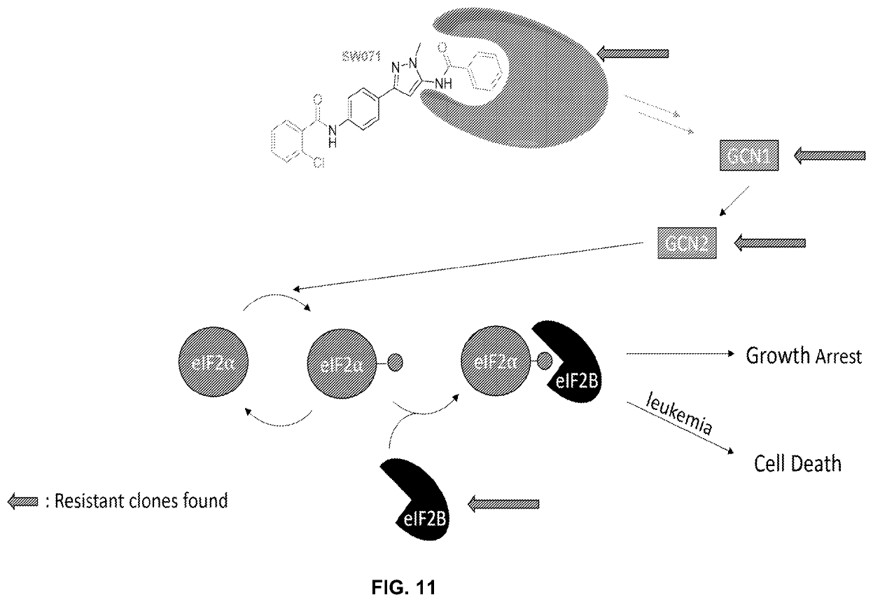

Based on the data so far, it was hypothesized that SW393071 activates intracellular stress response (ISR), leading to elF2α phosphorylation (see FIG. 11).

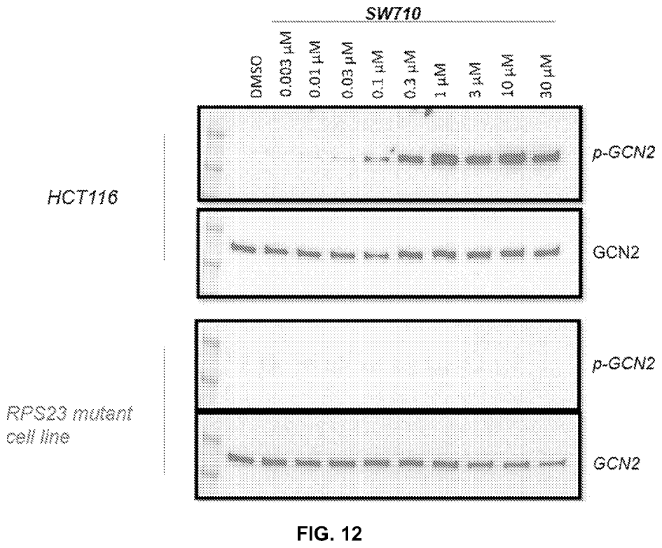

To confirm this hypothesis, wild-type HCT116 cells and HCT116 RPS23 del R107-G109 mutant cells were incubated with SW388710. Cells were lysed and evaluated via western blot for phospho-GCN2, a marker of activation of the integrated stress response. Wild-type but not RPS23 mutant cells, showed activation of the ISR as seen from phosphorylation of GCN2 (see FIG. 12).

### Example 10. SW388710 Inhibits Cell Proliferation Across Different Cancer Types

Known cancer cells lines were subjected to varying concentrations of SW388710 compound. IC₅₀ was calculated and is provided in Table 3. SW388710 was able to inhibit cell proliferation across multiple cell lines.

TABLE 3

| | | IC₅₀ to |
|---|---|---|
| Cell Line | Lineage | SW388719 (μM) |
| HCT116 | Colorectal | 0.14 |
| DLD1 | Colorectal | 0.06 |
| RKO | Colorectal | 2 |
| SUDHL5 | Lymphoma | 0.6 |
| Hela | Cervical | 0.54 |
| SUDHL-10 | Lymphoma | 0.065 |
| KBM-7 | Chronic Leukemia | 0.077 |
| MOLM-13 | Acute Leukemia | 0.34 |
| U937 | Acute Leukemia | 0.060 |

1. A compound of any one of Formulae I-VII: 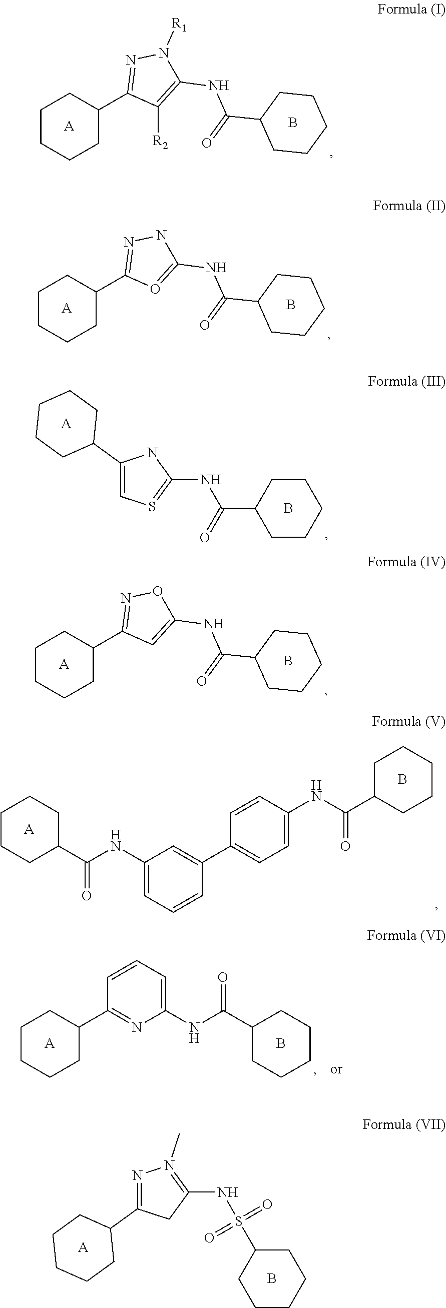
or a pharmaceutically acceptable salt thereof;
wherein,
Cycle A and Cycle B are each independently selected from —(C6-C10)-aryl, —(C6-C10)-substituted aryl, —(C6-C10)-heteroaryl, —(C6-C10)-substituted heteroaryl, —(C3-C10)-alkyl, or —(C3-C10)-substituted alkyl;
R1 is alkyl, aryl, or alkyl-aryl;
R2is hydrogen, halide, or alkyl;
wherein said heterocyclyl has 1-4 heteroatoms independently selected from N, NH, O, S, SO, and SO₂, and said heteroaryl has 1-4 heteroatoms independently selected from N, NH, O, and S.

2. The compound of claim 1, wherein Cycle A and Cycle B in Formula (I) are a C-6 substituted aryl as shown in Formula (Ia): 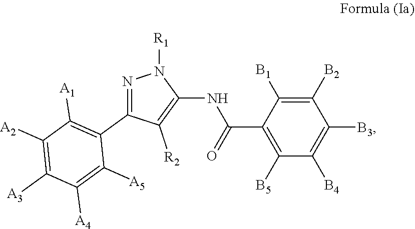
wherein
A₁-A₅ and B₁-B₅ are each independently selected from a halogen, —R, —OR, —NO₂, —NCS, —CN, —CF₃, —OCF₃, —SiR₃, —NH₂, —SR, —SOR, —SO2R, —SO2N(R)2, —SO3R, —(CR2)1-3R, —(CR2)1-3-OR, —(CR2)0-3-C(O)NR(CR2)0-3R, —(CR2)0-3—C(O)NR(CR2)0-3OR, —C(O)R, —C(O)C(O)R, —C(O)CH2C(O)R, —C(S)R, —C(S)OR, —C(O)OR, —C(O)C(O)OR, —C(O)C(O)N(R)2, —OC(O)R, —C(O)N(R)2, —OC(O)N(R)2, —C(S)N(R)2, —(CR2)0-3NHC(O)R, —N(R)N(R)COR, —N(R)N(R)C(O)OR, —N(R)N(R)CON(R)2, —N(R)SO2R, —N(R)SO2N(R)2, —N(R)C(O)OR, —N(R)C(O)R, —N(R)C(S)R, —N(R)C(O)N(R)2, —N(R)C(S)N(R)2, —N(COR)COR, —N(OR)R, —C(═NH)N(R)2, —C(O)N(OR)R, —C(═NOR)R, or —CF3;
wherein R is hydrogen, alkyl, alkyl amine, alkyl sulfonamide, aryl, substituted aryl, heterocyclic aryl amine, substituted heterocyclic aryl amine, cyclic aliphatic amine, heterocyclic aliphatic amine, substituted heterocyclic aliphatic amine, alkyne; or together with the carbon atom to form a cycloalkyl, a cycloalkenyl, or a heterocyclylalkyl ring;
R1 is alkyl, aryl, or alkyl-aryl; and
R2 is hydrogen, halogen, or alkyl.

3. The compound of claim 1, wherein in Formula (I) Cycle A is a substituted C6-aryl and Cycle B is a (C3-C10) cycloalkyl, a substituted (C3-C10) cycloalkyl, or a (C3-C10) cycloheteroalkyl as shown below:

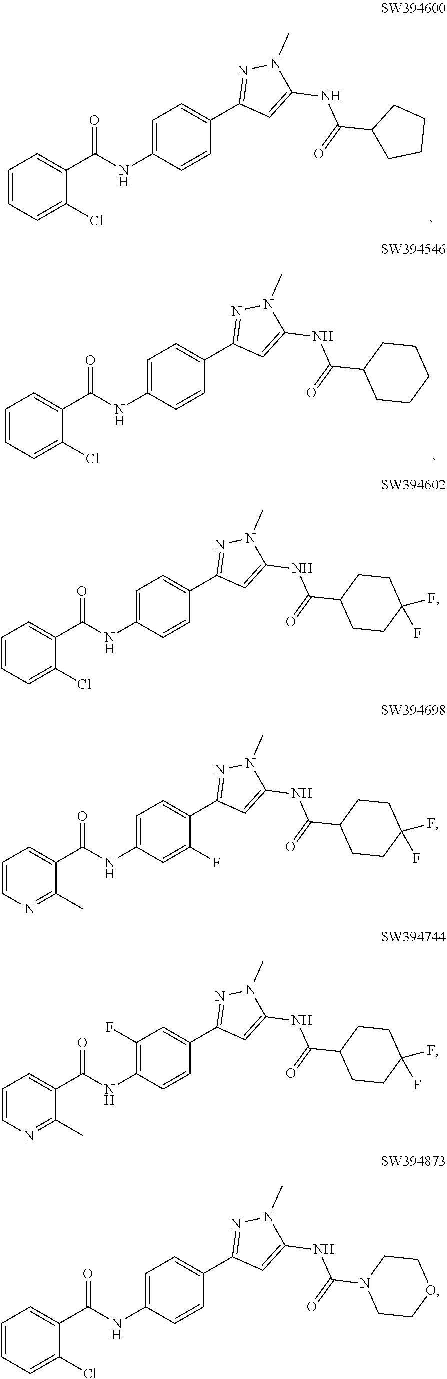 [SMILES: Cn1nc(-c2ccc(NC(=O)c3ccccc3Cl)cc2)cc1NC(=O)C1CCCC1 ; Cn1nc(-c2ccc(NC(=O)c3ccccc3Cl)cc2)cc1NC(=O)C1CCCCC1 ; Cn1nc(-c2ccc(NC(=O)c3ccccc3Cl)cc2)cc1NC(=O)C1CCC(F)(F)CC1 ; Cc1ncccc1C(=O)Nc1ccc(-c2cc(NC(=O)C3CCC(F)(F)CC3)n(C)n2)c(F)c1 ; Cc1ncccc1C(=O)Nc1ccc(-c2cc(NC(=O)C3CCC(F)(F)CC3)n(C)n2)cc1F ; Cn1nc(-c2ccc(NC(=O)c3ccccc3Cl)cc2)cc1NC(=O)N1CCOCC1]

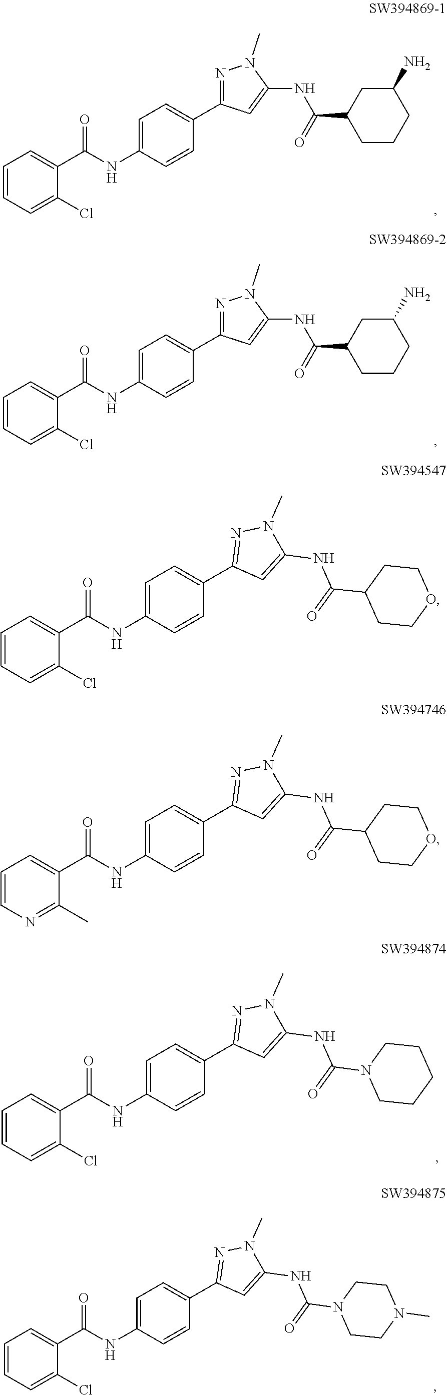

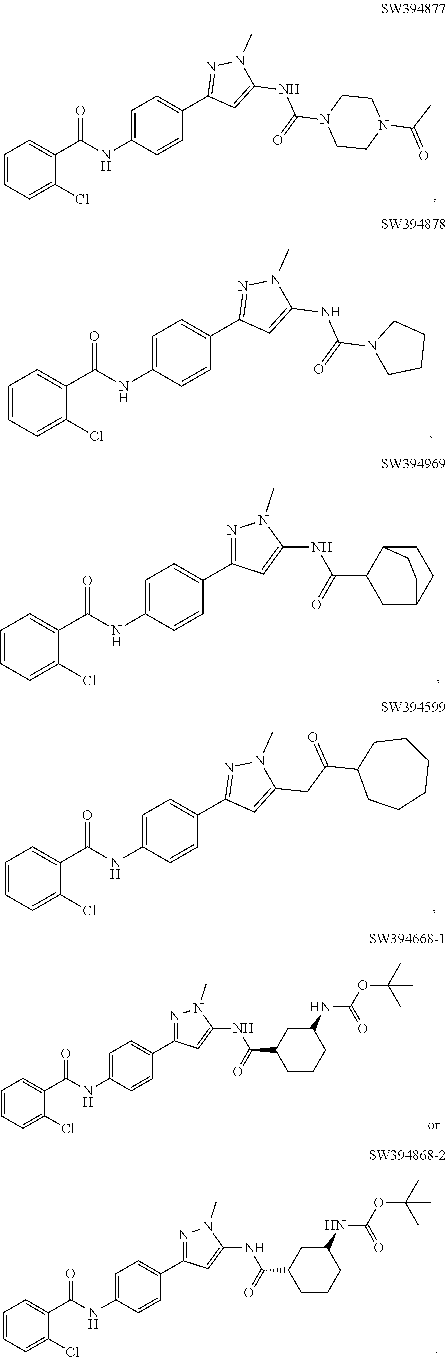

4. The compound of claim 1, wherein the compound is Formula (II) or Formula (III) and wherein in Formula (II) or Formula (III) cycle A and cycle B are C6-substituted aryl as shown in Formula (IIa)-(IIIa): 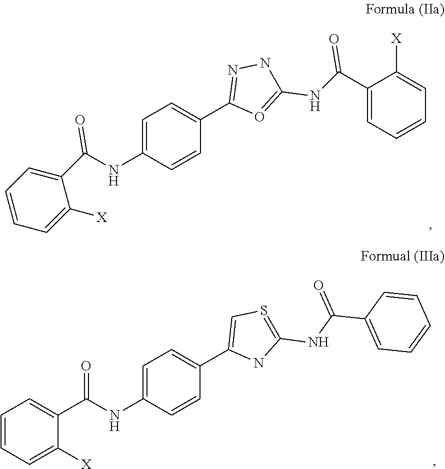
wherein X is a halogen.

5. The compound of claim 1, wherein the compound is Formula (IV) and wherein in Formula (IV) cycle A is a substituted C6-aryl and cycle B is a C6-aryl as shown in Formula (IVa):

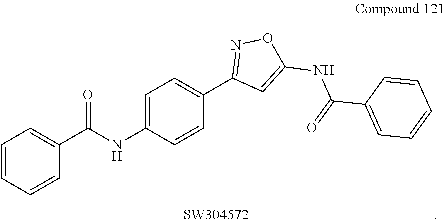 [SMILES: O=C(Nc1ccc(-c2cc(NC(=O)c3ccccc3)on2)cc1)c1ccccc1]

6. The compound of claim 1, wherein the compound is Formula (V) and wherein in Formula (V) cycle A is a C6-aryl and cycle B is a substituted C6-aryl as shown in Formula (Va):

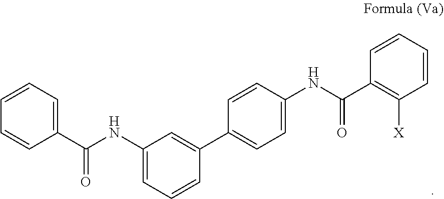

7. The compound of claim 1, wherein the compound is Formula (VI) and wherein in Formula (VI) cycle A is a substituted C6-aryl and cycle B is a C6-aryl as shown below:

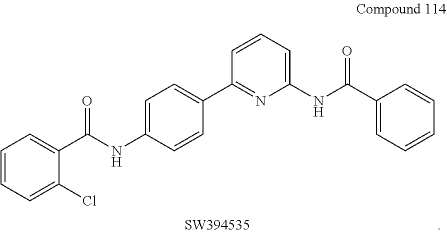 [SMILES: O=C(Nc1cccc(-c2ccc(NC(=O)c3ccccc3Cl)cc2)n1)c1ccccc1]

8. The compound of claim 1, wherein the compound is Formula (VII) and wherein in Formula (VII) cycle A is a substituted C6-aryl and cycle B is a C6-aryl as shown below:

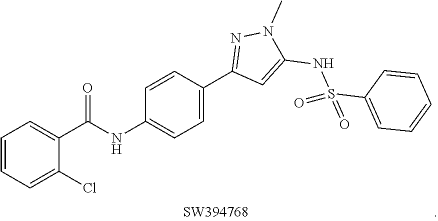 [SMILES: Cn1nc(-c2ccc(NC(=O)c3ccccc3Cl)cc2)cc1NS(=O)(=O)c1ccccc1]

9. (canceled)

10. (canceled)

11. A pharmaceutical composition comprising: one or more of the compounds of claim 1 and a pharmaceutically acceptable excipient.

12. (canceled)

13. A pharmaceutical composition of claim 11, wherein the composition does not comprise the following compounds:

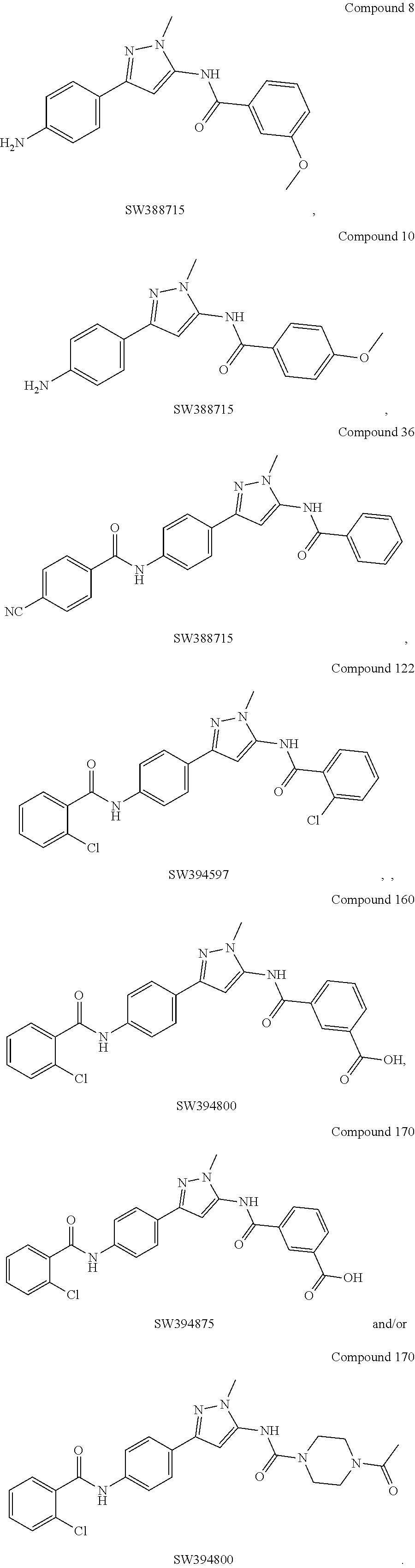

14. The pharmaceutical composition of claim 11, wherein the at least one pharmaceutically acceptable excipient is a liquid or solid filler, a diluent, a binder, a buffering agent, a pH modifying agent, a disintegrant, a dispersant, a preservative, a lubricant or wetting agent, taste-masking agent, an antioxidant, carrier, adjuvant, stabilizing agent, emulsifying agent, solution promoter, salt, solubilizer, antifoaming agent, surfactant, a flavoring agent, a coloring agent, solvent or encapsulating material or any combination thereof.

15. The pharmaceutical composition of claim 11, wherein the compound has an IC₅₀ value <20 nM against at least one type of cancer cell line.

16. The pharmaceutical composition of claim 11, further comprising additional active agents selected from a group consisting of NSAID, antibiotics, antimicrobial, anti-inflammatory, anticancer, theragnostic agent, and any combination thereof.

17. (canceled)

18. (canceled)

19. (canceled)

20. (canceled)

21. (canceled)

22. (canceled)

23. A method of treating cancer in a subject in need thereof, comprising:
administering an effective amount of one or more of the compounds of claim 1 and at least a pharmaceutically acceptable excipient to the subject.

24. The method of claim 23, wherein the compound is not SW388717, SW388715, SW393061, SW394597, SW394800, SW394875, and/or SW394877.

25. The method of claim 23, wherein the subject is diagnosed and/or suffering from a leukemia or colorectal cancer.

26. The method of claim 25, wherein the cancer is any one of acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, 10 gallbladder cancer, hairy cell leukemia, head and neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft 15 tissue sarcoma, squamous cell carcinomas, stomach cancer, testicular cancer, thyroid cancer, trophoblastic neoplasms, uterine cancer, vaginal cancer, cancer of the vulva and Wilma's tumor.

27. (canceled)

28. (canceled)

29. The method of claim 28, wherein the subject is a human.

30. The method of claim 28, wherein the administrating of the compound is done by a mode selected from parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal route.

31. (canceled)

32. (canceled)

33. (canceled)

34. (canceled)
